SEQUENCE LISTING (37 sequences)

SEQ ID NO: 1 - protein, 5 aa, synthetic construct
NYWMS

SEQ ID NO: 2 - protein, 17 aa, synthetic construct
DIHGNRGFNYHASWAKG

SEQ ID NO: 3 - protein, 9 aa, synthetic construct
ADDSGSHDI

SEQ ID NO: 4 - protein, 12 aa, synthetic construct
QSSQSVYSAKLS

SEQ ID NO: 5 - protein, 7 aa, synthetic construct
YGSTLAS

SEQ ID NO: 6 - protein, 12 aa, synthetic construct
QGTFYGPDWYFA

SEQ ID NO: 7 - protein, 116 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 8 - protein, 111 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 9 - protein, 446 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 218 aa, synthetic construct
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 242 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 16 - protein, 10 aa, synthetic construct
GYTFTNYYIH

SEQ ID NO: 17 - protein, 18 aa, synthetic construct
GWIYPGDGNTKYNEKFKG

SEQ ID NO: 18 - protein, 10 aa, synthetic construct
DSYSNYYFDY

SEQ ID NO: 19 - protein, 17 aa, synthetic construct
KSSQSLLNSRTRKNYLA

SEQ ID NO: 20 - protein, 7 aa, synthetic construct
WASTRES

SEQ ID NO: 21 - protein, 8 aa, synthetic construct
TQSFILRT

SEQ ID NO: 22 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 23 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 24 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 25 - protein, 503 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 26 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 446 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 497 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 30 - protein, 5 aa, synthetic construct
KYAMN

SEQ ID NO: 31 - protein, 19 aa, synthetic construct
RIRSKYNNYATYYADSVKD

SEQ ID NO: 32 - protein, 14 aa, synthetic construct
HGNFGNSYISYWAY

SEQ ID NO: 33 - protein, 14 aa, synthetic construct
GSSTGAVTSGNYPN

SEQ ID NO: 34 - protein, 7 aa, synthetic construct
GTKFLAP

SEQ ID NO: 35 - protein, 9 aa, synthetic construct
VLWYSNRWV

SEQ ID NO: 36 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 37 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

(19) United States
(12) Patent Application Publication
(10) Pub. No.: US 2026/0232834 A1
(43) Pub. Date: Aug. 13, 2026

(54) CYTOTOXICITY TARGETING CHIMERAS FOR ANTIBODY-DRUG CONJUGATES AND BISPECIFIC ANTIBODIES

(71) Applicant: GLAXOSMITHKLINE INTELLECTUAL PROPERTY DEVELOPMENT LIMITED, Brentford, Middlesex (GB)

(72) Inventors: Arthur Groy, Collegeville, PA (US); Aidan Hancock, Collegeville, PA (US); Matthew Robert Sender, Collegeville, PA (US); Brandon James Turunen, Boston, MA (US); Cunyu Zhang, Collegeville, PA (US); Craig Leach, Collegeville, PA (US)

(21) Appl. No.: 19/475,249

(22) PCT Filed: Apr. 19, 2024
(86) PCT No.: PCT/EP2024/060805

§ 371 Date: Oct. 14, 2025

Related U.S. Application Data
(60) Provisional application No. 63/461,171, filed on Apr. 21, 2023.

(51) Int. Cl. A61K 47/68; C07K 16/28; C07K 16/44

(52) U.S. Cl. CPC A61K 47/6879; A61K 47/68037; A61K 47/6873; C07K 16/2809; C07K 16/44; C07K 2317/31

(57) ABSTRACT

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer, inflammatory diseases, autoimmune diseases, viral infection, or bacterial infection.

### CROSS-REFERENCE TO RELATED APPLICATIONS

This application claims the benefit of and priority to United States Provisional Patent Application Ser. number 63/461,171, filed Apr. 21, 2023, the contents of which are hereby incorporated by reference in their entirety.

### REFERENCE TO AN ELECTRONIC SEQUENCE LISTING

This application contains a sequence listing which has been submitted electronically in ST.26 format and is hereby incorporated by reference in its entirety (said ST.26 copy, created on Apr. 17, 2024, is named “209280_seqlist.xml” and is 38, 133 bytes in size).

### FIELD OF THE DISCLOSURE

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein, such as an antibody drug conjugate or bispecific antibody or bispecific antigen binding fragment thereof. The present disclosure also relates to agents capable of binding to a receptor on a surface of a pathogenic cell and inducing the depletion of the pathogenic cell in a subject for use in the treatment of cancer, inflammatory diseases, autoimmune diseases, viral infection, or bacterial infection.

### BACKGROUND

Cell-surface proteins and their ligands play key roles in a range of inflammatory, infectious, and autoimmune diseases as well as tumor initiation, growth and metastasis. Antibody-based therapeutics have promising properties as drug candidates for these indications due to their selectivity for pathogenic cell-surface targets and their ability to direct immune surveillance to target-expressing tissues or cells to induce depletion of the pathogenic cells. Examples of such depletion mechanisms include antibody-dependent cellular cytotoxicity (ADCC), antibody-dependent cellular phagocytosis (ADCP), and complement-dependant cytotoxicity (CDC). However, antibody-based therapeutics often suffer from a lack of bioavailability, high cost, thermal instability, and difficult manufacturing due to their size, complexity and peptide based structures. Conversely, small molecule therapeutics often provide affordability, stability, and the convenience of oral dosing, but may suffer from poor selectivity and off-target effects, while also lacking the immune control of therapeutic antibodies.

Antibody-drug conjugates (ADCs) and bispecific antibodies or bispecific antigen binding fragments thereof, including bispecific T-cell engagers, have also shown promise in the treatment of a range of diseases. However, these therapeutics often suffer from high cost, instability, safety concerns, and difficult manufacturing due to their complexity. Conversely, small molecule therapeutics often provide affordability, stability, and the convenience of oral dosing, but may suffer from poor selectivity and off-target effects, while also lacking the immune control of antibody-based therapeutics.

Accordingly, a need exists for improved therapeutic approaches that target pathogenic cells for use in the treatment of disease. Such compositions and related methods are provided in the present disclosure.

### SUMMARY

In one aspect, the present disclosure provides an antibody-drug conjugate comprising an anti-cotinine antibody or antigen-binding fragment thereof covalently bound to a cytotoxic agent. In an embodiment, the anti-cotinine antibody or antigen-binding fragment thereof comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In an embodiment, the cytotoxic agent is bound to the antibody or antigen-binding fragment thereof via a linker. In an embodiment, the cytotoxic agent is Dxd (derivative of exatecan, also referred to as exatecan derivative for ADC). In an embodiment, the ratio of cytotoxic agent to antibody or antigen-binding fragment thereof is in a range of about 1:1 to about 10:1. In an embodiment, the antibody or antigen-binding fragment thereof comprises a heavy chain variable region (VH) as set forth in SEQ ID NO: 7 and a light chain variable region (VL) as set forth in SEQ ID NO: 8. In an embodiment, the antibody comprises a heavy chain as set forth in SEQ ID NO: 9 and a light chain as set forth in SEQ ID NO: 10.

In one aspect, the present disclosure provides a combination comprising the antibody-drug conjugate as disclosed herein and a heterobifunctional molecule comprising a moiety that binds a target cell-surface protein covalently linked to a cotinine moiety.

In a further aspect, the present disclosure provides a bispecific antibody or bispecific antigen binding fragment thereof comprising a cotinine binding domain and a CD3 binding domain. In an embodiment, the cotinine binding domain comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In an embodiment, the CD3 binding domain comprises: (i) a heavy chain CDR1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21; or (ii) a heavy chain CDR1 having SEQ ID NO: 30, a heavy chain CDR2 having SEQ ID NO: 31, a heavy chain CDR3 having SEQ ID NO: 32, a light chain CDR 1 having SEQ ID NO: 33, a light chain CDR2 having SEQ ID NO: 34, and a light chain CDR3 having SEQ ID NO: 35.

In an embodiment, the cotinine binding domain comprises a first single chain variable fragment (scFv) that binds a cotinine moiety, and the CD3 binding domain comprises a second scFv that binds CD3. In an embodiment, the scFv that binds a cotinine moiety comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In an embodiment, the scFv that binds a cotinine moiety comprises a heavy chain variable region (VH) and a light chain variable region (VL) joined by a first polypeptide linker. In an embodiment, the scFv that binds a cotinine moiety comprises a VH as set forth in SEQ ID NO: 7 and a VL as set forth in SEQ ID NO: 8. In an embodiment, the scFv that binds a cotinine moiety is as set forth in SEQ ID NO: 15. In an embodiment, the scFv that binds CD3 comprises (i) a heavy chain CDR1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21; or (ii) a heavy chain CDR1 having SEQ ID NO: 30, a heavy chain CDR2 having SEQ ID NO: 31, a heavy chain CDR3 having SEQ ID NO: 32, a light chain CDR 1 having SEQ ID NO: 33, a light chain CDR2 having SEQ ID NO: 34, and a light chain CDR3 having SEQ ID NO: 35. In an embodiment, the scFv that binds CD3 comprises a VH and a VL joined by a second polypeptide linker. In an embodiment, the scFv that binds CD3 comprises (i) a VH as set forth in SEQ ID NO: 22 and VL as set forth in SEQ ID NO: 23; or (ii) a VH as set forth in SEQ ID NO: 36 and VL as set forth in SEQ ID NO: 37. In an embodiment, the scFv that binds CD3 is as set forth in SEQ ID NO: 24. In an embodiment, the scFv that binds a cotinine moiety and the scFv that binds CD3 are joined by a third polypeptide linker. In an embodiment, the bispecific antibody or bispecific antigen binding fragment thereof is a bispecific T cell engager. In an embodiment, the bispecific antibody or bispecific antigen binding fragment thereof is a bispecific T cell engager is as set forth in SEQ ID NO: 25 or SEQ ID NO: 29.

In an embodiment, the bispecific antibody or bispecific antigen binding fragment thereof is a bispecific antibody. In an embodiment, the bispecific antibody comprises: a cotinine binding domain comprising a heavy chain comprising a heavy chain CDR1 having SEQ ID NO: 1, a heavy chain CDR2 having SEQ ID NO: 2, a heavy chain CDR3 having SEQ ID NO: 3, and a light chain comprising a light chain CDR1 having SEQ ID NO: 4, a light chain CDR2 having SEQ ID NO: 5, and a light chain CDR3 having SEQ ID NO: 6; and a CD3 binding domain comprising (i) a heavy chain comprising a heavy chain CDR1 having SEQ ID NO: 16, a heavy chain CDR2 having SEQ ID NO: 17, and a heavy chain CDR3 having SEQ ID NO: 18, and a light chain comprising a light chain CDR1 having SEQ ID NO: 19, a light chain CDR2 having SEQ ID NO: 20, and a light chain CDR3 having SEQ ID NO: 21, or (ii) a heavy chain comprising a heavy chain CDR1 having SEQ ID NO: 30, a heavy chain CDR2 having SEQ ID NO: 31, and a heavy chain CDR3 having SEQ ID NO: 32, and a light chain comprising a light chain CDR1 having SEQ ID NO: 33, a light chain CDR2 having SEQ ID NO: 34, and a light chain CDR3 having SEQ ID NO: 35. In an embodiment, the cotinine binding domain comprises a heavy chain comprising a heavy chain variable region (VH) as set forth in SEQ ID NO: 7 and a light chain comprising a light chain variable region (VL) as set forth in SEQ ID NO: 8. In an embodiment, the CD3 binding domain comprises (i) a heavy chain comprising a heavy chain variable region (VH) as set forth in SEQ ID NO: 22 and a light chain comprising a light chain variable region (VL) as set forth in SEQ ID NO: 23; or (ii) a heavy chain comprising a heavy chain variable region (VH) as set forth in SEQ ID NO: 36 and a light chain comprising a light chain variable region (VL) as set forth in SEQ ID NO: 37. In an embodiment, the cotinine binding domain comprises a heavy chain as set forth in SEQ ID NO: 28 and a light chain as set forth in SEQ ID NO: 10; and the CD3 binding domain comprises a heavy chain as set forth in SEQ ID NO: 26 and a light chain as set forth in SEQ ID NO: 27.

In one aspect, the present disclosure provides a combination comprising the bispecific antibody or bispecific antigen binding fragment thereof, including a bispecific T cell engager, as disclosed herein and a heterobifunctional molecule comprising a moiety that binds a target cell-surface protein covalently linked to a cotinine moiety.

In one aspect, the present disclosure provides a polynucleotide encoding the bispecific antibody or bispecific antigen binding fragment thereof, including a bispecific T cell engager, as disclosed herein. In one aspect, the present disclosure provides an expression vector comprising the polynucleotide encoding the bispecific antibody or bispecific antigen binding fragment thereof, including a bispecific T cell engager, as disclosed herein. In one aspect, the present disclosure provides a cell comprising the polynucleotide encoding the bispecific antibody or bispecific antigen binding fragment thereof, including a bispecific T cell engager, as disclosed herein. In one aspect, the present disclosure provides a cell comprising the expression vector comprising the polynucleotide encoding the bispecific antibody or bispecific antigen binding fragment thereof, including a bispecific T cell engager, as disclosed herein.

In one aspect, the present disclosure provides a method of treating and/or preventing a disease or disorder in a patient in need thereof, comprising: administering to the patient a therapeutically effective amount of a combination comprising an antibody-drug conjugate as disclosed herein and a heterobifunctional molecule as disclosed herein. In one aspect, the present disclosure provides a method of treating and/or preventing a disease or disorder in a patient in need thereof, comprising: administering to the patient a therapeutically effective amount of a combination comprising a bispecific antibody or bispecific antigen binding fragment thereof, including a bispecific T cell engager, as disclosed herein and a heterobifunctional molecule as disclosed herein.

In one aspect, the present disclosure provides a combination comprising an antibody-drug conjugate as disclosed herein and a heterobifunctional molecule as disclosed herein for use in therapy. In one aspect, the present disclosure provides a combination comprising a bispecific antibody or bispecific antigen binding fragment thereof, including a bispecific T cell engager, as disclosed herein and a heterobifunctional molecule as disclosed herein for use in therapy.

In one aspect, the present disclosure provides a combination comprising an antibody-drug conjugate as disclosed herein and a heterobifunctional molecule as disclosed herein for use in the treatment of a disease or disorder. In one aspect, the present disclosure provides a combination comprising a bispecific antibody or bispecific antigen binding fragment thereof, including a bispecific T cell engager, as disclosed herein and a heterobifunctional molecule as disclosed herein for use in the treatment of a disease or disorder.

In one aspect, the present disclosure provides use of a combination comprising an antibody-drug conjugate as disclosed herein and a heterobifunctional molecule as disclosed herein in the manufacture of a medicament for the treatment of a disease or disorder. In one aspect, the present disclosure provides use of a combination comprising a bispecific antibody or bispecific antigen binding fragment thereof, including a bispecific T cell engager, as disclosed herein and a heterobifunctional molecule as disclosed herein in the manufacture of a medicament for the treatment of a disease or disorder.

### BRIEF DESCRIPTION OF THE FIGURES

FIG. 1: Schematic representation of a combination of a cytotoxicity targeting chimera (CyTaC) and an antibody-drug conjugate.

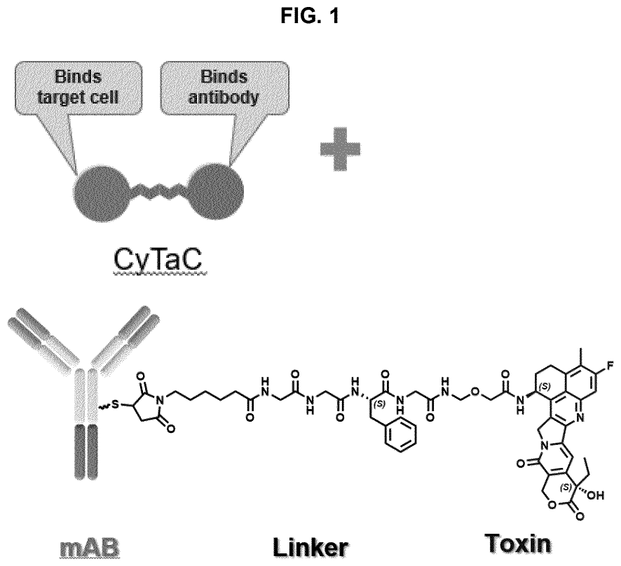

FIG. 2: LCMS chromatograms of unconjugated and conjugated anti-cotinine antibody. FIG. 2A shows a LCMS chromatogram of unconjugated anti-cotinine antibody. FIG. 2B shows a LCMS chromatogram of conjugated anti-cotinine antibody.

FIG. 3: Graphs showing results of PSMA targeting CyTaCs for antibody drug conjugate (ADC) cell killing assay described in Example 2. FIG. 3A shows % cell death of PSMA expressing cells (LNCAP) and control cells (CHO) treated with anti-cotinine targeting ADC (100 nM) and PSMA targeting CyTaC (varying concentrations). FIG. 3B shows % cell death of PSMA expressing cells (LNCAP) and control cells (CHO) each treated with (i) PSMA targeting CyTaC alone (20 μM), (ii) ADC alone (100 nM), or (iii) PSMA targeting CyTaC (20 μM) and ADC (100 nM).

FIG. 4: Graph showing results of T cell activation reporter assay using cotinine/CD3 bispecific antibody and CCR2 targeting CyTaC molecule as described in Example 3.

### DETAILED DESCRIPTION

### Definitions

As used herein and in the claims, the singular forms “a” and “the” include plural reference unless the context clearly dictates otherwise.

As used herein and in the claims, the term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “pathogenic cells” includes a cell subset that causes or is capable of causing disease. Examples of pathogenic cells include, but are not limited to, pathogenic immune cells, cancer or tumor cells, and stromal cells. A pathogenic cell can also be a pathogenic agent capable of causing an infection, such as a virus or a bacterial cell.

The term “pathogenic immune cells” includes a particular immune cell subset that causes or is capable of causing disease. These cellular subsets are resident cells or are recruited to particular locations and secrete cytokines, chemokines and other mediators and contribute to the persistence and progression of disease such as cancer in the case of a tumor microenvironment or chronic inflammation of the lung in the case of asthma. Examples of pathogenic immune cells include, but are not limited to myeloid-derived suppressor cells (MDSCs), T regulatory cells (Tregs), neutrophils, macrophages, B regulatory cells (Bregs), CD8 regulatory cells, (CD8regs), and exhausted T cells.

The term “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

The terms “effective amount” and “therapeutically effective amount” refer to an amount of a compound, or antibody, or antigen-binding portion thereof, according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The term “alkyl” represents a saturated, linear or branched hydrocarbon moiety having the specified number of carbon atoms. The term “C13 alkyl” refers to an unsubstituted alkyl moiety containing 1, 2 or 3 carbon atoms; exemplary alkyls include methyl, ethyl and propyl.

The term “alkylene” represents a saturated, linear or branched hydrocarbon moiety having the specified number of carbon atoms, with two points of attachment. The two points of attachment can be from the same or different carbon atoms. The term “C1-3 alkylene” refers to an unsubstituted alkylene moiety containing 1, 2 or 3 carbon atoms with two points of attachment; exemplary C1-3 alkylene groups include methylene, ethylene and propylene.

The term “alkenyl” represents an unsaturated, linear or branched hydrocarbon moiety having the specified number of carbon atoms. The term “C2-6 alkenyl” refers to an unsubstituted alkenyl moiety containing 2, 3, 4, 5, or 6 carbon atoms; exemplary alkenyls include propenyl, butenyl, pentenyl and hexenyl.

The term “alkenylene” represents an unsaturated, linear or branched hydrocarbon moiety having the specified number of carbon atoms, with two points of attachment. The two points of attachment can be from the same or different carbon atoms. The term “C2-6 alkenylene” refers to an unsubstituted alkenylene moiety containing 2, 3, 4, 5, or 6 carbon atoms with two points of attachment; exemplary C2-6 alkenylene groups include propenylene, butenylene, pentenylene and hexenylene.

The term “cycloalkyl” represents a saturated cyclic hydrocarbon moiety having the specified number of carbon atoms. The term “C3-6 cycloalkyl” refers to an unsubstituted cycloalkyl moiety containing 3, 4, 5 or 6 carbon atoms; exemplary cycloalkyls include cyclopropyl, cyclobutyl, cyclopentyl, and cyclohexyl.

The term “cycloalkylene” represents a saturated cyclic hydrocarbon moiety having the specified number of carbon atoms, with two points of attachment. The two points of attachment can be from the same or different carbon atoms. The term “C4-6 cycloalkylene” refers to an unsubstituted cycloalkylene moiety containing 4, 5, or 6 carbon atoms with two points of attachment. Exemplary cycloalkylene groups include cyclobutane-1,3-diyl, cyclopentane-1,3-diyl, cyclohexane-1,3-diyl, or cyclohexane-1,4-diyl.

The term “cycloalkenylene” represents an unsaturated cyclic hydrocarbon moiety having the specified number of carbon atoms, with two points of attachment. The two points of attachment can be from the same or different carbon atoms. The term “C3-6 cycloalkenylene” refers to an unsubstituted cycloalkenylene moiety containing 3, 4, 5, or 6 carbon atoms with two points of attachment.

The term “heterocycloalkylene” refers to a saturated cyclic hydrocarbon moiety containing 1 or 2 heteroatoms independently selected from oxygen, sulphur or nitrogen atoms, with two points of attachment. The two points of attachment can be from the same or different carbon atoms. The term “3- to 6-membered heterocycloalkylene” refers to a 3- to 6-membered saturated cyclic moiety containing 2, 3, 4 or 5 carbon atoms in addition to 1 or 2 oxygen, sulphur or nitrogen atoms, with two points of attachment. Suitably, the 3- to 6-membered heterocycloalkylene group contains 1 oxygen or nitrogen atom. Suitably such group contains 3 carbon atoms and 1 oxygen or nitrogen atom, such as azetidindiyl or oxetandiyl. Suitably such group contains 4 or 5 carbon atoms and 1 oxygen or nitrogen atom, such as tetrahydrofurandiyl, tetrahydropyrandiyl, pyrrolidindiyl or piperidindiyl.

The term “bridged bicyclic cycloalkylene” refers to a saturated bicyclic hydrocarbon moiety having at least one bridge, with two points of attachment. A “bridge” is an unbranched chain of atoms or an atom or a valence bond connecting two bridgeheads, where a “bridgehead” is any skeletal atom of the ring system which is bonded to three or more skeletal atoms (excluding hydrogen). The two points of attachment can be from the same or different carbon atoms. The term “C₇₋₉ bridged bicyclic cycloalkylene” refers to an unsubstituted bridged bicyclic cycloalkylene moiety containing 7, 8, or 9 carbon atoms with two points of attachment.

The term “arylene” refers to a monocyclic or bicyclic ring system wherein at least one ring in the system is aromatic, with two points of attachment. Exemplary arylene groups include phenylene, biphenylene, naphthylene, and anthracylene.

The term “heteroarylene” refers to a monocyclic or bicyclic ring system wherein at least one ring in the system is aromatic, and having, in addition to carbon atoms, from one to five heteroatoms independently selected from oxygen, sulphur or nitrogen atoms, with two points of attachment. The term “5- to 6-membered heteroarylene” refers to a 5- to 6-membered cyclic aromatic moiety containing 2, 3, 4 or 5 carbon atoms in addition to 1, 2, or 3 heteroatoms independently selected from oxygen, sulphur or nitrogen atoms, with two points of attachment.

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I). The invention is further directed to free or unsalted compounds of Formula (I).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, trifluoroacetate, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), b/s-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

A mixture of stereoisomers in which the relative configuration of all of the stereocenters is known may be depicted using the symbol “&” together with an index number (e.g., “&1”). For example, a group of two stereogenic centers labeled with the symbol “&1” represents a mixture of two possible stereoisomers in which the two stereogenic centers have a relative configuration as depicted.

Divalent groups are groups having two points of attachment. For all divalent groups, unless otherwise specified, the orientation of the group is implied by the direction in which the formula or structure of the group is written.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range.

Concentrations described herein are determined at ambient temperature and pressure. This may be, for example, the temperature and pressure at room temperature or in a particular portion of a process stream. Preferably, concentrations are determined at a standard state of 25° C. and 1 bar of pressure.

### Anti-Cotinine Antibodies

The present disclosure provides an antibody, or antigen-binding fragment thereof, that binds to a cotinine moiety. As used herein, the term “anti-cotinine antibody or antigen-binding fragment thereof” refers to an antibody, or antigen binding fragment thereof that binds to a cotinine moiety. Cotinine has the following structure:

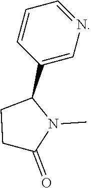

As used herein, the term “cotinine moiety” refers to cotinine or an analog of cotinine. Compounds of Formula (I) described herein comprise a cotinine moiety linked via a linker to a target-binding moiety, such as a PSMA-binding moiety. In one embodiment, the cotinine moiety has the following structure:

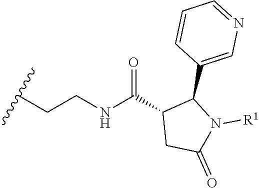

wherein R¹ is C₁₋₄ alkyl or C₃₋₆ cycloalkyl. In another embodiment, R¹ is methyl, ethyl, 1-propyl, 2-propyl, 1-butyl, 2-butyl, or t-butyl. In another embodiment, R¹ is methyl. In another embodiment, R¹ is ethyl. In another embodiment, R¹ is cyclopropyl, cyclobutyl, cyclopentyl, or cyclohexyl.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)2, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, 23 (9): 1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab′ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, δ and 0 respectively, each heavy chain can pair with either a K or A light chain. The majority of antibodies in the serum belong to the IgG class, there are four isotypes of human IgG (IgG1, IgG2, IgG3 and IgG4), the sequences of which differ mainly in their hinge region.

“CDRs” are defined as the complementarity determining region amino acid sequences of an antibody or antigen binding fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature, 1989, 342:877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 1 below represents one definition using each numbering convention for each CDR or binding unit. It should be noted that some of the CDR definitions may vary depending on the individual publication used.

TABLE 1

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

In a further embodiment, an antibody described herein is humanized. In a further embodiment, the Fc region of an antibody described herein is modified to increase ADCC activity, ADCP activity, and/or CDC activity, suitable modifications of which are provided below. In a further embodiment, the Fc region of an antibody described herein is modified to increase ADCC activity.

Fc engineering methods can be applied to modify the functional or pharmacokinetics properties of an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function. The in vivo half-life of an antibody can be altered by making mutations that affect binding of the Fc to the FcRn (neonatal Fc receptor).

The term “effector function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antibody, or antigen binding fragment thereof, of the present invention can be assessed for ADCC effector function in a Natural Killer cell assay. Examples of such assays can be found in Shields et al., The Journal of Biological Chemistry, 2001, 276:6591-6604; Chappel et al., The Journal of Biological Chemistry, 1993, 268:25124-25131; Lazar et al., PNAS, 2006, 103:4005-4010.

Examples of assays to determine CDC function include those described in J Imm Meth, 1995, 184:29-38.

The effects of mutations on effector functions (e.g., FcRn binding, FcγRs and C1q binding, CDC, ADCML, ADCC, ADCP) can be assessed, e.g., as described in Grevys et al., J Immunol., 2015, 194 (11): 5497-5508; Tam et al., Antibodies, 2017, 6 (3): 12; or Monnet et al., mAbs, 2014, 6 (2): 422-436.

Throughout this specification, amino acid residues in Fc regions, in antibody sequences or full-length antigen binding protein sequences, are numbered according to the EU index numbering convention.

Human IgG1 constant regions containing specific mutations have been shown to enhance binding to Fc receptors. In some cases these mutations have also been shown to enhance effector functions, such as ADCC and CDC, as described below. Antibodies, or antigen binding fragments thereof, of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science, 2014, 343:126O-1293) and E345R/E430G/S440Y results in preformed IgG hexamers (Wang et al., Protein Cell, 2018, 9 (1): 63-73).

Enhanced ADCC: Fc engineering can be used to enhance ADCC. For example (with reference to IgG1), F243L/R292P/Y³⁰⁰L/V305l/P396L; S239D/1332E; and S298A/E333A/K334A increase FcγRIIIa binding; S239D/1332E/A330L increases FcγRIIIa binding and decreases FcγRIIb binding; G236A/S239D/1332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., mAbs, 2013, 5 (2): 229-236).

Enhanced ADCP: Fc engineering can be used to enhance ADCP. For example (with reference to IgG1), G236A/S239D/1332E increases FcγRIIa binding and increases FcγRIIIa binding (Richards, J. et al., Mol. Cancer Ther., 2008, 7:2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding Wang et al., Protein Cell, 2018, 9 (1): 63-73).

In a further embodiment, an antibody, or antigen binding fragment thereof, of the present invention may comprise a heavy chain constant region with an altered glycosylation profile, such that the antibody, or antigen binding fragment thereof, has an enhanced effector function, e.g., enhanced ADCC, enhanced CDC, or both enhanced ADCC and CDC. Examples of suitable methodologies to produce an antibody, or antigen binding fragment thereof, with an altered glycosylation profile are described in WO 2003/011878, WO 2006/014679 and EP1229125.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al., J Biol Chem., 2002, 277 (30): 26733-40 and Monnet et al., mAbs, 2014, 6 (2): 422-436).

In one embodiment there is provided an antibody, or antigen binding fragment thereof, comprising a chimeric heavy chain constant region. In an embodiment, the antibody, or antigen binding fragment thereof, comprises an IgG1/IgG3 chimeric heavy chain constant region, such that the antibody, or antigen binding fragment thereof, has an enhanced effector function, for example enhanced ADCC or enhanced CDC, or enhanced ADCC and CDC functions. For example, a chimeric antibody, or antigen binding fragment thereof, of the invention may comprise at least one CH2 domain from IgG3. In one such embodiment, the antibody, or antigen binding fragment thereof, comprises one CH2 domain from IgG3 or both CH2 domains may be from IgG3. In a further embodiment, the chimeric antibody, or antigen binding fragment thereof, comprises an IgG1 CH1 domain, an IgG3 CH2 domain, and an IgG3 CH3 domain. In a further embodiment, the chimeric antibody, or antigen binding fragment thereof, comprises an IgG1 CH1 domain, an IgG3 CH2 domain, and an IgG3 CH3 domain except for position 435 that is histidine.

In a further embodiment, the chimeric antibody, or antigen binding fragment thereof, comprises an IgG1 CH1 domain and at least one CH2 domain from IgG3. In an embodiment, the chimeric antibody, or antigen binding fragment thereof, comprises an IgG1 CH1 domain and the following residues, which correspond to IgG3 residues, in a CH2 domain: 274Q, 276K, 296F, 300F and 339T. In an embodiment, the chimeric antibody, or antigen binding fragment thereof, also comprises 356E, which corresponds to an IgG3 residue, within a CH3 domain. In an embodiment, the antibody, or antigen binding fragment thereof, also comprises one or more of the following residues, which correspond to IgG3 residues within a CH3 domain: 358M, 384S, 392N, 397M, 4221, 435R, and 436F.

Also provided is a method of producing an antibody, or antigen binding fragment thereof, according to the invention comprising the steps of:
a) culturing a recombinant host cell comprising an expression vector comprising a nucleic acid sequence encoding a chimeric Fc region having both IgG1 and IgG3 Fc region amino acid residues (e.g. as described above); and
b) recovering the antibody, or antigen binding fragment thereof.

Such methods for the production of antibody, or antigen binding fragment thereof, with chimeric heavy chain constant regions can be performed, for example, using the COMPLEGENT technology system available from BioWa, Inc. (Princeton, NJ) and Kyowa Hakko Kirin Co., Ltd. The COMPLEGENT system comprises a recombinant host cell comprising an expression vector in which a nucleic acid sequence encoding a chimeric Fc region having both IgG1 and IgG3 Fc region amino acid residues is expressed to produce an antibody, or antigen binding fragment thereof, having enhanced CDC activity, i.e. CDC activity is increased relative to an otherwise identical antibody, or antigen binding fragment thereof, lacking such a chimeric Fc region, as described in WO 2007/011041 and US 2007/0148165, each of which are incorporated herein by reference. In an alternative embodiment, CDC activity may be increased by introducing sequence specific mutations into the Fc region of an IgG chain. Those of ordinary skill in the art will also recognize other appropriate systems.

The present invention also provides a method of producing an antibody, or antigen binding fragment thereof, according to the invention comprising the steps of:
a) culturing a recombinant host cell comprising an expression vector comprising a nucleic acid encoding the antibody, or antigen binding fragment thereof, optionally wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO 00/61739 and WO 02/31240, all of which are incorporated herein by reference. Those of ordinary skill in the art will also recognize other appropriate systems.

In one embodiment, the antibody, or antigen binding fragment thereof, is produced in a host cell in which the FUT8 gene has been inactivated. In a further embodiment, the antibody, or antigen binding fragment thereof, is produced in a−/− FUT8 host cell. In a further embodiment, the antibody, or antigen binding fragment thereof, is afucosylated at Asn297 (IgG1).

It will be apparent to those skilled in the art that such modifications may not only be used alone but may be used in combination with each other in order to further enhance effector function.

In one such embodiment, there is provided an antibody, or antigen binding fragment thereof, comprising a heavy chain constant region that comprises a both a mutated and chimeric heavy chain constant region, individually described above. For example, an antibody, or antigen binding fragment thereof, comprising at least one CH2 domain from IgG3 and one CH2 domain from IgG1, and wherein the IgG1 CH2 domain has one or more mutations at positions selected from 239, 332 and 330 (for example the mutations may be selected from S239D, 1332E and A330L), such that the antibody, or antigen binding fragment thereof, has enhanced effector function, e.g. enhanced ADCC or enhanced CDC, or enhanced ADCC and enhanced CDC in comparison to an equivalent antibody, or antigen binding fragment thereof, with an IgG1 heavy chain constant region lacking said mutations. In one embodiment, the IgG1 CH2 domain has the mutations S239D and 1332E. In another embodiment, the IgG1 CH2 domain has the mutations S239D, A330L, and 1332E.

In an alternative embodiment, there is provided an antibody, or antigen binding fragment thereof, comprising both a chimeric heavy chain constant region and an altered glycosylation profile, as individually described above. In an embodiment, the antibody, or antigen binding fragment thereof, comprises an altered glycosylation profile such that the ratio of fucose to mannose is 0.8:3 or less. In one such embodiment, the heavy chain constant region comprises at least one CH2 domain from IgG3 and one CH2 domain from IgG1 and has an altered glycosylation profile such that the ratio of fucose to mannose is 0.8:3 or less, for example wherein the antibody, or antigen binding fragment thereof, is defucosylated. Said antibody, or antigen binding fragment thereof, has an enhanced effector function, e.g. enhanced ADCC or enhanced CDC, or enhanced ADCC and enhanced CDC, in comparison to an equivalent antibody, or antigen binding fragment thereof, with an IgG1 heavy chain constant region lacking said glycosylation profile.

In an alternative embodiment, the antibody, or antigen binding fragment thereof, has at least one IgG3 heavy chain CH2 domain and at least one heavy chain constant domain from IgG1 wherein both IgG CH2 domains are mutated in accordance with the limitations described herein.

In one aspect, there is provided a method of producing an antibody, or antigen binding fragment thereof, according to the invention described herein comprising the steps of:
a) culturing a recombinant host cell containing an expression vector comprising a nucleic acid sequence encoding a chimeric Fc domain having both IgG1 and IgG3 Fc domain amino acid residues (e.g. as described above); and wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the ACCRETAMAB technology system available from BioWa, Inc. (Princeton, NJ) that combines the POTELLIGENT and COMPLEGENT technology systems to produce an antibody, or antigen binding fragment thereof, having both enhanced ADCC and CDC activity relative to an otherwise identical monoclonal antibody that lacks a chimeric Fc domain and that is fucosylated.

In another embodiment, there is provided an antibody, or antigen binding fragment thereof, comprising a mutated and chimeric heavy chain constant region wherein said antibody, or antigen binding fragment thereof, has an altered glycosylation profile such that the antibody, or antigen binding fragment thereof, has enhanced effector function, e.g. enhanced ADCC or enhanced CDC, or both enhanced ADCC and CDC. In one embodiment the mutations are selected from positions 239, 332 and 330, e.g. S239D, 1332E and A330L. In a further embodiment the heavy chain constant region comprises at least one CH2 domain from IgG3 and one CH1 domain from IgG1. In one embodiment the heavy chain constant region has an altered glycosylation profile such that the ratio of fucose to mannose is 0.8:3 or less, e.g. the antibody, or antigen binding fragment thereof, is defucosylated, such that said antibody, or antigen binding fragment thereof, has an enhanced effector function in comparison with an equivalent non-chimeric antibody, or antigen binding fragment thereof, lacking said mutations and lacking said altered glycosylation profile.

In a further embodiment, the anti-cotinine antibody, or antigen binding fragment thereof, comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In a further embodiment, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/1332E or S239D/1332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/1332E, wherein residue numbering is according to the EU Index.

In a further embodiment, the anti-cotinine antibody, or antigen binding fragment thereof, comprises a heavy chain variable region (VH) having SEQ ID NO: 7 and a light chain variable region (VL) having SEQ ID NO: 8. In a further embodiment, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/1332E or S239D/1332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/1332E, wherein residue numbering is according to the EU Index.

In a further embodiment, the anti-cotinine antibody has a heavy chain comprising SEQ ID NO: 9 and a light chain comprising SEQ ID NO: 10.

### Antibody-Drug Conjugates (ADCs)

The present disclosure also provides an immunoconjugate (interchangeably referred to as an “antibody-drug conjugate,” “ADC” or “antigen binding protein-drug conjugate”) comprising an antibody or antigen-binding fragment thereof as disclosed herein conjugated to one or more drugs, such as a cytotoxic agent, such as a chemotherapeutic agent, an immunotherapeutic agent, a growth inhibitory agent, a toxin (e.g., a protein toxin, such as an enzymatically active toxin of bacterial, fungal, plant, or animal origin, or fragments thereof), an antiviral agent, a radioactive isotope (i.e., a radioconjugate), an antibiotic, or a small interfering RNA (siRNA). Immunoconjugates have been used for the local delivery of cytotoxic agents, i.e., drugs that kill or inhibit the growth or proliferation of cells, in the treatment of cancer (Lambert, J. (2005) Curr. Opinion in Pharmacology 5:543-549; Wu et al. (2005) Nature Biotechnology 23 (9): 1137-1146; Payne, G. (2003) Cancer Cell 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278). Immunoconjugates allow for, inter alia, the targeted delivery of a drug moiety to a tumor, and intracellular accumulation therein, where systemic administration of unconjugated drugs may result in unacceptable levels of toxicity to normal cells (Tsuchikama and An, Protein and Cell, (2018)9:33-46). Immunoconjugates can enable selective delivery of a potent cytotoxic payload to target cancer cells, resulting in improved efficacy, reduced systemic toxicity, and preferable pharmacokinetics (PK)/pharmacodynamics (PD) and biodistribution compared to traditional chemotherapy (Tsuchikama and An 2018); Beck A. et al. (2017) Nature Rev. Drug Disc. 16:315-337). Both polyclonal antibodies and monoclonal antibodies have been reported as useful in these strategies (Rowland et al., (1986) Cancer Immunol. Immunother. 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al. (2000) J. Nat. Cancer Inst. 92 (19): 1573-1581; Mandler et al. (2000) Bioorganic & Med. Chem. Letters 10:1025-1028; Mandler et al. (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al. (1993) Cancer Res. 53:3336-3342).

In certain embodiments, an immunoconjugate comprises an antigen binding protein, such as an antibody or antigen binding fragment thereof, and a drug, such as toxin, such as a chemotherapeutic agent. The drug can be modified (e.g., via standard synthetic chemistry) to allow its chemical attachment (e.g., to contain a reaction handle to allow its chemical attachment) to a reactive end of a linker that joins the drug to the antigen binding protein. Immunoconjugate drugs, such as chemotherapeutic agents, useful in the generation of immunoconjugates are described herein. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct.28, 1993. In addition to toxins, a radioactive material, such as a radionucleotide, may be used as the drug in an ADC. A variety of radionucleotides are available for the production of radioconjugated antibodies. Examples include 212Bi, 1311, 131 In, 90Y, and 186Re.

Antigen binding proteins (such as antibodies or antigen binding fragments thereof) of the present disclosure may also be conjugated to one or more toxins, including, but not limited to, a calicheamicin, a maytansinoid, a dolastatin, an aurostatin, a trichothecene, and CC1065, and a derivative of these toxins that have toxin activity. Suitable cytotoxic agents include, but are not limited to, an auristatin including dovaline-valine-dolaisoleunine-dolaproine-phenylalanine (MMAF) and monomethyl auristatin E (MMAE) as well as an ester form of MMAE, a DNA minor groove binding agent, a DNA minor groove alkylating agent, an enediyne, a lexitropsin, a duocarmycin, a taxane (such as paclitaxel and docetaxel), a puromycin, a dolastatin, a maytansinoid, and a vinca alkaloid. Specific cytotoxic agents include exatecan, Dxd (derivative of exatecan), topotecan, morpholino-doxorubicin, rhizoxin, cyanomorpholino-doxorubicin, dolastatin-10, echinomycin, combretatstatin, chalicheamicin, maytansine, DM-1, DM-4, and netropsin. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, or a dolastatin. Antitubulin agents include dimethylvaline-valine-dolaisoleuine-dolaproine-phenylalanine-p-phenylenediamine (AFP), MMAF, MMAE, auristatin E, vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansine, DM-1, DM-4, and eleutherobin.

Antibody drug conjugates can be produced by conjugating a cytotoxic agent to an antigen binding protein (such as an antibody or antigen binding fragment thereof). In some embodiments, the linker comprises a thiol-reactive maleimide. In the case of monomethylauristatin E (MMAE), the linker can consist of a thiol-reactive maleimide, a caproyl spacer, the dipeptide valine-citrulline, or p-aminobenzyloxycarbonyl, a self-immolative fragmenting group. In the case of monomethylauristatin F (MMAF), a protease-resistant maleimidocaproyl linker can be used. The conjugation process may lead to heterogeneity in drug-antibody attachment, varying in both the number of drugs bound to each antibody molecule (mole ratio [MR]), and the site of attachment. The drug-to-antibody MR is typically between 0 and 10, such as 2, 4, 6, or 8.

Auristatins and Dolastatins: In some embodiments, the immunoconjugate comprises an antigen binding protein (such as an antibody) conjugated to a dolastatin or a dolostatin peptidic analog or derivative, an auristatin (U.S. Pat. Nos. 5,635,483; 5,780,588). Dolastatins and auristatins have been shown to interfere with microtubule dynamics, GTP hydrolysis, and nuclear and cellular division (Woyke et al. (2001) Antimicrob. Agents and Chemother. 45 (12): 358O-3584) and have anticancer (U.S. Pat. No. 5,663,149) and antifungal activity (Pettit et al. (1998) Antimicrob. Agents Chemother. 42:2961-2965). The dolastatin or auristatin (a pentapeptide derivative of dolastatin) drug moiety may be attached to the antibody through the N (amino) terminus or the C (carboxyl) terminus of the peptidic drug moiety (WO 02/088172). Exemplary auristatin embodiments include the N-terminus linked monomethylauristatin drug moieties DE and DF, disclosed in “Monomethylvaline Compounds Capable of Conjugation to Ligands,” U.S. Pat. No. 7,498,298. As used herein, the abbreviation “MMAE” refers to monomethyl auristatin E. As used herein the abbreviation “MMAF” refers to dovaline-valine-dolaisoleuine-dolaproine-phenylalanine. Typically, peptide-based drug moieties can be prepared by forming a peptide bond between two or more amino acids and/or peptide fragments. Such peptide bonds can be prepared, for example, according to the liquid phase synthesis method (see E. Schroder and K. Lubke, “The Peptides,” volume 1, pp 76-136, 1965, Academic Press) that is well known in the field of peptide chemistry. The auristatin/dolastatin drug moieties may be prepared according to the methods of: U.S. Pat. Nos. 5,635,483; 5,780,588; Pettit et al. (1989) J. Am. Chem. Soc. 111:5463-5465; Pettit et al. (1998) Anti-Cancer Drug Design 13:243-277; Pettit, G. R., et al. Synthesis, 1996, 719-725; and Pettit et al. (1996) J. Chem. Soc. Perkin Trans. 15:859-863. See also Doronina (2003) Nat Biotechnol 21 (7): 778-784; “Monomethylvaline Compounds Capable of Conjugation to Ligands,” U.S. Pat. No. 7,498,298, (disclosing, e.g., linkers and methods of preparing monomethylvaline compounds such as MMAE and MMAF conjugated to linkers). Biologically active organic compounds that act as cytotoxic agents, specifically pentapeptides, are disclosed in U.S. Pat. Nos. 6,884,869; 7,498,298; 7,098,308; 7,256,257; and 7,423, 116. Maytansine and Maytansinoids: Maytansinoids are mitototic inhibitors that act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Highly cytotoxic maytansinoid drugs can be prepared from ansamitocin precursors produced by fermentation of microorganisms such as Actinosynnema. Methods for isolating ansamitocins are described in U.S. Pat. No. 6,573,074. Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Antibody-maytansinoid conjugates are prepared by chemically linking an antigen binding protein (such as an antibody) to a maytansinoid molecule without significantly diminishing the biological activity of either the antibody or the maytansinoid molecule. See, e.g., U.S. Pat. No. 5,208,020. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Maytansinoids are maytansinol and maytansinol analogues modified in the aromatic ring or at other positions of the maytansinol molecule, such as various maytansinol esters. Methods for preparing maytansinoids for linkage with antibodies are disclosed, e.g., in U.S. Pat. Nos. 6,570,024 and 6,884,874.

Calicheamicin: The calicheamicin family of antibiotics is capable of producing double-stranded DNA breaks at sub-picomolar concentrations. For the preparation of conjugates of the calicheamicin family, see, e.g., U.S. Pat. Nos. 5,712,374, 5,714,586, 5,739,116, 5,767,285, 5,770,701, 5,770,710, 5,773,001, and 5,877,296. Structural analogues of calicheamicin that may be used include, but are not limited to, γ₁′, α₂′, α₃′, N-acetyl-yl, PSAG and θ₁′ (Hinman et al., Cancer Research 53:3336-3342 (1993), Lode et al., Cancer Research 58:2925-2928 (1998) and the aforementioned U.S. patents). Another anti-tumor drug that the antibody can be conjugated to is QFA, which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents: Other cytotoxic agents, such as antitumor agents, that can be conjugated to an antigen binding protein (such as an antibody or antigen binding fragment thereof) include BCNU, streptozoicin, vincristine and 5-fluorouracil, the family of agents known collectively LL-E33288 complex described in U.S. Pat. Nos. 5,053,394 and 5,770,710, as well as esperamicins (U.S. Pat. No. 5,877,296).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

The present disclosure further contemplates an immunoconjugate formed between an antigen binding protein (such as an antibody or antigen binding fragment thereof) and a compound with nucleolytic activity (e.g., a ribonuclease or a DNA endonuclease such as a deoxyribonuclease; DNase).

For selective destruction of the tumor, the antigen binding protein (such as an antibody or antigen binding fragment thereof) may comprise a highly radioactive atom. A variety of radioactive isotopes are available for the production of radioconjugated antibodies. Examples include At211, 1131, 1125, Y⁹⁰, Re186, Re188, Sm153, Bi212, P32, Pb212 and radioactive isotopes of Lu. When the conjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc99m or 1123, Re186, Re188 and In111 can be attached via a cysteine residue in the peptide. Yttrium-90 can be attached via a lysine residue. The IODOGEN method (Fraker et al. (1978) Biochem. Biophys. Res. Commun. 80:49-57) can be used to incorporate iodine-123. “Monoclonal Antibodies in Immunoscintigraphy” (Chatal, CRC Press 1989) describes other methods in detail.

In some cases, an anti-cotinine antigen binding protein (such as an antibody or antigen binding fragment thereof) disclosed herein is an immunoconjugate comprising an antigen binding protein, such as an antibody or antigen binding fragment thereof according to the disclosure as herein described including, but not limited to, an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate). In some cases, the anti-cotinine antibody or antigen binding fragment thereof is conjugated to a toxin such as an auristatin, e.g., monomethyl auristatin E (MMAE) or monomethyl auristatin F (MMAF). In some embodiments, the anti-cotinine antibody or antigen binding fragment thereof is conjugated to AFP, MMAF, MMAE, AEB, AEVB or auristatin E. In some embodiments, the anti-cotinine antibody or antigen binding fragment thereof is conjugated to paclitaxel, docetaxel, CC-1065, SN-38, Dxd (derivative of exatecan), exatecan, topotecan, morpholino-doxorubicin, rhizoxin, cyanomorpholino-doxorubicin, dolastatin-10, echinomycin, combretatstatin, calicheamicin, or netropsin. In some embodiments, the anti-cotinine antibody or antigen binding fragment thereof is conjugated to an auristatin, a maytansinoid, or calicheamicin. In some embodiments, the anti-cotinine antibody or antigen binding fragment thereof is conjugated to AFP, MMAP, MMAE, AEB, AEVB, auristatin E, vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansinol, maytansine, DM1, DM2, DM3, DM4 or eleutherobin. In some embodiments, the anti-cotinine antibody or antigen binding fragment thereof is conjugated to a topoisomerase inhibitor. In some embodiments, the anti-cotinine antibody or antigen binding fragment thereof is conjugated to a topoisomerase inhibitor selected from exatecan or Dxd (derivative of exatecan).

In some embodiments, the antibody-drug conjugate comprises an anti-cotinine antibody or antigen-binding fragment thereof covalently bound to a cytotoxic agent. In some embodiments, the anti-cotinine antibody or antigen-binding fragment thereof covalently bound to a cytotoxic agent comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In some embodiments, the anti-cotinine antibody or antigen-binding fragment thereof covalently bound to a cytotoxic agent comprises a heavy chain variable region (VH) as set forth in SEQ ID NO: 7 and a light chain variable region (VL) as set forth in SEQ ID NO: 8. In some embodiments, the anti-cotinine antibody covalently bound to a cytotoxic agent comprises a heavy chain as set forth in SEQ ID NO: 9 and a light chain as set forth in SEQ ID NO: 10.

In some embodiments, the disclosure provides antibody-drug conjugates having the following general structure:

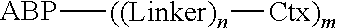

wherein ABP is an antigen binding protein, such as an anti-cotinine antibody or antigen-binding fragment thereof;
Linker is either absent or a cleavable or non-cleavable linker;
Ctx is any cytotoxic agent described herein;
n is 0, 1, 2, or 3; and
m is 1, 2, 3, 4, 5, 6, 7, 8, 9 or 10.

Exemplary linkers include 6-maleimidocaproyl (MC), maleimidopropanoyl (MP), valine-citrulline (val-cit), alanine-phenylalanine (ala-phe), glycine-glycine-phenylalanine-glycine (gly-gly -phe-gly), p-aminobenzyloxycarbonyl (PAB), N-Succinimidyl 4-(2-pyridylthio) pentanoate (SPP), N-succinimidyl 4-(N-maleimidomethyl)cyclohexane-1 carboxylate (SMCC), and N-succinimidyl (4-iodo-acetyl)aminobenzoate (SIAB). In some embodiments, the antibody-drug conjugate comprises an anti-cotinine antibody or antigen-binding fragment thereof covalently bound to Dxd. In some embodiments, the antibody-drug conjugate comprises an anti-cotinine antibody or antigen-binding fragment thereof covalently bound to Dxd via a linker. In some embodiments, the linker comprises gly-gly-phe-gly. In some embodiments, the antibody-drug conjugate comprises an anti-cotinine antibody or antigen-binding fragment thereof covalently bound to deruxtecan. In some embodiments, the ratio of cytotoxic agent to antibody or antigen-binding fragment thereof is in a range of about 1:1 to about 10:1. In some embodiments, the ratio of cytotoxic agent to antibody or antigen-binding fragment thereof is about 1:1, about 2:1, about 3:1, about 4:1, about 5:1, about 6:1, about 7:1, about 8:1, about 9:1, or about 10:1. In another embodiment, the antibody-drug conjugate comprises an anti-cotinine antibody or antigen-binding fragment thereof covalently bound to Dxd via a linker comprising gly-gly-phe-gly. In some embodiments, the linker is as depicted in the following structure:

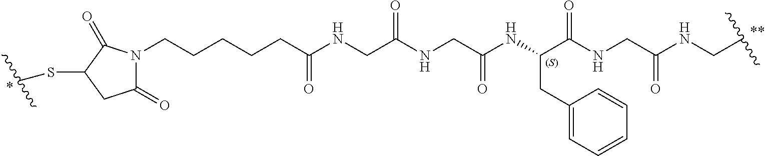

wherein

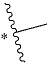

indicates the point of attachment to the antibody or antigen-binding fragment thereof and

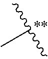

indicates the point of attachment to the cytotoxic agent, such as Dxd.

In some embodiments, the antibody-drug conjugate comprises a Dxd moiety as depicted in the following structure:

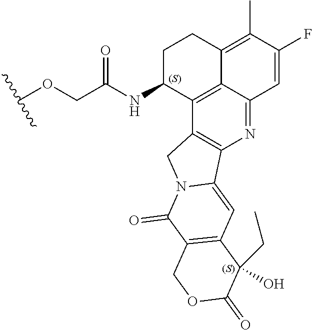

wherein the wavy line indicates the position of binding to the linker, if the linker is present, or the antibody or antigen-binding fragment thereof, if the linker is absent.

In another embodiment, the antibody-drug conjugate comprises an anti-cotinine antibody or antigen-binding fragment thereof covalently bound to deruxtecan as depicted in the following structure:

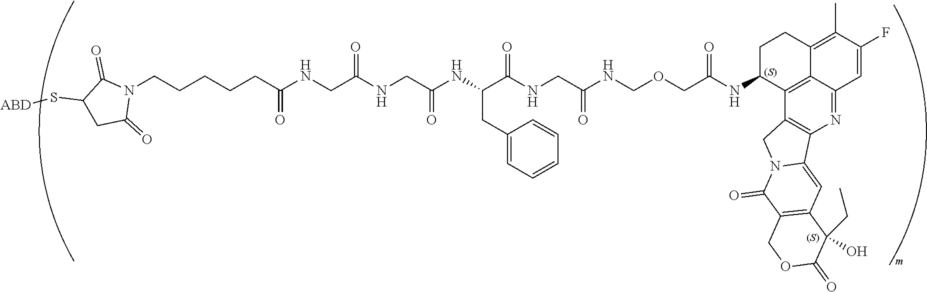

wherein m is 1, 2, 3, 4, 5, 6, 7, 8, 9, or 10.

The present disclosure also provides a pharmaceutical composition comprising an antibody-drug conjugate as disclosed herein, and a pharmaceutically acceptable excipient, carrier, or diluent.

### Bispecific Antibodies and Bispecific Antigen Binding Fragments Thereof

The present disclosure also provides a bispecific antibody or bispecific antigen binding fragment thereof, including a bispecific T-cell engager, that binds to CD3 and a cotinine moiety. In some cases, CD3 is an activating T cell antigen. An “activating T cell antigen” as used herein can refer to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In some cases, a T cell engager disclosed herein is capable of inducing T cell activation. “T cell activation” as used herein can refer to one or more cellular response of a T lymphocyte, particularly a cytotoxic T lymphocyte, selected from: proliferation, differentiation, cytokine secretion, cytotoxic effector molecule release, cytotoxic activity, and expression of activation markers.

In some cases, the bispecific T-cell engager binds to both the CD3 antigen on cytotoxic T lymphocytes (CTLs) and a target cell-surface protein found on target-expressing cells with binding to the target cell-surface protein mediated by a heterobivalent molecule comprising a cotinine moiety covalently linked to a moiety that binds the target cell-surface protein. In some cases, this activates and crosslinks CTLs with target-expressing cells, which results in the CTL-mediated cell death of the target-expressing cells.

In some embodiments, a bispecific antibody or bispecific antigen binding fragment thereof provided herein comprises a cotinine binding domain and a CD3 binding domain.

The term “cotinine binding domain” as used herein refers to antibodies and other protein constructs (e.g., single chain variable fragments or scFvs), which are capable of binding to a cotinine moietyor a derivative thereof. In one embodiment, the cotinine binding domain is an antibody. In another embodiment, the cotinine binding domain is a scFv.

The term “CD3 binding domain” as used herein refers to antibodies and other protein constructs (e.g., single chain variable fragments or scFvs), which are capable of binding to CD3. This does not include the natural cognate receptor. In one embodiment, the CD3 binding domain is an antibody. In another embodiment, the CD3 binding domain is a scFv.

In some embodiments, a cotinine binding domain comprises a single chain variable fragment (scFv). In some embodiments, a cotinine binding domain comprises an antibody heavy chain and an antibody light chain. In some embodiments, a CD3 binding domain comprises a single chain variable fragment (scFv). In some embodiments, a CD3 binding domain comprises an antibody heavy chain and an antibody light chain that specifically binds CD3.

In one embodiment, a bispecific antibody or bispecific antigen binding fragment thereof comprising a cotinine binding domain and a CD3 binding domain is a bispecific antibody. In one embodiment, a bispecific antibody or bispecific antigen binding fragment thereof comprising a cotinine binding domain and a CD3 binding domain is a bispecific antibody with an immunoglobulin format.

In some embodiments, a bispecific antibody or bispecific antigen binding fragment thereof comprises a cotinine binding domain comprising a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6.

In some embodiments, a bispecific antibody or bispecific antigen binding fragment thereof comprises a CD3 binding domain comprising a heavy chain CDR1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21.

In some embodiments, a bispecific antibody or bispecific antigen binding fragment thereof comprises a CD3 binding domain comprising a heavy chain CDR1 having SEQ ID NO: 30, a heavy chain CDR2 having SEQ ID NO: 31, a heavy chain CDR3 having SEQ ID NO: 32, a light chain CDR1 having SEQ ID NO: 33, a light chain CDR2 having SEQ ID NO: 34, and a light chain CDR3 having SEQ ID NO: 35.

In some embodiments, a bispecific antibody or bispecific antigen binding fragment thereof comprises a cotinine binding domain and a CD3 binding domain, wherein the cotinine binding domain comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6; and the CD3 binding domain comprises a heavy chain CDR1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21.

In some embodiments, a bispecific antibody or bispecific antigen binding fragment thereof comprises a cotinine binding domain and a CD3 binding domain, wherein the cotinine binding domain comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6; and the CD3 binding domain comprises a heavy chain CDR1 having SEQ ID NO: 30, a heavy chain CDR2 having SEQ ID NO: 31, a heavy chain CDR3 having SEQ ID NO: 32, a light chain CDR1 having SEQ ID NO: 33, a light chain CDR2 having SEQ ID NO: 34, and a light chain CDR3 having SEQ ID NO: 35.

In some embodiments, the bispecific antibody or bispecific antigen binding fragment thereof comprises a first single chain variable fragment (scFv) that binds a cotinine moiety, and a second scFv that binds CD3. In some embodiments, the scFv that binds a cotinine moiety comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In some embodiments, the scFv that binds a cotinine moiety comprises a heavy chain variable region (VH) and a light chain variable region (VL) joined by a first polypeptide linker. In some embodiments, the scFv that binds a cotinine moiety comprises a VH as set forth in SEQ ID NO: 7 and a VL as set forth in SEQ ID NO: 8. In some embodiments, the scFv that binds a cotinine moiety comprises a VH as set forth in SEQ ID NO: 7 and a VL as set forth in SEQ ID NO: 8 joined by a first polypeptide linker. In some embodiments, the scFv that binds a cotinine moiety is as set forth in SEQ ID NO: 15.

In some embodiments, the scFv that binds CD3 comprises a heavy chain CDR 1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21. In some embodiments, the scFv that binds CD3 comprises a VH and a VL joined by a second polypeptide linker. In some embodiments, the scFv that binds CD3 comprises a VH as set forth in SEQ ID NO: 22 and VL as set forth in SEQ ID NO: 23. In some embodiments, the scFv that binds CD3 comprises a VH as set forth in SEQ ID NO: 22 and VL as set forth in SEQ ID NO: 23 joined by a second polypeptide linker.

In some embodiments, the scFv that binds CD3 comprises a heavy chain CDR1 having SEQ ID NO: 30, a heavy chain CDR2 having SEQ ID NO: 31, a heavy chain CDR3 having SEQ ID NO: 32, a light chain CDR1 having SEQ ID NO: 33, a light chain CDR2 having SEQ ID NO: 34, and a light chain CDR3 having SEQ ID NO: 35. In some embodiments, the scFv that binds CD3 comprises a VH and a VL joined by a second polypeptide linker. In some embodiments, the scFv that binds CD3 comprises a VH as set forth in SEQ ID NO: 36 and a VL as set forth in SEQ ID NO: 37. In some embodiments, the scFv that binds CD3 comprises a VH as set forth in SEQ ID NO: 36 and a VL as set forth in SEQ ID NO: 37 joined by a second polypeptide linker. In some embodiments, the scFv that binds CD3 is as set forth in SEQ ID NO: 24.

In some embodiments, the bispecific antibody or bispecific antigen binding fragment thereof (or bispecific T-cell engager) comprises a first scFv that binds a cotinine moiety, and a second scFv that binds CD3, wherein the first scFv and the second scFv are joined by a third polypeptide linker. In some embodiments, the bispecific antibody or bispecific antigen binding fragment thereof is a bispecific T-cell engager is as set forth in SEQ ID NO: 25. In some embodiments, the bispecific antibody or bispecific antigen binding fragment thereof is a bispecific T-cell engager is as set forth in SEQ ID NO: 29.

In some embodiments, the bispecific antibody or bispecific antigen binding fragment thereof is a bispecific antibody that binds a cotinine moiety and CD3. In some embodiments, the bispecific antibody comprises a cotinine binding domain comprising a heavy chain and a light chain that bind a cotinine moiety; and a CD3 binding domain comprising a heavy chain and a light chain that bind CD3. In some embodiments, the cotinine binding domain comprises a heavy chain comprising a heavy chain CDR1 having SEQ ID NO: 1, a heavy chain CDR2 having SEQ ID NO: 2, and a heavy chain CDR3 having SEQ ID NO: 3, and a light chain comprising a light chain CDR1 having SEQ ID NO: 4, a light chain CDR2 having SEQ ID NO: 5, and a light chain CDR3 having SEQ ID NO: 6. In some embodiments, the cotinine binding domain comprises a heavy chain comprising a heavy chain variable region (VH) as set forth in SEQ ID NO: 7 and a light chain comprising a light chain variable region (VL) as set forth in SEQ ID NO: 8. In some embodiments, the cotinine binding domain comprises a heavy chain as set forth in SEQ ID NO: 28 and a light chain as set forth in SEQ ID NO: 10. In some embodiments, the CD3 binding domain comprises a heavy chain comprising a heavy chain CDR1 having SEQ ID NO: 16, a heavy chain CDR2 having SEQ ID NO: 17, and a heavy chain CDR3 having SEQ ID NO: 18, and a light chain comprising a light chain CDR1 having SEQ ID NO: 19, a light chain CDR2 having SEQ ID NO: 20, and a light chain CDR3 having SEQ ID NO: 21. In some embodiments, the CD3 binding domain comprises a heavy chain comprising a heavy chain variable region (VH) as set forth in SEQ ID NO: 22 and a light chain comprising a light chain variable region (VL) as set forth in SEQ ID NO: 23. In some embodiments, the CD3 binding domain comprises a heavy chain as set forth in SEQ ID NO: 26 and a light chain as set forth in SEQ ID NO: 27.

In some embodiments, the bispecific antibody or bispecific antigen binding fragment thereof is a bispecific antibody that binds a cotinine moiety and CD3, wherein the bispecific antibody comprises a cotinine binding domain comprising a heavy chain comprising a heavy chain variable region (VH) as set forth in SEQ ID NO: 7 and a light chain comprising a light chain variable region (VL) as set forth in SEQ ID NO: 8; and a CD3 binding domain comprising a heavy chain comprising a heavy chain variable region (VH) as set forth in SEQ ID NO: 22 and a light chain comprising a light chain variable region (VL) as set forth in SEQ ID NO: 23.

In some embodiments, the bispecific antibody or bispecific antigen binding fragment thereof is a bispecific antibody that binds a cotinine moiety and CD3, wherein the bispecific antibody comprises a cotinine binding domain comprising a heavy chain as set forth in SEQ ID NO: 28 and a light chain as set forth in SEQ ID NO: 10; and a CD3 binding domain comprising a heavy chain as set forth in SEQ ID NO: 26 and a light chain as set forth in SEQ ID NO: 27.

The present disclosure also provides a pharmaceutical composition comprising a bispecific antibody or bispecific antigen binding fragment thereof (e.g., bispecific T-cell engager) as disclosed herein, and a pharmaceutically acceptable excipient, carrier, or diluent.

### Polynucleotides

In another aspect, a polynucleotide encoding one or more bispecific antibodies or bispecific antigen binding fragments thereof, including bispecific T cell engagers, as described herein is provided. As used herein, the terms “polynucleotide” or “nucleic acid” refer to messenger RNA (mRNA), RNA, genomic RNA (gRNA), plus strand RNA (RNA (+)), minus strand RNA (RNA (−)), genomic DNA (gDNA), complementary DNA (cDNA) or recombinant DNA. Polynucleotides include single and double stranded polynucleotides.

In various illustrative embodiments, polynucleotides include expression vectors, viral vectors, and transfer plasmids, and compositions and cells comprising the same. In various illustrative embodiments, polynucleotides encode a bispecific antibody or bispecific antigen binding fragment thereof (e.g., bispecific T cell engager) or polypeptide contemplated herein, including, but not limited to a bispecific T cell engager having the sequence of SEQ ID NO: 25 or a polynucleotide sequence encoding SEQ ID NO: 25.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

In a further aspect, the present disclosure provides a cell comprising the polynucleotide encoding the bispecific antibody or bispecific antigen binding fragment thereof (e.g., bispecific T cell engager) as disclosed herein.

### Vectors

In another aspect, the present invention provides vectors which comprise a polynucleotide encoding one or more bispecific antibodies or bispecific antigen binding fragments thereof (e.g., bispecific T cell engagers) as described herein.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes and viral vectors. Useful viral vectors include, e.g., replication defective retroviruses and lentiviruses.

In particular embodiments, the vectors are expression vectors. Expression vectors may be used to produce bispecific antibodies or bispecific antigen binding fragments thereof (e.g., bispecific T cell engagers) and polypeptides contemplated herein. In addition, expression vectors may include additional components which allow for the production of viral vectors, which in turn comprise a polynucleotide contemplated herein. Viral vectors may be used for delivery of the polynucleotides contemplated herein to a subject or a subject's cells. Examples of expression vectors include, but are not limited to, plasmids, autonomously replicating sequences and transposable elements. Additional exemplary vectors include, without limitation, plasmids, phagemids, cosmids, transposons, artificial chromosomes such as yeast artificial chromosome (YAC), bacterial artificial chromosome (BAC), or PI-derived artificial chromosome (PAC), bacteriophages such as lambda phage or MI 3 phage, and animal viruses.

Additional examples of expression vectors are pClneo vectors (Promega) for expression in mammalian cells; pLenti4/V5-DEST™ pLenti6/V5-DESTTM and pLenti6.2/V5 -GW/IacZ (Invitrogen)) for lentivirus-mediated gene transfer and expression in mammalian cells. In particular embodiments, the coding sequences of the bispecific antibodies or bispecific antigen binding fragments thereof (e.g., bispecific T cell engagers) and polypeptides disclosed herein can be ligated into such expression vectors for the expression of the bispecific antibodies or bispecific antigen binding fragments thereof (e.g., bispecific T cell engagers) and/or polypeptides in mammalian cells.

In particular embodiments, the expression vectors provided herein are BACs which comprise a polynucleotide as described herein. In particular embodiments, the BACs additionally comprise one or more polynucleotides encoding for proteins necessary to allow the production of a viral vector when expressed in a producer or packaging cell line. By way of example, PCT applications WO2017/089307 and WO2017/089308 describe expression vectors used to produce retroviral vectors, in particular lentiviral vectors. In a particular embodiment, the expression vectors described in WO2017/089307 and WO2017/089308, comprising a polynucleotide as described herein are provided.

The “control elements” or “regulatory sequences” present in an expression vector are those non-translated regions of the vector-origin of replication, selection cassettes, promoters, enhancers, translation initiation signals (Shine Dalgarno sequence or Kozak sequence), introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In a further aspect, the present disclosure provides a cell comprising the expression vector comprising the polynucleotide encoding the bispecific antibodies or bispecific antigen binding fragments thereof (e.g., bispecific T cell engager) as disclosed herein.

### Heterobifunctional Molecules

In another embodiment, the disclosure provides a combination comprising an antibody-drug conjugate as disclosed herein and a heterobivalent molecule comprising a cotinine moiety covalently linked to a target binding moiety. In another embodiment, the combination comprises an antibody-drug conjugate as disclosed herein and a heterobifunctional molecule comprising a moiety that binds a target cell-surface protein covalently linked to a cotinine moiety.

In another embodiment, the disclosure provides a combination comprising a bispecific antibody or bispecific antigen binding fragment thereof (e.g., bispecific T cell engager) as disclosed herein and a heterobivalent molecule comprising a cotinine moiety covalently linked to a target binding moiety. In another embodiment, the combination comprises a bispecific antibody or bispecific antigen binding fragment thereof (e.g., bispecific T cell engager) as disclosed herein and a heterobifunctional molecule comprising a moiety that binds a target cell-surface protein covalently linked to a cotinine moiety.

In another embodiment, the heterobifunctional molecule is a compound of Formula (I):

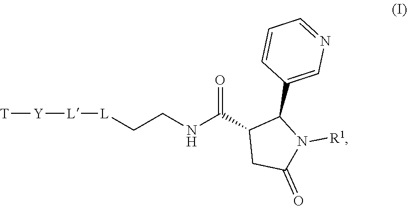

or a pharmaceutically acceptable salt thereof,
wherein:
T is a target binding moiety;
R¹ is C₁₋₄ alkyl or C₃₋₆ cycloalkyl;

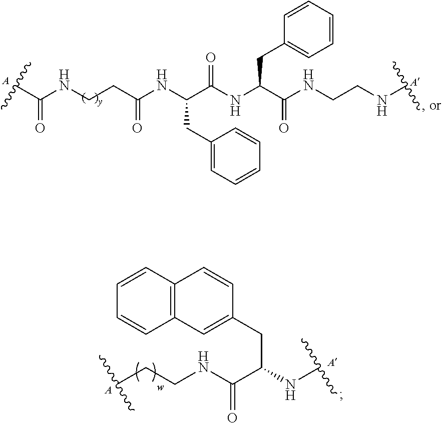

L′ is a bond,
y is an integer of 1 to 9;
w is an integer of 0 to 5;
Y is a bond or a divalent spacer moiety of one to twelve atoms in length; and
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), (L-n-iv), (L-p), (L-q), (L-r), or (L-s);
wherein each

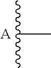

represents a covalent bond to the Y group of Formula (I), or when Y is a bond, a covalent bond to the T group of Formula (I), and each

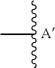

represents a covalent bond to the L group of Formula (I).

In another embodiment, L is a divalent linker of Formula (L-a):

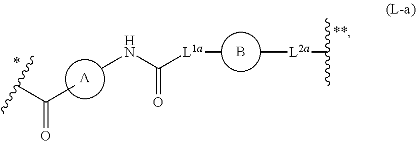

or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O—or —NR^{a}−;
each R^{a} is independently hydrogen or C₁₋₃ alkyl; and
L^{2a} is —O—, —NHC(O)—, or —CH₂—O—;
wherein

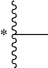

represents a covalent bond to the L′ group of Formula (I), or when L′ is a bond, a covalent bond to the Y group of Formula (I), or when both L′ and Y are a bond, a covalent bond to the T group of Formula (I), and

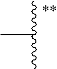

represents a covalent bond to the methylene group of Formula (I).

In another embodiment, Ring A and Ring B of Formula (L-a) are each independently

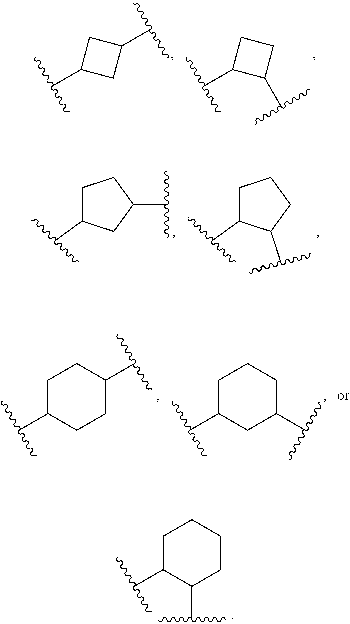

In another embodiment, L is a divalent linker of Formula (L-a-i):

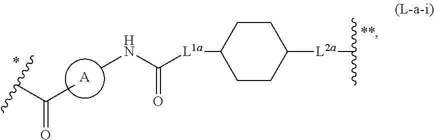

or a stereoisomer thereof,
wherein:
Ring A is C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
   wherein

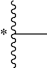

represents a covalent bond to the L′ group of Formula (I), or when L′ is a bond, a covalent bond to the Y group of Formula (I), or when both L′ and Y are a bond, a covalent bond to the T group of Formula (I), and

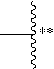

represents a covalent bond to the methylene group of Formula (I).

In another embodiment, Ring A of Formula (L-a-i) is

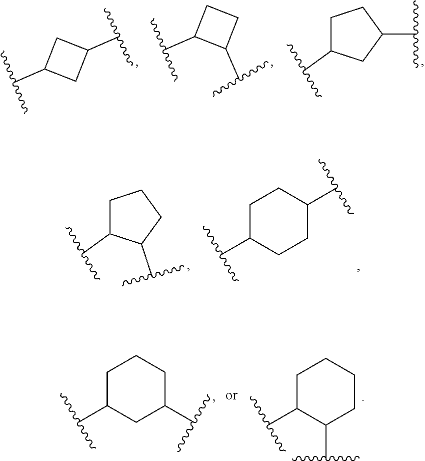

In another embodiment, L is a divalent linker of Formula (L-a-ii):

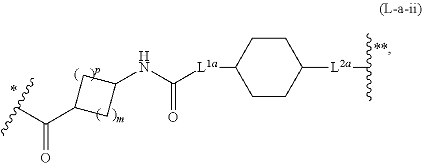

or a stereoisomer thereof,
wherein:

L^{1a} is C₃₋₅ linear alkylene, wherein 1 or 2 methylene units are replaced with —O— or —NR^{a}-; each R^{a} is independently hydrogen or C₁₋₃ alkyl;
L^{2a} is —O—, —NHC(O)—, or —CH₂—O—;
p is 1 or 2; and
m is 1 or 2;
wherein

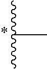

represents a covalent bond to the L′ group of Formula (I), or when L′ is a bond, a covalent bond to the Y group of Formula (I), or when both L′ and Y are a bond, a covalent bond to the T group of Formula (I), and

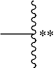

represents a covalent bond to the methylene bond to the T group of Formula (I), and group of Formula (I).

In another embodiment, L^{1a} of Formula (L-a), (L-a-i), or (L-a-ii) is selected from

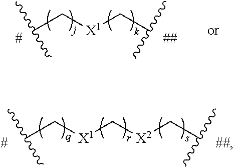

wherein:

j is 1, 2, 3, or 4;
k is 0, 1, 2, or 3;
the sum of j and k is 2, 3, or 4;
q is 1 or 2;
r is 1 or 2;
s is 0 or 1;
the sum of q, r, and s is 2 or 3;
X¹ and X² are independently —O— or NR^{a}; and
each R^{a} is independently hydrogen or C₁₋₃ alkyl;
wherein

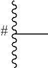

represents a covalent bond to the C(O) group of Formula (L-a), (L-a-i), or (L-a-ii), and

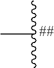

represents a covalent bond to Ring B of Formula (L-a) or to the cyclohexylene group of Formula (L-a-i) or (L-a-ii).

In another embodiment, L^{1a} of Formula (L-a), (L-a-i), or (L-a-ii) is selected from —(CH₂)₂O—, —(CH₂)₃O—, —(CH₂)₄O—, —(CH₂)₂OCH2—, —(CH₂)₃OCH2—, —(CH₂)₂O(CH₂)₂—, —CH₂OCH2—, —CH₂O(CH₂)₂—, —CH₂O(CH₂)₃—, —CH₂OCH₂O—, or —CH₂OCH₂OCH₂—. In another embodiment, L^{1a} of Formula (L-a), (L-a-i), or (L-a-ii) is selected from —(CH₂)₂O—, —(CH₂)₃O—, —(CH₂)₂OCH₂—, or —(CH₂)₃OCH₂—. In another embodiment, L^{1a} of Formula (L-a), (L-a-i), or (L-a-ii) is selected from (CH₂)₂NR^{a}—, —(CH₂)₃NR^{a}—, —(CH₂)₄NR^{a}—, —(CH₂)₂NR^{a}CH₂—, —(CH₂)₃NR^{a}CH₂—, —(CH₂)₂NR^{a}(CH₂)₂—, —CH₂NR^{a}CH₂—, —CH₂NR^{a}(CH₂)₂—, —CH₂NR^{a}(CH₂)₃—, —CH₂NR^{a}CH₂NR^{a}—, or CH₂NR^{a}CH₂NR^{a}CH₂—, wherein each R^{a} is independently hydrogen or C₁₋₃ alkyl. In another embodiment, L^{1a} of Formula (L-a), (L-a-i), or (L-a-ii) is selected from—(CH₂)₂NR^{a}—, —(CH₂)₃NR^{a}—, —(CH₂)₂NR^{a}CH₂—, or —(CH₂)₃NR^{a}CH₂—, wherein R^{a} is hydrogen or C₁₋₃ alkyl. In another embodiment, L^{1a} of Formula (L-a), (L-a-i), or (L-a-ii) is selected from—(CH₂)₂NH—, —(CH₂)₃NH—, (CH₂)₄NH—, —(CH₂)₂NHCH₂—, —(CH₂)₃NHCH₂—, —(CH₂)₂NH(CH₂)₂—, —CH₂NHCH₂—, —CH₂NH(CH₂)₂—, —CH₂NH(CH₂)₃—, —CH₂NHCH₂NH—, or —CH₂NHCH₂NHCH₂—. In another embodiment, L^{1a} of Formula (L-a), (L-a-i), or (L-a-ii) is selected from—(CH₂)₂NH—, —(CH₂)₃NH—, —(CH₂)₂NHCH₂—, or —(CH₂)₃NHCH₂—. In another embodiment, L^{1a} of Formula (L-a), (L-a-i), or (L-a-ii) is selected from —CH₂OCH₂NR^{a}—, —CH₂NR^{a}CH₂O—, —CH₂OCH₂NR^{a}CH₂—, —CH₂NR^{a}CH₂OCH₂—, wherein R^{a} is independently hydrogen or C₁₋₃ alkyl. In another embodiment, L^{1a} of Formula (L-a), (L-a-i), or (L-a-ii) is selected from-CH₂OCH₂NH—, —CH₂NHCH₂O—, —CH₂OCH₂NHCH₂—, —CH₂NHCH₂OCH₂—.

In another embodiment, L is a divalent linker of Formula (L-a-iii):

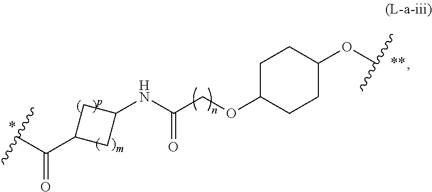

or a stereoisomer thereof,
wherein:
p is 1 or 2;
m is 1 or 2; and
n is 1, 2, or 3;
wherein

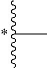

represents a covalent bond to the L′ group of Formula (I), or when L′ is a bond, a covalent bond to the Y group of Formula (I), or when both L′ and Y are a bond, a covalent bond to the T group of Formula (I), and

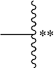

represents a covalent bond to the methylene bond to the T group of Formula (I), and group of Formula (I).

In another embodiment, L is a divalent linker of Formula (L-a) selected from the group consisting of:

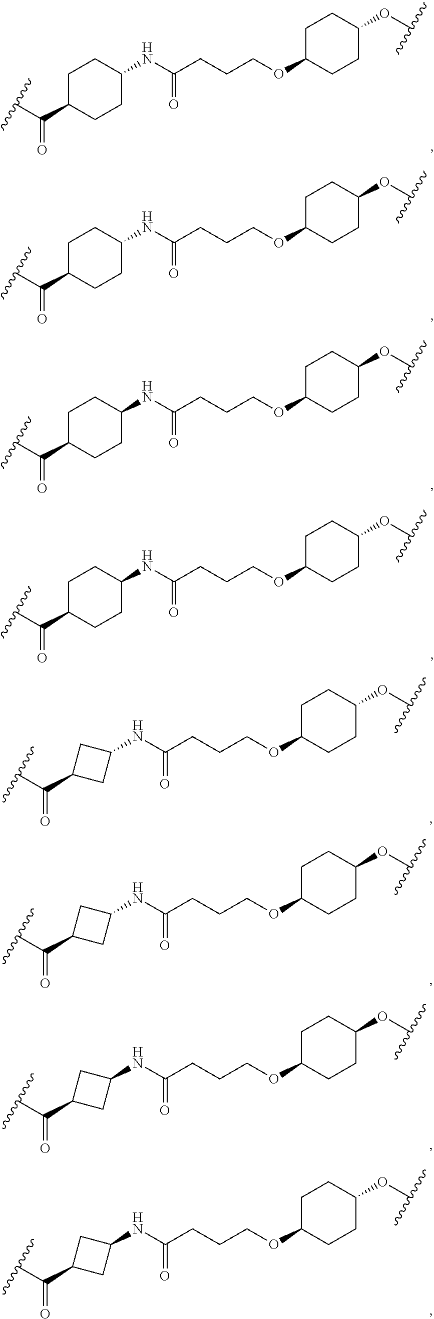

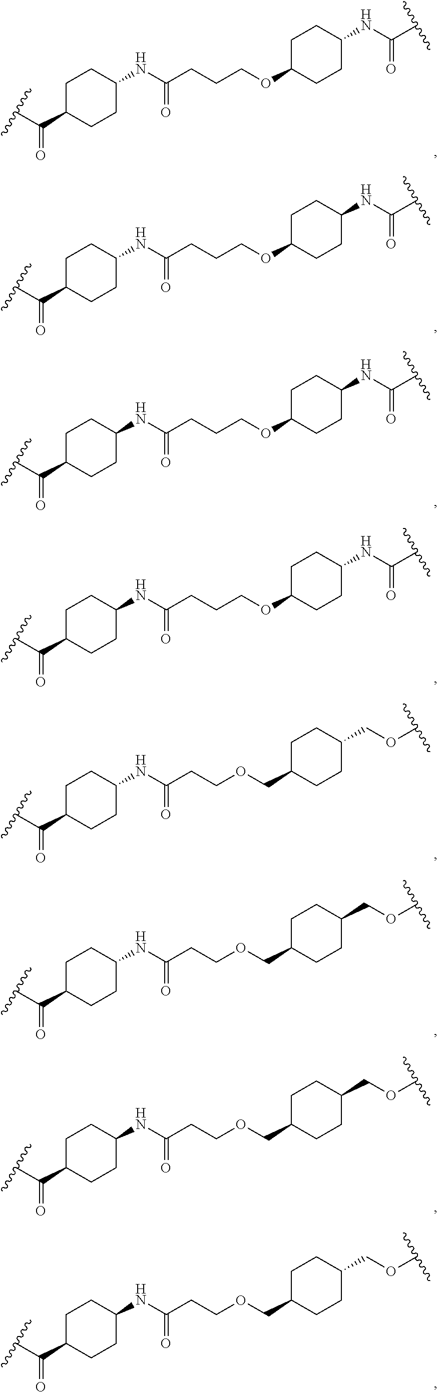

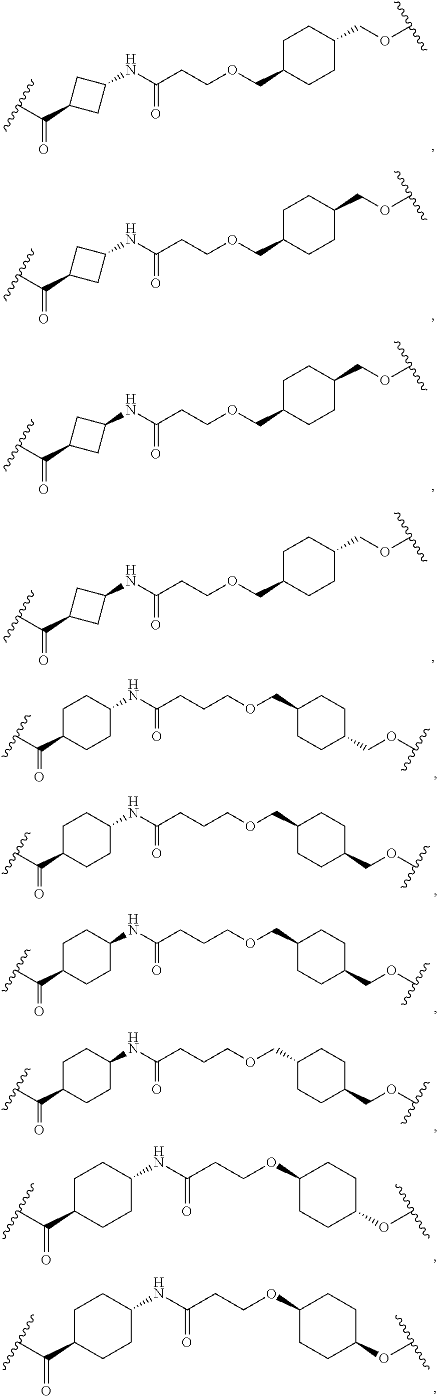

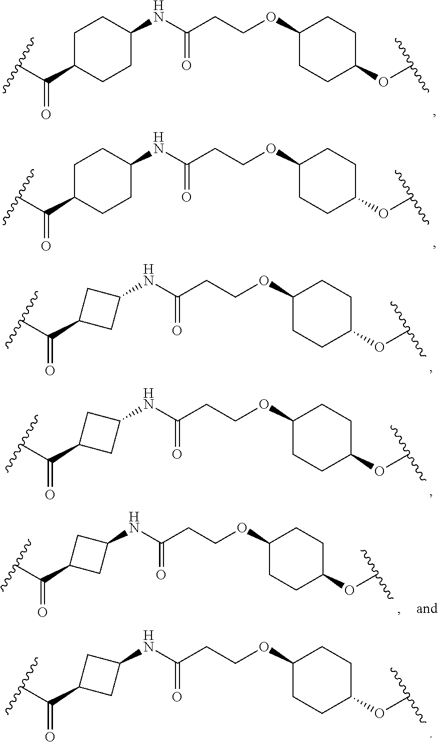 [SMILES: *O[C@H]1CC[C@@H](OCCC(=O)N[C@H]2CC[C@@H](C(*)=O)CC2)CC1 ; *O[C@H]1CC[C@H](OCCC(=O)N[C@H]2CC[C@@H](C(*)=O)CC2)CC1 ; *O[C@H]1CC[C@H](OCCC(=O)N[C@H]2C[C@H](C(*)=O)C2)CC1 ; *O[C@H]1CC[C@@H](OCCC(=O)N[C@H]2C[C@H](C(*)=O)C2)CC1 ; *O[C@H]1CC[C@@H](OCCC(=O)N[C@H]2C[C@@H](C(*)=O)C2)CC1 ; *O[C@H]1CC[C@H](OCCC(=O)N[C@H]2C[C@@H](C(*)=O)C2)CC1]

In another embodiment, L is a divalent linker of Formula (L-b):

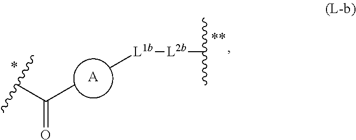

or a stereoisomer thereof,
wherein:
Ring A is C₄₋₆ cycloalkylene or C₇₋₉ bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is

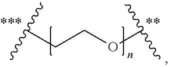

wherein n is 1, 2, 3, or 4, and

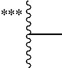

represents a covalent bond to L^{1b};
each R^{1b} is independently hydrogen or C₁₋₃ alkyl;
wherein

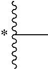

represents a covalent bond to the L′ group of Formula (I), or when L′ is a bond, a covalent bond to the Y group of Formula (I), or when both L′ and Y are a bond, a covalent bond to the T group of Formula (I), and

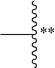

represents a covalent bond to the methylene group of Formula (I).

In another embodiment, Ring A of Formula (L-b) is

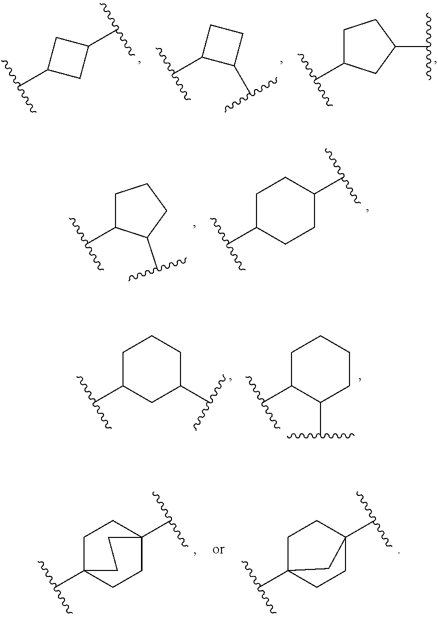

In another embodiment, L is a divalent linker of Formula (L-b-i):

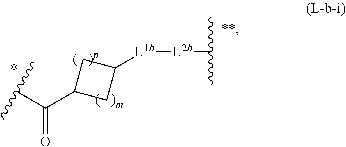

or a stereoisomer thereof,
wherein:
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—C(O)NR^{1b}—, or —NR^{1b}C(O)—; or
L^{2b} is

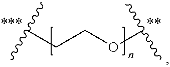

wherein n is 1, 2, 3, or 4, and

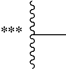

represents a covalent bond to L^{1b};
each R^{1b} is independently hydrogen or C₁₋₃ alkyl;
p is 1 or 2; and
m is 1 or 2;
wherein

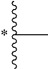

represents a covalent bond to the L′ group of Formula (I), or when L′ is a bond, a covalent bond to the Y group of Formula (I), or when both L′ and Y are a bond, a covalent bond to the T group of Formula (I), and

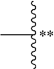

represents a covalent bond to the methylene bond to the T group of Formula (I), and group of Formula (I).

In another embodiment, L^{2b} of Formula (L-b) or (L-b-i) is selected from

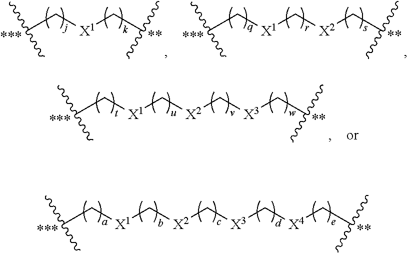

wherein:
j is 1, 2, 3, 4, 5, 6, 7, 8, 9, or 10;
k is 1, 2, 3, 4, 5, 6, 7, 8, 9, or 10;
the sum of j and k is 5, 6, 7, 8, 9, 10, or 11;
q is 1, 2, 3, 4, 5, 6, 7, 8, or 9;
r is 1, 2, 3, 4, 5, 6, 7, 8, or 9;
s is 0, 1, 2, 3, 4, 5, 6, 7, or 8;
the sum of q, r, and s is 4, 5, 6, 7, 8, 9, or 10;
tis 1, 2, 3, 4, 5, 6, or 7;
u is 1, 2, 3, 4, 5, 6, or 7;
v is 1, 2, 3, 4, 5, 6, or 7;
w is 0, 1, 2, 3, 4, 5, or 6;
the sum of t, u, v, and w is 3, 4, 5, 6, 7, 8, or 9;
a is 1, 2, 3, 4, or 5;
bis 1, 2, 3, 4, or 5;
c is 1, 2, 3, 4, or 5;
d is 1, 2, 3, 4, or 5;
e is 0, 1, 2, 3, or 4;
the sum of a, b, c, d, and e is 4, 5, 6, 7, or 8;
X¹, X², X³, and X⁴ are independently —O—, —NR^{1b}—, —C(O)NR^{1b}—, or —NR^{1b}C(O)—; and
each R^{1b} is independently hydrogen or C₁₋₃ alkyl;
wherein

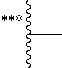

represents a covalent bond to L^{1b} of Formula (L-b) or (L-b-i), and

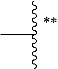

represents a covalent bond to the methylene group of Formula (I).

In another embodiment, L is a divalent linker of Formula (L-b) selected from the group consisting of:

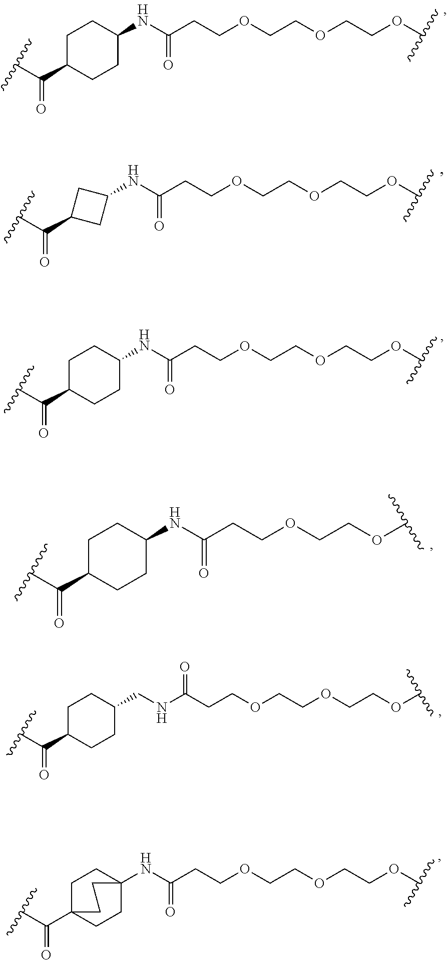 [SMILES: *OCCOCCOCCC(=O)N[C@H]1CC[C@@H](C(*)=O)CC1 ; *OCCOCCOCCC(=O)N[C@H]1C[C@H](C(*)=O)C1 ; *OCCOCCOCCC(=O)N[C@H]1CC[C@H](C(*)=O)CC1 ; *OCCOCCC(=O)N[C@H]1CC[C@@H](C(*)=O)CC1 ; *OCCOCCOCCC(=O)NC[C@H]1CC[C@H](C(*)=O)CC1 ; *OCCOCCOCCC(=O)NC12CCC(C(*)=O)(CC1)CC2]

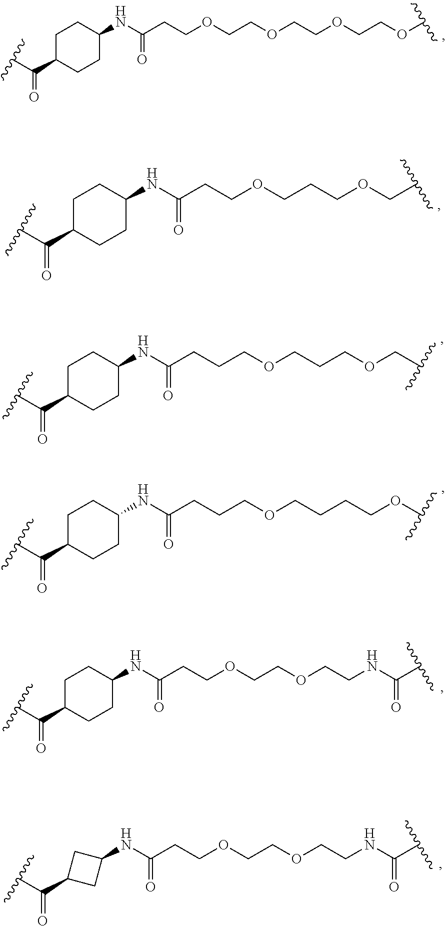 [SMILES: *OCCOCCOCCOCCC(=O)N[C@H]1CC[C@@H](C(*)=O)CC1 ; *COCCCOCCC(=O)N[C@H]1CC[C@@H](C(*)=O)CC1 ; *COCCCOCCCC(=O)N[C@H]1CC[C@@H](C(*)=O)CC1 ; *OCCCCOCCCC(=O)N[C@H]1CC[C@H](C(*)=O)CC1 ; *C(=O)NCCOCCOCCC(=O)N[C@H]1CC[C@@H](C(*)=O)CC1 ; *C(=O)NCCOCCOCCC(=O)N[C@H]1C[C@@H](C(*)=O)C1]

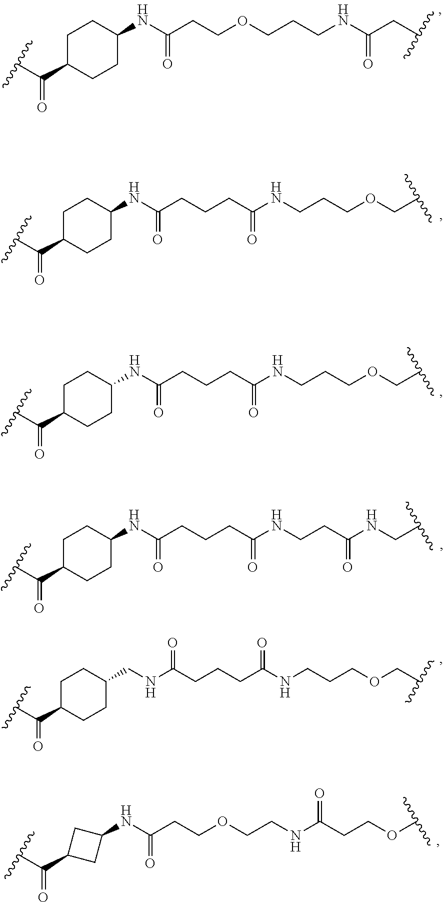 [SMILES: *CC(=O)NCCCOCCC(=O)N[C@H]1CC[C@@H](C(*)=O)CC1 ; *COCCCNC(=O)CCCC(=O)N[C@H]1CC[C@@H](C(*)=O)CC1 ; *COCCCNC(=O)CCCC(=O)N[C@H]1CC[C@H](C(*)=O)CC1 ; *CNC(=O)CCNC(=O)CCCC(=O)N[C@H]1CC[C@@H](C(*)=O)CC1 ; *COCCCNC(=O)CCCC(=O)NC[C@H]1CC[C@H](C(*)=O)CC1 ; *OCCC(=O)NCCOCCC(=O)N[C@H]1C[C@@H](C(*)=O)C1]

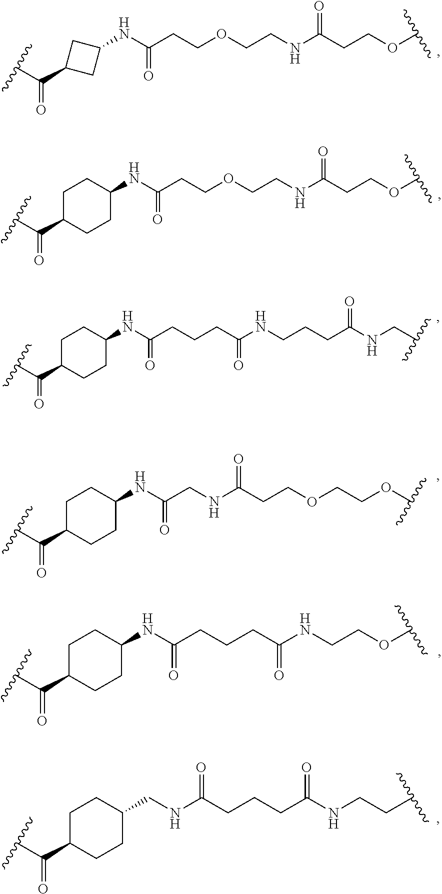 [SMILES: *OCCC(=O)NCCOCCC(=O)N[C@H]1C[C@H](C(*)=O)C1 ; *OCCC(=O)NCCOCCC(=O)N[C@H]1CC[C@@H](C(*)=O)CC1 ; *CNC(=O)CCCNC(=O)CCCC(=O)N[C@H]1CC[C@@H](C(*)=O)CC1 ; *OCCOCCC(=O)NCC(=O)N[C@H]1CC[C@@H](C(*)=O)CC1 ; *OCCNC(=O)CCCC(=O)N[C@H]1CC[C@@H](C(*)=O)CC1 ; *CCNC(=O)CCCC(=O)NC[C@H]1CC[C@H](C(*)=O)CC1]

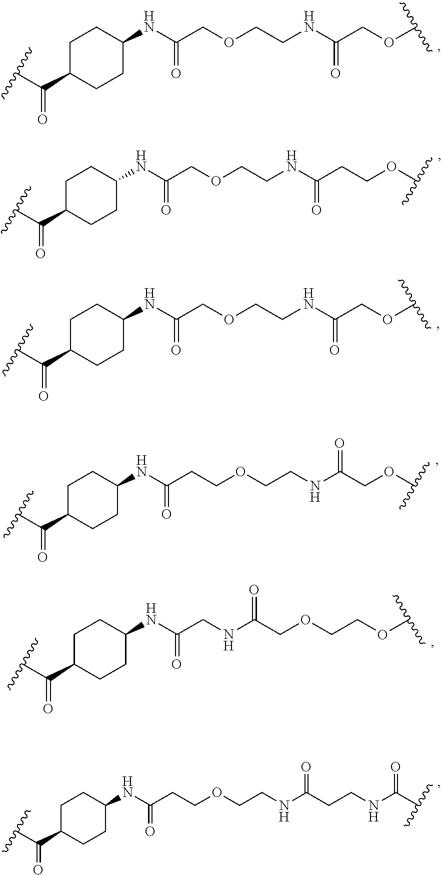 [SMILES: *OCC(=O)NCCOCC(=O)N[C@H]1CC[C@@H](C(*)=O)CC1 ; *OCCC(=O)NCCOCC(=O)N[C@H]1CC[C@H](C(*)=O)CC1 ; *OCC(=O)NCCOCC(=O)N[C@H]1CC[C@@H](C(*)=O)CC1 ; *OCC(=O)NCCOCCC(=O)N[C@H]1CC[C@@H](C(*)=O)CC1 ; *OCCOCC(=O)NCC(=O)N[C@H]1CC[C@@H](C(*)=O)CC1 ; *C(=O)NCCC(=O)NCCOCCC(=O)N[C@H]1CC[C@@H](C(*)=O)CC1]

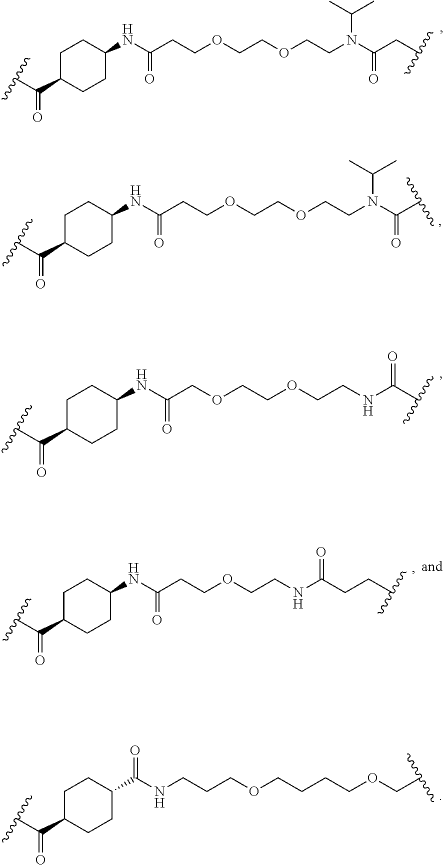 [SMILES: *CC(=O)N(CCOCCOCCC(=O)N[C@H]1CC[C@@H](C(*)=O)CC1)C(C)C ; *C(=O)N(CCOCCOCCC(=O)N[C@H]1CC[C@@H](C(*)=O)CC1)C(C)C ; *C(=O)NCCOCCOCC(=O)N[C@H]1CC[C@@H](C(*)=O)CC1 ; *CCC(=O)NCCOCCC(=O)N[C@H]1CC[C@@H](C(*)=O)CC1 ; *COCCCCOCCCNC(=O)[C@H]1CC[C@H](C(*)=O)CC1]

In another embodiment, L is a divalent linker of Formula (L-c):

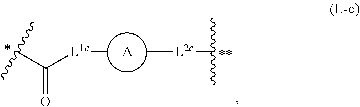

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇₋₉ bridged bicyclic cycloalkylene; and
L²c is —O— or a saturated C₂₋₁₀ linear alkylene, wherein 1, 2, or 3 methylene units are replaced with —O—, —NH—, —NHC(O)—, or —C(O)NH—;
wherein

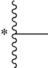

represents a covalent bond to the L′ group of Formula (I), or when L′ is a bond, a covalent bond to the Y group of Formula (I), or when both L′ and Y are a bond, a covalent bond to the T group of Formula (I), and

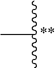

represents a covalent bond to the methylene group of Formula (I).

In another embodiment, Ring A of Formula (L-c) is

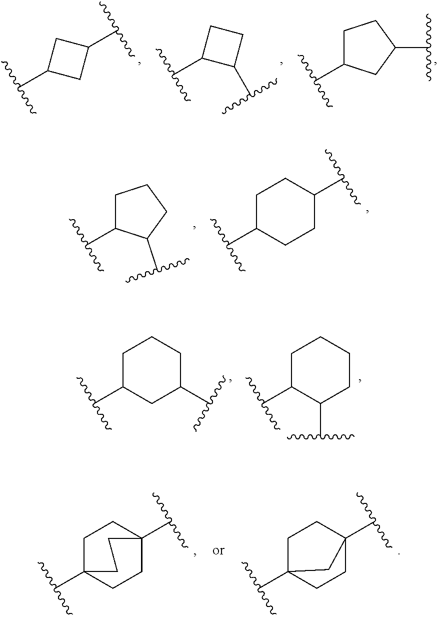

In another embodiment, L is a divalent linker of Formula (L-c-i):

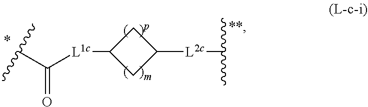

or a stereoisomer thereof,
wherein:
L¹c is C₂₋₁₀ linear alkylene, wherein 1, 2, or 3 methylene units are replaced with —O—, —NH—, —NHC(O)—, or —C(O)NH—;
L²c is —O— or a saturated C₂₋₁₀ linear alkylene, wherein 1, 2, or 3 methylene units are replaced with —O—, —NH—, —NHC(O)—, or —C(O)NH—;
p is 1 or 2; and
m is 1 or 2;
wherein

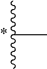

represents a covalent bond to the L′ group of Formula (I), or when L′ is a bond, a covalent bond to the Y group of Formula (I), or when both L′ and Y are a bond, a covalent bond to the T group of Formula (I), and

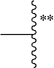

represents a covalent bond to the methylene group of Formula (I).

In another embodiment, L¹° of Formula (L-c) or (L-c-i) is selected from

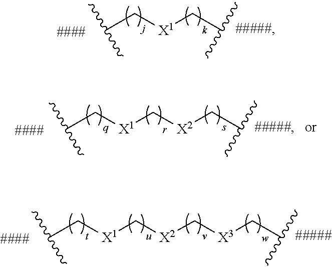

wherein:
j is 1, 2, 3, 4, 5, 6, 7, 8, or 9;
k is 0, 1, 2, 3, 4, 5, 6, 7, or 8;
the sum of j and k is 1, 2, 3, 4, 5, 6, 7, 8, or 9;
q is 1, 2, 3, 4, 5, 6, or 7;
r is 1, 2, 3, 4, 5, 6, or 7;
s is 0, 1, 2, 3, 4, 5, or 6;
the sum of q, r, and s is 2, 3, 4, 5, 6, 7, or 8;
t is 1, 2, 3, 4, or 5;
u is 1, 2, 3, 4, or 5;
v is 1, 2, 3, 4, or 5;
w is 0, 1, 2, 3, or 4;
the sum of t, u, v, and w is 3, 4, 5, 6, or 7; and
X¹, X² and X³ are independently —O—, —NH—, —NHC(O)—, or —C(O)NH—;
wherein

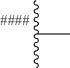

represents a covalent bond to the C(O) group of Formula (L-c) or (L-c-i), and

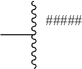

represents a covalent bond to the ring of Formula (L-c) or (L-c-i).

In another embodiment, L^{2c} of Formula (L-c) or (L-c-i) is selected from

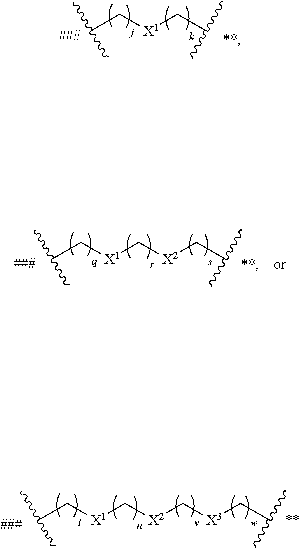

wherein:
j is 0, 1, 2, 3, 4, 5, 6, 7, 8, or 9;
k is 0, 1, 2, 3, 4, 5, 6, 7, 8, or 9;
the sum of j and k is 1, 2, 3, 4, 5, 6, 7, 8, or 9;
q is 0, 2, 3, 4, 5, 6, or 7;
r is 1, 2, 3, 4, 5, 6, 7, or 8;
s is 0, 1, 2, 3, 4, 5, 6, or 7;
the sum of q, r, and s is 1, 2, 3, 4, 5, 6, 7, or 8;
t is 0, 1, 2, 3, 4, or 5;
u is 1, 2, 3, 4, 5, or 6;
v is 1, 2, 3, 4, 5, or 6;
w is 0, 1, 2, 3, 4, or 5;
the sum of t, u, v, and w is 2, 3, 4, 5, 6, or 7; and
X¹, X² and X³ are independently —O—, —NH—, —NHC(O)—, or —C(O)NH—;
wherein

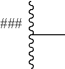

represents a covalent bond to the ring of Formula (L-c) or (L-c-i), and

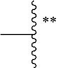

represents a covalent bond to the methylene group of Formula (I).

In another embodiment, L is a divalent linker of Formula (L-c) selected from the group consisting of:

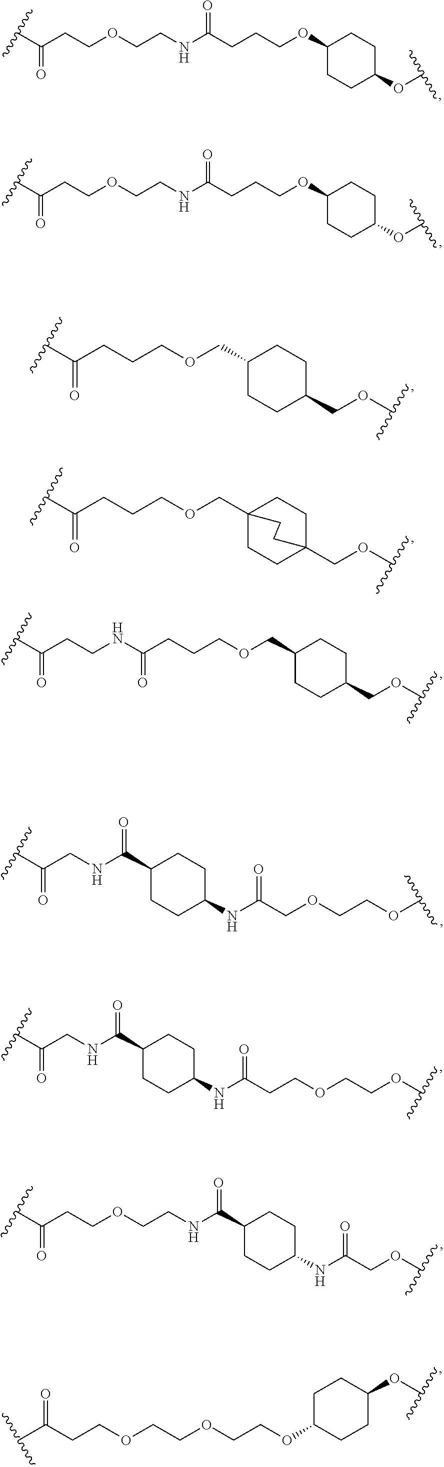

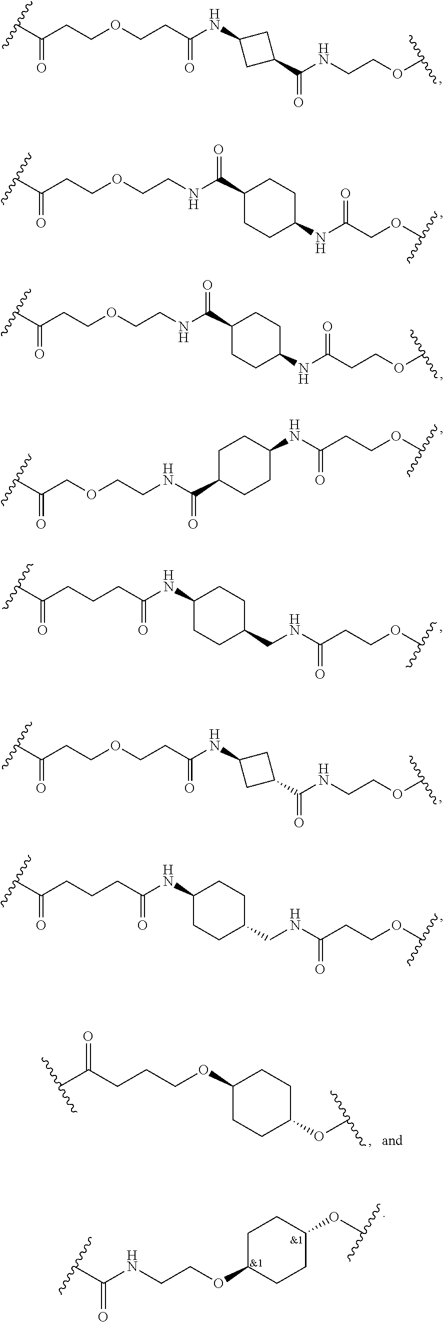

In another embodiment, L is a divalent linker of Formula (L-d):

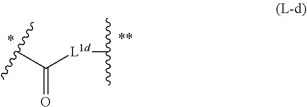

wherein:
L¹d is C₁₂₋₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—;
wherein

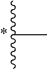

represents a covalent bond to the L′ group of Formula (I), or when L′ is a bond, a covalent bond to the Y group of Formula (I), or when both L′ and Y are a bond, a covalent bond to the T group of Formula (I), and

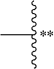

represents a covalent bond to the methylene group of Formula (I). In another embodiment, L¹d is a C₁₂, C₁₋₃, C₁₋₄, C₁₅, C₁₋₆, C₁₇, C₁₈, C₁₉, C₂₀, C₂₁, C₂₂, C₂₃, C₂₄, C₂₅, C₂₆, C₂₇, C₂₈, C₂₉, C₃₀, or C₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—. In another embodiment, L^{1d} is C₁₂₋₂₂ linear alkylene, for example, C₁₂, C₁₋₃, C₁₋₄, C₁₅, C₁₆, C₁₇, C₁₈, C₁₉, C₂₀, C₂₁, or C₂₂, wherein 1, 2, 3, 4, or 5 methylene units are replaced with —NH—, —O—, —C(O)NH—, —NHC(O)—, or —NHC(O)—NH—.

In another embodiment, L¹d of Formula (L-d) is selected from

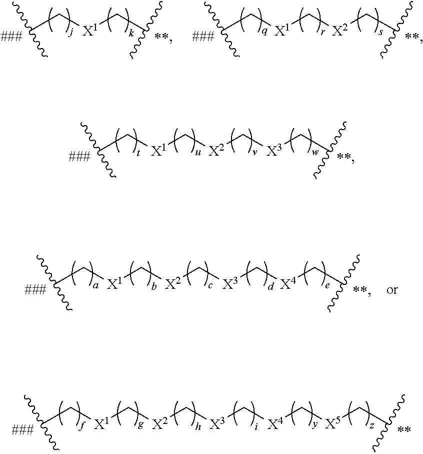

wherein:
j is 1, 2, 3, 4, 5, 6, 7, 8, 9, 10, 11, 12, 13, 14, 15, 16, 17, 18, 19, or 20;
k is 1, 2, 3, 4, 5, 6, 7, 8, 9, 10, 11, 12, 13, 14, 15, 16, 17, 18, 19, or 20;
the sum of j and k is 11, 12, 13, 14, 15, 16, 17, 18, 19, 20, or 21;
q is 1, 2, 3, 4, 5, 6, 7, 8, 9, 10, 11, 12, 13, 14, 15, 16, 17, 18, or 19;
r is 1, 2, 3, 4, 5, 6, 7, 8, 9, 10, 11, 12, 13, 14, 15, 16, 17, 18, or 19;
s is 0, 1, 2, 3, 4, 5, 6, 7, 8, 9, 10, 11, 12, 13, 14, 15, 16, 17, or 18;
the sum of q, r, and s is 10, 11, 12, 13, 14, 15, 16, 17, 18, 19, or 20;
tis 1, 2, 3, 4, 5, 6, 7, 8, 9, 10, 11, 12, 13, 14, 15, 16, or 17;
u is 1, 2, 3, 4, 5, 6, 7, 8, 9, 10, 11, 12, 13, 14, 15, 16, or 17;
v is 1, 2, 3, 4, 5, 6, 7, 8, 9, 10, 11, 12, 13, 14, 15, 16, or 17;
w is 0, 1, 2, 3, 4, 5, 6, 7, 8, 9, 10, 11, 12, 13, 14, 15, or 16;
the sum of t, u, v, and w is 9, 10, 11, 12, 13, 14, 15, 16, 17, 18, or 19;
a is 1, 2, 3, 4, 5, 6, 7, 8, 9, 10, 11, 12, 13, 14, or 15;
b is 1, 2, 3, 4, 5, 6, 7, 8, 9, 10, 11, 12, 13, 14, or 15;
c is 1, 2, 3, 4, 5, 6, 7, 8, 9, 10, 11, 12, 13, 14, or 15;
d is 1, 2, 3, 4, 5, 6, 7, 8, 9, 10, 11, 12, 13, 14, or 15;
e is 0, 1, 2, 3, 4, 5, 6, 7, 8, 9, 10, 11, 12, 13, or 14;
the sum of a, b, c, d, and e is 8, 9, 10, 11, 12, 13, 14, 15, 16, 17, or 18;
f is 1, 2, 3, 4, 5, 6, 7, 8, 9, 10, 11, 12, or 13;
g is 1, 2, 3, 4, 5, 6, 7, 8, 9, 10, 11, 12, or 13;
h is 1, 2, 3, 4, 5, 6, 7, 8, 9, 10, 11, 12, or 13;
i is 1, 2, 3, 4, 5, 6, 7, 8, 9, 10, 11, 12, or 13;
y is 1, 2, 3, 4, 5, 6, 7, 8, 9, 10, 11, 12, or 13;
z is 0, 1, 2, 3, 4, 5, 6, 7, 8, 9, 10, 11, or 12;
the sum of f, g, h, i, y, and z is 7, 8, 9, 10, 11, 12, 13, 14, 15, 16, or 17; and
X¹, X², X³, X⁴, and X⁵ are independently —NH—, —O—, —C(O)NH—, —NHC(O)—, or —NHC(O)—NH—;
wherein

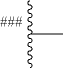

represents a covalent bond to the C(O) group of Formula (L-d), and

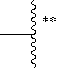

represents a covalent bond to the methylene group of Formula (I).

In another embodiment, L¹d of Formula (L-d) is

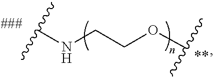

wherein n is 4, 5, 6, 7, 8, 9, or 10;
wherein

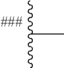

represents a covalent bond to the C(O) group of Formula (L-d), and

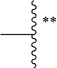

represents a covalent bond to the methylene group of Formula (I).

In another embodiment, L is a divalent linker of Formula (L-d) selected from the group consisting of:

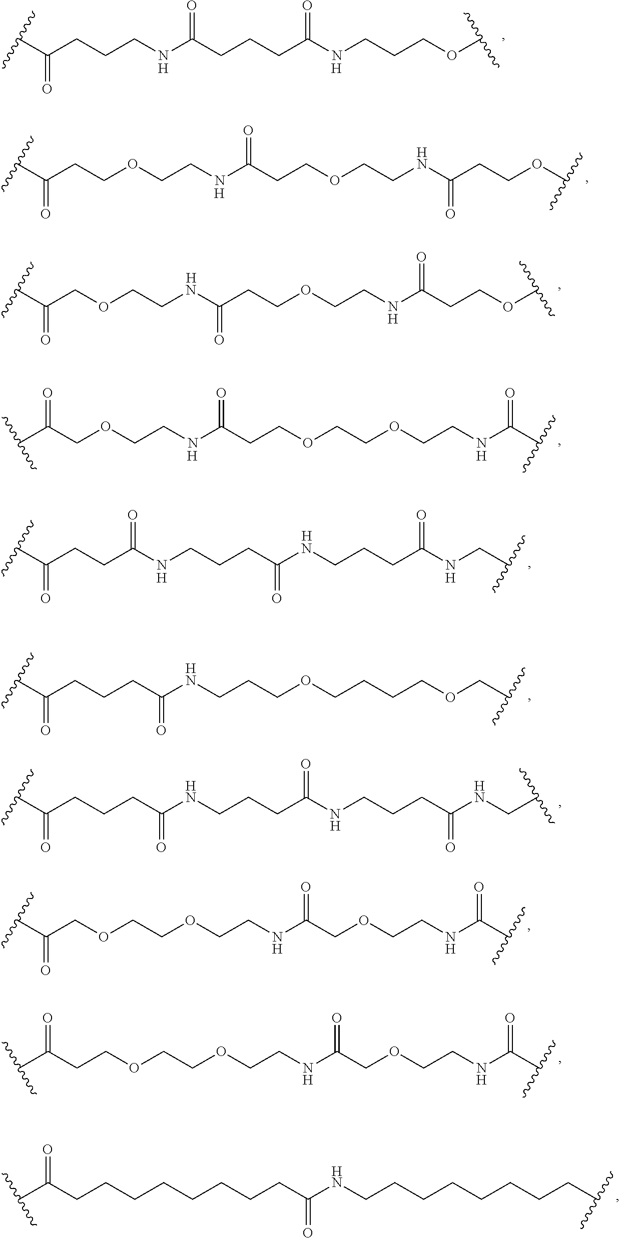

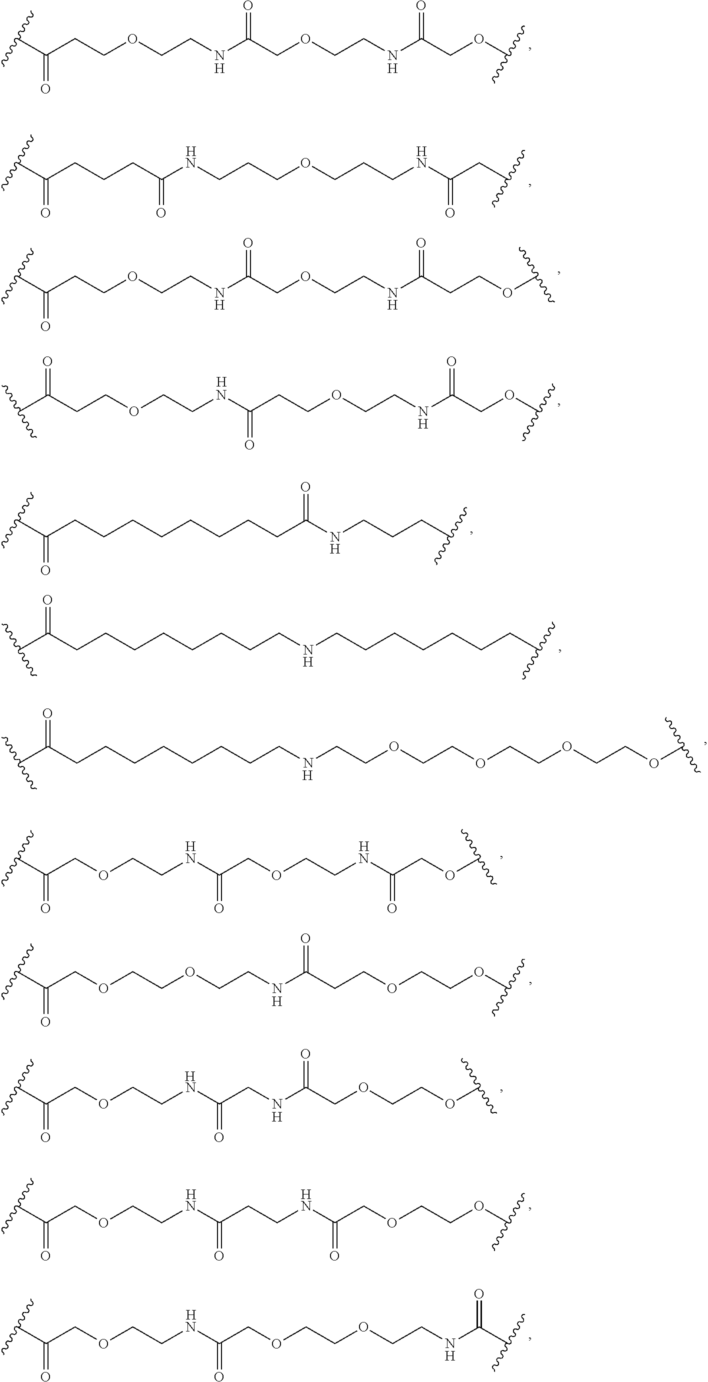

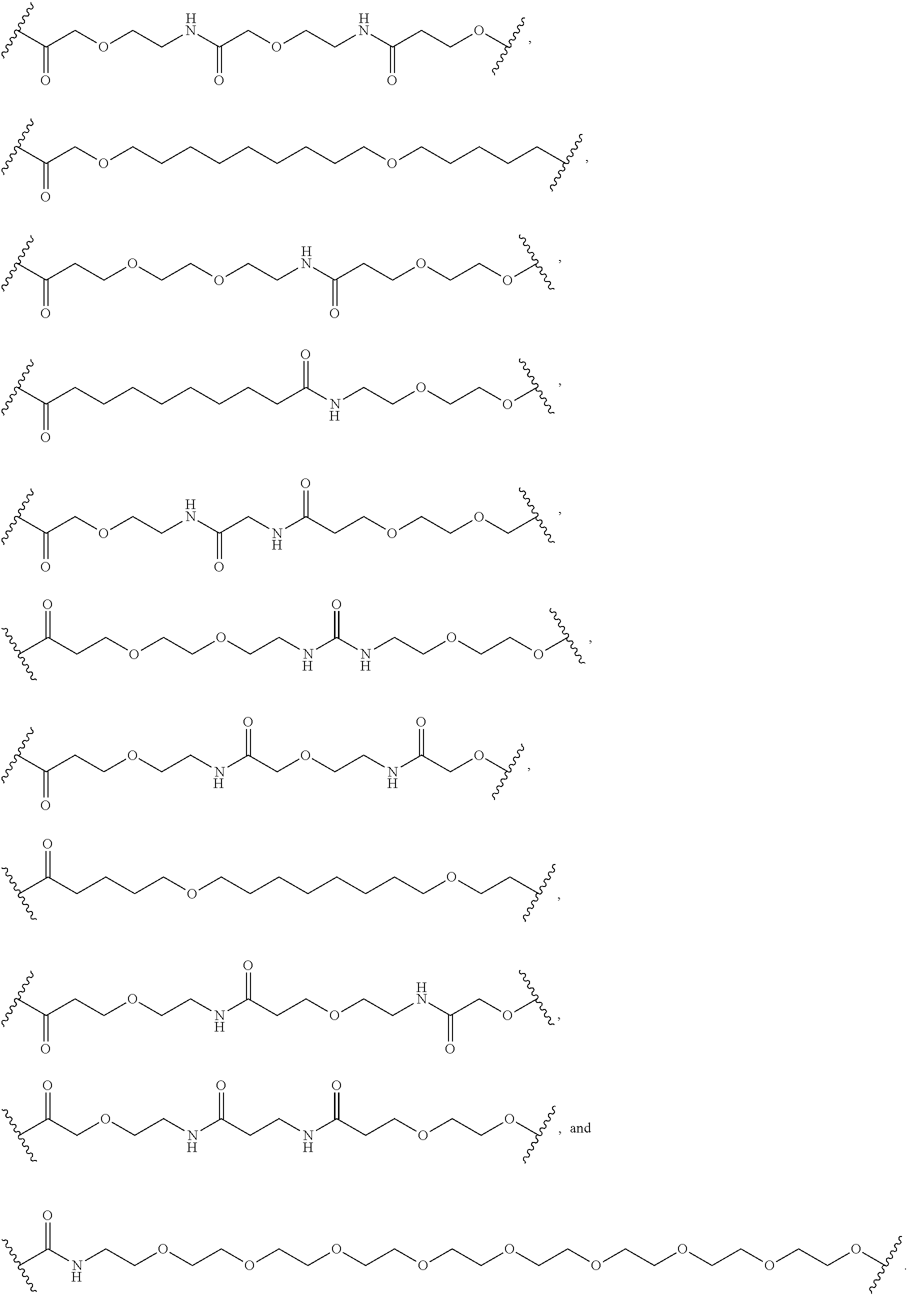

In another embodiment, L is a divalent linker of Formula (L-e):

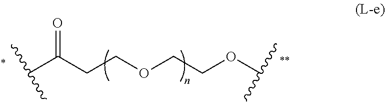

wherein:
n is an integer of 3 to 50;
wherein

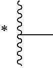

represents a covalent bond to the L′ group of Formula (I), or when L′ is a bond, a covalent bond to the Y group of Formula (I), or when both L′ and Y are a bond, a covalent bond to the T group of Formula (I), and

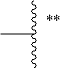

represents a covalent bond to the methylene bond to the T group of Formula (I), and group of Formula (I).

In another embodiment, n of Formula (L-e) is 3 to 25, 3 to 10, 3 to 8, 3 to 7, 3 to 5, or 3 to 4. In another embodiment, n of Formula (L-e) is 5 to 22, 7 to 15, or 9 to 13. In another embodiment, n of Formula (L-e) is 3, 4, 5, 7, 8, 11, 22, or 50.

In another embodiment, n of Formula (L-e) is 12 to 50, 15 to 30, 17 to 25, 18 to 24, 18 to 20, 20 to 22, or 22 to 24. In another embodiment, n of Formula (L-e) is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50. In another embodiment, n of Formula (L-e) is 19 or 23.

In another embodiment, L is a divalent linker of Formula (L-f):

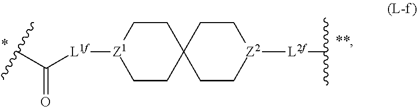

or a stereoisomer thereof, wherein:
L¹f is a bond; C₁₋₆ linear alkylene, wherein 0, 1, or 2 methylene units are replaced with —O—, —NH—, or —C(O)—; or —(C₃₋₆ cycloalkylene)—NHC(O)—;
L²f is a bond, —NHC(O)—, —C(O)NH—, or a C₁₋₆ linear alkylene, wherein 0, 1, or 2 methylene units are replaced with —O—; and
each of Z¹ and Z² is independently N or CH;
wherein

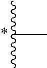

represents a covalent bond to the L′ group of Formula (I), or when L′ is a bond, a covalent bond to the Y group of Formula (I), or when both L′ and Y are a bond, a covalent bond to the T group of Formula (I), and

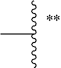

represents a covalent bond to the methylene group of Formula (I).

In another embodiment, L¹f of Formula (L-f) is selected from

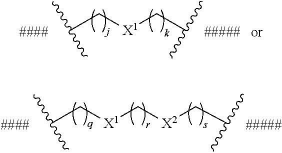

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2;
the sum of q, r, and s is 2, 3, or 4; and
X¹ and X² are independently —O—, —NH—, or —C(O)—; or —(C₃₋₆ cycloalkylene)—NHC(O)—;
wherein

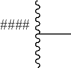

represents a covalent bond to the C(O) group of Formula (L-f), and

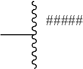

represents a covalent bond to the ring of Formula (L-f).

In another embodiment, L²f of Formula (L-f) is selected from

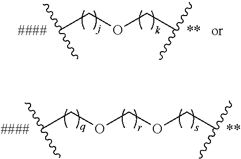

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2; and
the sum of q, r, and s is 2, 3, or 4;
wherein

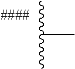

represents a covalent bond to the ring of Formula (L-f), and

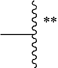

represents a covalent bond to the methylene group of Formula (I).

In another embodiment, L is a divalent linker of Formula (L-f) selected from the group consisting of:

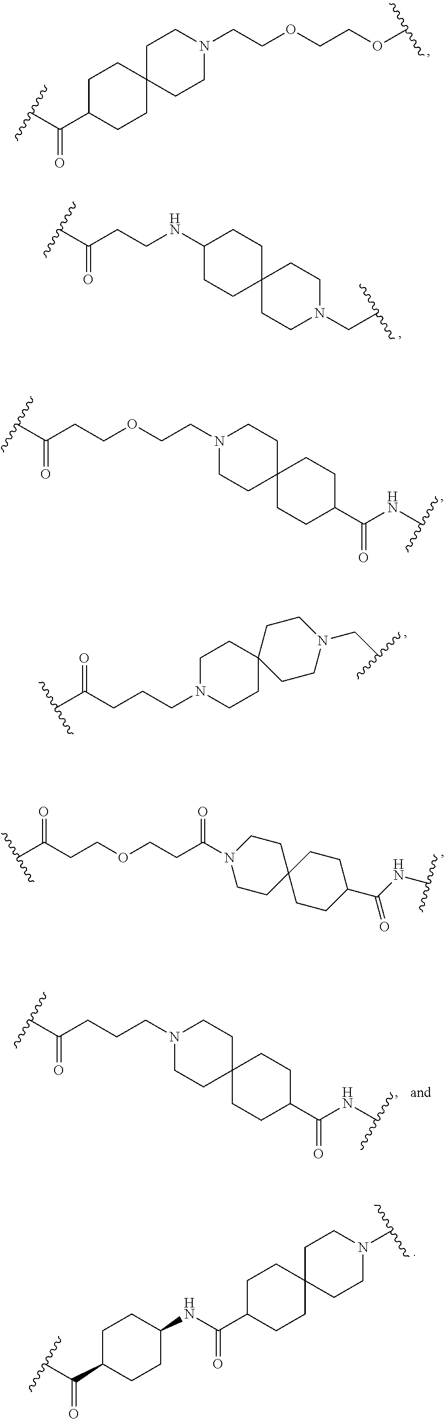

In another embodiment, L is a divalent linker of Formula (L-g):

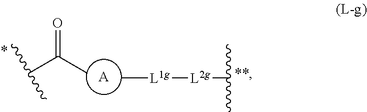

wherein:
Ring A is a 5 to 6 membered heteroarylene having 1 or 2 nitrogen ring atoms;
L¹g is a bond, —CH₂—, —NH—, or —O—; and
L²g is

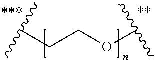

wherein n is 1, 2, 3, 4, or 5, and

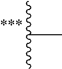

represents a covalent bond to L¹g;
wherein

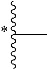

represents a covalent bond to the L′ group of Formula (I), or when L′ is a bond, a covalent bond to the Y group of Formula (I), or when both L′ and Y are a bond, a covalent bond to the T group of Formula (I), and

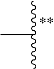

represents a covalent bond to the methylene group of Formula (I).

In another embodiment, L is a divalent linker of Formula (L-g-i):

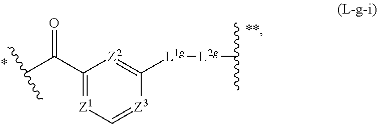

wherein:
L¹g is a bond, —CH₂—, —NH—, or —O—;
L²g is

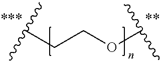

wherein n is 1, 2, 3, 4, or 5, and

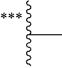

represents a covalent bond to L¹g;
Z¹, Z², and Z³ are each independently selected from N or CH, provided that one or two of Z¹, Z², and Z³ is N;
wherein

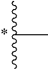

represents a covalent bond to the L′ group of Formula (I), or when L′ is a bond, a covalent bond to the Y group of Formula (I), or when both L′ and Y are a bond, a covalent bond to the T group of Formula (I), and

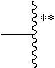

represents a covalent bond to the methylene bond to the T group of Formula (I), and group of Formula (I).

In another embodiment, L is a divalent linker of Formula (L-g) selected from the group consisting of:

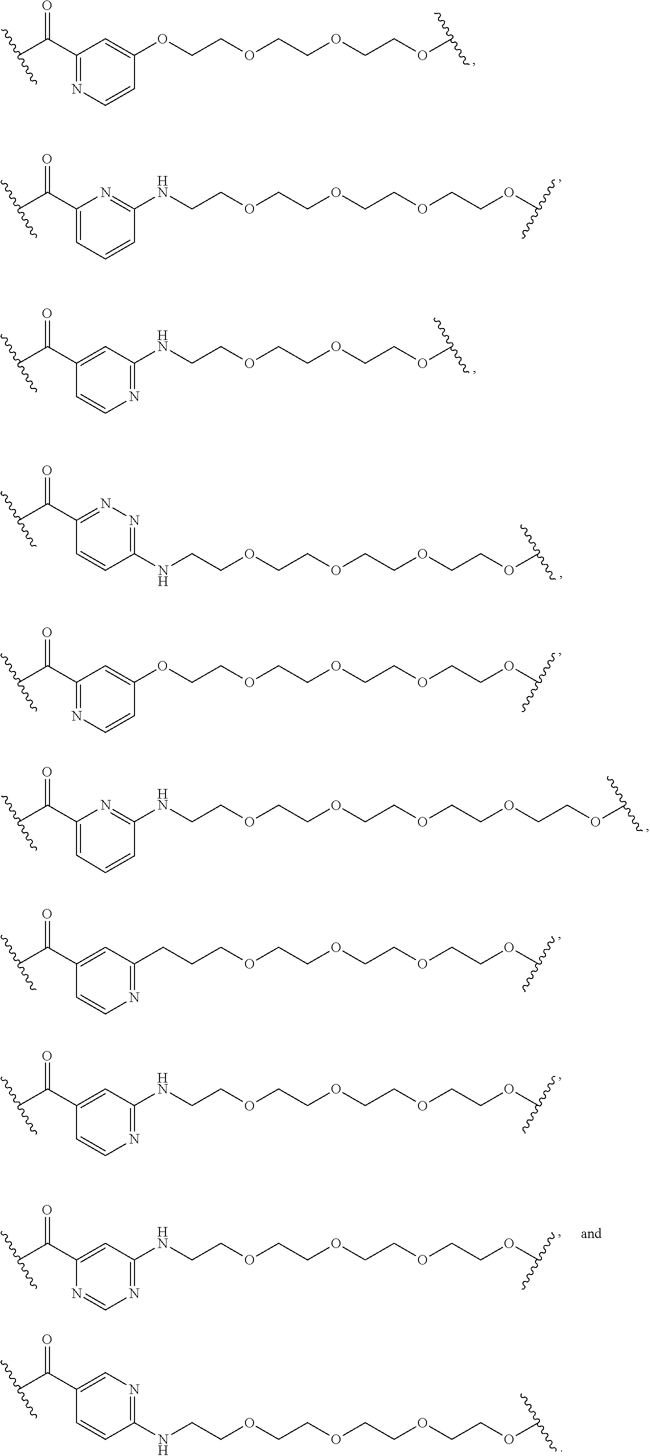

In another embodiment, L is a divalent linker of Formula (L-h):

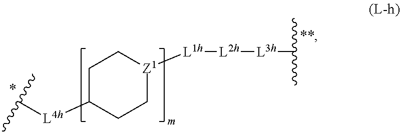

or a stereoisomer thereof,
wherein:
each Z¹ is independently N or CH;
L¹ h is a bond, —C(O)—, —C(O)—NH—, or —NHC(O)—;
L² h is C₂₋₁₀ linear alkylene or

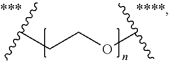

wherein n is 1, 2, 3, or 4, and

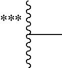

represents
a covalent bond to L¹ h and

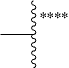

represents a covalent bond to L³h;
L³ h is a bond, —C(O) CH₂—, —O—(C₃₋₆ cycloalkylene)—O—, or —C(O)NH(CH₂)₃OCH₂—;
L⁴ h is a bond, —C(O)—, —CH₂C(O)—, or —C(O) CH₂—; and
m is 1, 2, or 3;
wherein

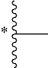

represents a covalent bond to the L′ group of Formula (I), or when L′ is a bond, a covalent bond to the Y group of Formula (I), or when both L′ and Y are a bond, a covalent bond to the T group of Formula (I), and

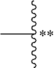

represents a covalent bond to the methylene group of Formula (I).

In another embodiment, L is a divalent linker of Formula (L-h) selected from the group consisting of:

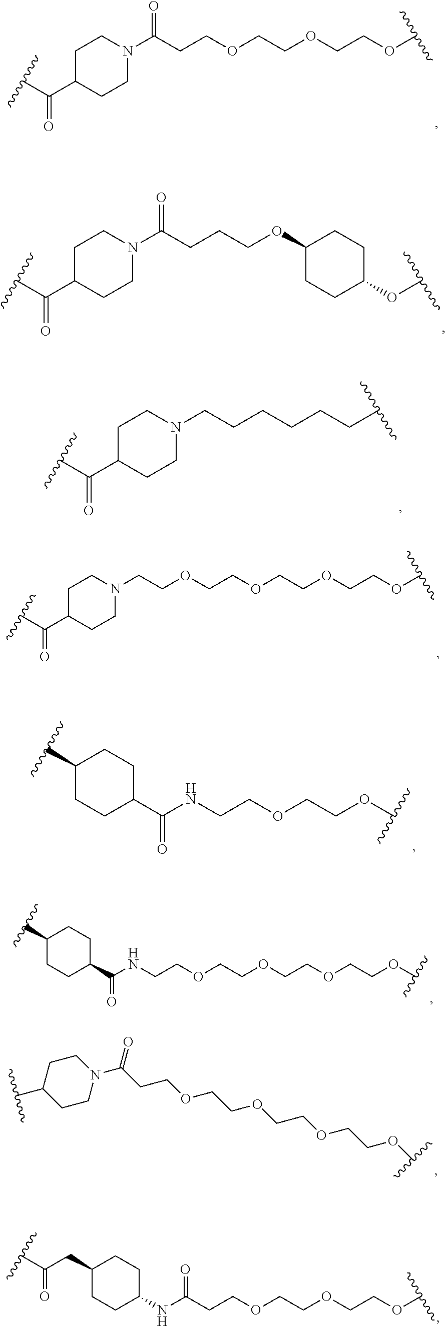

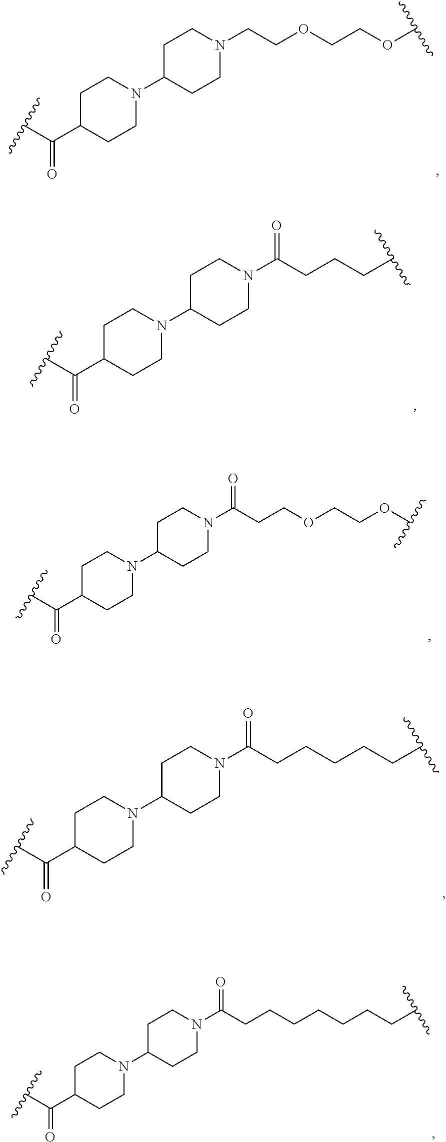 [SMILES: *OCCOCCN1CCC(N2CCC(C(*)=O)CC2)CC1 ; *CCCC(=O)N1CCC(N2CCC(C(*)=O)CC2)CC1 ; *OCCOCCC(=O)N1CCC(N2CCC(C(*)=O)CC2)CC1 ; *CCCCCC(=O)N1CCC(N2CCC(C(*)=O)CC2)CC1 ; *CCCCCCCC(=O)N1CCC(N2CCC(C(*)=O)CC2)CC1]

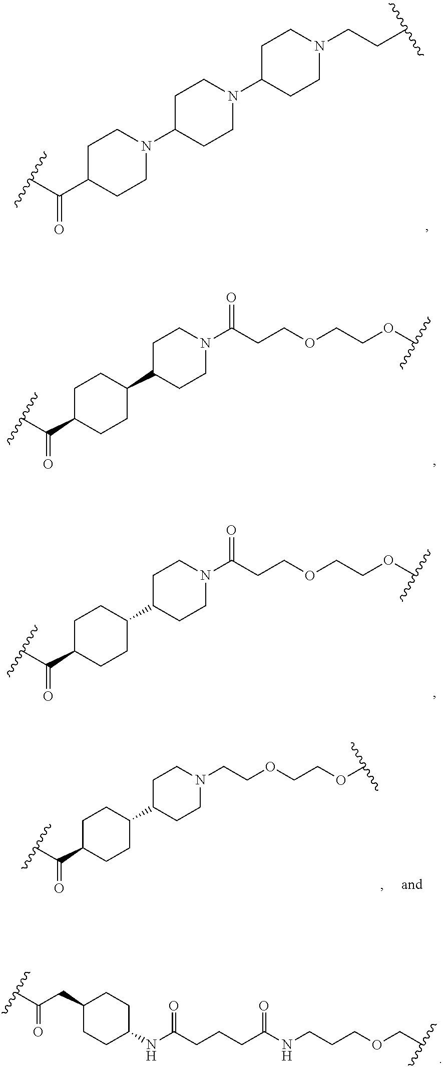 [SMILES: *CCN1CCC(N2CCC(N3CCC(C(*)=O)CC3)CC2)CC1 ; *OCCOCCC(=O)N1CCC([C@H]2CC[C@@H](C(*)=O)CC2)CC1 ; *OCCOCCC(=O)N1CCC([C@H]2CC[C@H](C(*)=O)CC2)CC1 ; *OCCOCCN1CCC([C@H]2CC[C@H](C(*)=O)CC2)CC1 ; *COCCCNC(=O)CCCC(=O)N[C@H]1CC[C@H](CC(*)=O)CC1]

In another embodiment, L is a divalent linker of Formula (L-i):

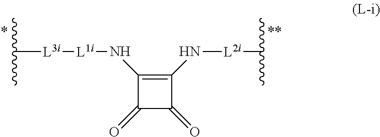

wherein:
L¹ⁱ is a bond, C₁₋₁₂ linear alkylene, or

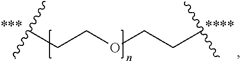

wherein n is 1, 2, 3, 4, or 5, and

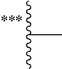

represents a covalent bond to L³ⁱ and

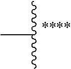

represents a covalent bond to NH;
L²ⁱ is a bond, C₁₋₁₂ linear alkylene, or

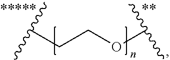

wherein n is 1, 2, 3, 4, or 5, and

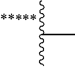

represents a covalent bond to HN; and
L³ⁱ is a bond or —C(O)—;
wherein

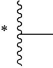

represents a covalent bond to the L′ group of Formula (I), or when L′ is a bond, a covalent bond to the Y group of Formula (I), or when both L′ and Y are a bond, a covalent bond to the T group of Formula (I), and

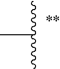

represents a covalent bond to the methylene group of Formula (I).

In another embodiment, L is a divalent linker of Formula (L-i) selected from the group consisting of:

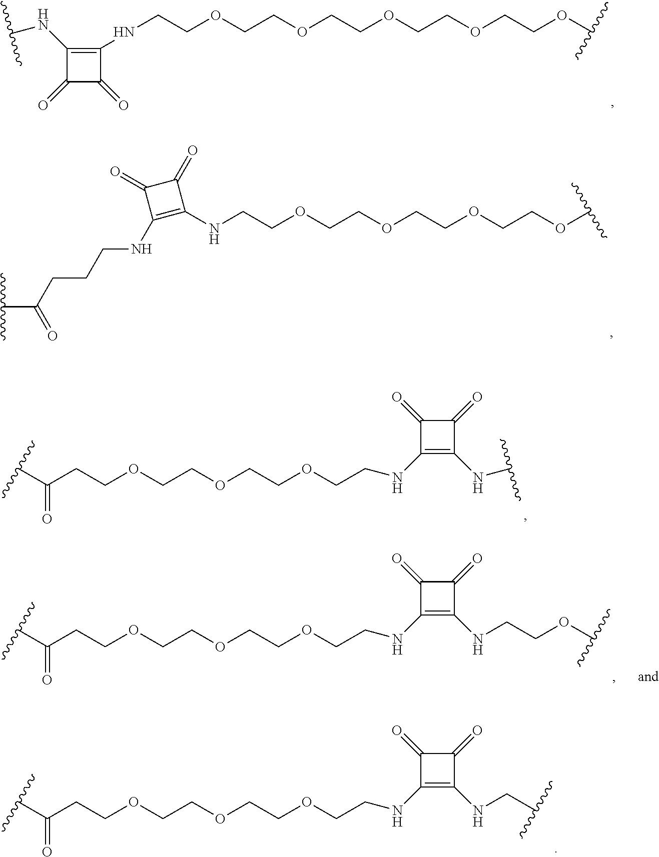 [SMILES: *Nc1c(NCCOCCOCCOCCOCCO*)c(=O)c1=O ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

In another embodiment, L is a divalent linker of Formula (L-j):

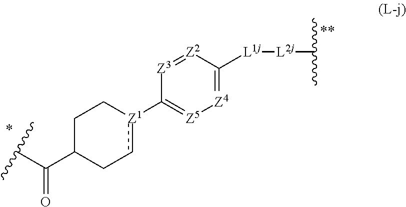

or a stereoisomer thereof,
wherein:
Z¹ is C, CH, or N;
each of Z², Z³, Z⁴ and Z⁵ is independently CH or N, provided that no more than two of Z², Z³, Z⁴ and Z⁵ are N;
L¹j is —NH—, —C(O)NH—, —NHC(O)—, or —O—;
L²j is C₁₋₆ linear alkylene or

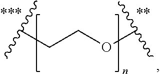

wherein n is 1 or 2, and

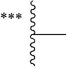

represents a covalent bond to L¹j; and
represents a single bond or a double bond;
wherein

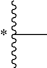

represents a covalent bond to the L′ group of Formula (I), or when L′ is a bond, a covalent bond to the Y group of Formula (I), or when both L′ and Y are a bond, a covalent bond to the T group of Formula (I), and

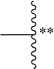

represents a covalent bond to the methylene group of Formula (I).

In another embodiment, L is a divalent linker of Formula (L-j) selected from the group consisting of:

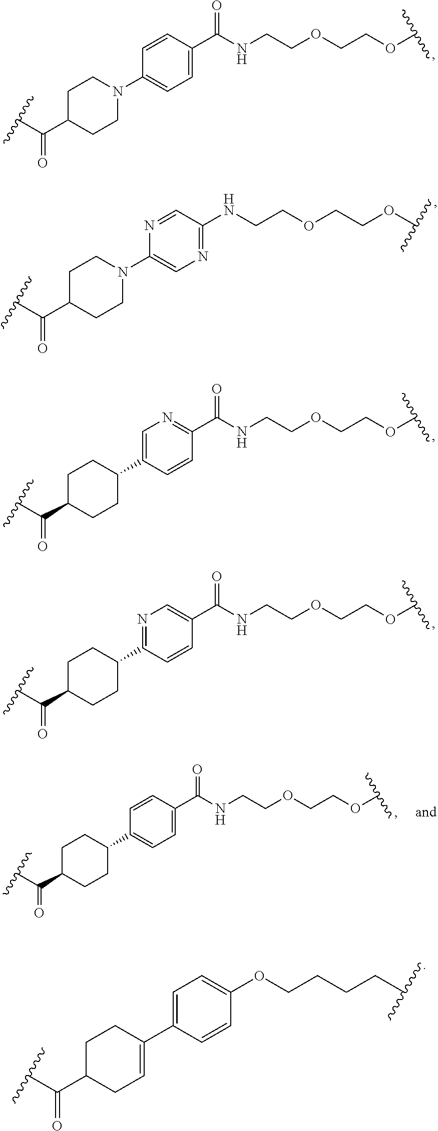 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

In another embodiment, L is a divalent linker of Formula (L-k):

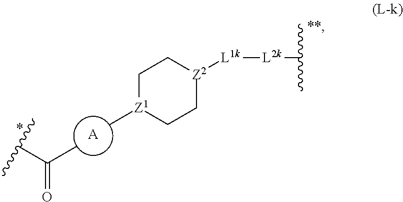

or a stereoisomer thereof,
wherein:
Ring A is phenyl or a 5 or 6 membered heteroarylene having 1 or 2 nitrogen ring atoms; each of Z¹ and Z² is independently CH or N;
L¹k is a bond, —C(O)—, —C(O)NH— or —NHC(O)—; and
L²k is a C₃₋₈ straight chain alkylene or

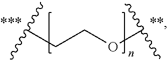

wherein n is 1, 2, or 3, and

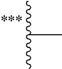

represents a covalent bond to L¹k;
wherein

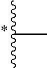

represents a covalent bond to the L′ group of Formula (I), or when L′ is a bond, a covalent bond to the Y group of Formula (I), or when both L′ and Y are a bond, a covalent bond to the T group of Formula (I), and

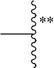

represents a covalent bond to the methylene group of Formula (I).

In another embodiment, L is a divalent linker of Formula (L-k) selected from the group consisting of:

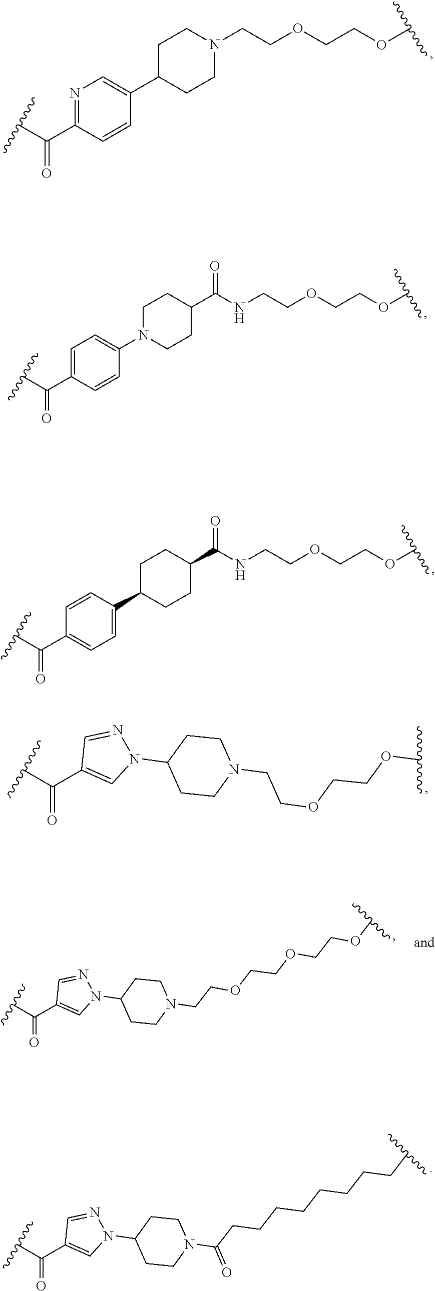 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

In another embodiment, L is a divalent linker of Formula (L-m):

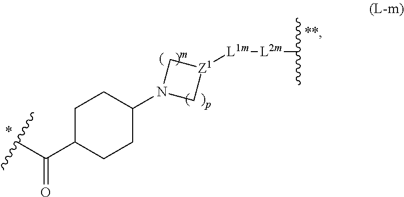

or a stereoisomer thereof,
wherein:
Z¹ is CH or N;
m is 1 or 2;
p is 1 or 2;
0, 1, or 2 hydrogen atoms of

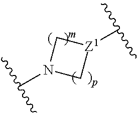

are replaced with F;
L^{1m} is a bond, —C(O)—, —C(O)NH—, —NHC(O)—, —S(O)₂NH— or —NHS(O)₂—; and
L²m is C₃₋₆ linear alkylene, C₃₋₆ cycloalkylene, or

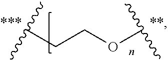

wherein n is 1 or 2, and

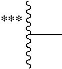

represents a covalent bond to L¹m;
wherein

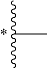

represents a covalent bond to the L′ group of Formula (I), or when L′ is a bond, a covalent bond to the Y group of Formula (I), or when both L′ and Y are a bond, a covalent bond to the T group of Formula (I), and

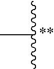

represents a covalent bond to the methylene group of Formula (I).

In another embodiment, L is a divalent linker of Formula (L-m) selected from the group consisting of:

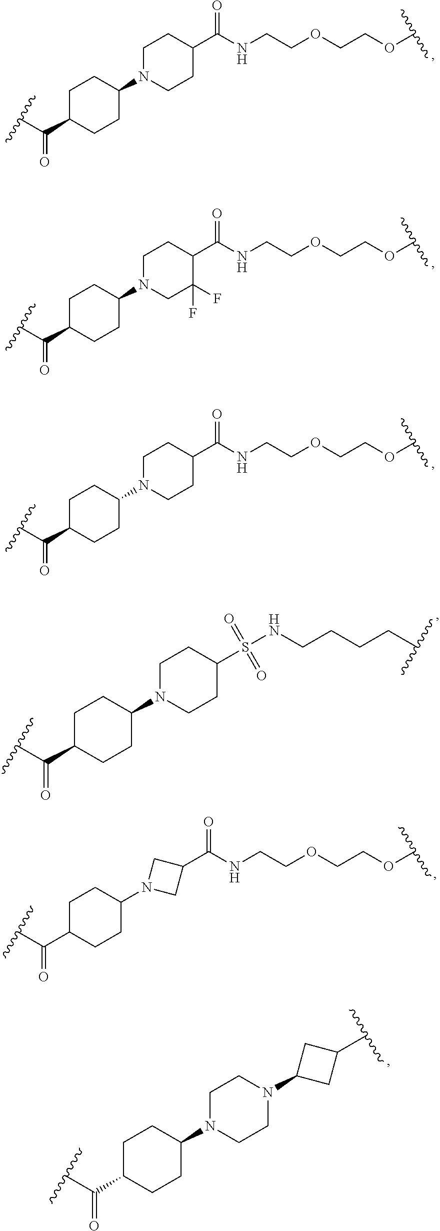

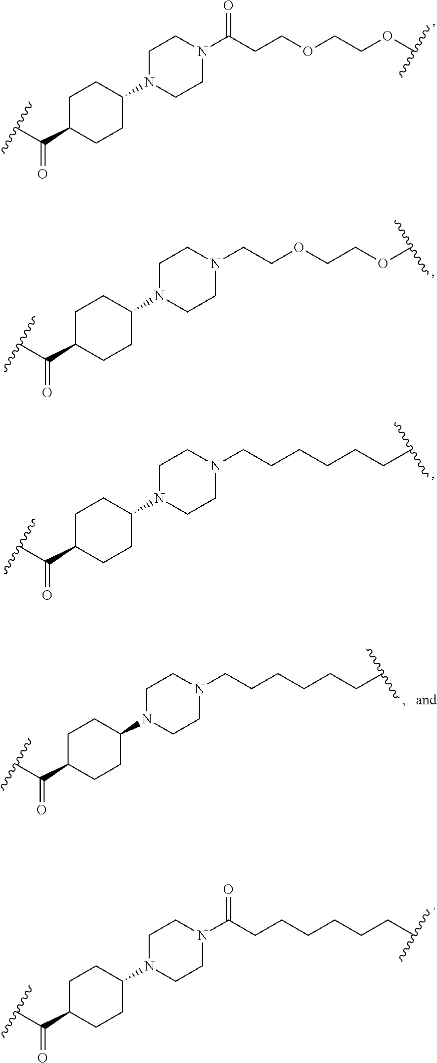

In another embodiment, L is a divalent linker of Formula (L-n-i):

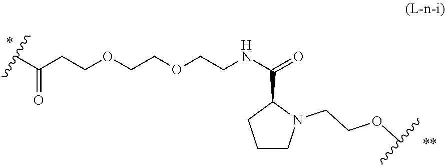 [SMILES: *OCCN1CCC[C@H]1C(=O)NCCOCCOCCC(*)=O]

wherein

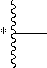

represents a covalent bond to the L′ group of Formula (I), or when L′ is a bond, a covalent bond to the Y group of Formula (I), or when both L′ and Y are a bond, a covalent bond to the T group of Formula (I), and

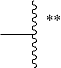

represents a covalent bond to the methylene group of Formula (I).

In another embodiment, L is a divalent linker of Formula (L-n-ii):

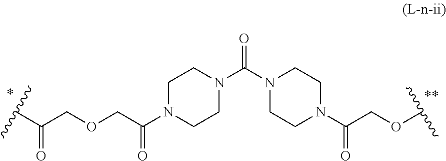 [SMILES: *OCC(=O)N1CCN(C(=O)N2CCN(C(=O)COCC(*)=O)CC2)CC1]

wherein

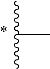

represents a covalent bond to the L′ group of Formula (I), or when L′ is a bond, a covalent bond to the Y group of Formula (I), or when both L′ and Y are a bond, a covalent to the T group of Formula (I), and

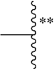

represents a covalent bond to the methylene bond group of Formula (I).

In another embodiment, L is a divalent linker of Formula (L-n-iii):

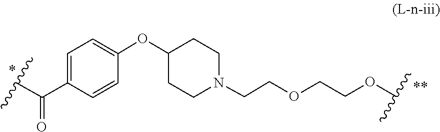 [SMILES: *OCCOCCN1CCC(Oc2ccc(C(*)=O)cc2)CC1]

wherein

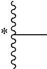

represents a covalent bond to the L′ group of Formula (I), or when L′ is a bond, a covalent bond to the Y group of Formula (I), or when both L′ and Y are a bond, a covalent bond to the T group of Formula (I), and

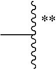

represents a covalent bond to the methylene group of Formula (I).

In another embodiment, L is a divalent linker of Formula (L-n-iv):

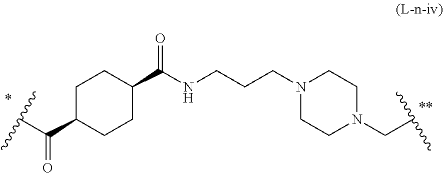 [SMILES: *CN1CCN(CCCNC(=O)[C@H]2CC[C@@H](C(*)=O)CC2)CC1]

wherein

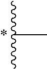

represents a covalent bond to the L′ group of Formula (I), or when L′ is a bond, a covalent bond to the Y group of Formula (I), or when both L′ and Y are a bond, a covalent

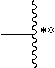

represents a covalent bond to the methylene bond to the T group of Formula (I), and group of Formula (I).

In another embodiment, L is a divalent linker of Formula (L-p):

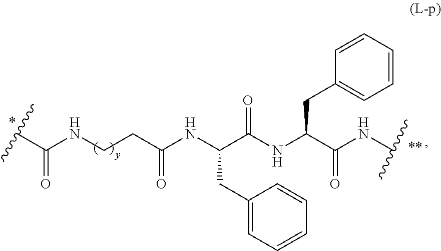

or a stereoisomer thereof, wherein y is an integer of 1 to 9;
wherein

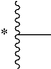

represents a covalent bond to the L′ group of Formula (I), or when L′ is a bond, a covalent bond to the Y group of Formula (I), or when both L′ and Y are a bond, a covalent bond to the T group of Formula (I), and

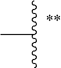

represents a covalent bond to the methylene group of Formula (I).

In another embodiment, L is a divalent linker of Formula (L-q):

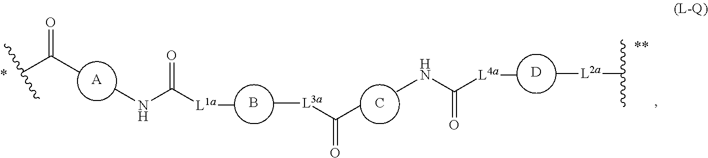

or a stereoisomer thereof,
wherein:
Ring A, Ring B, Ring C, and Ring D are each independently C₄₋₆ cycloalkylene;
L^{1a}, L³a, and L^{4a} are each independently C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

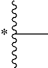

represents a covalent bond to the L′ group of Formula (I), or when L′ is a bond, a covalent bond to the Y group of Formula (I), or when both L′ and Y are a bond, a covalent bond to the T group of Formula (I), and

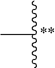

represents a covalent bond to the methylene bond to the T group of Formula (I), and group of Formula (I).

In another embodiment, L is a divalent linker of Formula (L-q-i):

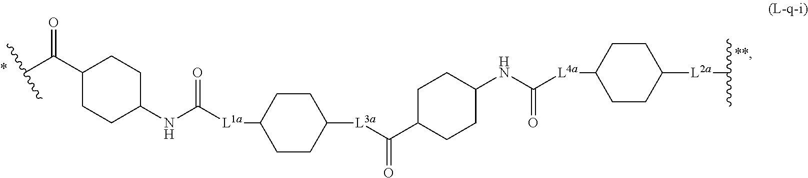

or a stereoisomer thereof,
wherein:
L^{1a}, L³a, and L⁴a are each independently C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

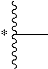

represents a covalent bond to the L′ group of Formula (I), or when L′ is a bond, a covalent bond to the Y group of Formula (I), or when both L′ and Y are a bond, a covalent bond to the T group of Formula (I), and

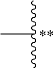

represents a covalent bond to the methylene group of Formula (I).

In another embodiment, L is a divalent linker of Formula (L-q-ii):

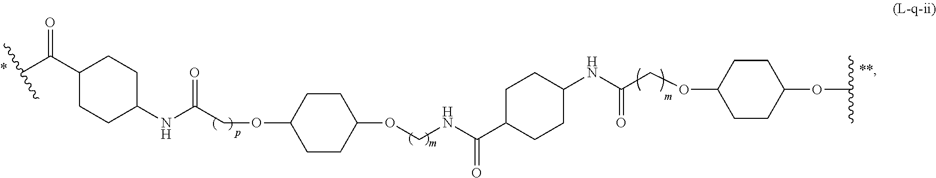

or a stereoisomer thereof,
wherein:
p is 1, 2, or 3;
m is 1, 2, or 3; and
n is 1, 2, or 3;
wherein

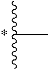

represents a covalent bond to the L′ group of Formula (I), or when L′ is a bond, a covalent bond to the Y group of Formula (I), or when both L′ and Y are a bond, a covalent bond to the T group of Formula (I), and

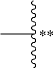

represents a covalent bond to the methylene group of Formula (I).

In another embodiment, L is a divalent linker of Formula (L-q) having the following structure:

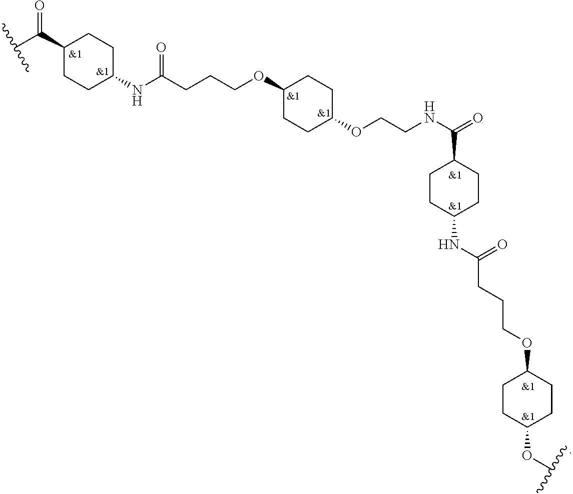 [SMILES: *O[C@H]1CC[C@H](OCCCC(=O)N[C@H]2CC[C@H](C(=O)NCCO[C@H]3CC[C@H](OCCCC(=O)N[C@H]4CC[C@H](C(*)=O)CC4)CC3)CC2)CC1]

In another embodiment, L is a divalent linker of Formula (L-r):

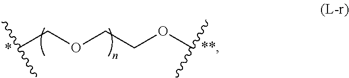

wherein
n is an integer of 10 to 30;
wherein

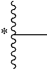

represents a covalent bond to the L′ group of Formula (I), or when L′ is a bond, a covalent bond to the Y group of Formula (I), or when both L′ and Y are a bond, a covalent bond to the T group of Formula (I), and

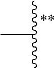

represents a covalent bond to the methylene group of Formula (I).

In another embodiment, n of Formula (L-r) is 10 to 20, 10 to 18, 12 to 16, or 13 to 15. In another embodiment, n of Formula (L-r) is 10, 11, 12, 13, 14, 15, 16, 17, 18, 19, 20, 21, 22, 23, 24, 25, 26, 27, 28, 29, or 30. In another embodiment, n of Formula (L-r) is 14.

In another embodiment, L is a divalent linker of Formula (L-s):

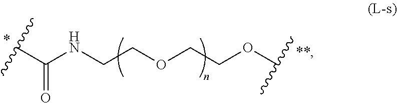

wherein
n is an integer of 10 to 30;
wherein

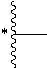

represents a covalent bond to the L′ group of Formula (I), or when L′ is a bond, a covalent bond to the Y group of Formula (I), or when both L′ and Y are a bond, a covalent bond to the T group of Formula (I), and

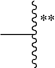

represents a covalent bond to the methylene group of Formula (I).

In another embodiment, n of Formula (L-s) is 10 to 20, 10 to 18, 12 to 16, or 13 to 15. In another embodiment, n of Formula (L-s) is 15 to 30, 17 to 28, 18 to 26, 19 to 25, 20 to 24, or 21 to 23. In another embodiment, n of Formula (L-s) is 10, 11, 12, 13, 14, 15, 16, 17, 18, 19, 20, 21, 22, 23, 24, 25, 26, 27, 28, 29, or 30. In another embodiment, n of Formula (L-s) is 14 or 22.

In one embodiment of the disclosure, Y is selected from a bond; —NH—; —(C₁₋₁₂ alkylene)—, wherein 1, 2, or 3 methylene units are replaced with —O—, —NH—, —N(CH₃)—, —C(O)—, —NHC(O)—, —C(O)NH—, —(C₃₋₆ cycloalkylene)—, —(C₃₋₆ cycloalkenylene)—, 3- to 1O-membered heterocycloalkylene, arylene, or heteroarylene; or —(C₂₋₁₂ alkenylene)—, wherein 1, 2, or 3 methylene units are replaced with —O—, —NH—, —N(CH₃)—, —C(O)—, —NHC(O)—, —C(O)NH—, —(C₃₋₆ cycloalkylene)—, —(C₃₋₆ cycloalkenylene)—, 3- to 1O-membered heterocycloalkylene, arylene, or heteroarylene.

In another embodiment, Y is selected from a bond; —NH—; —(C₁₋₆ alkylene)—O —; —O—(C₁-6 alkylene)—; —(C₂₋₆ alkenylene)—O—; —(C₁₋₆ alkylene)—C(O)—; —(C₂₋₆ alkenylene)—C(O)—; phenylene; piperidinylene; hydroxypiperidinylene; fluoropiperidinylene; azetidinylene; —C(O)-piperazinylene-; —(C₁₋₆ alkylene)-oxopiperazinylene-; pyrrolidinylene; 7- to 9-membered bridged bicyclic heterocycloalkylene; -(C₁₋₆ alkylene)—O-phenylene-; -(C₂₋₆ alkenylene)—O-piperidinylene; -(C₁₋₅ alkylene)—NH—, wherein 0, 1, or 2 methylene units are replaced with —O—; —NH-(C₁₋₅ alkylene)—NH—; —N(CH₃)-(C₁₋₅ alkylene)—NH—; —NH-(C₁₋₅ alkylene)—N(CH₃)-; —N(CH₃)-(C₁₋₅ alkylene)—N(CH₃)-; -(C₃₋₆ cycloalkylene)—NH—; —C(O)NH-(C₁₋₅ alkylene)—NH—; —C(O)NH-(C₃₋₆ cycloalkylene)—NH—; -(C₁₋₅ alkylene)—O-(C₃₋₆ cycloalkylene)—NH—; -(C₃₋₆ cycloalkenylene)—NH—;
or

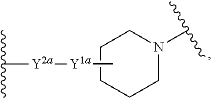

wherein Y^{1a} is a bond, —O—, —NH—, —NHC(O)—, —C(O)NH—, or C₁₋₃ alkylene; and Y^{2a} is a bond, —O—, —NH—, —NHC(O)—, —C(O)NH—, or C₁₋₃ alkylene. In another embodiment, Y is —NH—.

In another embodiment, Y is selected from the group consisting of:

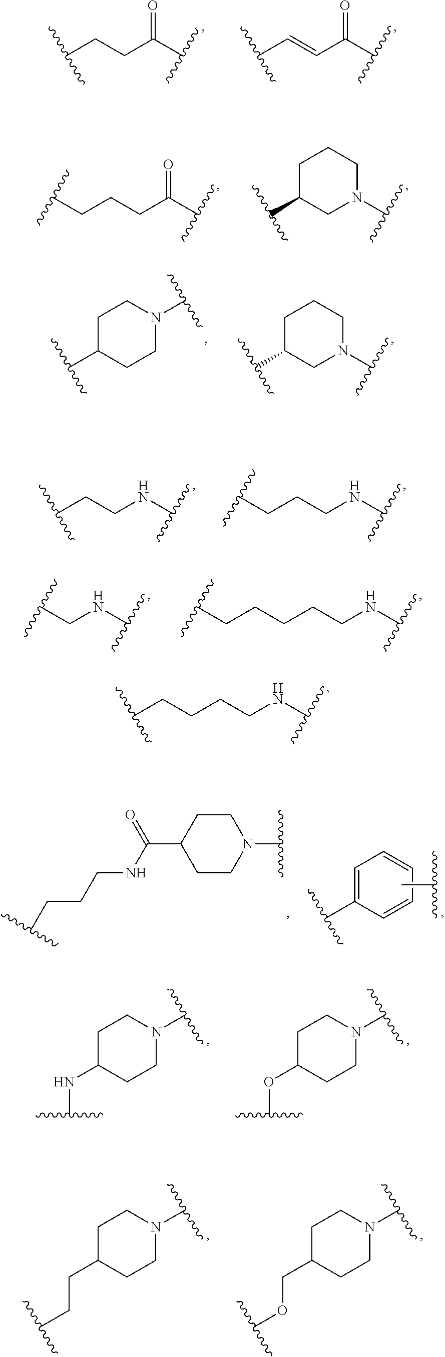

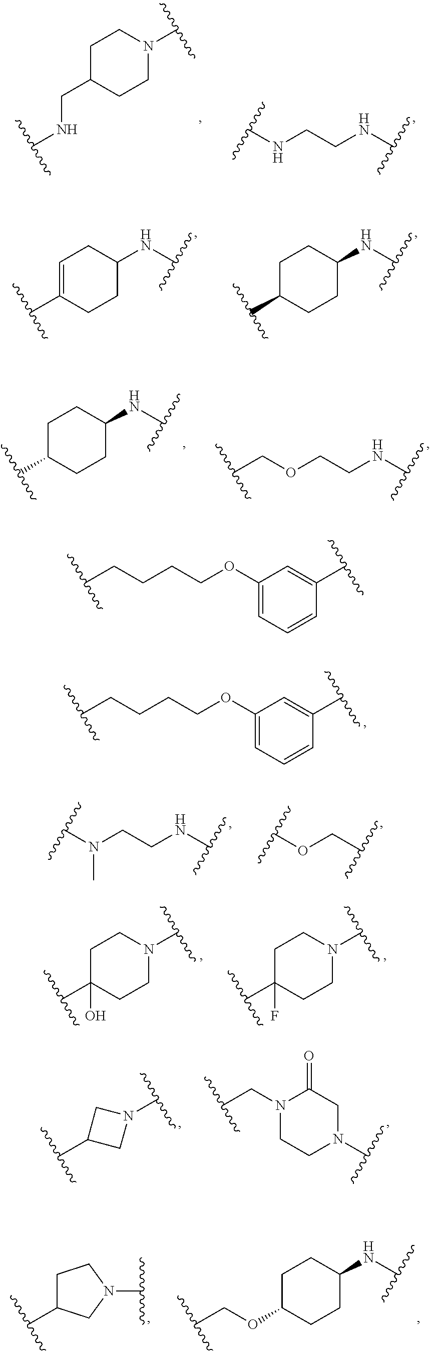

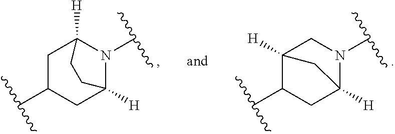 [SMILES: *C1C[C@H]2CC[C@@H](C1)N2* ; *C1C[C@H]2C[C@@H]1CN2*]

In another embodiment, Y is a bond. In another embodiment, Y is

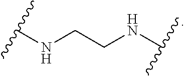 [SMILES: *NCCN*]

In another embodiment, Y is:

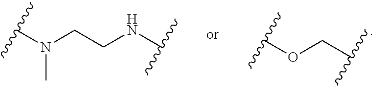

In another embodiment, R¹ is methyl, ethyl, 1-propyl, 2-propyl, 1-butyl, 2-butyl, or t-butyl. In another embodiment, R¹ is methyl. In another embodiment, R¹ is ethyl. In another embodiment, R¹ is cyclopropyl, cyclobutyl, cyclopentyl, or cyclohexyl.

In another embodiment, y of L′ is 2 to 8, 3 to 7, 4 to 7, or 5 to 7. In another embodiment, y of L′ is 1, 2, 3, 4, 5, 6, 7, 8, or 9.

In another embodiment, w of L′ is 0 to 4, 0 to 3, 0 to 2, or 1 to 2. In another embodiment, w of L′ is 0, 1, 2, 3, 4, or 5.

In another embodiment, L′ is

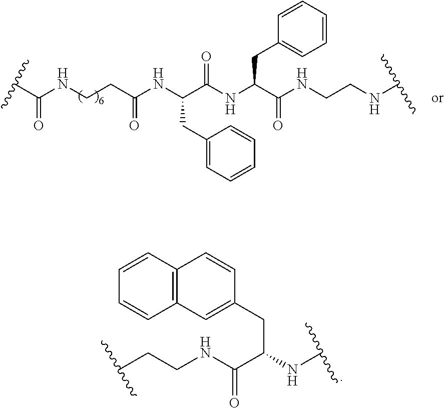

In another embodiment, L′ is a bond.

In another embodiment, T is

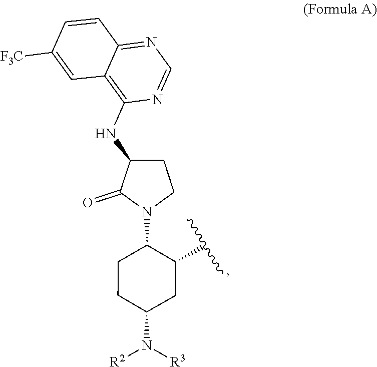

wherein R² of Formula A is hydrogen or C₁₋₄ alkyl; and R³ of Formula A is hydrogen or C₁₋₄ alkyl. In another embodiment, R² and R³ of Formula A are each independently hydrogen, methyl, ethyl, 1-propyl, 2-propyl, 1-butyl, 2-butyl, or t-butyl. In another embodiment, R² of Formula A is isopropyl and R³ of Formula A is methyl. In another embodiment, R² of Formula A is t-butyl and R³ of Formula A is hydrogen. In another embodiment, T is

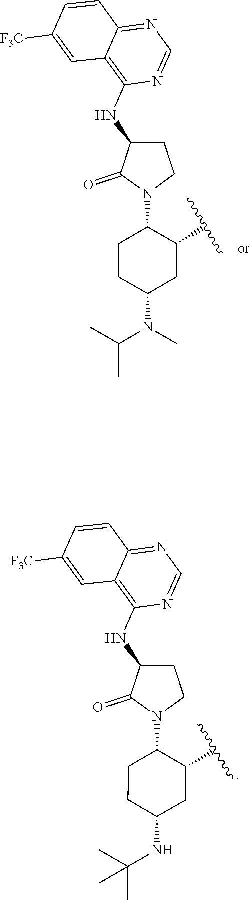 [SMILES: *[C@@H]1C[C@H](N(C)C(C)C)CC[C@@H]1N1CC[C@H](Nc2ncnc3ccc(C(F)(F)F)cc23)C1=O ; *[C@@H]1C[C@H](NC(C)(C)C)CC[C@@H]1N1CC[C@H](Nc2ncnc3ccc(C(F)(F)F)cc23)C1=O]

In another embodiment, T is:

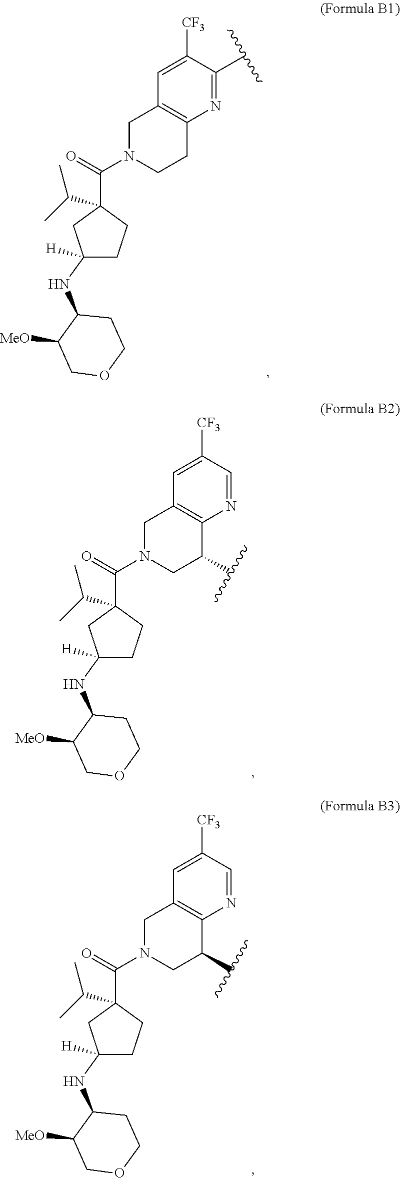 [SMILES: *c1nc2c(cc1C(F)(F)F)CN(C(=O)[C@@]1(C(C)C)CC[C@@H](N[C@H]3CCOC[C@H]3OC)C1)CC2 ; *[C@@H]1CN(C(=O)[C@@]2(C(C)C)CC[C@@H](N[C@H]3CCOC[C@H]3OC)C2)Cc2cc(C(F)(F)F)cnc21 ; *[C@H]1CN(C(=O)[C@@]2(C(C)C)CC[C@@H](N[C@H]3CCOC[C@H]3OC)C2)Cc2cc(C(F)(F)F)cnc21]

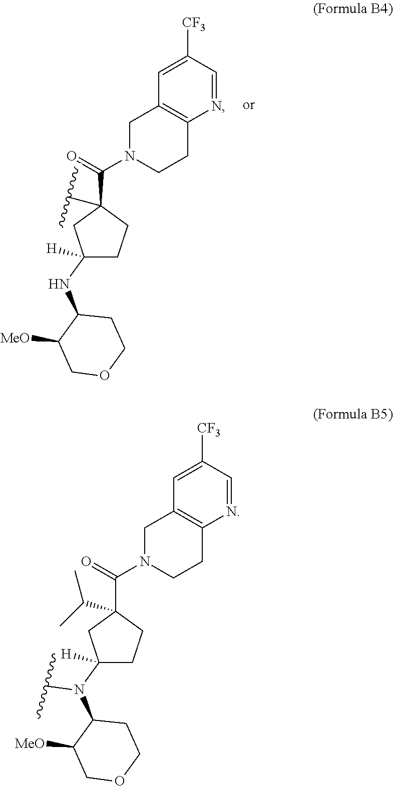 [SMILES: *[C@]1(C(=O)N2CCc3ncc(C(F)(F)F)cc3C2)CC[C@@H](N[C@H]2CCOC[C@H]2OC)C1 ; "or" ; *N([C@@H]1CC[C@@](C(=O)N2CCc3ncc(C(F)(F)F)cc3C2)(C(C)C)C1)[C@H]1CCOC[C@H]1OC]

In another embodiment, T is

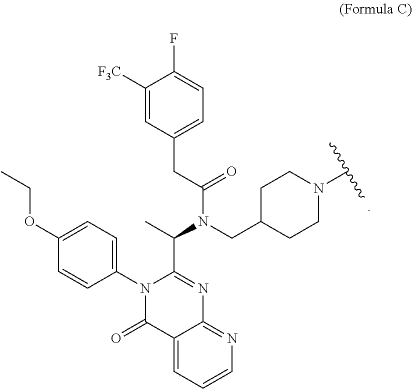 [SMILES: *N1CCC(CN(C(=O)Cc2ccc(F)c(C(F)(F)F)c2)[C@H](C)c2nc3ncccc3c(=O)n2-c2ccc(OCC)cc2)CC1]

In another embodiment, T is

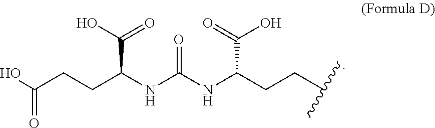 [SMILES: *CC[C@H](NC(=O)N[C@@H](CCC(=O)O)C(=O)O)C(=O)O]

In another embodiment, T is

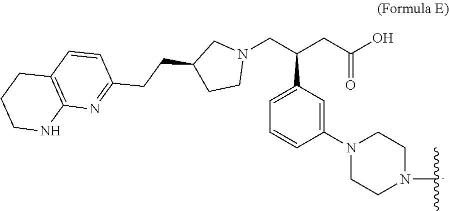 [SMILES: *N1CCN(c2cccc([C@H](CC(=O)O)CN3CC[C@@H](CCc4ccc5c(n4)NCCC5)C3)c2)CC1]

In another embodiment, T is

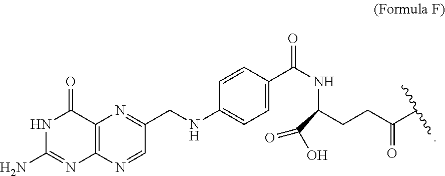 [SMILES: *C(=O)CC[C@H](NC(=O)c1ccc(NCc2cnc3nc(N)[nH]c(=O)c3n2)cc1)C(=O)O]

In another embodiment, T is:

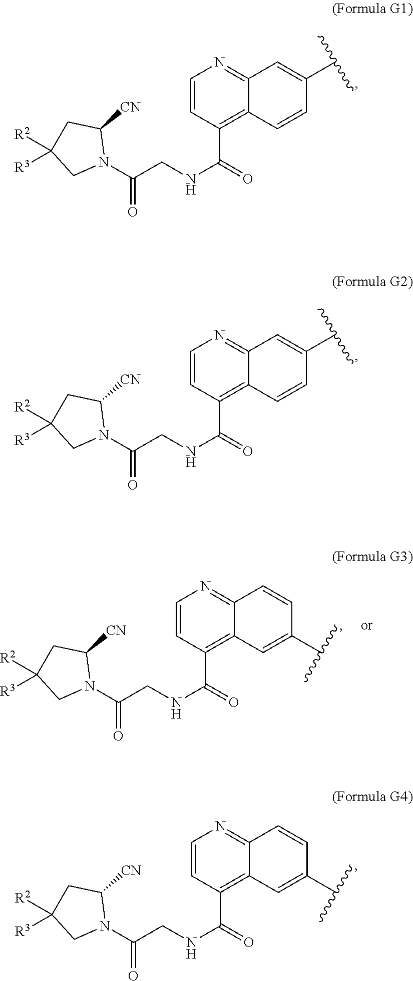

wherein R² and R³ of Formula G1, Formula G2, Formula G3, and Formula G4 are each independently F or H.

In another embodiment, T is

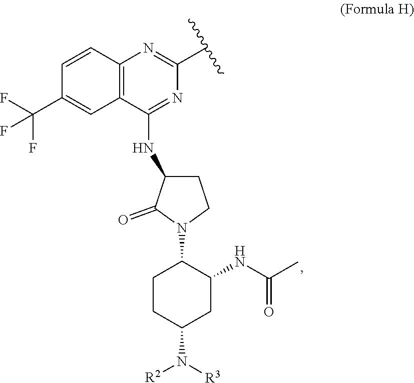

wherein R² of Formula H is hydrogen or C₁₋₄ alkyl;
and R³ of Formula H is hydrogen or C₁₋₄ alkyl. In another embodiment, R² and R³ of Formula H are each independently hydrogen, methyl, ethyl, 1-propyl, 2-propyl, 1-butyl, 2-butyl, or t-butyl. In another embodiment, R² of Formula H is isopropyl and R³ of Formula H is methyl. In another embodiment, R² of Formula H is t-butyl and R³ of Formula H is hydrogen.

In another embodiment, T is

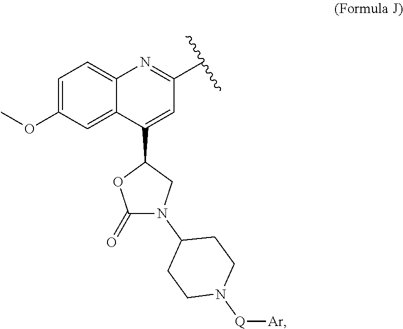

wherein Q is C₁₋₅ alkylene, wherein 0, 1, or 2 methylene units are replaced with —O—; and Ar is an optionally substituted 5- to 1O-membered aromatic ring or 9- or 1O-membered unsaturated fused bicyclic ring.

In another embodiment, Q is —CH₂—, —CH(CH₃)—, —CH₂CH₂CH₂—, —CH(CH₂CH₃)—, or —CH₂CH₂O—. In another embodiment, Q is —CH₂— or —CH(CH₃)—. In another embodiment, Q is —CH(CH₃)—.

In another embodiment, Ar is an optionally substituted 5—, 6—, 7—, 8—, 9—, or 1O-membered aromatic ring. In another embodiment, Ar is an optionally substituted 6-membered aromatic ring. In another embodiment, Ar is an optionally substituted 9-membered aromatic ring. In another embodiment, Ar is an optionally substituted 9- or 1O-membered unsaturated fused bicyclic ring. In another embodiment, Ar is an optionally substituted 9-membered unsaturated fused bicyclic ring.

In another embodiment, Ar is phenyl, pyridinyl, indolyl, indolinyl, dihydrobenzofuranyl, or benzofuranyl, and each Ar is substituted with 0, 1, or 2 substituent groups. In another embodiment, Ar is phenyl, 2-pyridinyl, 3-pyridinyl, 4-pyridinyl, 2-indolyl, 3-indolyl, 4-indolyl, 5-indolyl, 6-indolyl, 7-indolyl, 2-indolinyl, 3-indolinyl, 4-indolinyl, 5-indolinyl, 6-indolinyl, 7-indolinyl, 2-dihydrobenzofuranyl, 3-dihydrobenzofuranyl, 4-dihydrobenzofuranyl, 5-dihydrobenzofuranyl, 6-dihydrobenzofuranyl, 7-dihydrobenzofuranyl, 2-benzofuranyl, 3-benzofuranyl, 4-benzofuranyl, 5-benzofuranyl, 6-benzofuranyl, or 7-benzofuranyl, and each Ar is substituted with 0, 1, or 2 substituent groups.

In another embodiment, the Ar substituent groups are independently selected from C₁-3 alkyl, C₁₋₃ alkoxy, C₁₋₃ haloalkyl, C₁-3 haloalkoxy, or halo. In another embodiment, the Ar substituent groups are independently selected from methyl, ethyl, methoxy, ethoxy, bromo, chloro, or trifluoromethyl.

In another embodiment, Ar is

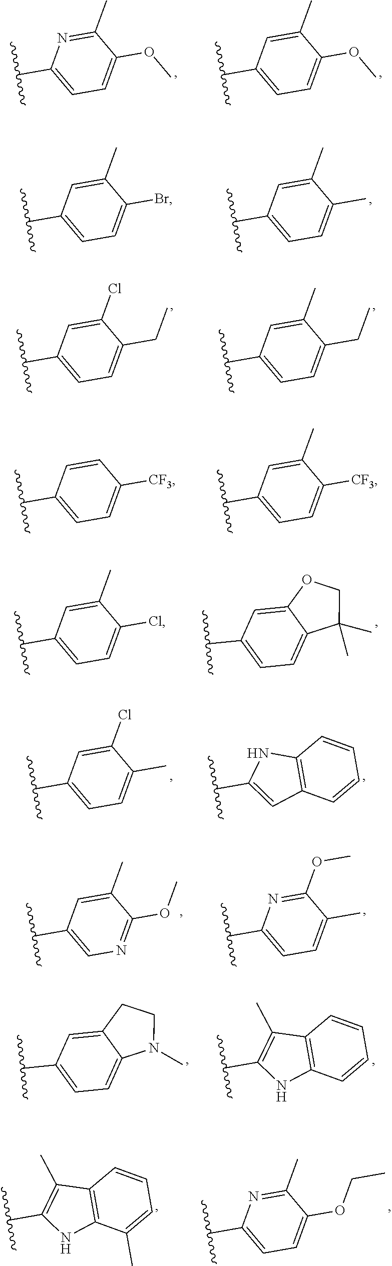

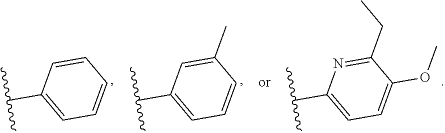

In another embodiment, the compound of Formula (I) is selected from a compound as listed in Table 2:

TABLE 2

| Cpd. | |
|---|---|
| No. | Structure |
| 1 | 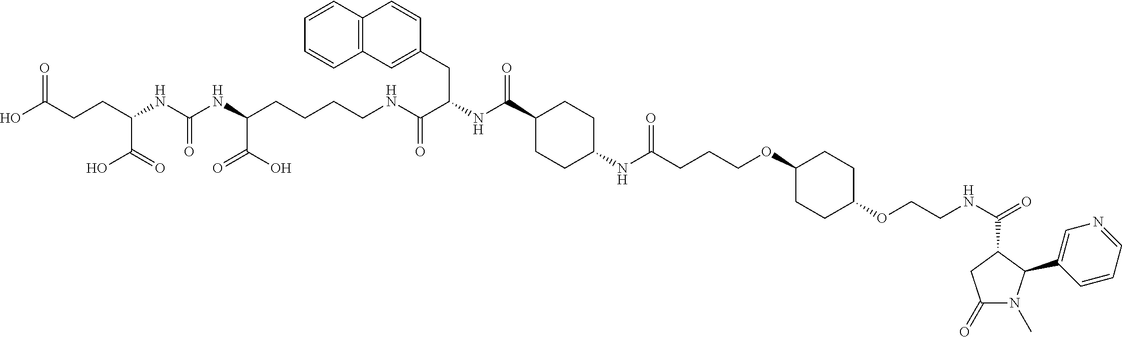 [SMILES: CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1] |
| 2 | 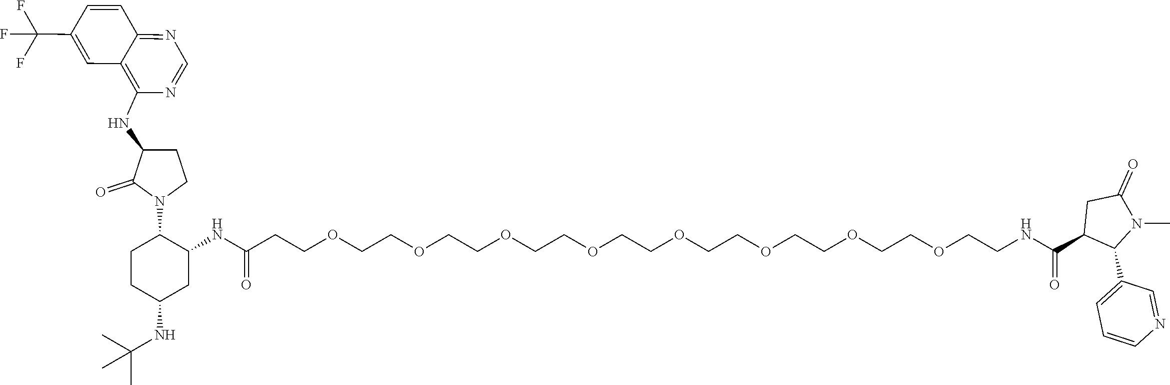 [SMILES: CN1C(=O)C[C@H](C(=O)NCCOCCOCCOCCOCCOCCOCCOCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |

In another embodiment, the compound of Formula (I) is selected from a compound as listed in Table 3:

TABLE 3

| Com- | |
|---|---|
| pound | Structure |
| A1 | 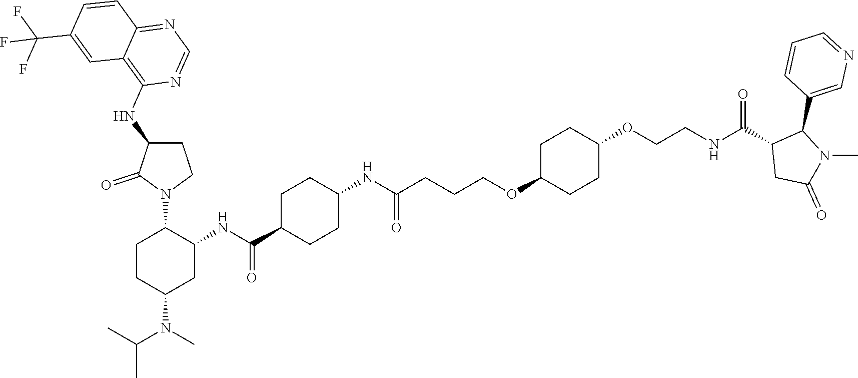 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C)[C@@H]4c4cccnc4)CC3)CC2)C1] |
| A2 | 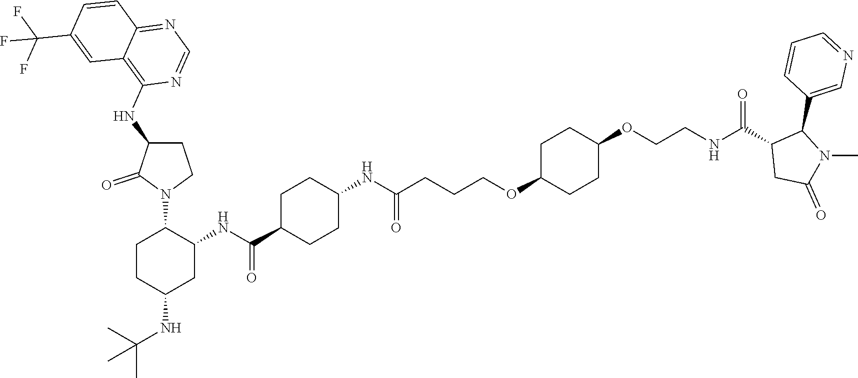 [SMILES: CN1C(=O)C[C@H](C(=O)NCCO[C@H]2CC[C@@H](OCCCC(=O)N[C@H]3CC[C@H](C(=O)N[C@@H]4C[C@H](NC(C)(C)C)CC[C@@H]4N4CC[C@H](Nc5ncnc6ccc(C(F)(F)F)cc56)C4=O)CC3)CC2)[C@H]1c1cccnc1] |
| A3 | 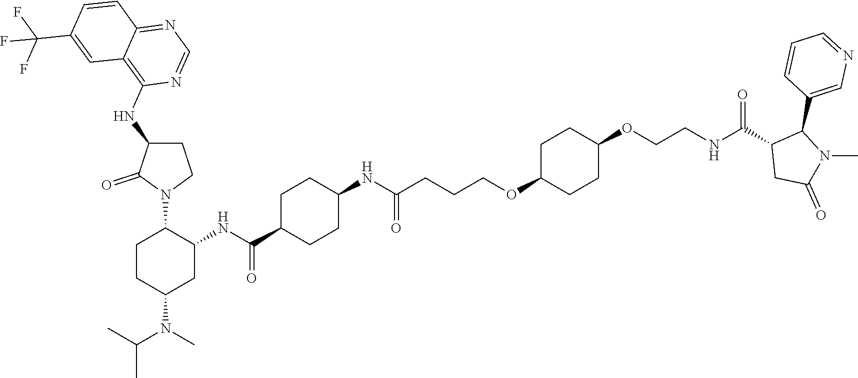 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)C1] |
| A4 | 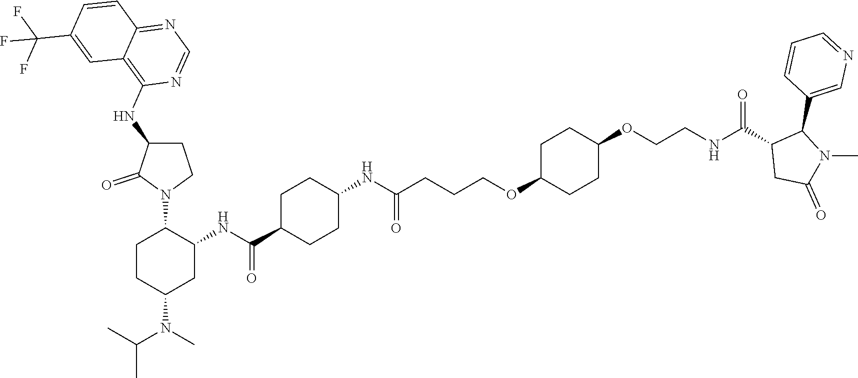 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)C1] |
| A5 | 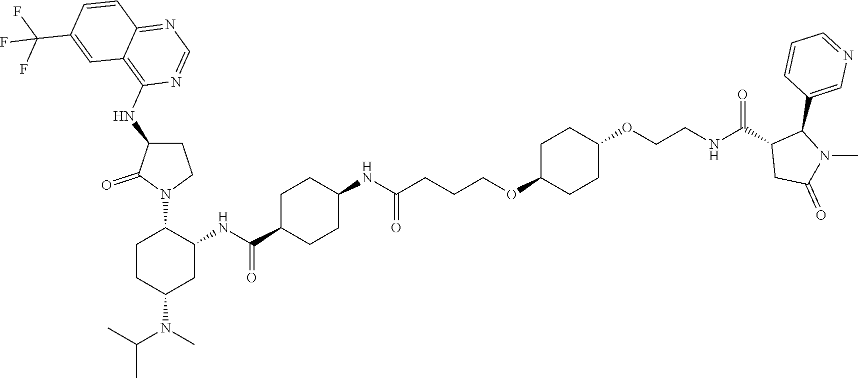 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)C1] |
| A6 | 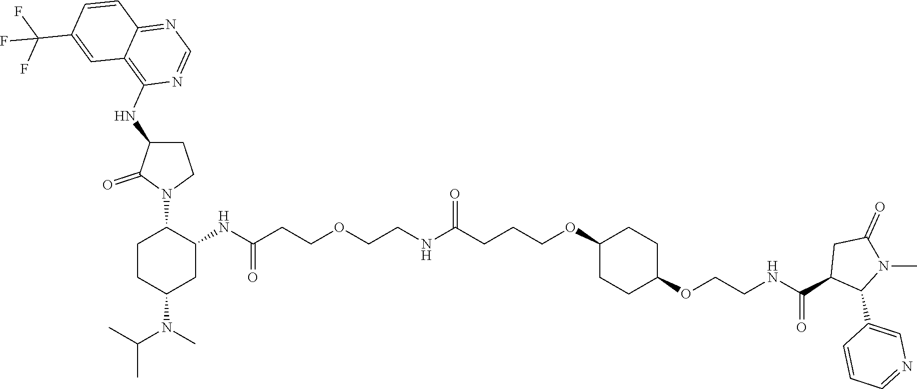 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCCO[C@H]2CC[C@@H](OCCNC(=O)[C@H]3CC(=O)N(C)[C@@H]3c3cccnc3)CC2)C1] |
| A7 | 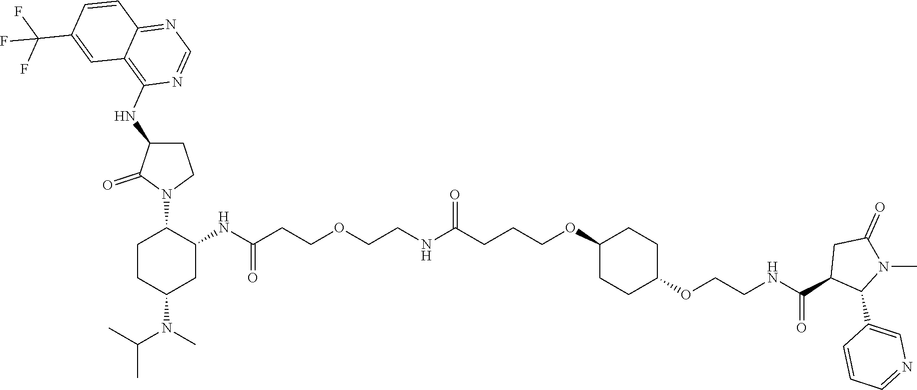 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCCO[C@H]2CC[C@H](OCCNC(=O)[C@H]3CC(=O)N(C)[C@@H]3c3cccnc3)CC2)C1] |
| A8 | 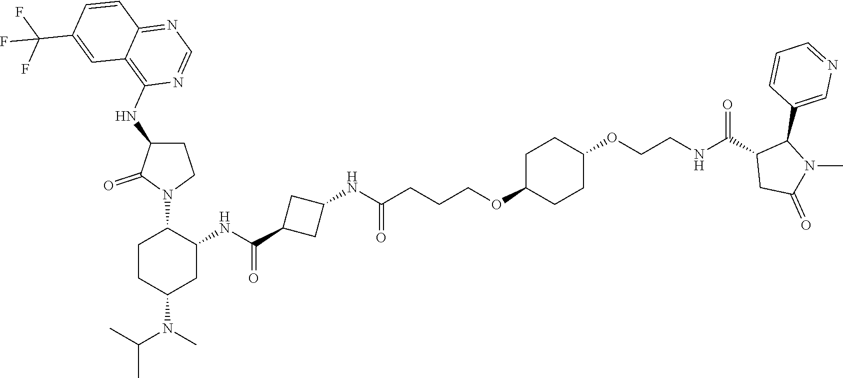 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCCO[C@H]3CC[C@H](OCCNC(=O)[C@H]4CC(=O)N(C)[C@@H]4c4cccnc4)CC3)C2)C1] |
| A9 | 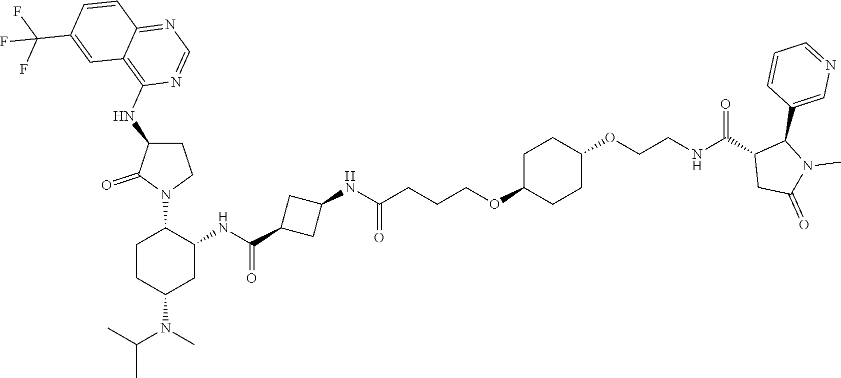 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCCO[C@H]3CC[C@H](OCCNC(=O)[C@H]4CC(=O)N(C)[C@@H]4c4cccnc4)CC3)C2)C1] |
| A10 | 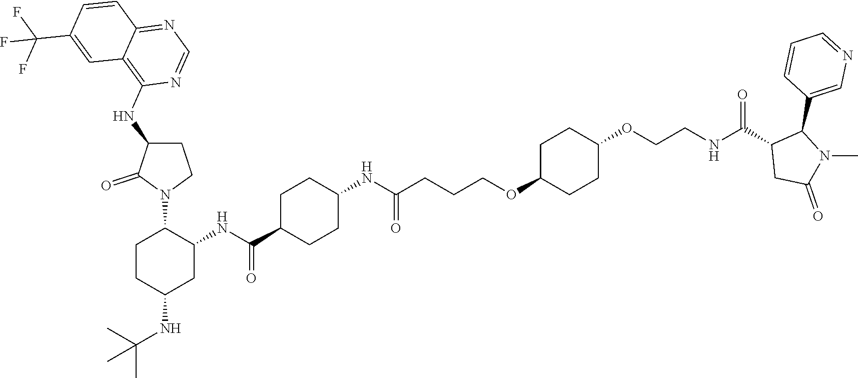 [SMILES: CN1C(=O)C[C@H](C(=O)NCCO[C@H]2CC[C@H](OCCCC(=O)N[C@H]3CC[C@H](C(=O)N[C@@H]4C[C@H](NC(C)(C)C)CC[C@@H]4N4CC[C@H](Nc5ncnc6ccc(C(F)(F)F)cc56)C4=O)CC3)CC2)[C@H]1c1cccnc1] |
| A11 | 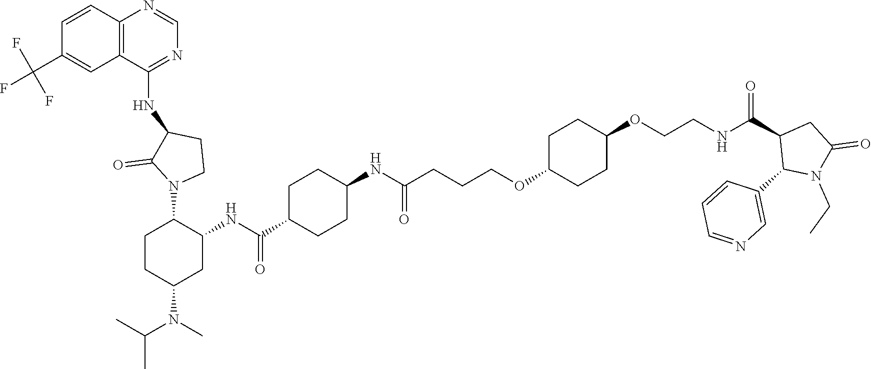 [SMILES: CCN1C(=O)C[C@H](C(=O)NCCO[C@H]2CC[C@H](OCCCC(=O)N[C@H]3CC[C@H](C(=O)N[C@@H]4C[C@H](N(C)C(C)C)CC[C@@H]4N4CC[C@H](Nc5ncnc6ccc(C(F)(F)F)cc56)C4=O)CC3)CC2)[C@H]1c1cccnc1] |
| A12 | 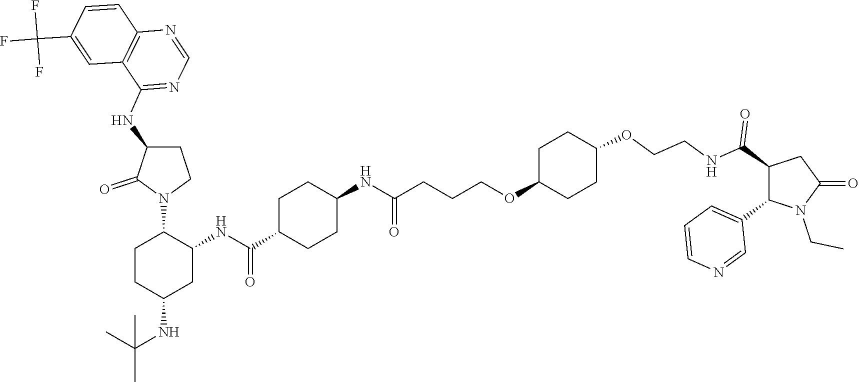 [SMILES: CCN1C(=O)C[C@H](C(=O)NCCO[C@H]2CC[C@H](OCCCC(=O)N[C@H]3CC[C@H](C(=O)N[C@@H]4C[C@H](NC(C)(C)C)CC[C@@H]4N4CC[C@H](Nc5ncnc6ccc(C(F)(F)F)cc56)C4=O)CC3)CC2)[C@H]1c1cccnc1] |
| A13 | 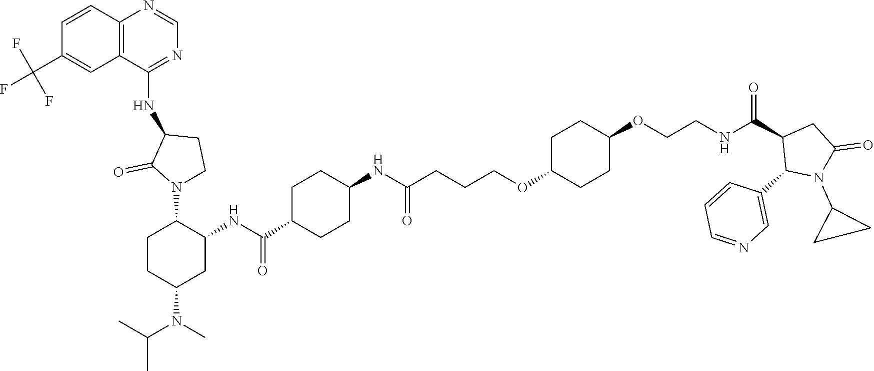 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C5CC5)[C@@H]4c4cccnc4)CC3)CC2)C1] |
| A14 | 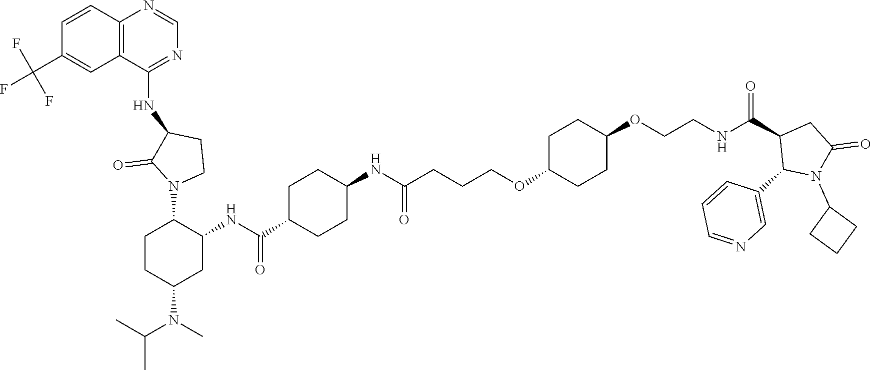 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C5CCC5)[C@@H]4c4cccnc4)CC3)CC2)C1] |
| A15 | 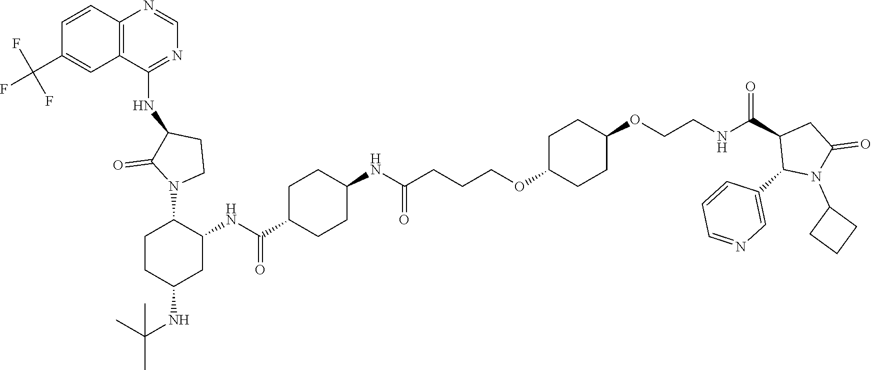 [SMILES: CC(C)(C)N[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C5CCC5)[C@@H]4c4cccnc4)CC3)CC2)C1] |
| A15a | 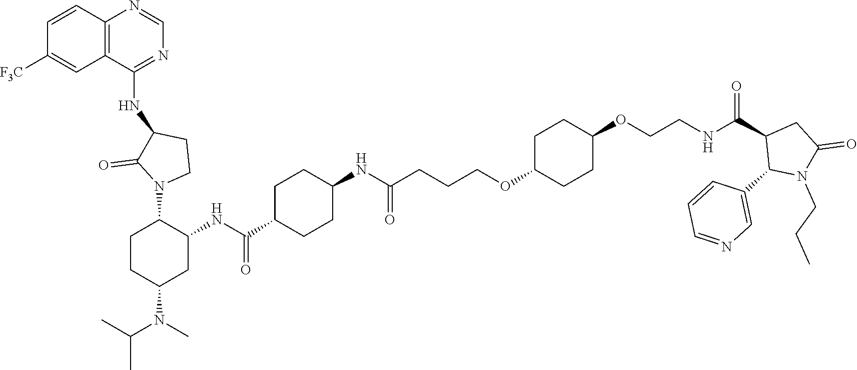 [SMILES: CCCN1C(=O)C[C@H](C(=O)NCCO[C@H]2CC[C@H](OCCCC(=O)N[C@H]3CC[C@H](C(=O)N[C@@H]4C[C@H](N(C)C(C)C)CC[C@@H]4N4CC[C@H](Nc5ncnc6ccc(C(F)(F)F)cc56)C4=O)CC3)CC2)[C@H]1c1cccnc1] |
| A16 | 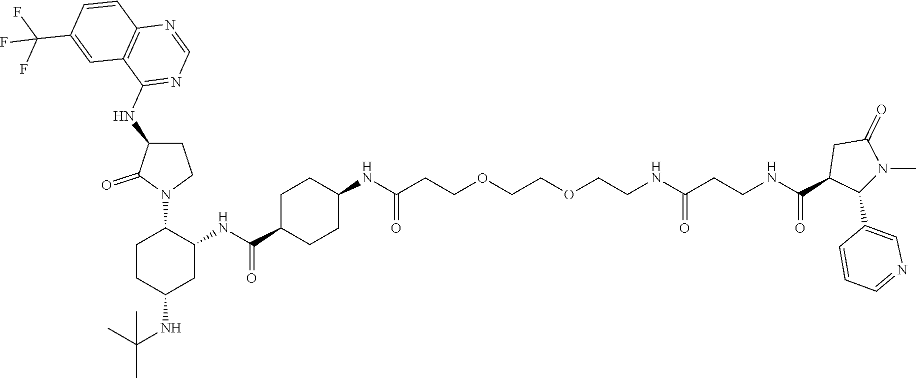 [SMILES: CN1C(=O)C[C@H](C(=O)NCCC(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A17 | 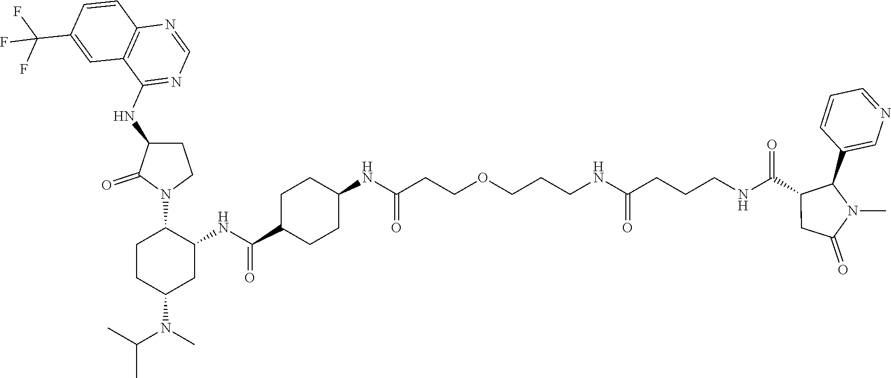 |
| A18 | 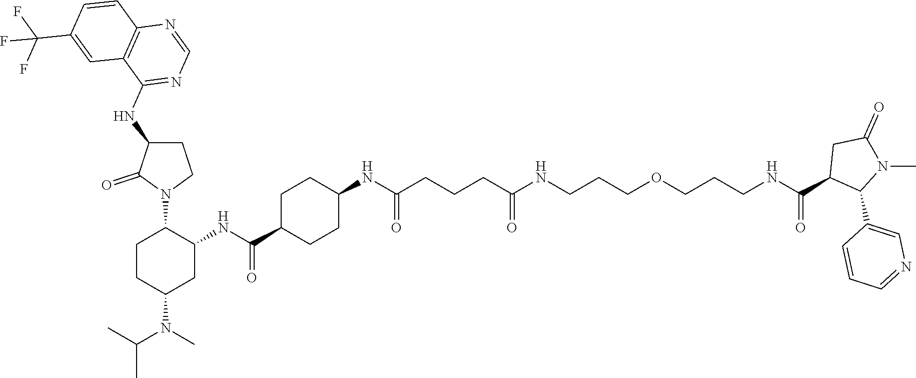 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCC(=O)NCCCOCCCNC(=O)[C@H]3CC(=O)N(C)[C@@H]3c3cccnc3)CC2)C1] |
| A19 | 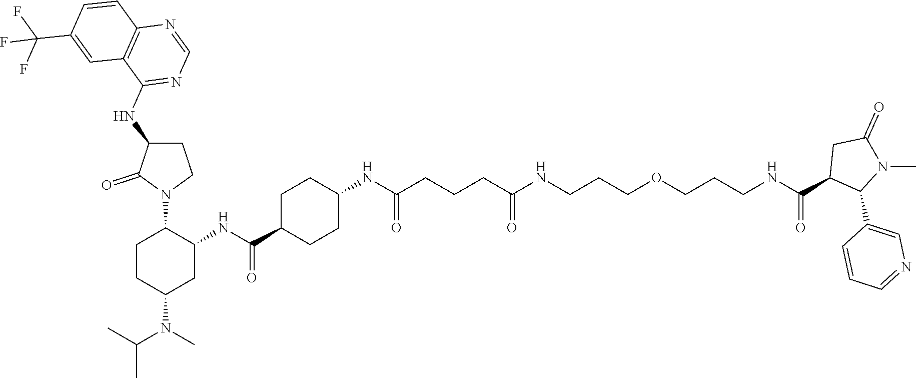 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCC(=O)NCCCOCCCNC(=O)[C@H]3CC(=O)N(C)[C@@H]3c3cccnc3)CC2)C1] |
| A20 | 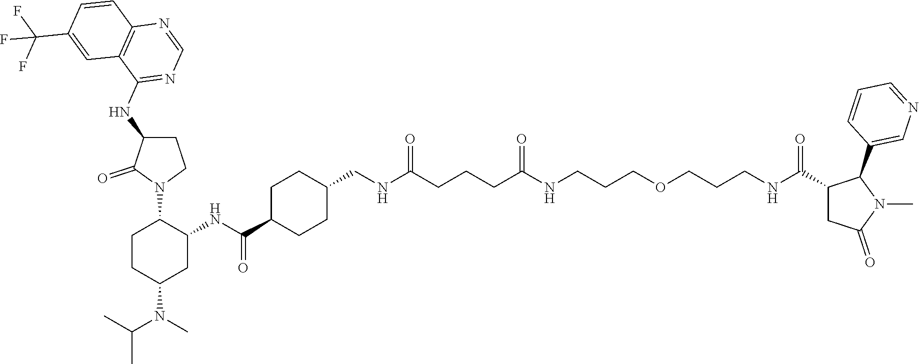 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](CNC(=O)CCCC(=O)NCCCOCCCNC(=O)[C@H]3CC(=O)N(C)[C@@H]3c3cccnc3)CC2)C1] |
| A21 | 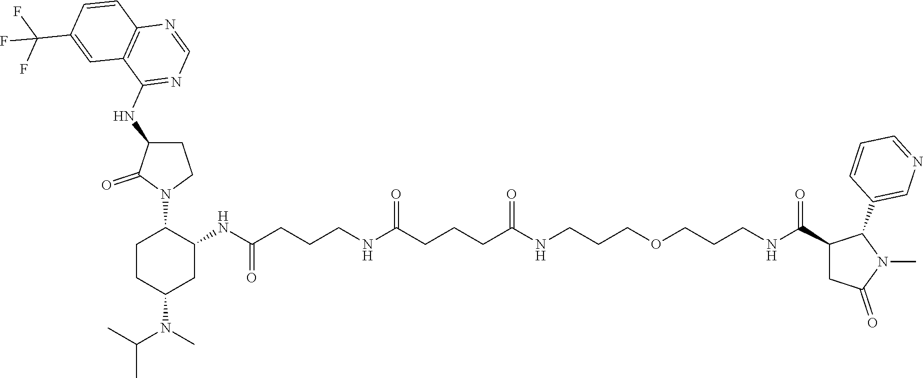 [SMILES: CC(C)N(C)[C@@H]1CC[C@H](N2CC[C@H](Nc3ncnc4ccc(C(F)(F)F)cc34)C2=O)[C@H](NC(=O)CCCNC(=O)CCCC(=O)NCCCOCCCNC(=O)[C@@H]2CC(=O)N(C)[C@H]2c2cccnc2)C1] |
| A22 | 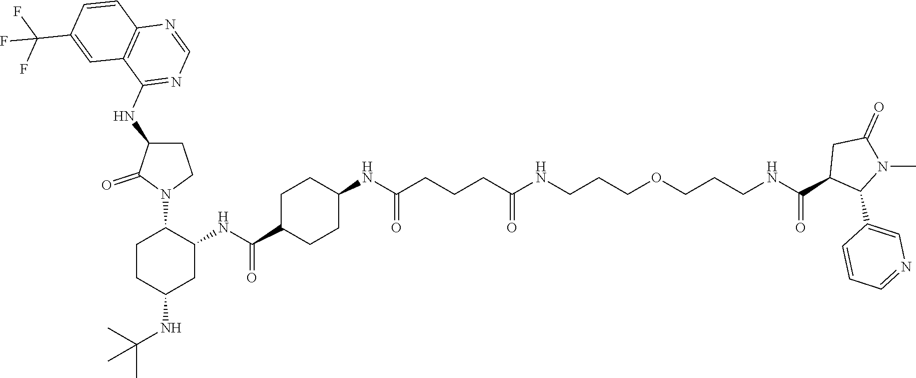 |
| A23 | 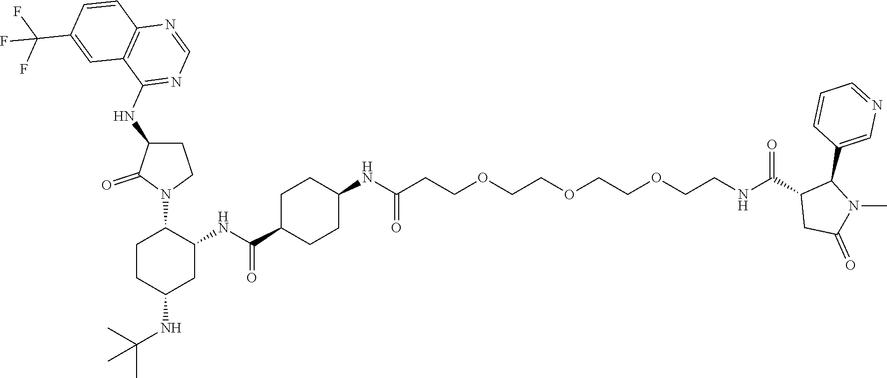 [SMILES: CN1C(=O)C[C@H](C(=O)NCCOCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A24 | 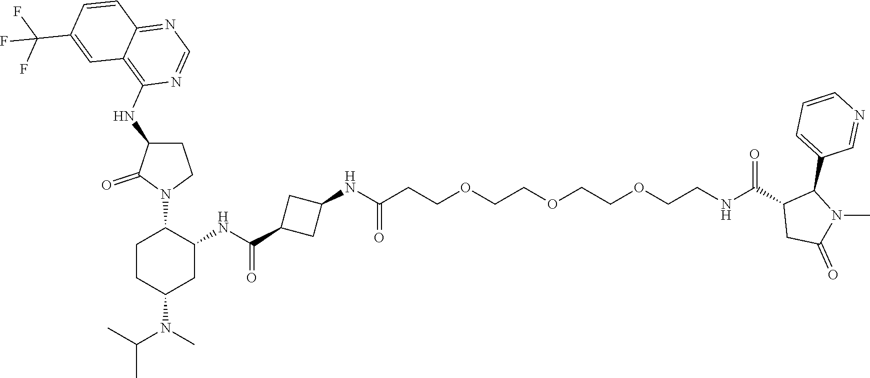 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCOCCOCCNC(=O)[C@H]3CC(=O)N(C)[C@@H]3c3cccnc3)C2)C1] |
| A25 | 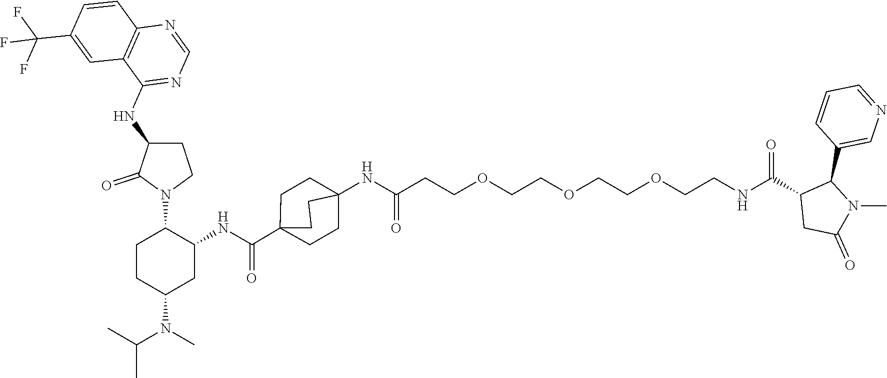 [SMILES: CC(C)N(C)[C@@H]1CC[C@H](N2CC[C@H](Nc3ncnc4ccc(C(F)(F)F)cc34)C2=O)[C@H](NC(=O)C23CCC(NC(=O)CCOCCOCCOCCNC(=O)[C@H]4CC(=O)N(C)[C@@H]4c4cccnc4)(CC2)CC3)C1] |
| A26 | 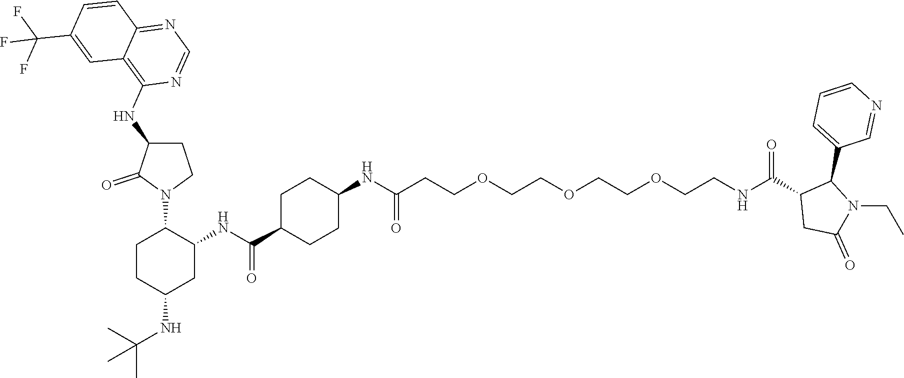 [SMILES: CCN1C(=O)C[C@H](C(=O)NCCOCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A27 | 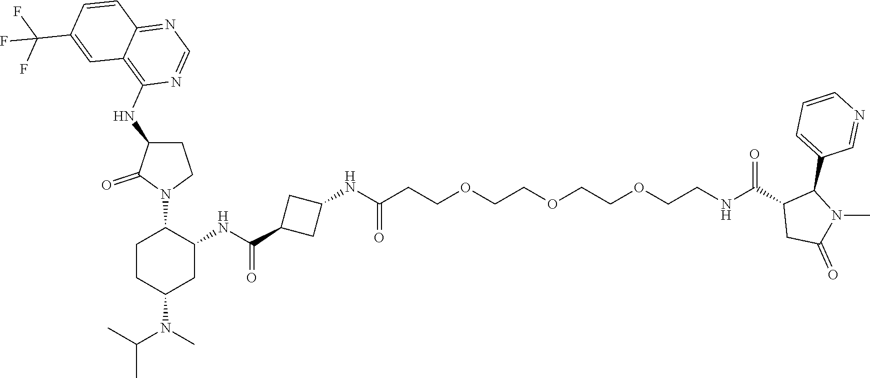 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCOCCOCCOCCNC(=O)[C@H]3CC(=O)N(C)[C@@H]3c3cccnc3)C2)C1] |
| A28 | 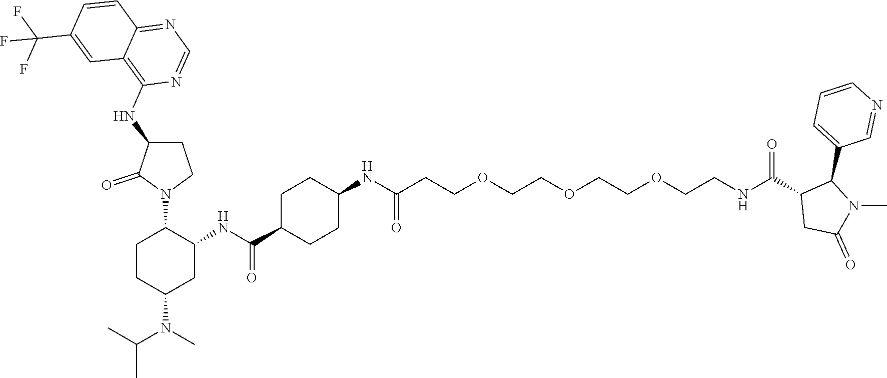 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCOCCNC(=O)[C@H]3CC(=O)N(C)[C@@H]3c3cccnc3)CC2)C1] |
| A29 | 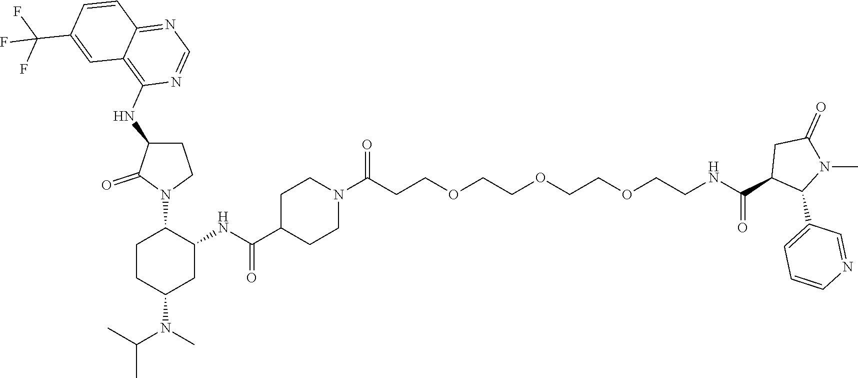 [SMILES: CC(C)N(C)[C@@H]1CC[C@H](N2CC[C@H](Nc3ncnc4ccc(C(F)(F)F)cc34)C2=O)[C@H](NC(=O)C2CCN(C(=O)CCOCCOCCOCCNC(=O)[C@H]3CC(=O)N(C)[C@@H]3c3cccnc3)CC2)C1] |
| A30 | 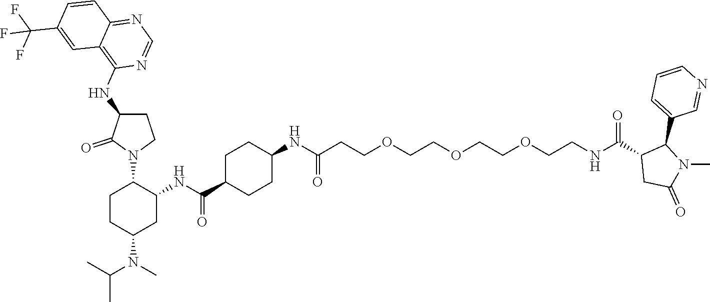 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCOCCNC(=O)[C@H]3CC(=O)N(C)[C@@H]3c3cccnc3)CC2)C1] |
| A31 | 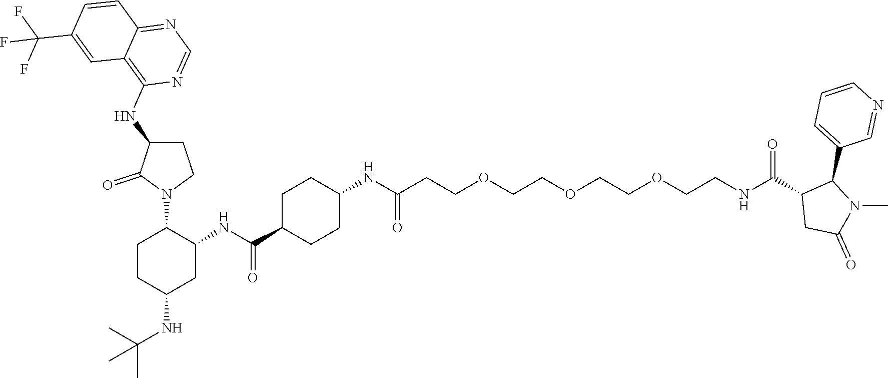 [SMILES: CN1C(=O)C[C@H](C(=O)NCCOCCOCCOCCC(=O)N[C@H]2CC[C@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A32 | 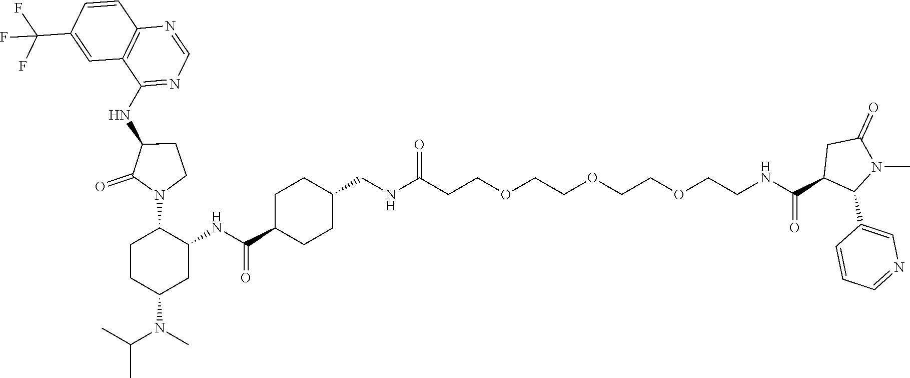 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](CNC(=O)CCOCCOCCOCCNC(=O)[C@H]3CC(=O)N(C)[C@@H]3c3cccnc3)CC2)C1] |
| A33 | 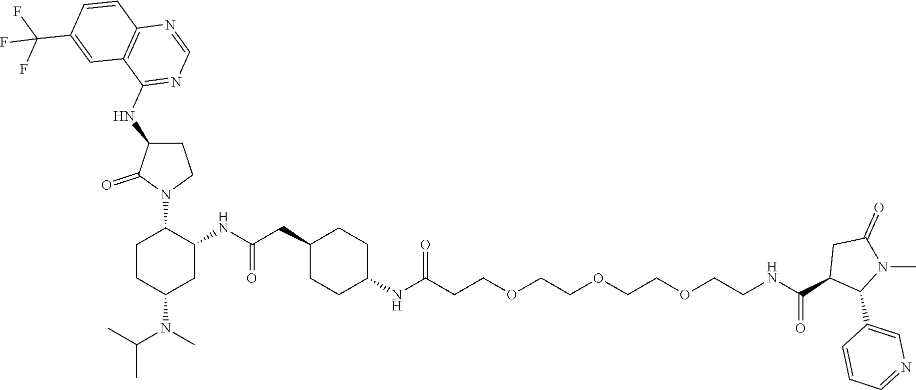 [SMILES: CC(C)N(C)[C@@H]1CC[C@H](N2CC[C@H](Nc3ncnc4ccc(C(F)(F)F)cc34)C2=O)[C@H](NC(=O)C[C@H]2CC[C@H](NC(=O)CCOCCOCCOCCNC(=O)[C@H]3CC(=O)N(C)[C@@H]3c3cccnc3)CC2)C1] |
| A34 | 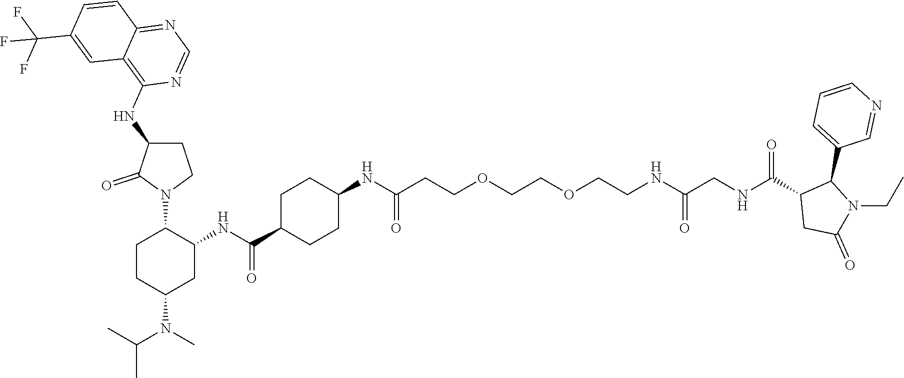 [SMILES: CCN1C(=O)C[C@H](C(=O)NCC(=O)NCCOCCOCCC(=O)N[C@H]2CC[C@@H](C(=O)N[C@@H]3C[C@H](N(C)C(C)C)CC[C@@H]3N3CC[C@H](Nc4ncnc5ccc(C(F)(F)F)cc45)C3=O)CC2)[C@H]1c1cccnc1] |
| A35 | 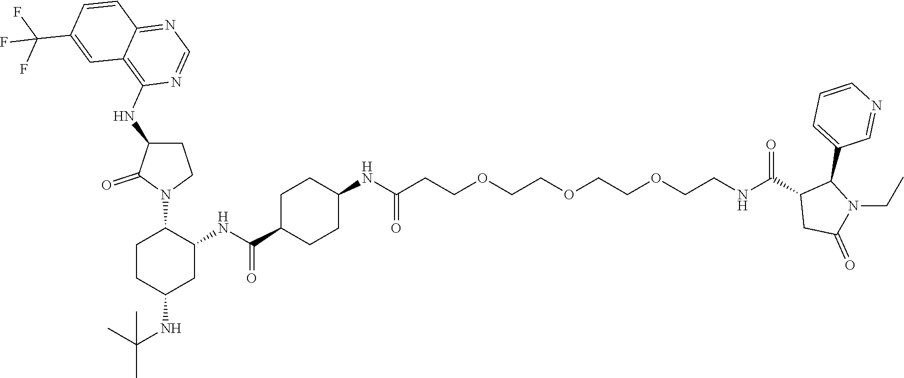 [SMILES: CCN1C(=O)C[C@H](C(=O)NCCOCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A36 | 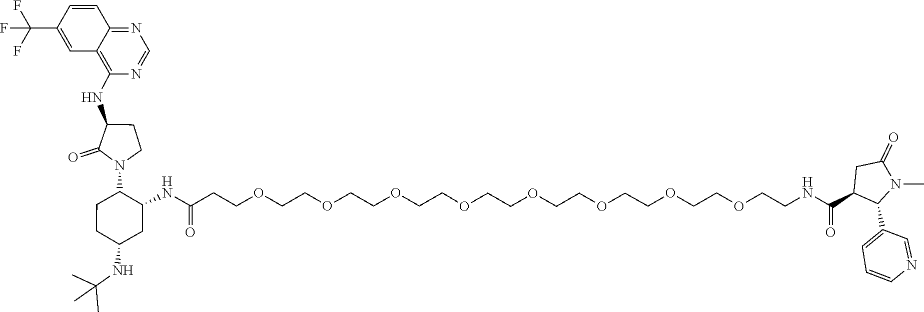 |
| A37 | 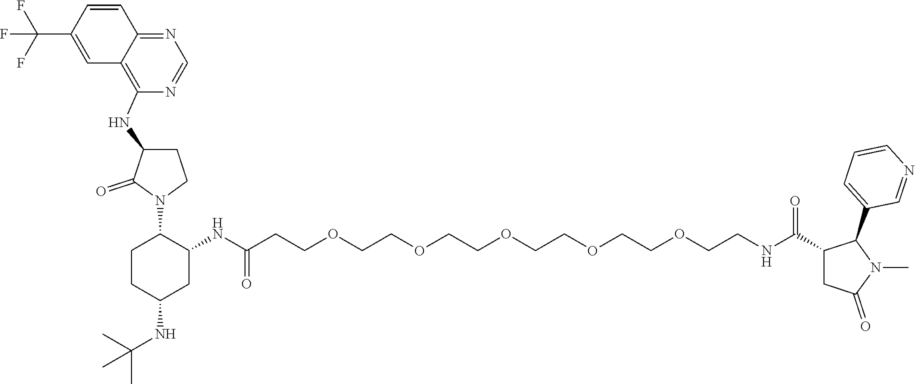 [SMILES: CN1C(=O)C[C@H](C(=O)NCCOCCOCCOCCOCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |
| A38 | 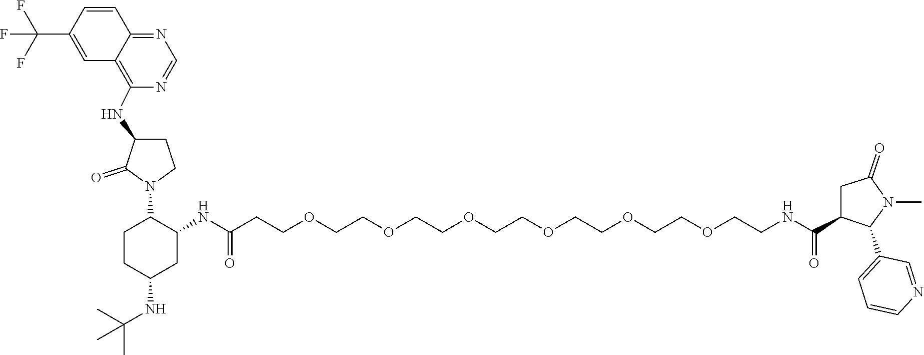 |
| A39 | 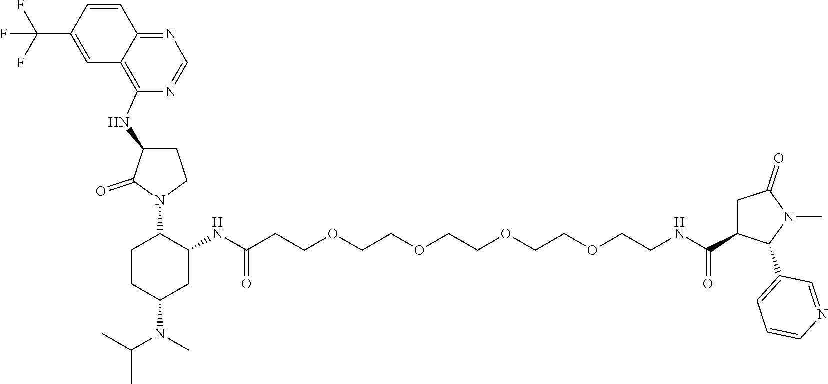 |
| A40 | 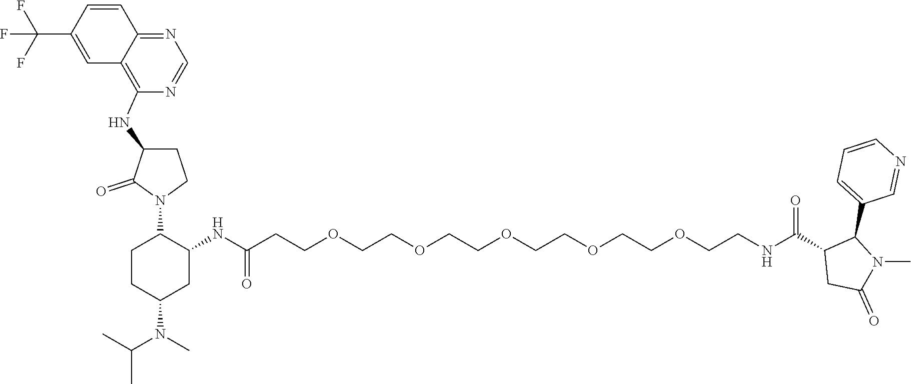 |
| A41 | 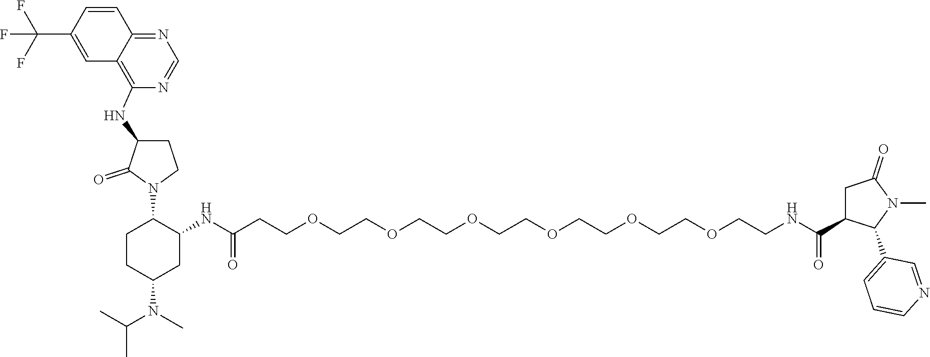 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCOCCOCCOCCNC(=O)[C@H]2CC(=O)N(C)[C@@H]2c2cccnc2)C1] |
| A42 | 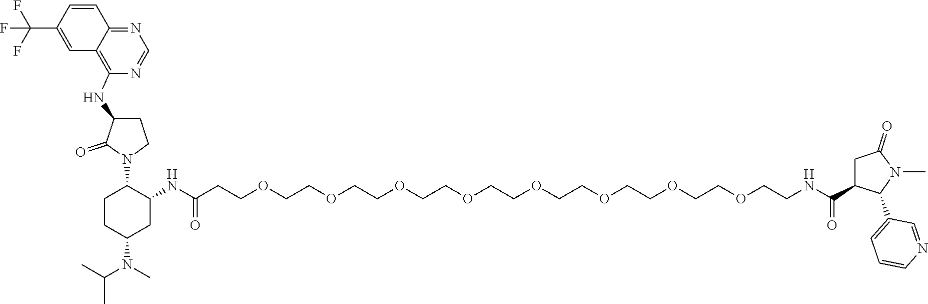 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCOCCOCCOCCOCCOCCNC(=O)[C@H]2CC(=O)N(C)[C@@H]2c2cccnc2)C1] |
| A43 | 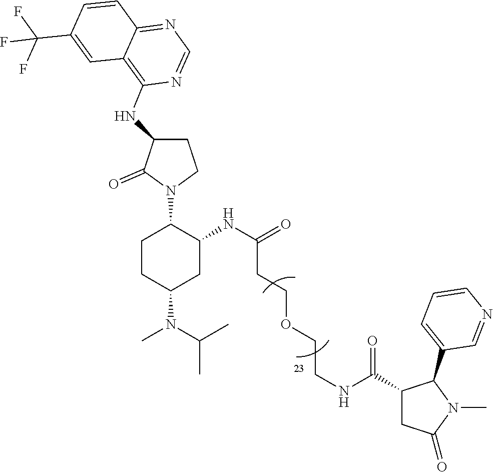 |
| A44 | 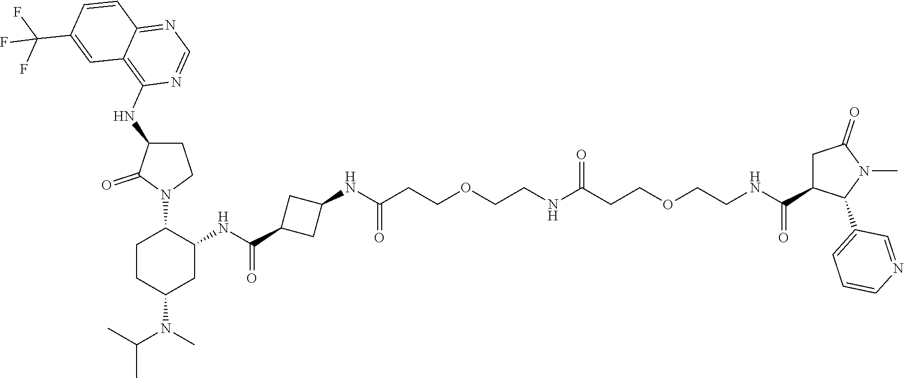 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCNC(=O)CCOCCNC(=O)[C@H]3CC(=O)N(C)[C@@H]3c3cccnc3)C2)C1] |
| A45 | 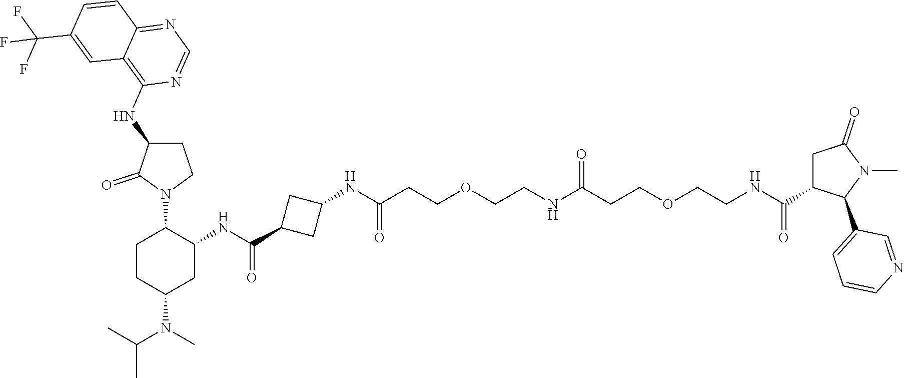 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCOCCNC(=O)CCOCCNC(=O)[C@@H]3CC(=O)N(C)[C@H]3c3cccnc3)C2)C1] |
| A46 | 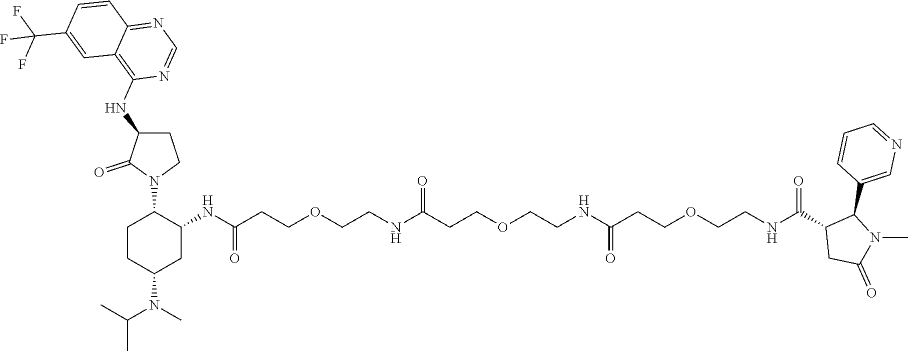 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCOCCNC(=O)CCOCCNC(=O)[C@H]2CC(=O)N(C)[C@@H]2c2cccnc2)C1] |
| A47 | 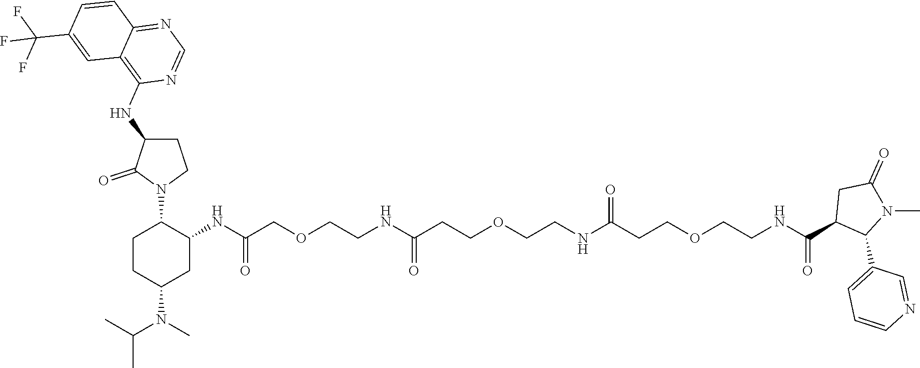 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCOCCNC(=O)CCOCCNC(=O)[C@H]2CC(=O)N(C)[C@@H]2c2cccnc2)C1] |
| A48 | 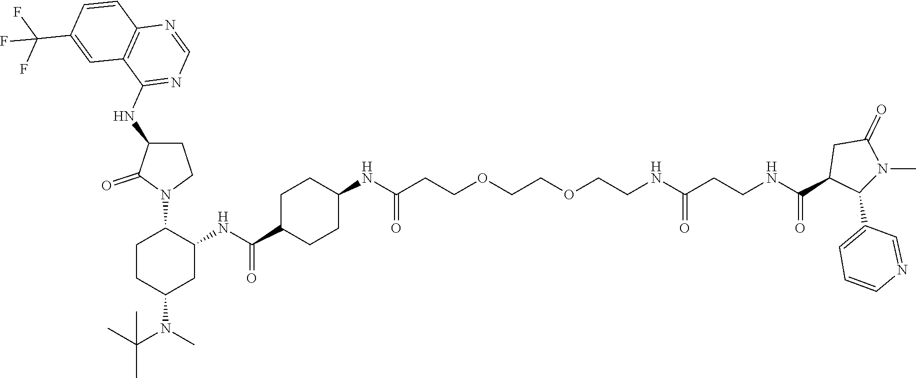 |
| A49 | 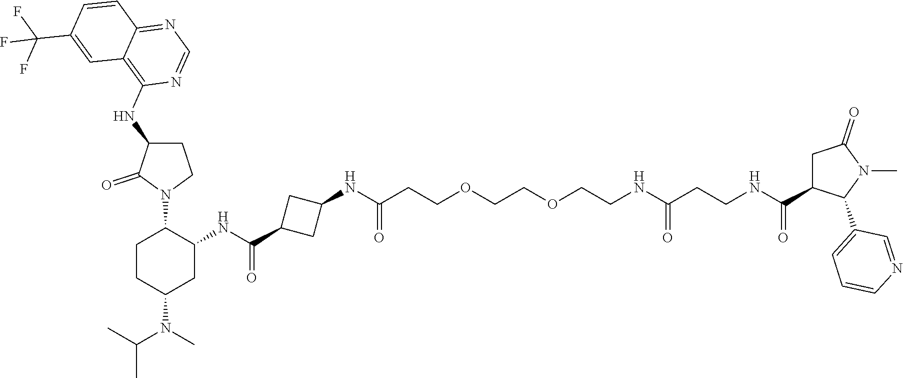 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCOCCNC(=O)CCNC(=O)[C@H]3CC(=O)N(C)[C@@H]3c3cccnc3)C2)C1] |
| A50 | 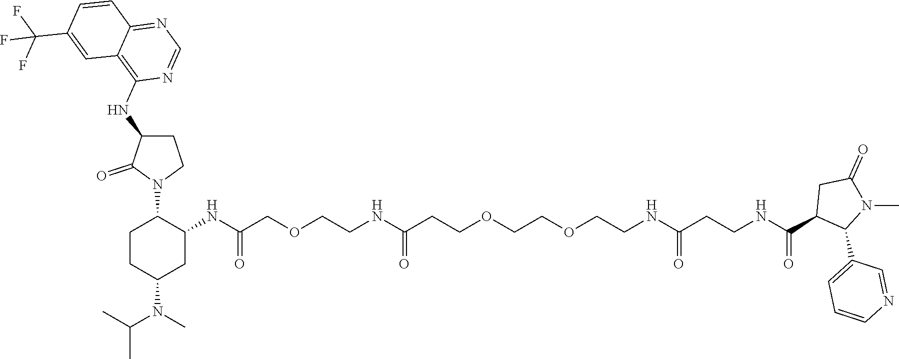 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCOCCOCCNC(=O)CCNC(=O)[C@H]2CC(=O)N(C)[C@@H]2c2cccnc2)C1] |
| A51 | 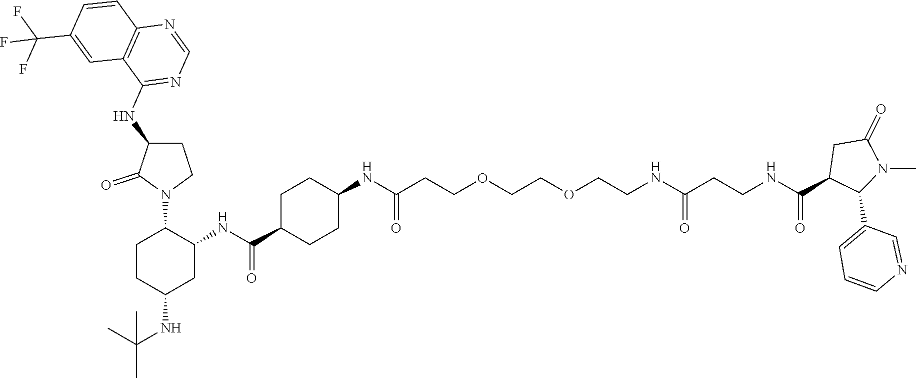 [SMILES: CN1C(=O)C[C@H](C(=O)NCCC(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A52 | 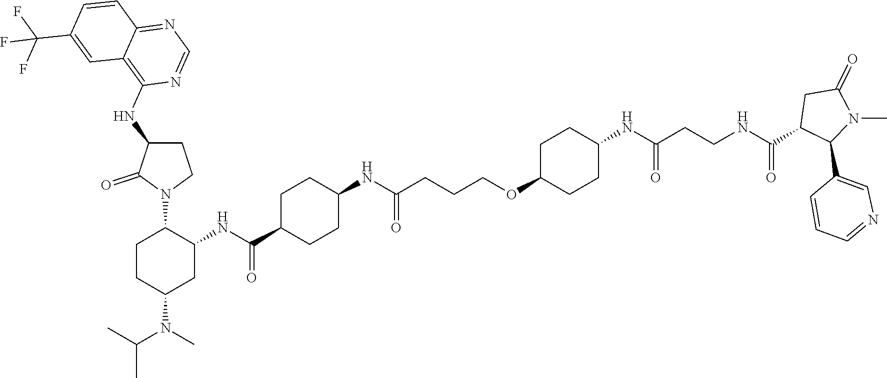 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@H](NC(=O)CCNC(=O)[C@@H]4CC(=O)N(C)[C@H]4c4cccnc4)CC3)CC2)C1] |
| A53 | 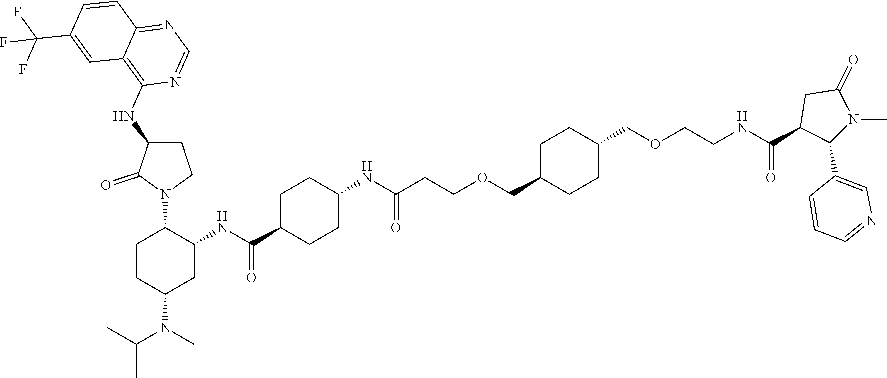 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCOC[C@H]3CC[C@H](COCCNC(=O)[C@H]4CC(=O)N(C)[C@@H]4c4cccnc4)CC3)CC2)C1] |
| A54 | 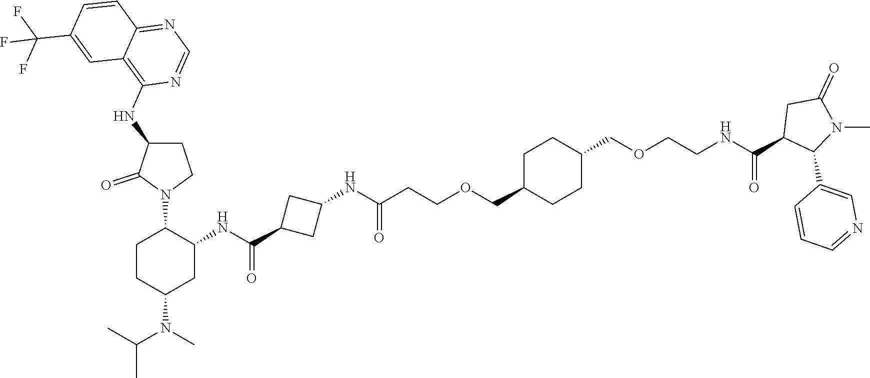 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCOC[C@H]3CC[C@H](COCCNC(=O)[C@H]4CC(=O)N(C)[C@@H]4c4cccnc4)CC3)C2)C1] |
| A55 | 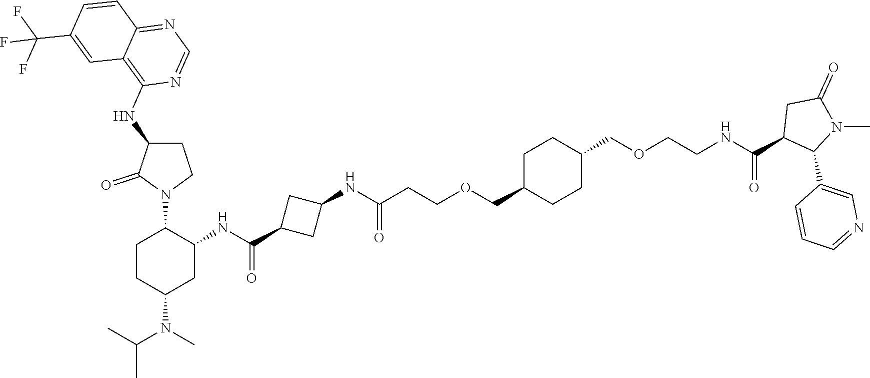 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOC[C@H]3CC[C@H](COCCNC(=O)[C@H]4CC(=O)N(C)[C@@H]4c4cccnc4)CC3)C2)C1] |
| A56 | 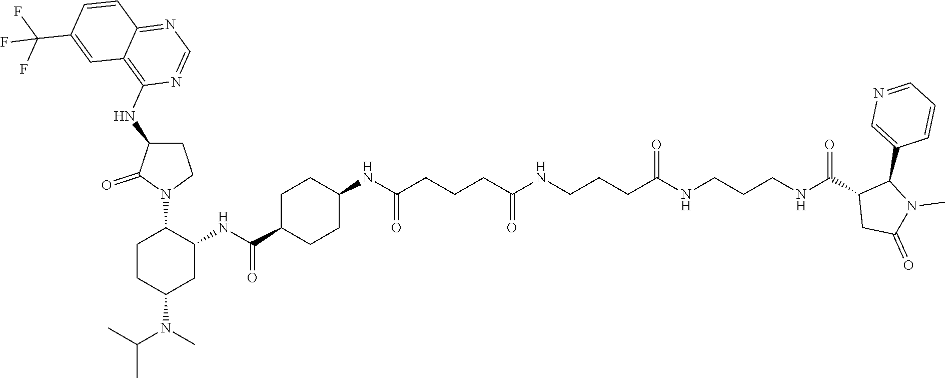 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCC(=O)NCCCC(=O)NCCCNC(=O)[C@H]3CC(=O)N(C)[C@@H]3c3cccnc3)CC2)C1] |
| A57 | 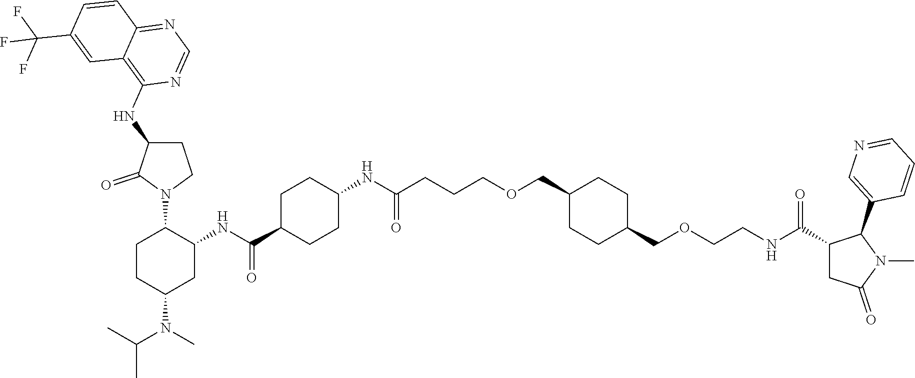 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCOC[C@H]3CC[C@@H](COCCNC(=O)[C@H]4CC(=O)N(C)[C@@H]4c4cccnc4)CC3)CC2)C1] |
| A58 | 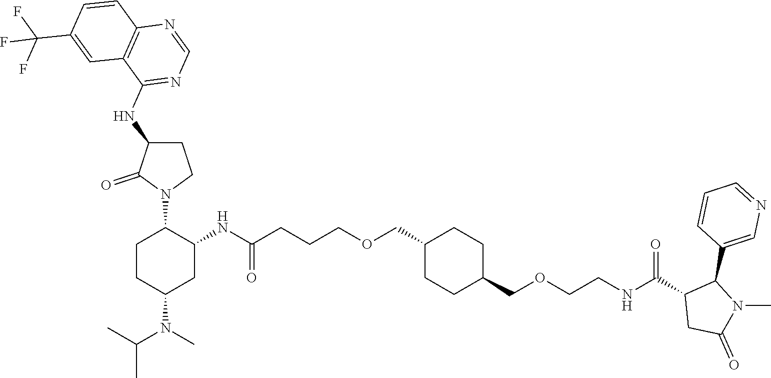 [SMILES: CC(C)N(C)[C@@H]1CC[C@H](N2CC[C@H](Nc3ncnc4ccc(C(F)(F)F)cc34)C2=O)[C@H](NC(=O)CCCOC[C@H]2CC[C@H](COCCNC(=O)[C@H]3CC(=O)N(C)[C@@H]3c3cccnc3)CC2)C1] |
| A59 | 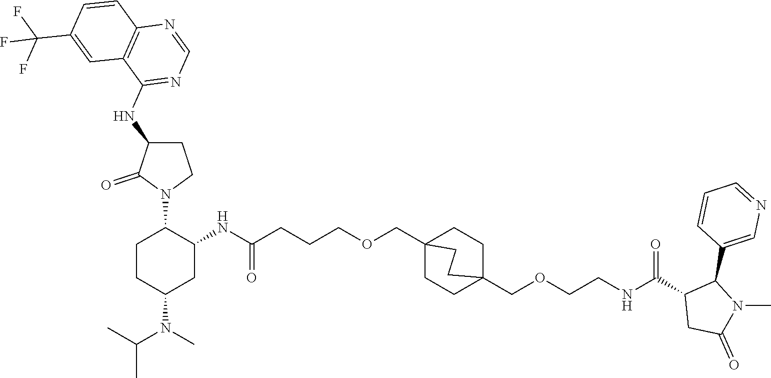 [SMILES: CC(C)N(C)[C@@H]1CC[C@H](N2CC[C@H](Nc3ncnc4ccc(C(F)(F)F)cc34)C2=O)[C@H](NC(=O)CCCOCC23CCC(COCCNC(=O)[C@H]4CC(=O)N(C)[C@@H]4c4cccnc4)(CC2)CC3)C1] |
| A60 | 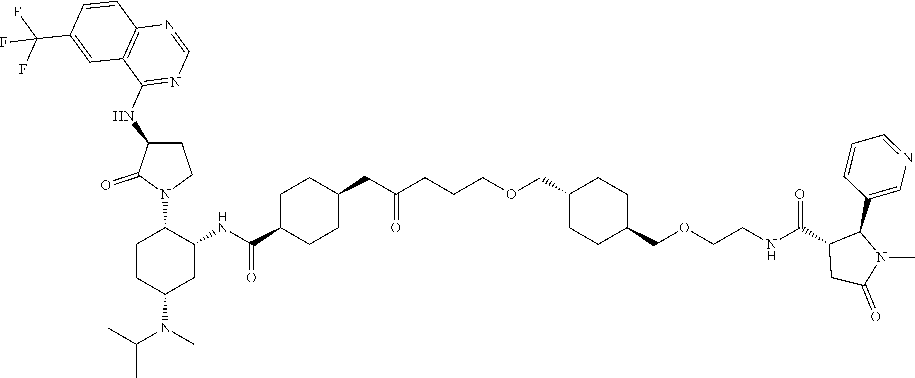 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](CC(=O)CCCOC[C@H]3CC[C@H](COCCNC(=O)[C@H]4CC(=O)N(C)[C@@H]4c4cccnc4)CC3)CC2)C1] |
| A61 | 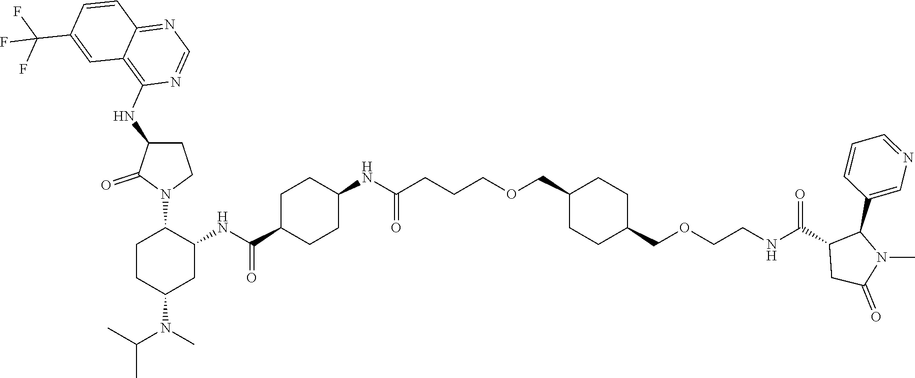 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCOC[C@H]3CC[C@@H](COCCNC(=O)[C@H]4CC(=O)N(C)[C@@H]4c4cccnc4)CC3)CC2)C1] |
| A62 | 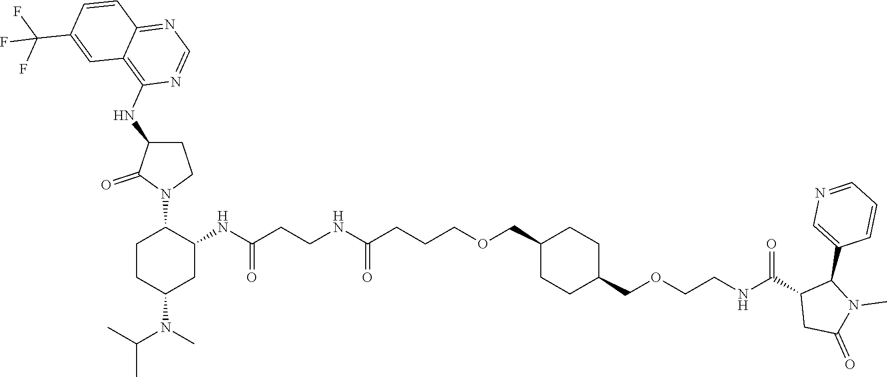 [SMILES: CC(C)N(C)[C@@H]1CC[C@H](N2CC[C@H](Nc3ncnc4ccc(C(F)(F)F)cc34)C2=O)[C@H](NC(=O)CCNC(=O)CCCOC[C@H]2CC[C@@H](COCCNC(=O)[C@H]3CC(=O)N(C)[C@@H]3c3cccnc3)CC2)C1] |
| A63 | 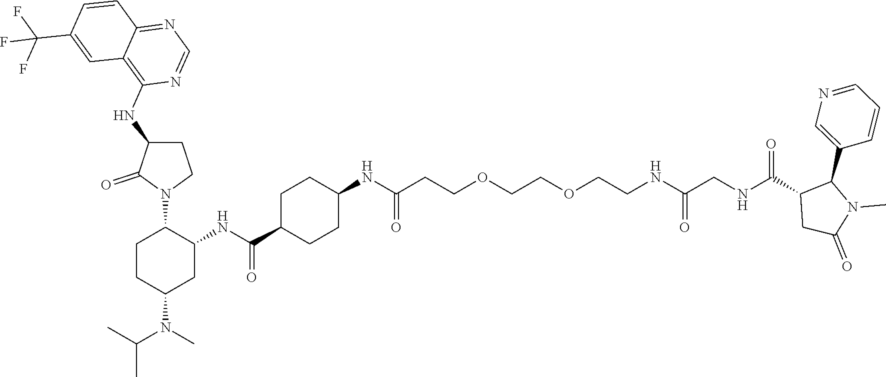 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCNC(=O)CNC(=O)[C@H]3CC(=O)N(C)[C@@H]3c3cccnc3)CC2)C1] |
| A64 | 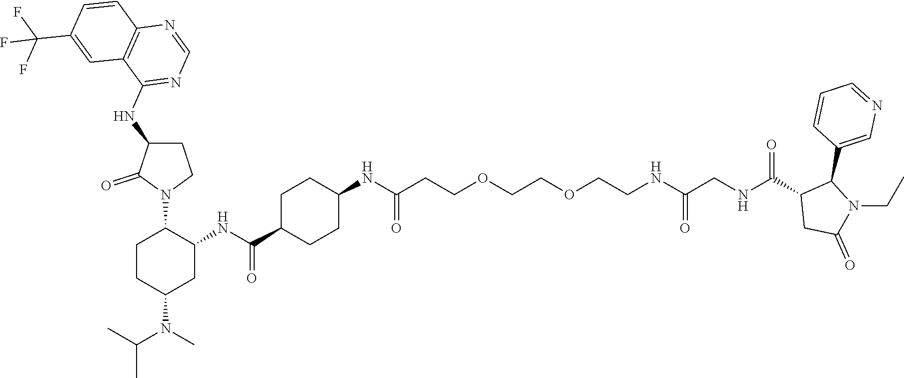 [SMILES: CCN1C(=O)C[C@H](C(=O)NCC(=O)NCCOCCOCCC(=O)N[C@H]2CC[C@@H](C(=O)N[C@@H]3C[C@H](N(C)C(C)C)CC[C@@H]3N3CC[C@H](Nc4ncnc5ccc(C(F)(F)F)cc45)C3=O)CC2)[C@H]1c1cccnc1] |
| A65 | 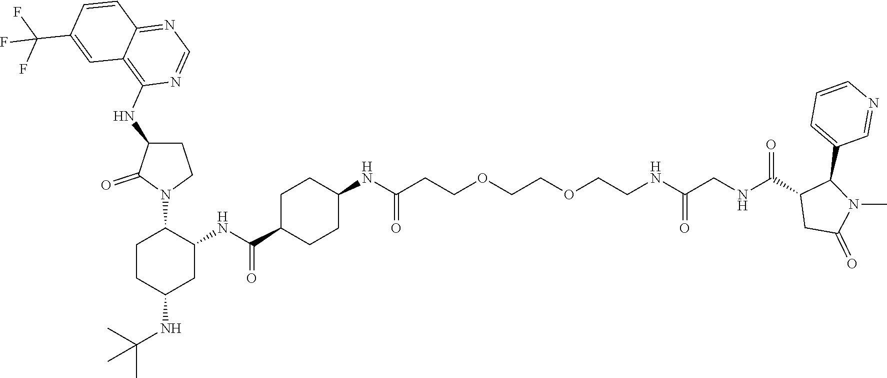 [SMILES: CN1C(=O)C[C@H](C(=O)NCC(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A66 | 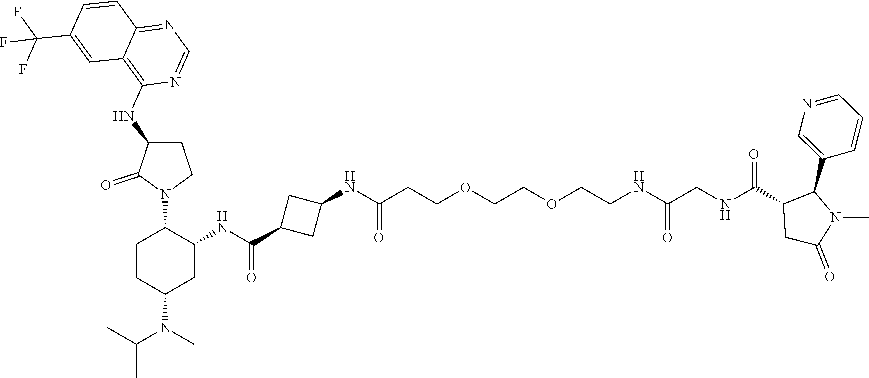 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCOCCNC(=O)CNC(=O)[C@H]3CC(=O)N(C)[C@@H]3c3cccnc3)C2)C1] |
| A67 | 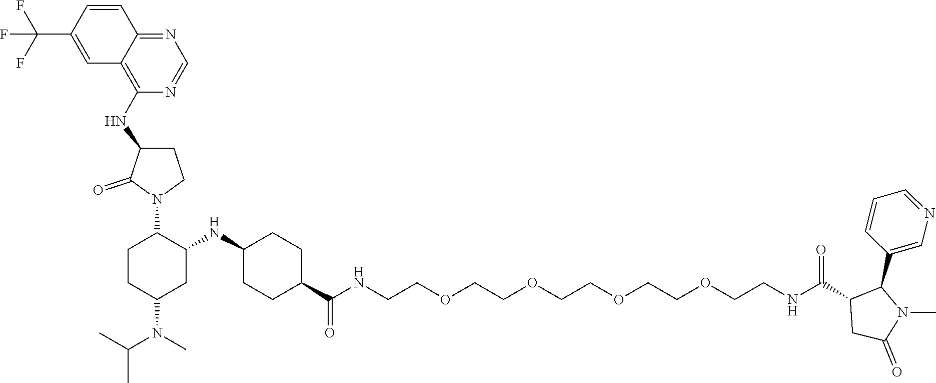 |
| A68 | 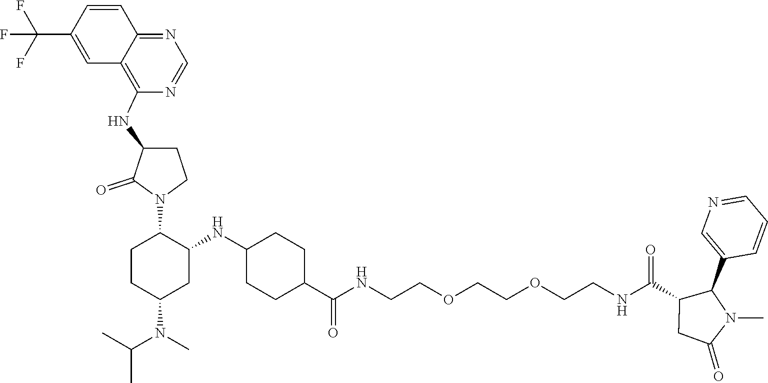 [SMILES: CC(C)N(C)[C@@H]1CC[C@H](N2CC[C@H](Nc3ncnc4ccc(C(F)(F)F)cc34)C2=O)[C@H](NC2CCC(C(=O)NCCOCCOCCNC(=O)[C@H]3CC(=O)N(C)[C@@H]3c3cccnc3)CC2)C1] |
| A69 | 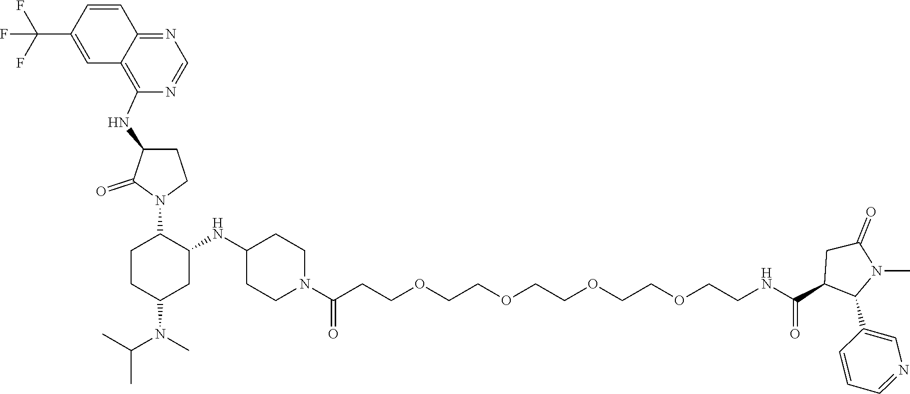 |
| A70 | 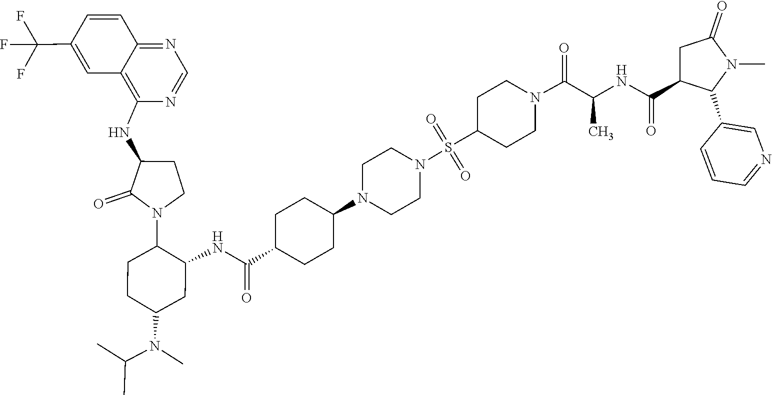 |
| A71 | 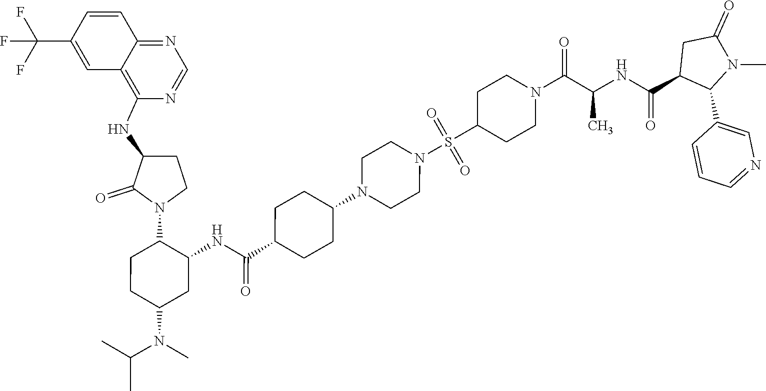 |
| A72 | 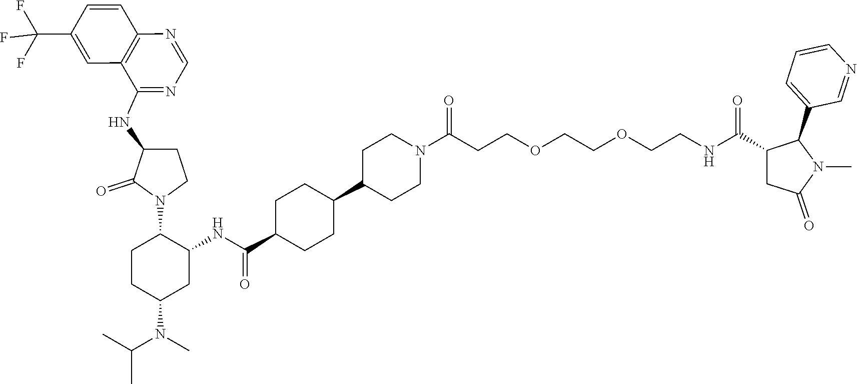 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](C3CCN(C(=O)CCOCCOCCNC(=O)[C@H]4CC(=O)N(C)[C@@H]4c4cccnc4)CC3)CC2)C1] |
| A73 | 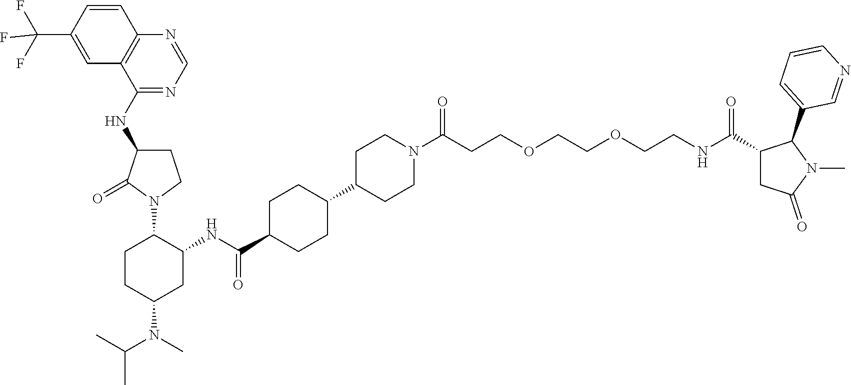 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](C3CCN(C(=O)CCOCCOCCNC(=O)[C@H]4CC(=O)N(C)[C@@H]4c4cccnc4)CC3)CC2)C1] |
| A74 | 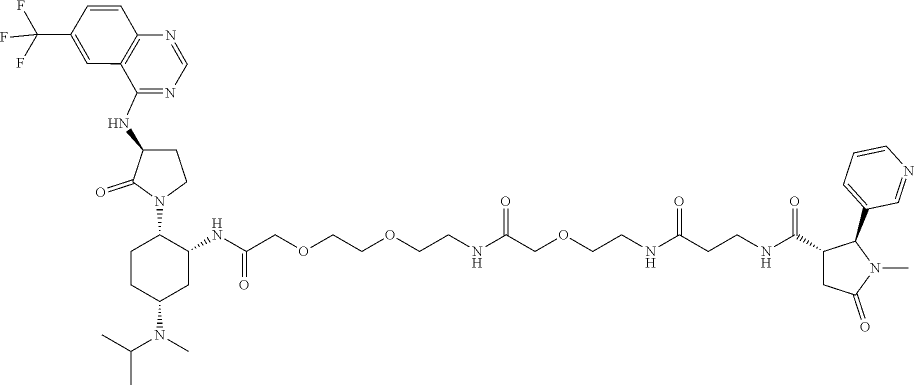 [SMILES: CC(C)N(C)[C@@H]1CC[C@H](N2CC[C@H](Nc3ncnc4ccc(C(F)(F)F)cc34)C2=O)[C@H](NC(=O)COCCOCCNC(=O)COCCNC(=O)CCNC(=O)[C@H]2CC(=O)N(C)[C@@H]2c2cccnc2)C1] |
| A75 | 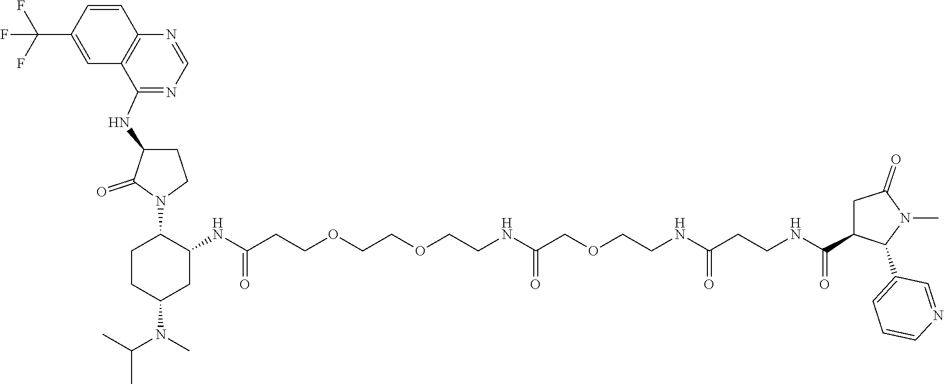 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)COCCNC(=O)CCNC(=O)[C@H]2CC(=O)N(C)[C@@H]2c2cccnc2)C1] |
| A76 | 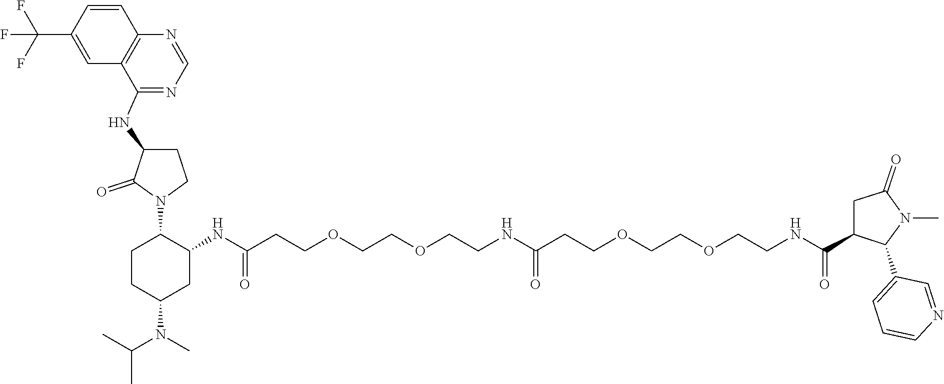 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)CCOCCOCCNC(=O)[C@H]2CC(=O)N(C)[C@@H]2c2cccnc2)C1] |
| A77 | 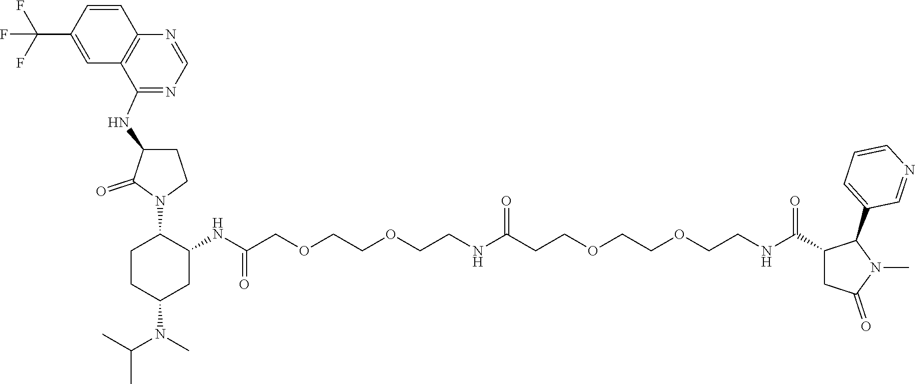 [SMILES: CC(C)N(C)[C@@H]1CC[C@H](N2CC[C@H](Nc3ncnc4ccc(C(F)(F)F)cc34)C2=O)[C@H](NC(=O)COCCOCCNC(=O)CCOCCOCCNC(=O)[C@H]2CC(=O)N(C)[C@@H]2c2cccnc2)C1] |
| A78 | 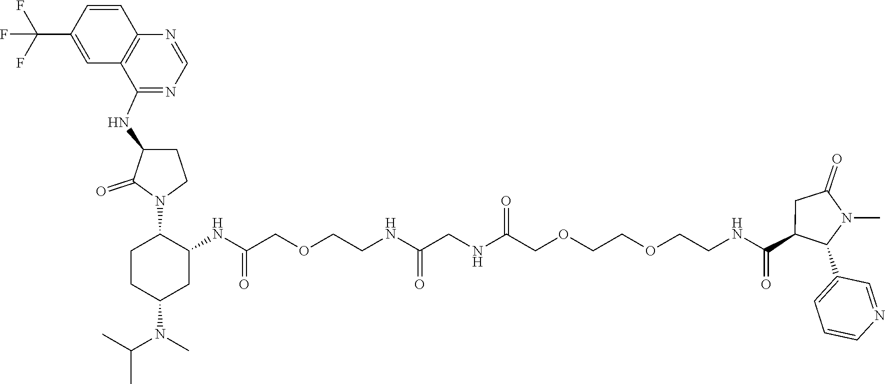 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CNC(=O)COCCOCCNC(=O)[C@H]2CC(=O)N(C)[C@@H]2c2cccnc2)C1] |
| A79 | 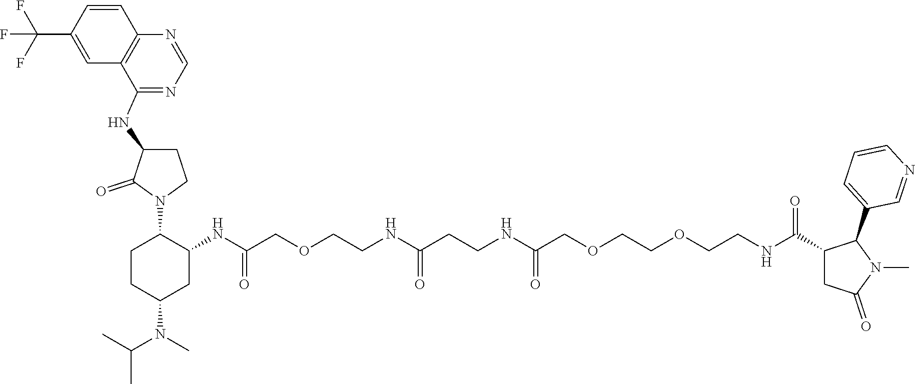 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCNC(=O)COCCOCCNC(=O)[C@H]2CC(=O)N(C)[C@@H]2c2cccnc2)C1] |
| A80 | 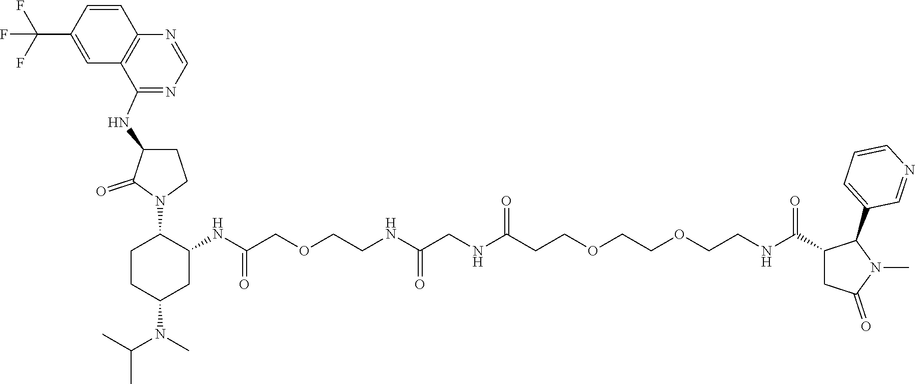 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CNC(=O)CCOCCOCCNC(=O)[C@H]2CC(=O)N(C)[C@@H]2c2cccnc2)C1] |
| A81 | 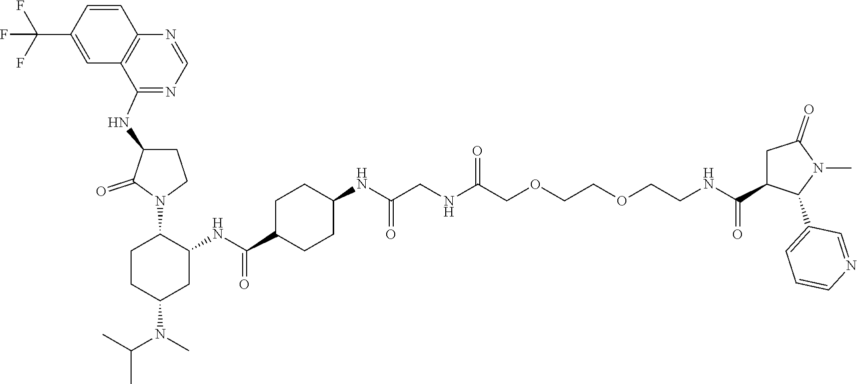 |
| A82 | 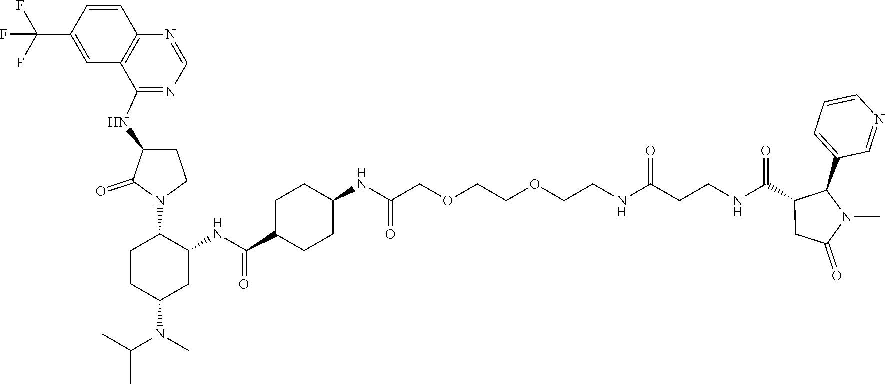 |
| A83 | 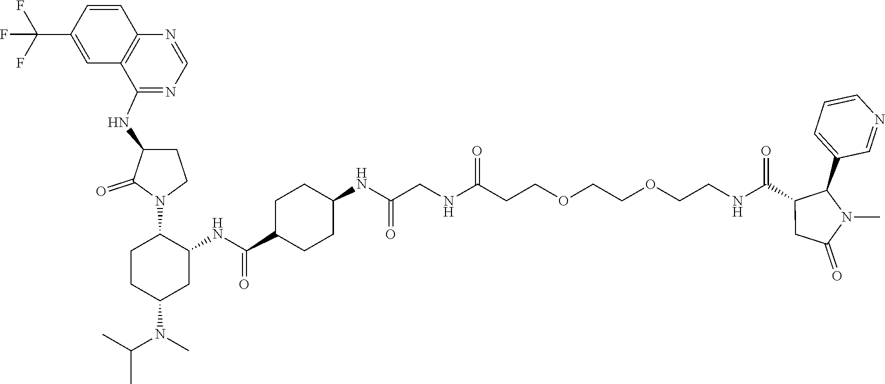 |
| A84 | 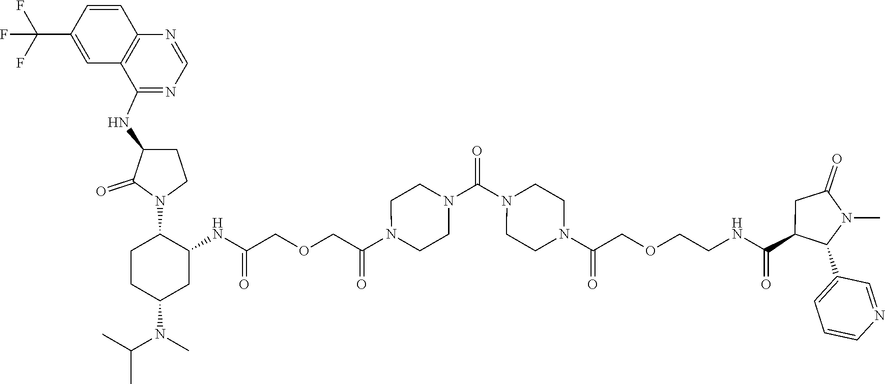 [SMILES: CC(C)N(C)[C@@H]1CC[C@H](N2CC[C@H](Nc3ncnc4ccc(C(F)(F)F)cc34)C2=O)[C@H](NC(=O)COCC(=O)N2CCN(C(=O)N3CCN(C(=O)COCCNC(=O)[C@H]4CC(=O)N(C)[C@@H]4c4cccnc4)CC3)CC2)C1] |
| A85 | 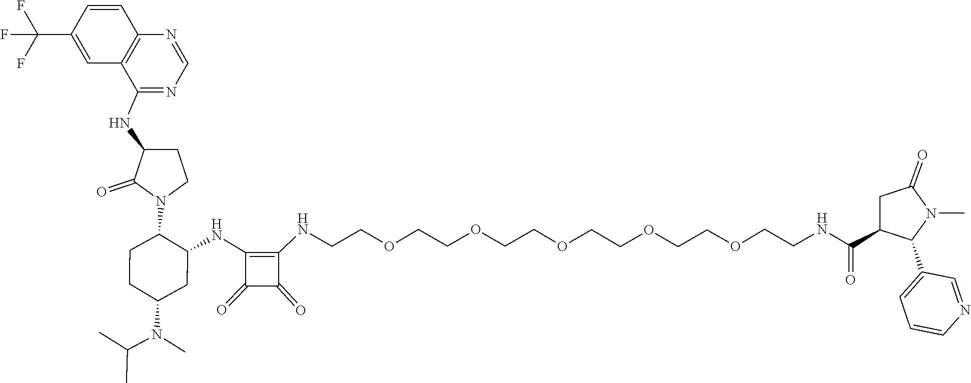 [SMILES: CC(C)N(C)[C@@H]1CC[C@H](N2CC[C@H](Nc3ncnc4ccc(C(F)(F)F)cc34)C2=O)[C@H](Nc2c(NCCOCCOCCOCCOCCOCCNC(=O)[C@H]3CC(=O)N(C)[C@@H]3c3cccnc3)c(=O)c2=O)C1] |
| A86 | 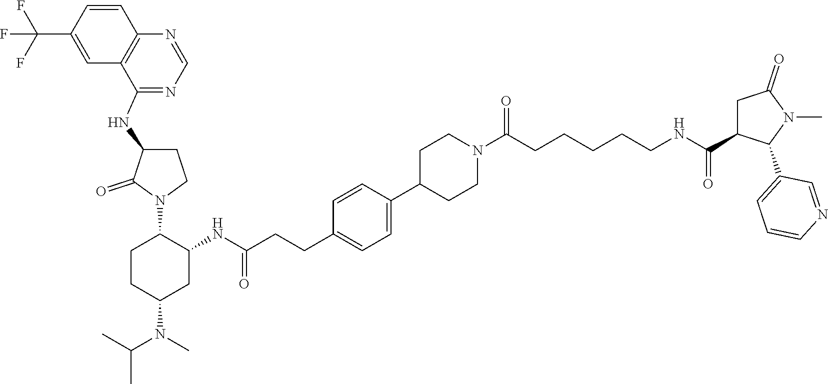 [SMILES: CC(C)N(C)[C@@H]1CC[C@H](N2CC[C@H](Nc3ncnc4ccc(C(F)(F)F)cc34)C2=O)[C@H](NC(=O)CCc2ccc(C3CCN(C(=O)CCCCCNC(=O)[C@H]4CC(=O)N(C)[C@@H]4c4cccnc4)CC3)cc2)C1] |
| A87 | 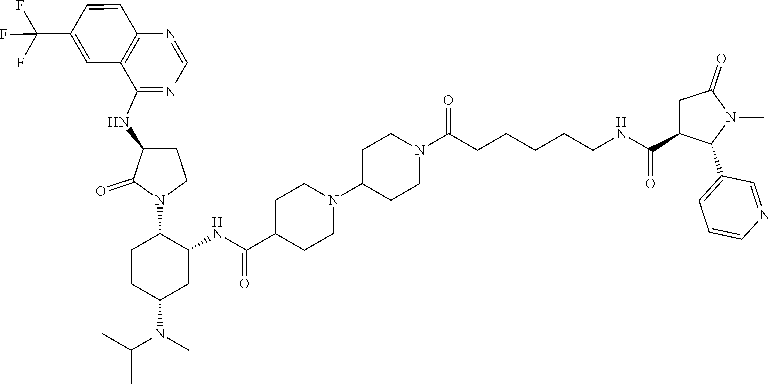 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCCCCNC(=O)[C@H]4CC(=O)N(C)[C@@H]4c4cccnc4)CC3)CC2)C1] |
| A88 | 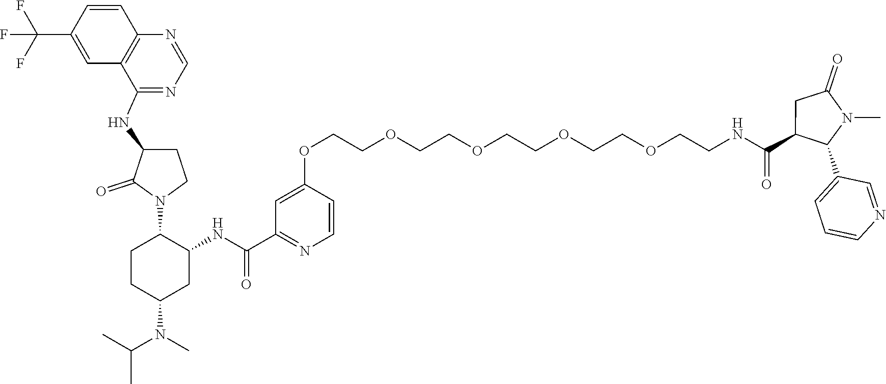 [SMILES: CC(C)N(C)[C@@H]1CC[C@H](N2CC[C@H](Nc3ncnc4ccc(C(F)(F)F)cc34)C2=O)[C@H](NC(=O)c2cc(OCCOCCOCCOCCOCCNC(=O)[C@H]3CC(=O)N(C)[C@@H]3c3cccnc3)ccn2)C1] |
| A89 | 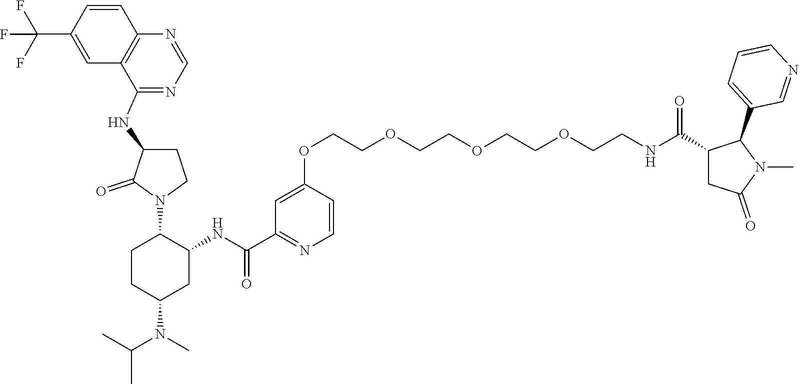 [SMILES: CC(C)N(C)[C@@H]1CC[C@H](N2CC[C@H](Nc3ncnc4ccc(C(F)(F)F)cc34)C2=O)[C@H](NC(=O)c2cc(OCCOCCOCCOCCNC(=O)[C@H]3CC(=O)N(C)[C@@H]3c3cccnc3)ccn2)C1] |
| A90 | 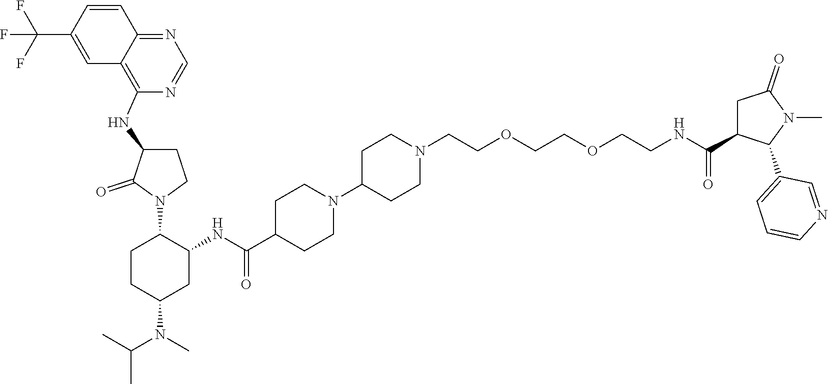 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(CCOCCOCCNC(=O)[C@H]4CC(=O)N(C)[C@@H]4c4cccnc4)CC3)CC2)C1] |
| A91 | 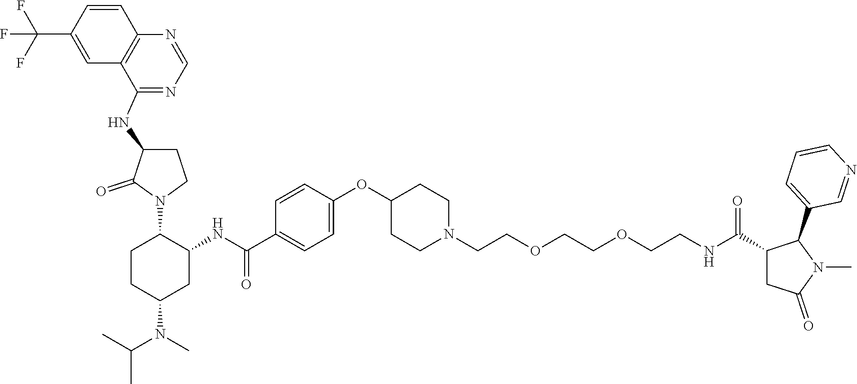 [SMILES: CC(C)N(C)[C@@H]1CC[C@H](N2CC[C@H](Nc3ncnc4ccc(C(F)(F)F)cc34)C2=O)[C@H](NC(=O)c2ccc(OC3CCN(CCOCCOCCNC(=O)[C@H]4CC(=O)N(C)[C@@H]4c4cccnc4)CC3)cc2)C1] |
| A92 | 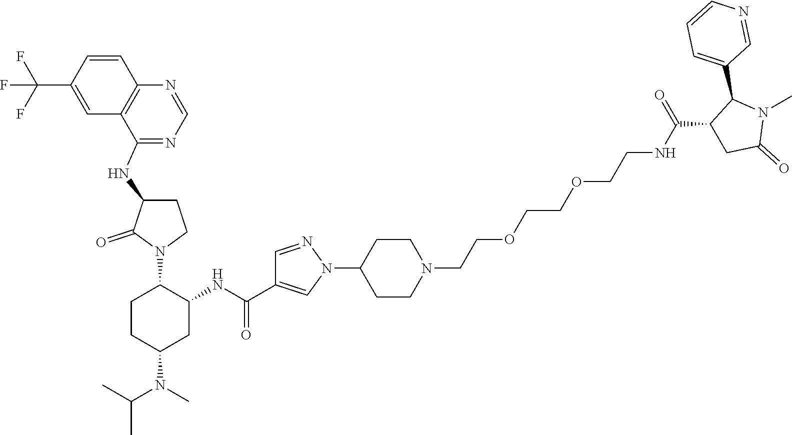 [SMILES: CC(C)N(C)[C@@H]1CC[C@H](N2CC[C@H](Nc3ncnc4ccc(C(F)(F)F)cc34)C2=O)[C@H](NC(=O)c2cnn(C3CCN(CCOCCOCCNC(=O)[C@H]4CC(=O)N(C)[C@@H]4c4cccnc4)CC3)c2)C1] |
| A93 | 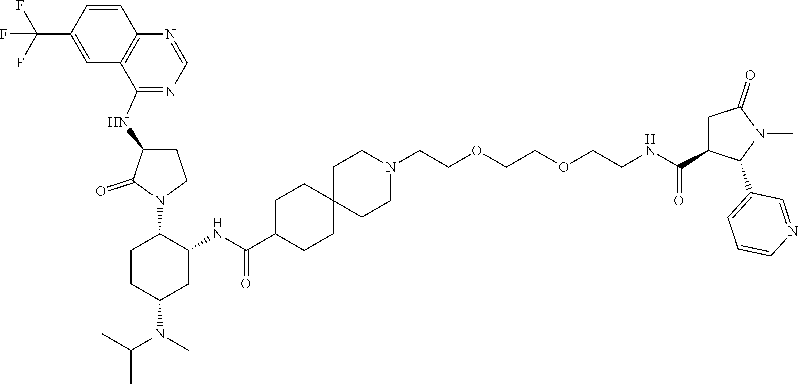 [SMILES: CC(C)N(C)[C@@H]1CC[C@H](N2CC[C@H](Nc3ncnc4ccc(C(F)(F)F)cc34)C2=O)[C@H](NC(=O)C2CCC3(CC2)CCN(CCOCCOCCNC(=O)[C@H]2CC(=O)N(C)[C@@H]2c2cccnc2)CC3)C1] |
| A94 | 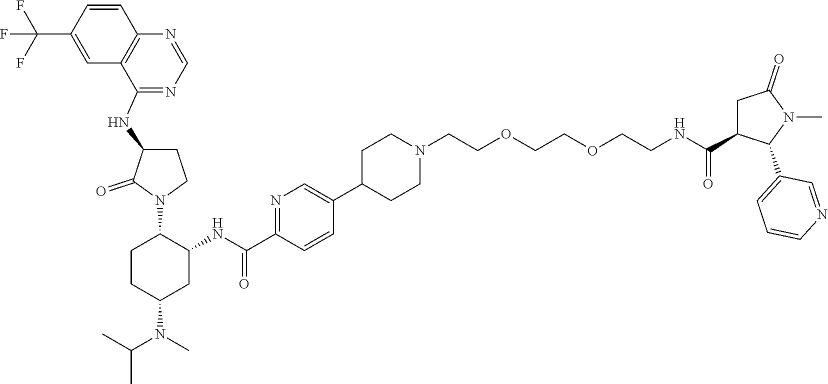 [SMILES: CC(C)N(C)[C@@H]1CC[C@H](N2CC[C@H](Nc3ncnc4ccc(C(F)(F)F)cc34)C2=O)[C@H](NC(=O)c2ccc(C3CCN(CCOCCOCCNC(=O)[C@H]4CC(=O)N(C)[C@@H]4c4cccnc4)CC3)cn2)C1] |
| A95 | 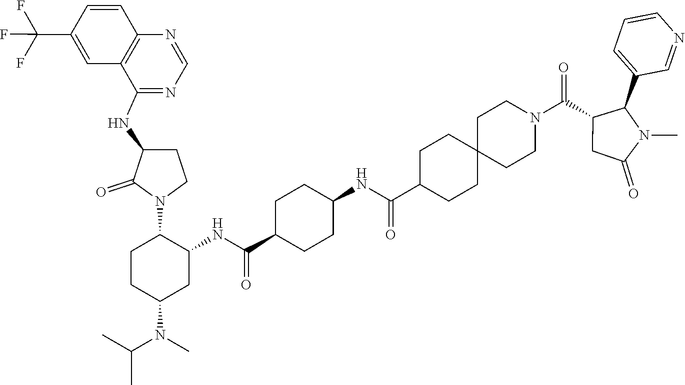 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)C3CCC4(CC3)CCN(C(=O)[C@H]3CC(=O)N(C)[C@@H]3c3cccnc3)CC4)CC2)C1] |
| A96 | 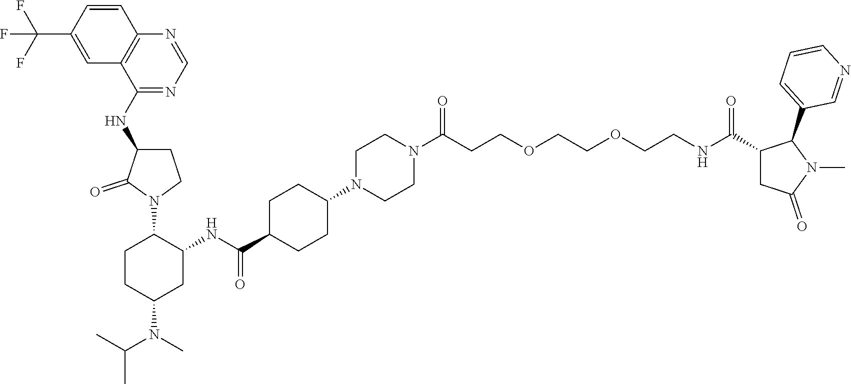 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C(=O)CCOCCOCCNC(=O)[C@H]4CC(=O)N(C)[C@@H]4c4cccnc4)CC3)CC2)C1] |
| A97 | 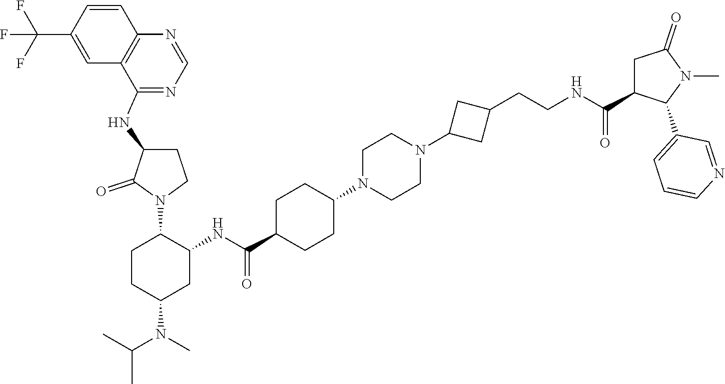 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C4CC(CCNC(=O)[C@H]5CC(=O)N(C)[C@@H]5c5cccnc5)C4)CC3)CC2)C1] |
| A98 | 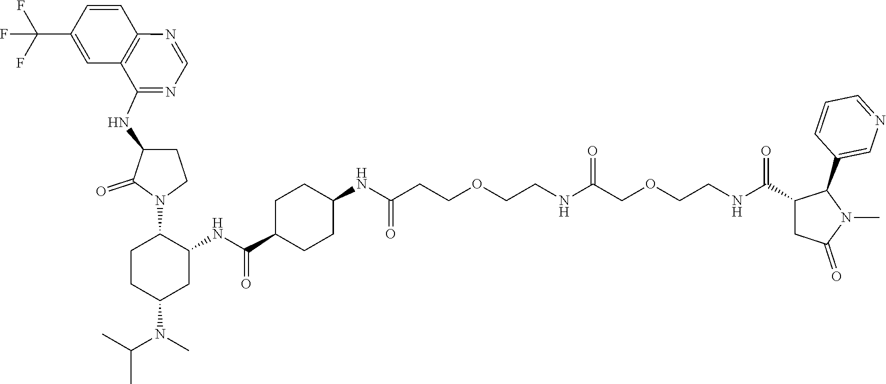 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)COCCNC(=O)[C@H]3CC(=O)N(C)[C@@H]3c3cccnc3)CC2)C1] |
| A99 | 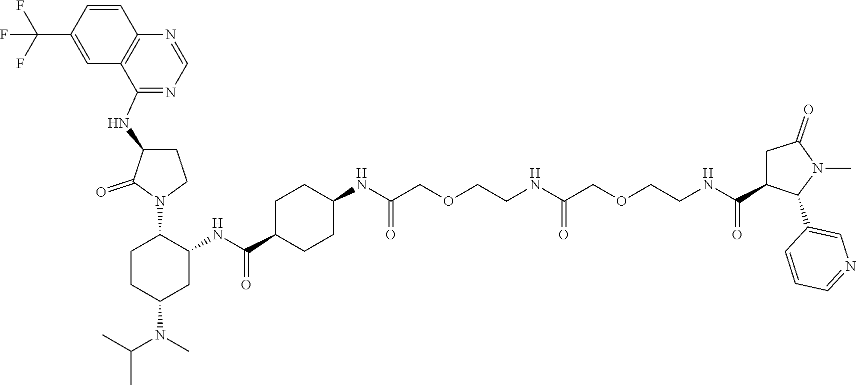 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCNC(=O)COCCNC(=O)[C@H]3CC(=O)N(C)[C@@H]3c3cccnc3)CC2)C1] |
| A100 | 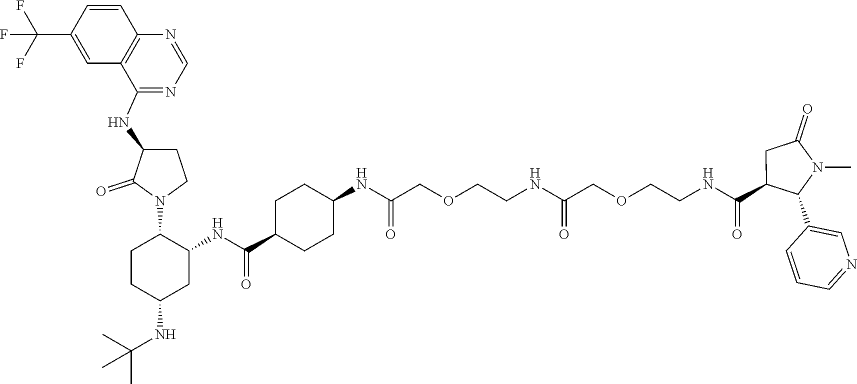 [SMILES: CN1C(=O)C[C@H](C(=O)NCCOCC(=O)NCCOCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A101 | 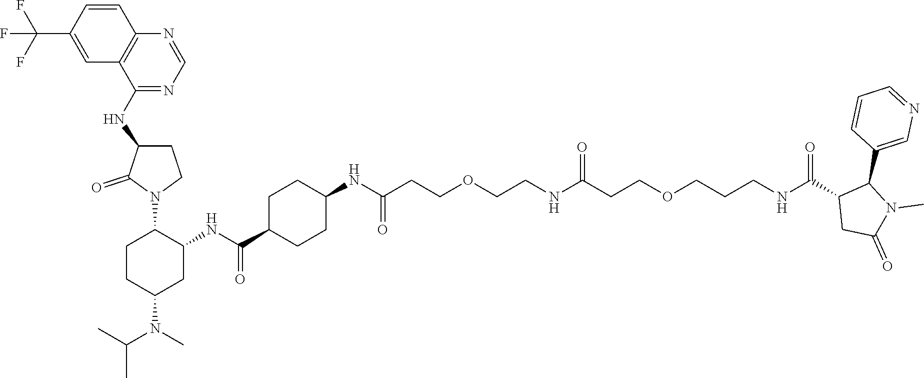 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCOCCCNC(=O)[C@H]3CC(=O)N(C)[C@@H]3c3cccnc3)CC2)C1] |
| A102 | 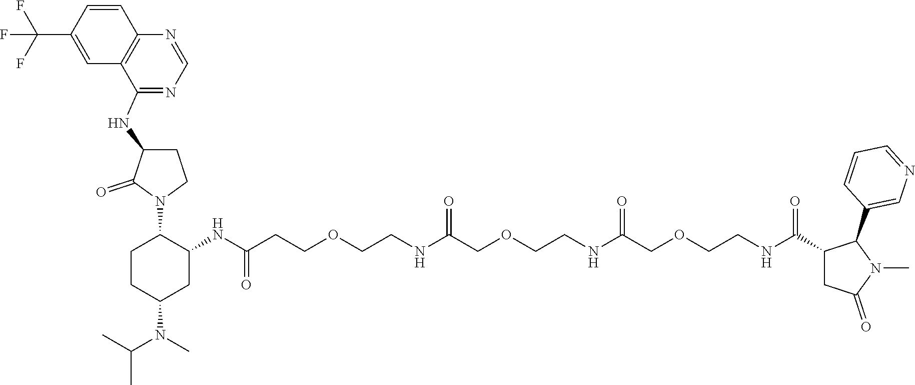 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)COCCNC(=O)COCCNC(=O)[C@H]2CC(=O)N(C)[C@@H]2c2cccnc2)C1] |
| A103 | 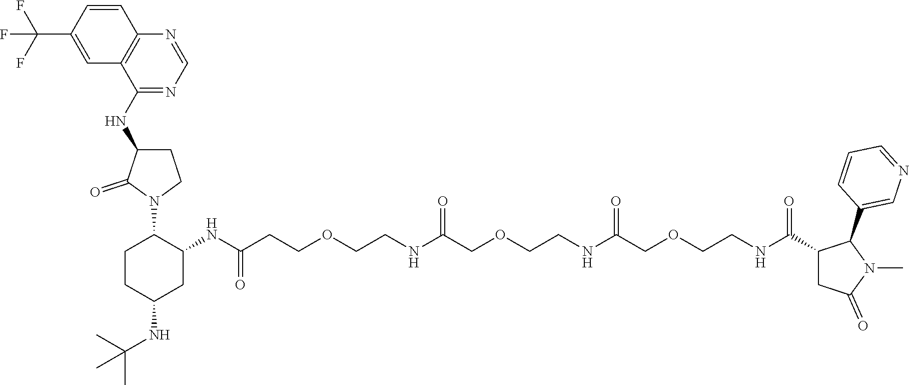 [SMILES: CN1C(=O)C[C@H](C(=O)NCCOCC(=O)NCCOCC(=O)NCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |
| A104 | 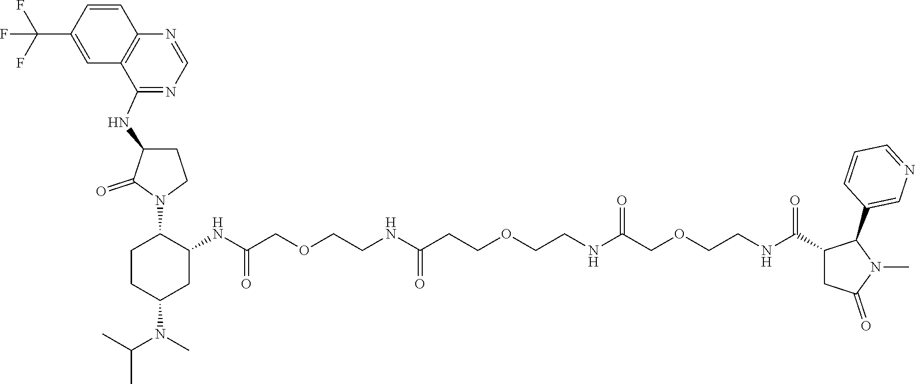 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCOCCNC(=O)COCCNC(=O)[C@H]2CC(=O)N(C)[C@@H]2c2cccnc2)C1] |
| A105 | 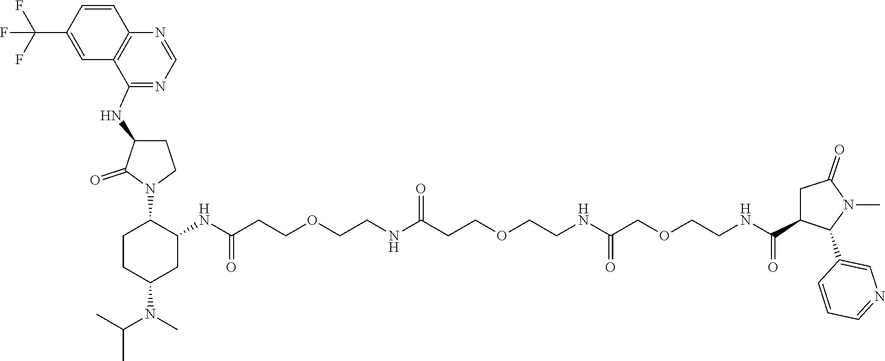 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCOCCNC(=O)COCCNC(=O)[C@H]2CC(=O)N(C)[C@@H]2c2cccnc2)C1] |
| A106 | 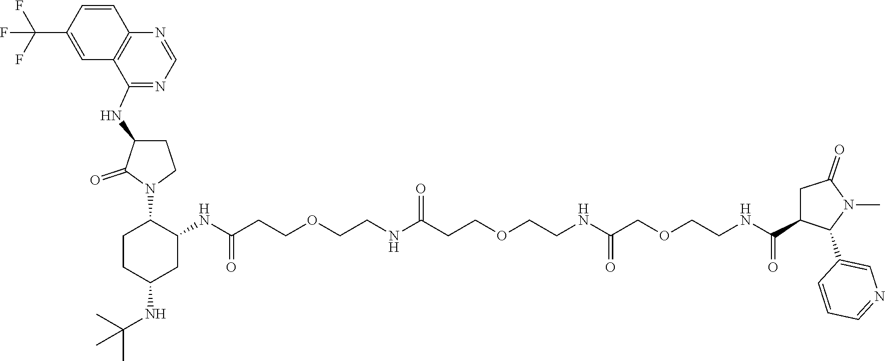 [SMILES: CN1C(=O)C[C@H](C(=O)NCCOCC(=O)NCCOCCC(=O)NCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |
| A107 | 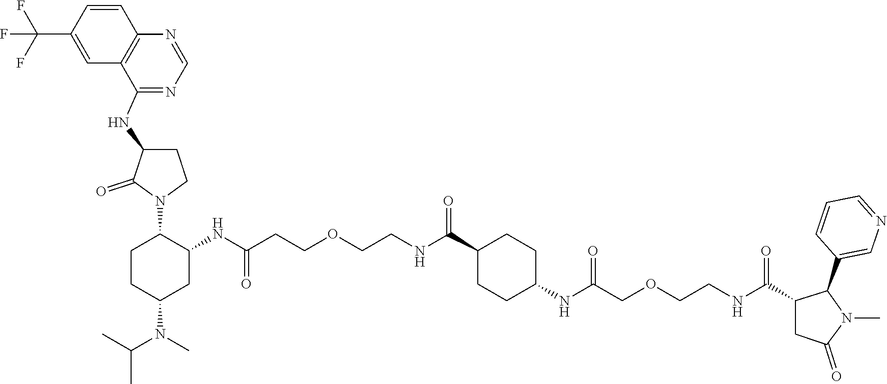 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)[C@H]2CC[C@H](NC(=O)COCCNC(=O)[C@H]3CC(=O)N(C)[C@@H]3c3cccnc3)CC2)C1] |
| A108 | 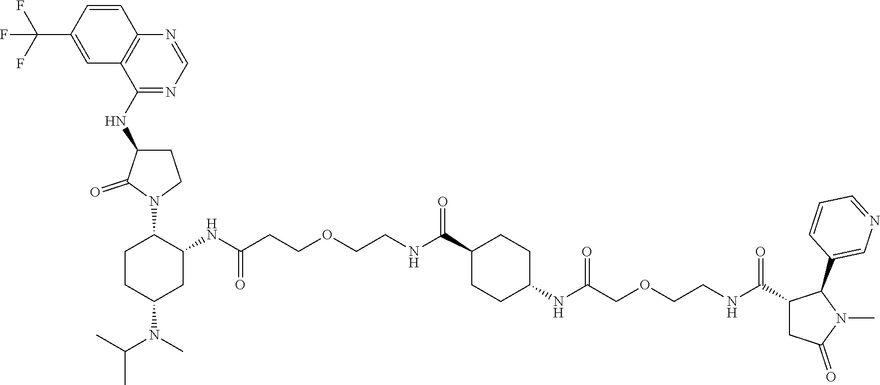 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)[C@H]2CC[C@H](NC(=O)COCCNC(=O)[C@H]3CC(=O)N(C)[C@@H]3c3cccnc3)CC2)C1] |
| A109 | 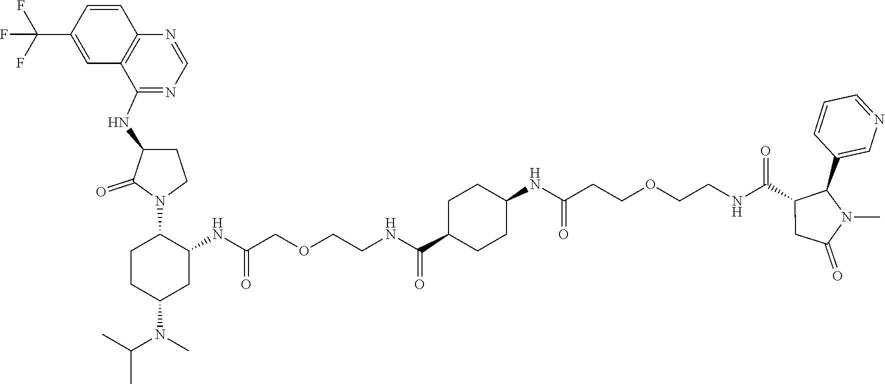 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)[C@H]2CC[C@@H](NC(=O)CCOCCNC(=O)[C@H]3CC(=O)N(C)[C@@H]3c3cccnc3)CC2)C1] |
| A110 | 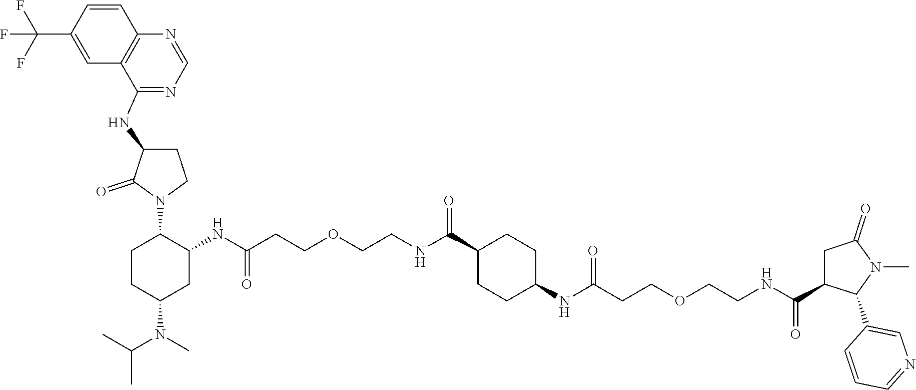 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)[C@H]2CC[C@@H](NC(=O)CCOCCNC(=O)[C@H]3CC(=O)N(C)[C@@H]3c3cccnc3)CC2)C1] |
| A111 | 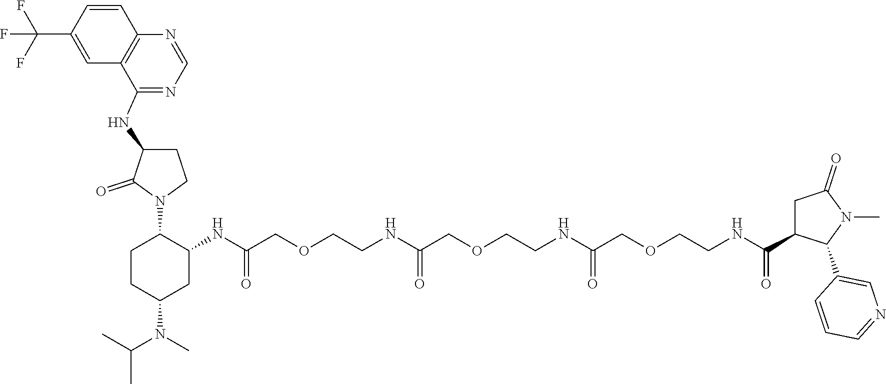 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)COCCNC(=O)COCCNC(=O)[C@H]2CC(=O)N(C)[C@@H]2c2cccnc2)C1] |
| A112 | 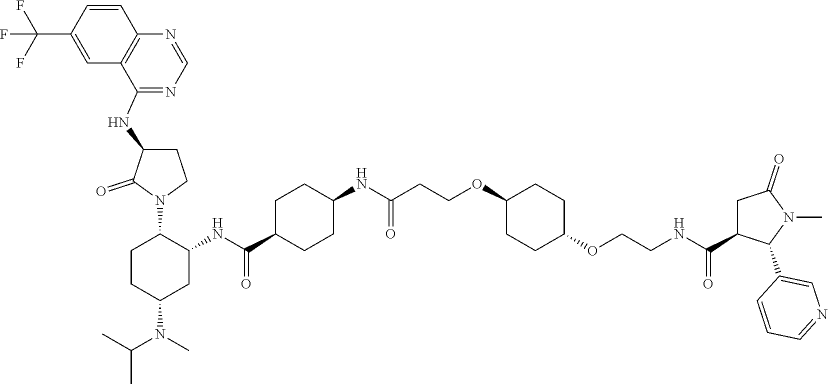 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCO[C@H]3CC[C@H](OCCNC(=O)[C@H]4CC(=O)N(C)[C@@H]4c4cccnc4)CC3)CC2)C1] |
| A113 | 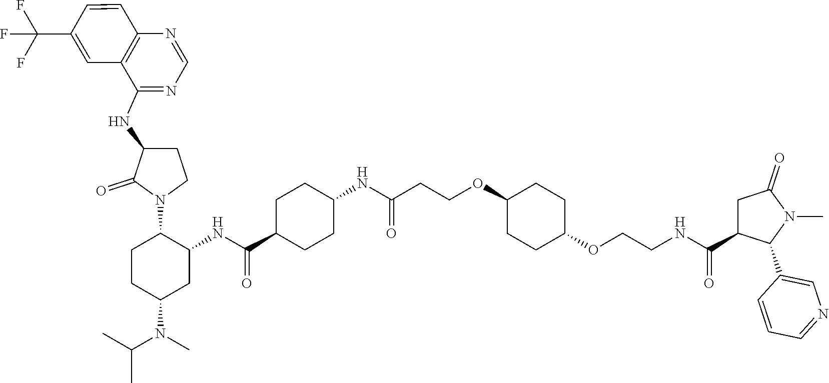 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCO[C@H]3CC[C@H](OCCNC(=O)[C@H]4CC(=O)N(C)[C@@H]4c4cccnc4)CC3)CC2)C1] |
| A114 | 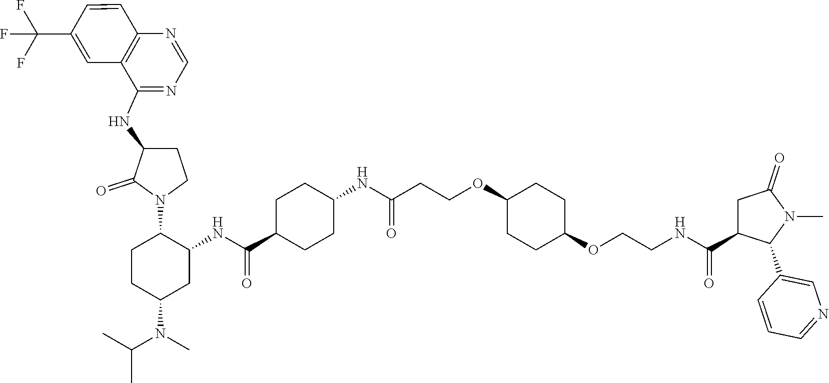 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCO[C@H]3CC[C@@H](OCCNC(=O)[C@H]4CC(=O)N(C)[C@@H]4c4cccnc4)CC3)CC2)C1] |
| A115 | 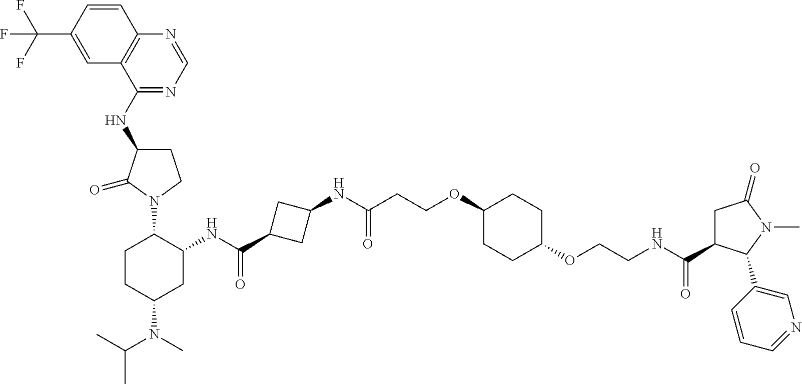 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCO[C@H]3CC[C@H](OCCNC(=O)[C@H]4CC(=O)N(C)[C@@H]4c4cccnc4)CC3)C2)C1] |
| A116 | 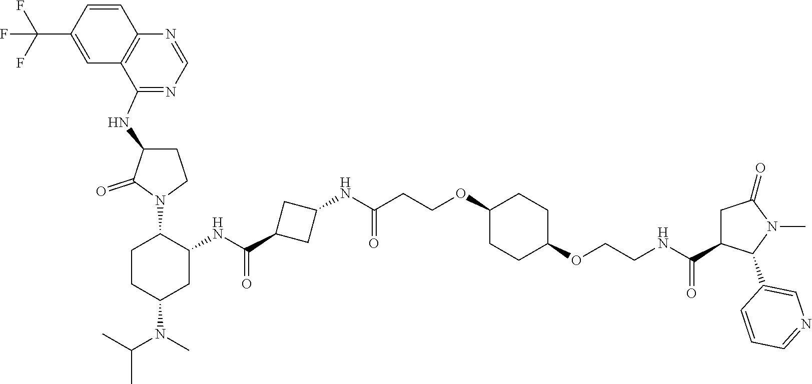 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCO[C@H]3CC[C@@H](OCCNC(=O)[C@H]4CC(=O)N(C)[C@@H]4c4cccnc4)CC3)C2)C1] |
| A117 | 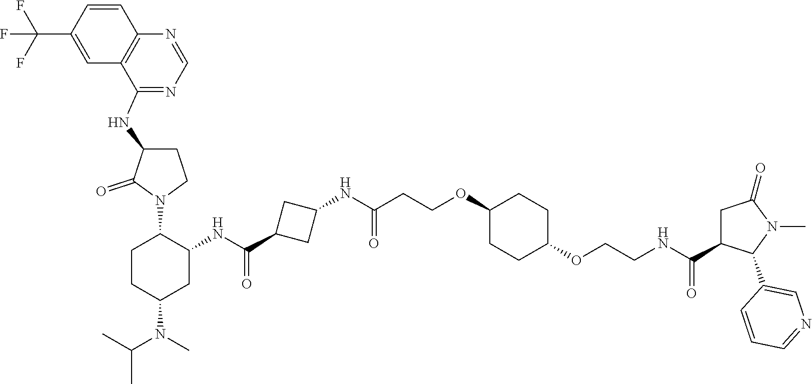 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCO[C@H]3CC[C@H](OCCNC(=O)[C@H]4CC(=O)N(C)[C@@H]4c4cccnc4)CC3)C2)C1] |
| A118 | 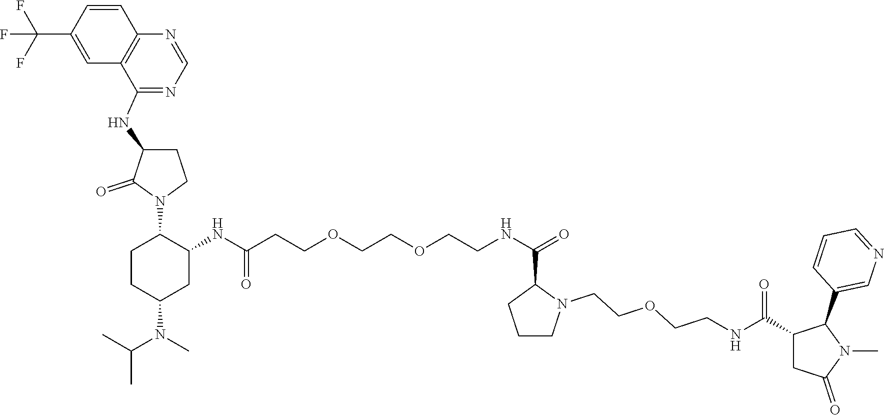 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)[C@@H]2CCCN2CCOCCNC(=O)[C@H]2CC(=O)N(C)[C@@H]2c2cccnc2)C1] |
| A119 | 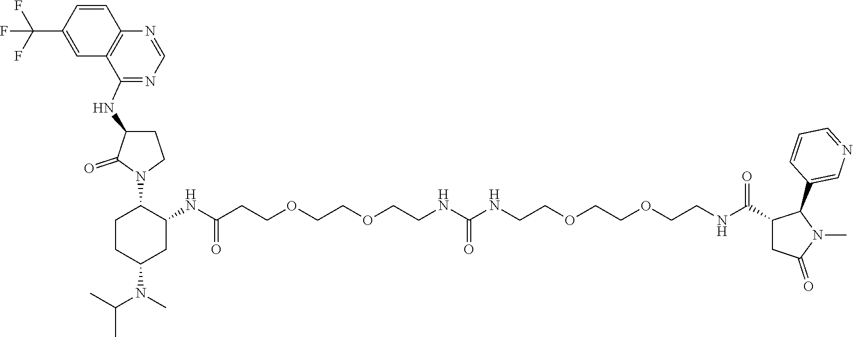 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)NCCOCCOCCNC(=O)[C@H]2CC(=O)N(C)[C@@H]2c2cccnc2)C1] |
| A120 | 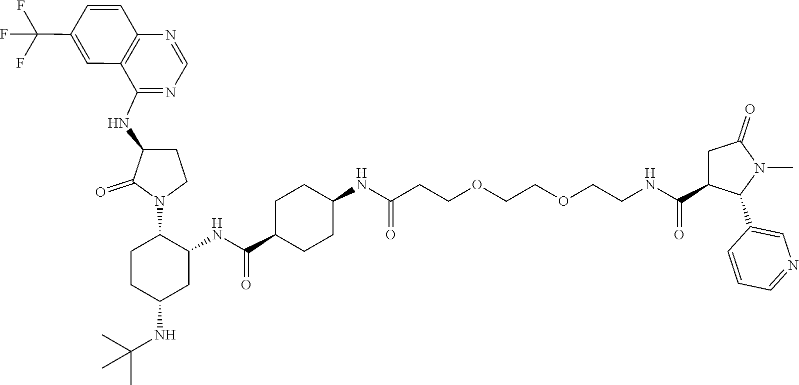 [SMILES: CN1C(=O)C[C@H](C(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A121 | 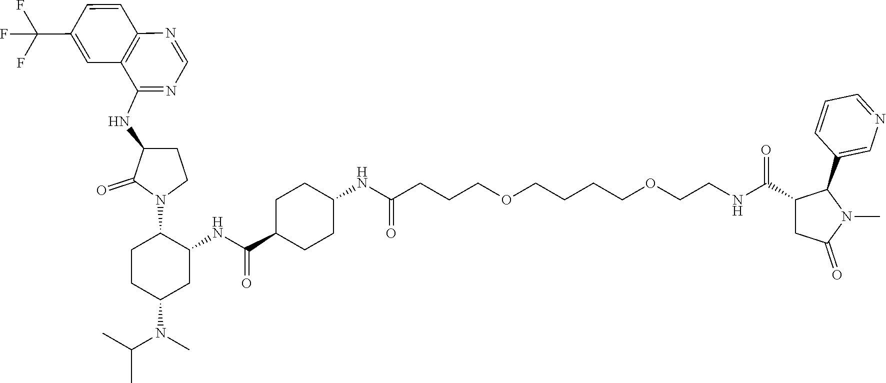 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCOCCCCOCCNC(=O)[C@H]3CC(=O)N(C)[C@@H]3c3cccnc3)CC2)C1] |
| A122 | 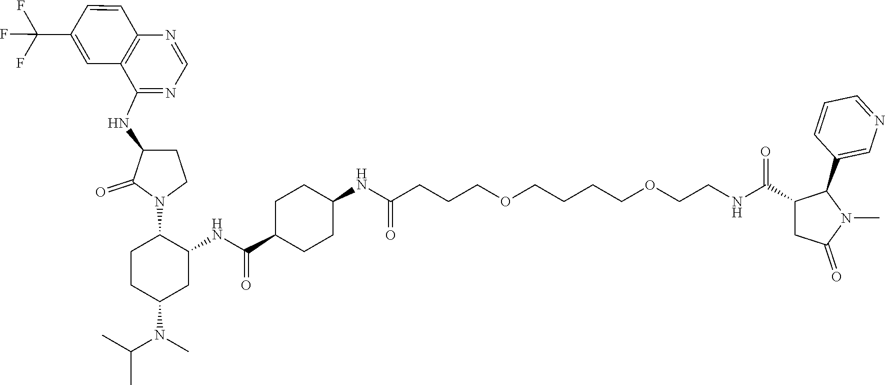 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCOCCCCOCCNC(=O)[C@H]3CC(=O)N(C)[C@@H]3c3cccnc3)CC2)C1] |
| A123 | 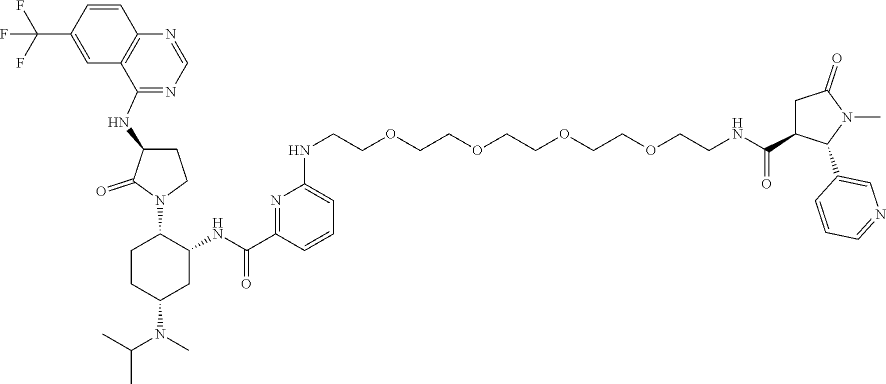 [SMILES: CC(C)N(C)[C@@H]1CC[C@H](N2CC[C@H](Nc3ncnc4ccc(C(F)(F)F)cc34)C2=O)[C@H](NC(=O)c2cccc(NCCOCCOCCOCCOCCNC(=O)[C@H]3CC(=O)N(C)[C@@H]3c3cccnc3)n2)C1] |
| A124 | 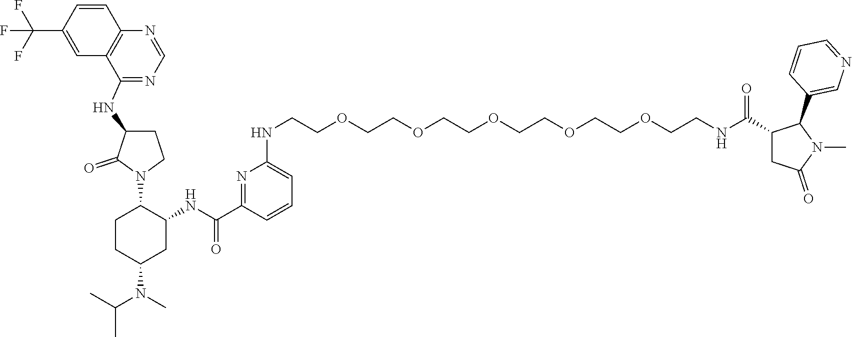 [SMILES: CC(C)N(C)[C@@H]1CC[C@H](N2CC[C@H](Nc3ncnc4ccc(C(F)(F)F)cc34)C2=O)[C@H](NC(=O)c2cccc(NCCOCCOCCOCCOCCOCCNC(=O)[C@H]3CC(=O)N(C)[C@@H]3c3cccnc3)n2)C1] |
| A125 | 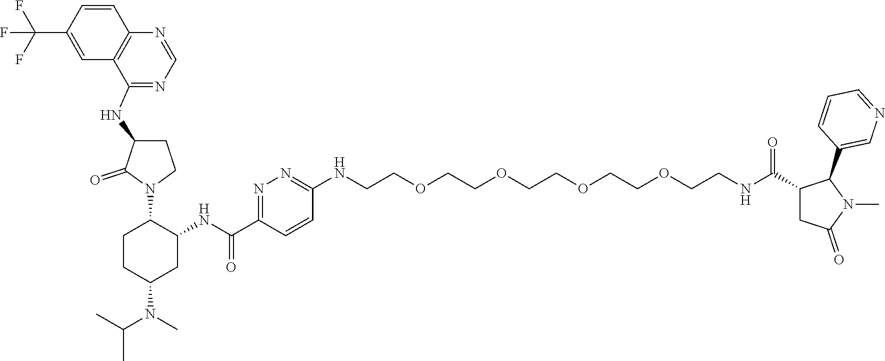 [SMILES: CC(C)N(C)[C@@H]1CC[C@H](N2CC[C@H](Nc3ncnc4ccc(C(F)(F)F)cc34)C2=O)[C@H](NC(=O)c2ccc(NCCOCCOCCOCCOCCNC(=O)[C@H]3CC(=O)N(C)[C@@H]3c3cccnc3)nn2)C1] |
| A126 | 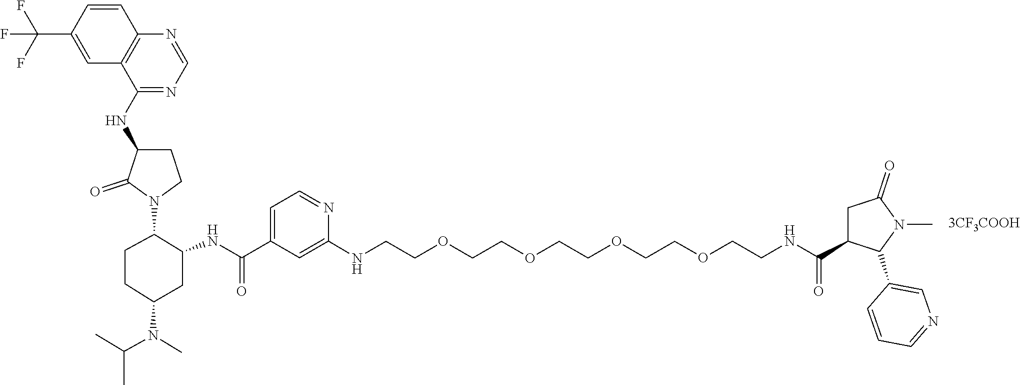 |
| A127 | 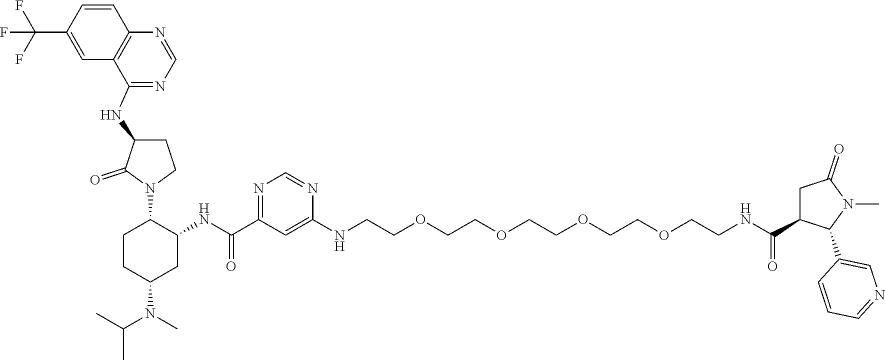 [SMILES: CC(C)N(C)[C@@H]1CC[C@H](N2CC[C@H](Nc3ncnc4ccc(C(F)(F)F)cc34)C2=O)[C@H](NC(=O)c2cc(NCCOCCOCCOCCOCCNC(=O)[C@H]3CC(=O)N(C)[C@@H]3c3cccnc3)ncn2)C1] |
| A128 | 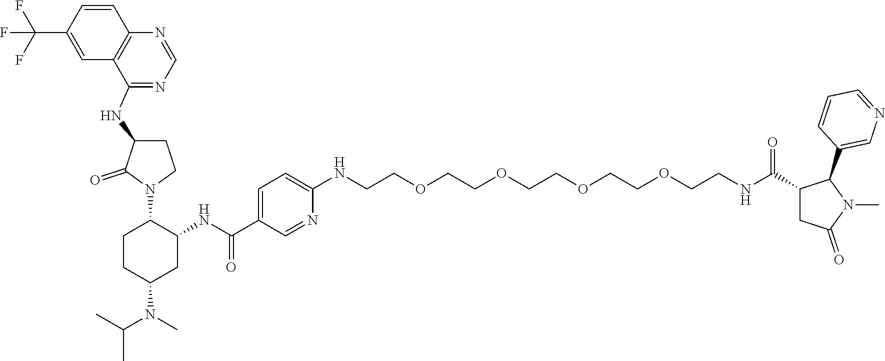 [SMILES: CC(C)N(C)[C@@H]1CC[C@H](N2CC[C@H](Nc3ncnc4ccc(C(F)(F)F)cc34)C2=O)[C@H](NC(=O)c2ccc(NCCOCCOCCOCCOCCNC(=O)[C@H]3CC(=O)N(C)[C@@H]3c3cccnc3)nc2)C1] |
| A129 | 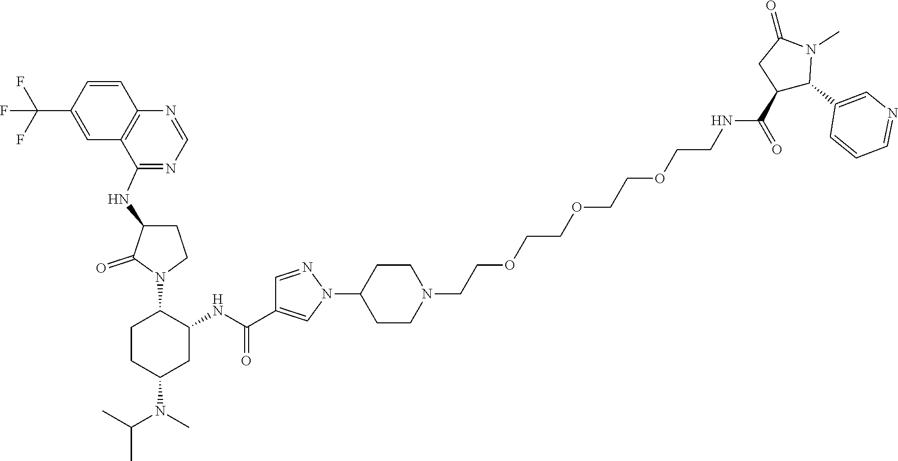 [SMILES: CC(C)N(C)[C@@H]1CC[C@H](N2CC[C@H](Nc3ncnc4ccc(C(F)(F)F)cc34)C2=O)[C@H](NC(=O)c2cnn(C3CCN(CCOCCOCCOCCNC(=O)[C@H]4CC(=O)N(C)[C@@H]4c4cccnc4)CC3)c2)C1] |
| A130 | 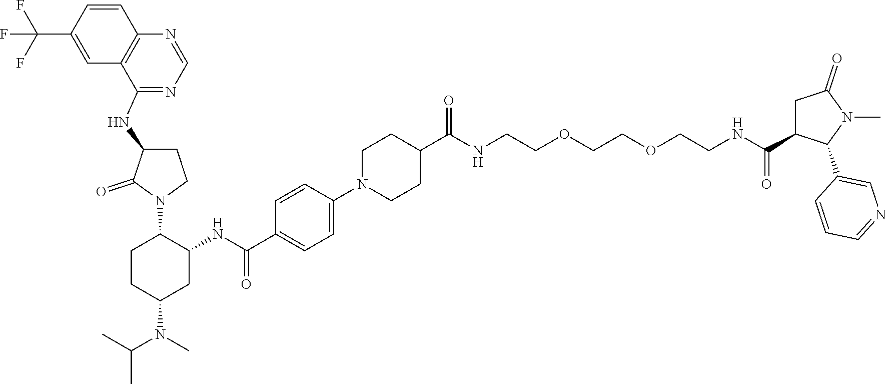 [SMILES: CC(C)N(C)[C@@H]1CC[C@H](N2CC[C@H](Nc3ncnc4ccc(C(F)(F)F)cc34)C2=O)[C@H](NC(=O)c2ccc(N3CCC(C(=O)NCCOCCOCCNC(=O)[C@H]4CC(=O)N(C)[C@@H]4c4cccnc4)CC3)cc2)C1] |
| A131 | 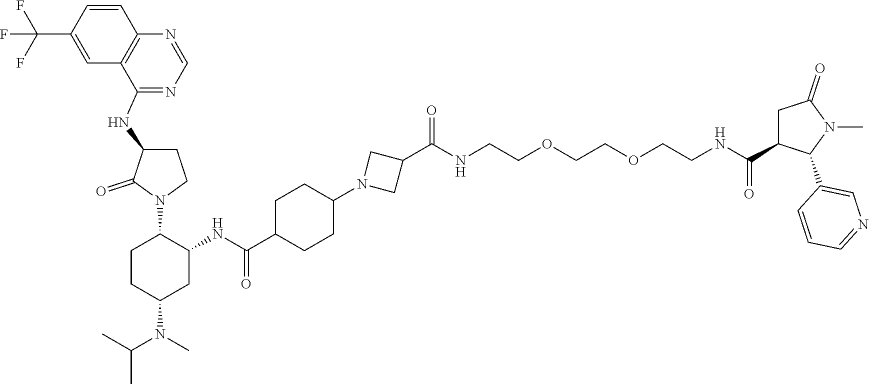 [SMILES: CC(C)N(C)[C@@H]1CC[C@H](N2CC[C@H](Nc3ncnc4ccc(C(F)(F)F)cc34)C2=O)[C@H](NC(=O)C2CCC(N3CC(C(=O)NCCOCCOCCNC(=O)[C@H]4CC(=O)N(C)[C@@H]4c4cccnc4)C3)CC2)C1] |
| A132 | 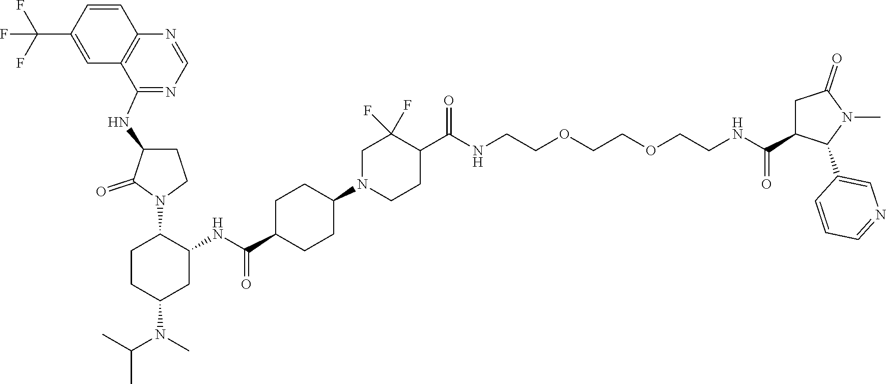 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](N3CCC(C(=O)NCCOCCOCCNC(=O)[C@H]4CC(=O)N(C)[C@@H]4c4cccnc4)C(F)(F)C3)CC2)C1] |
| A133 | 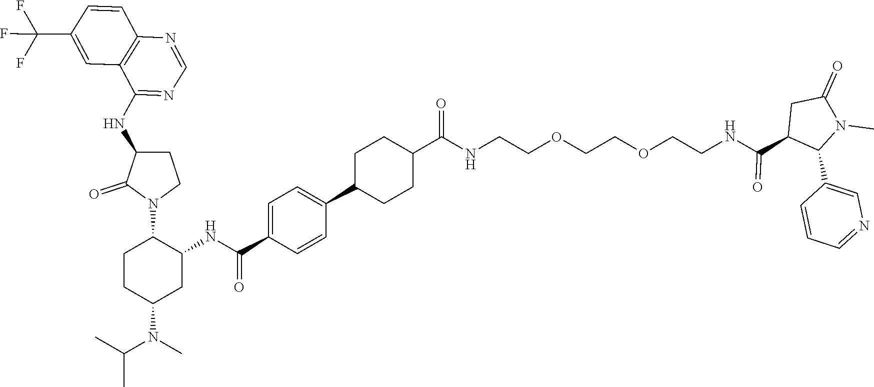 |
| A134 | 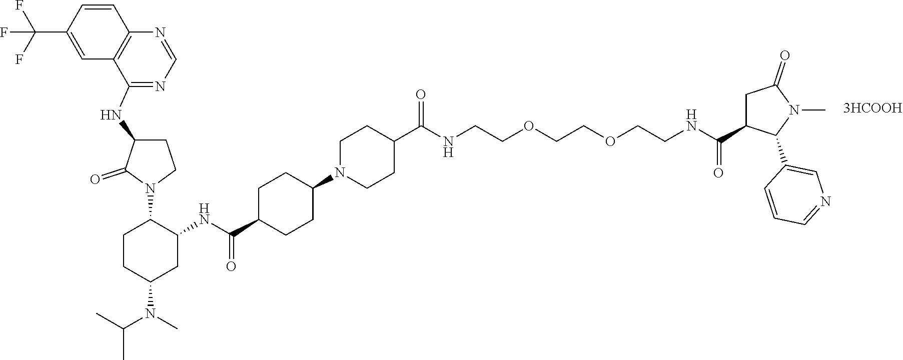 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](N3CCC(C(=O)NCCOCCOCCNC(=O)[C@H]4CC(=O)N(C)[C@@H]4c4cccnc4)CC3)CC2)C1] |
| A135 | 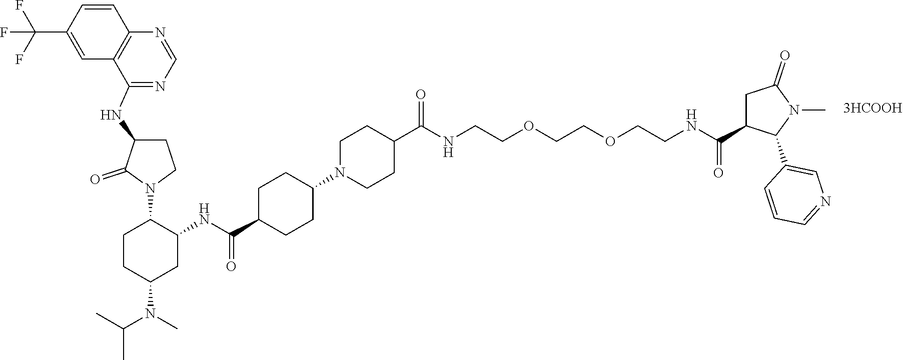 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCC(C(=O)NCCOCCOCCNC(=O)[C@H]4CC(=O)N(C)[C@@H]4c4cccnc4)CC3)CC2)C1] |
| A136 | 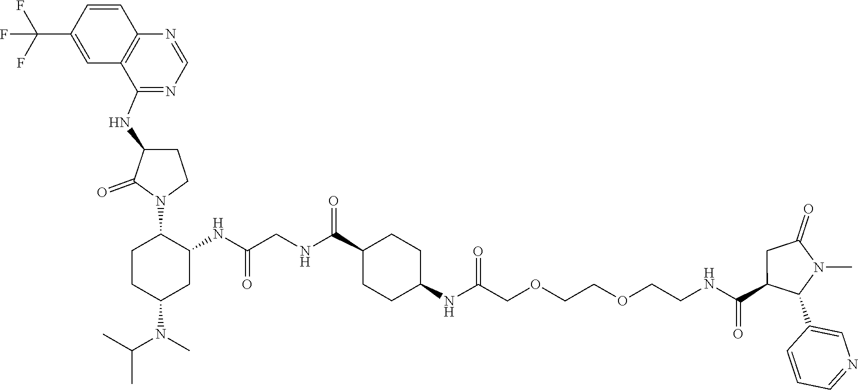 [SMILES: CC(C)N(C)[C@@H]1CC[C@H](N2CC[C@H](Nc3ncnc4ccc(C(F)(F)F)cc34)C2=O)[C@H](NC(=O)CNC(=O)[C@H]2CC[C@@H](NC(=O)COCCOCCNC(=O)[C@H]3CC(=O)N(C)[C@@H]3c3cccnc3)CC2)C1] |
| A137 | 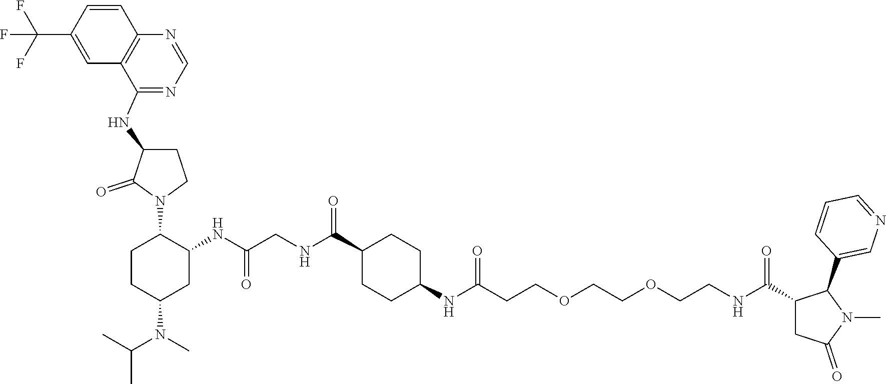 [SMILES: CC(C)N(C)[C@@H]1CC[C@H](N2CC[C@H](Nc3ncnc4ccc(C(F)(F)F)cc34)C2=O)[C@H](NC(=O)CNC(=O)[C@H]2CC[C@@H](NC(=O)CCOCCOCCNC(=O)[C@H]3CC(=O)N(C)[C@@H]3c3cccnc3)CC2)C1] |
| A138 | 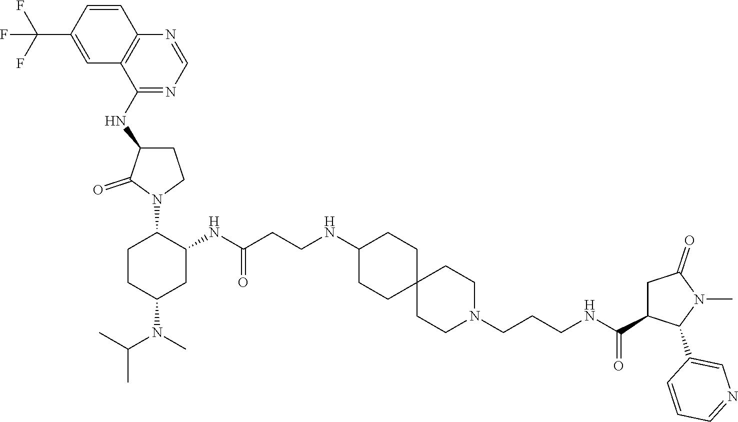 [SMILES: CC(C)N(C)[C@@H]1CC[C@H](N2CC[C@H](Nc3ncnc4ccc(C(F)(F)F)cc34)C2=O)[C@H](NC(=O)CCNC2CCC3(CC2)CCN(CCCNC(=O)[C@H]2CC(=O)N(C)[C@@H]2c2cccnc2)CC3)C1] |
| A139 | 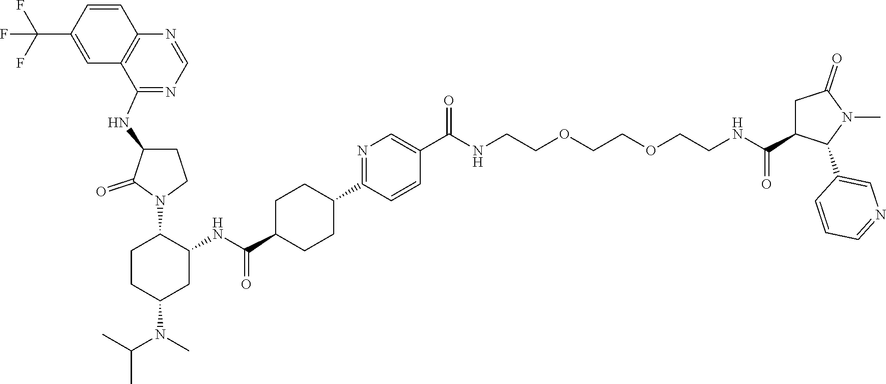 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](c3ccc(C(=O)NCCOCCOCCNC(=O)[C@H]4CC(=O)N(C)[C@@H]4c4cccnc4)cn3)CC2)C1] |
| A140 | 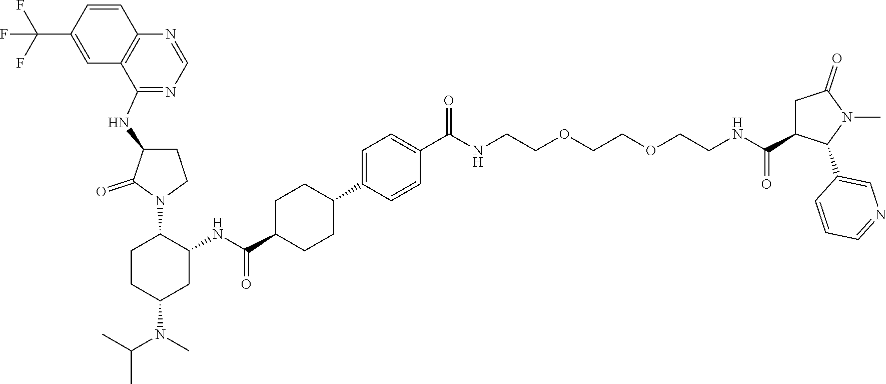 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](c3ccc(C(=O)NCCOCCOCCNC(=O)[C@H]4CC(=O)N(C)[C@@H]4c4cccnc4)cc3)CC2)C1] |
| A141 | 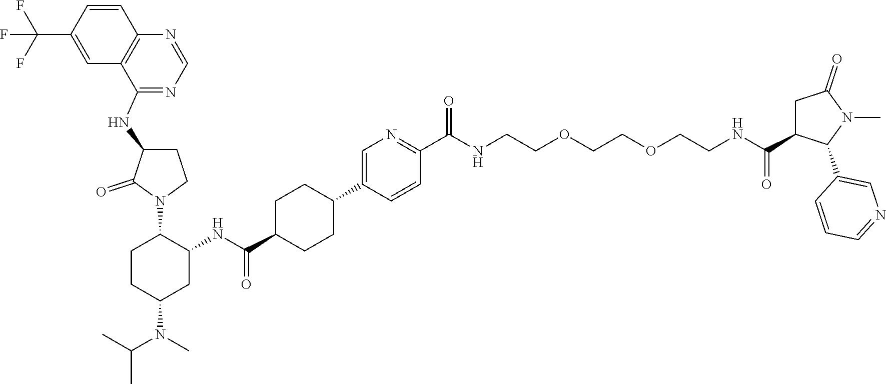 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](c3ccc(C(=O)NCCOCCOCCNC(=O)[C@H]4CC(=O)N(C)[C@@H]4c4cccnc4)nc3)CC2)C1] |
| A142 | 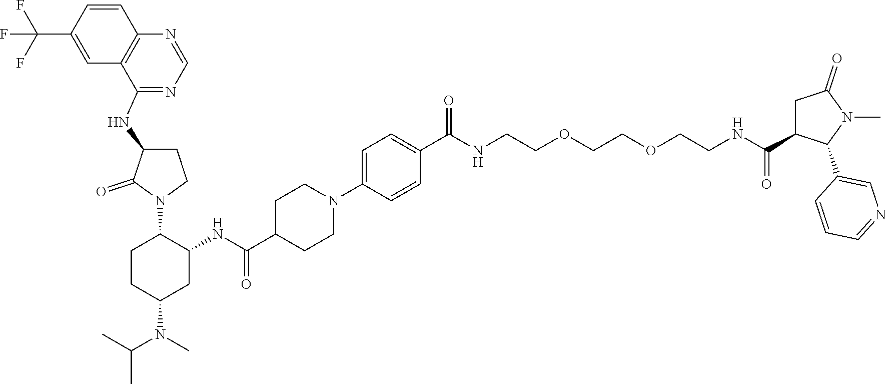 [SMILES: CC(C)N(C)[C@@H]1CC[C@H](N2CC[C@H](Nc3ncnc4ccc(C(F)(F)F)cc34)C2=O)[C@H](NC(=O)C2CCN(c3ccc(C(=O)NCCOCCOCCNC(=O)[C@H]4CC(=O)N(C)[C@@H]4c4cccnc4)cc3)CC2)C1] |
| A143 | 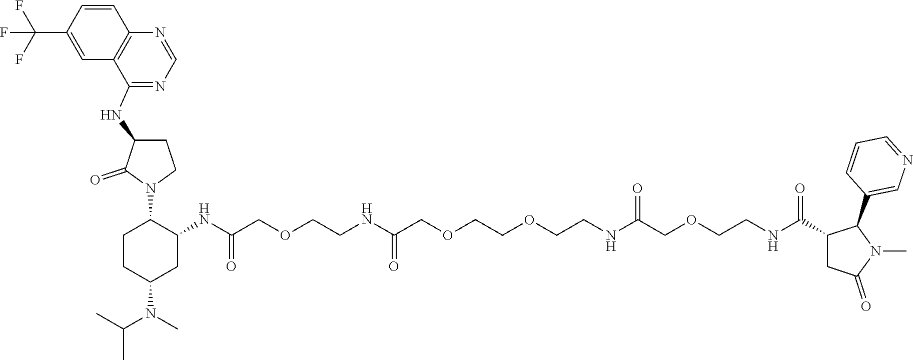 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)COCCOCCNC(=O)COCCNC(=O)[C@H]2CC(=O)N(C)[C@@H]2c2cccnc2)C1] |
| A144 | 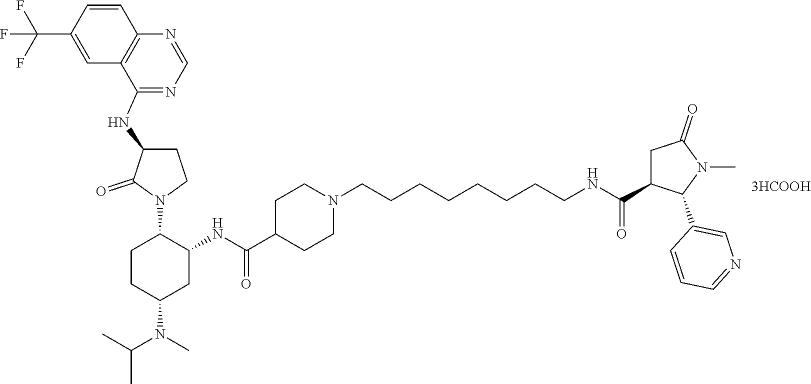 [SMILES: CC(C)N(C)[C@@H]1CC[C@H](N2CC[C@H](Nc3ncnc4ccc(C(F)(F)F)cc34)C2=O)[C@H](NC(=O)C2CCN(CCCCCCCCNC(=O)[C@H]3CC(=O)N(C)[C@@H]3c3cccnc3)CC2)C1] |
| A145 | 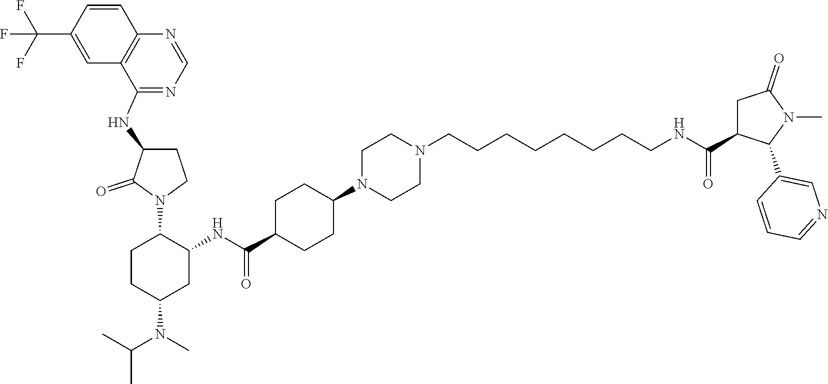 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](N3CCN(CCCCCCCCNC(=O)[C@H]4CC(=O)N(C)[C@@H]4c4cccnc4)CC3)CC2)C1] |
| A146 | 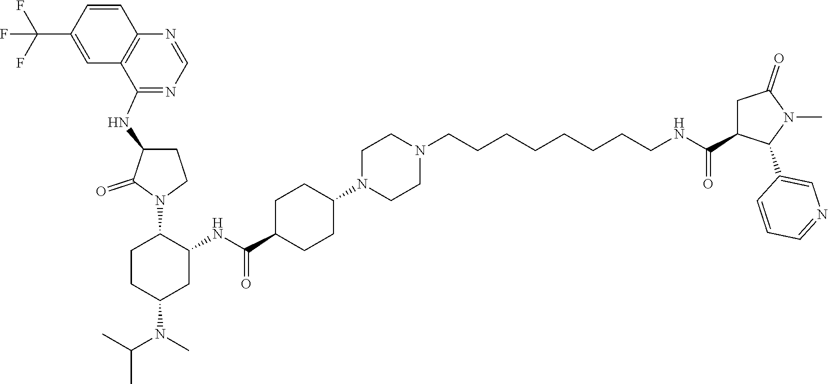 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(CCCCCCCCNC(=O)[C@H]4CC(=O)N(C)[C@@H]4c4cccnc4)CC3)CC2)C1] |
| A147 | 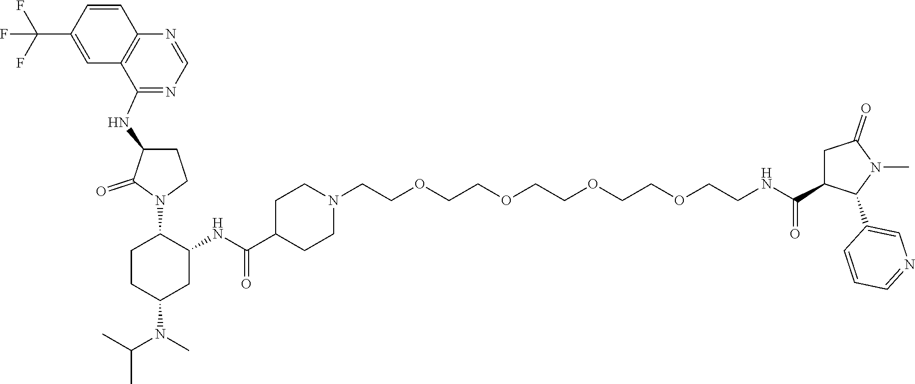 [SMILES: CC(C)N(C)[C@@H]1CC[C@H](N2CC[C@H](Nc3ncnc4ccc(C(F)(F)F)cc34)C2=O)[C@H](NC(=O)C2CCN(CCOCCOCCOCCOCCNC(=O)[C@H]3CC(=O)N(C)[C@@H]3c3cccnc3)CC2)C1] |
| A148 | 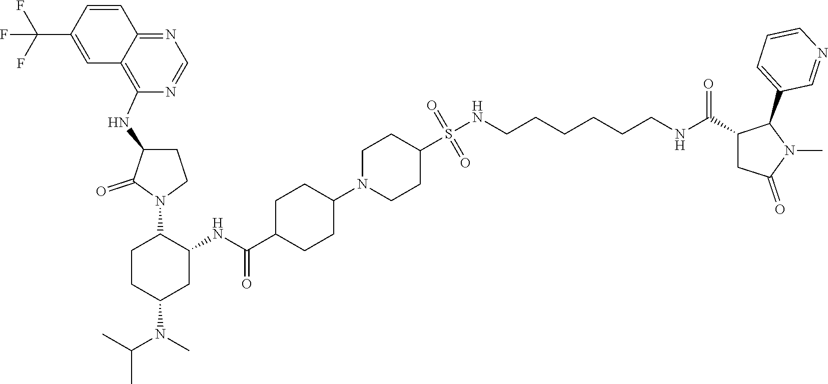 |
| A149 | 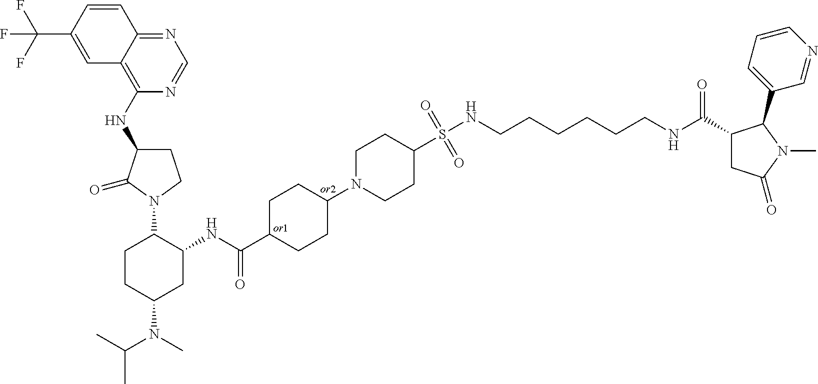 |
| A150 | 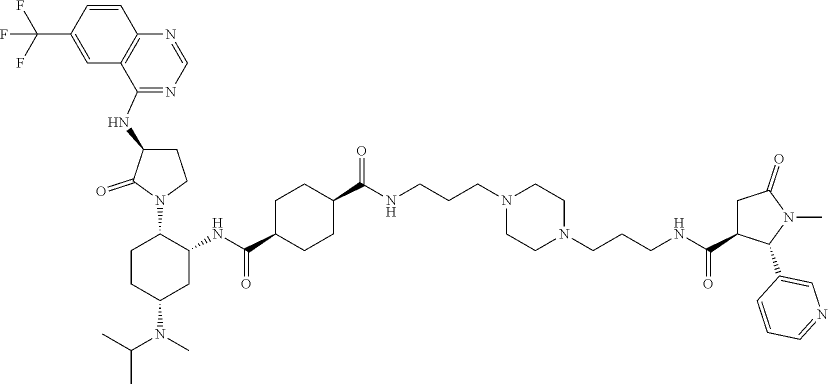 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](C(=O)NCCCN3CCN(CCCNC(=O)[C@H]4CC(=O)N(C)[C@@H]4c4cccnc4)CC3)CC2)C1] |
| A151 | 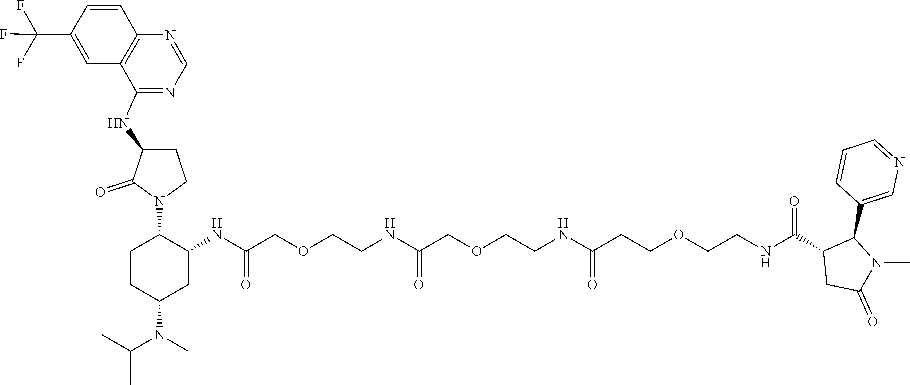 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)COCCNC(=O)CCOCCNC(=O)[C@H]2CC(=O)N(C)[C@@H]2c2cccnc2)C1] |
| A152 | 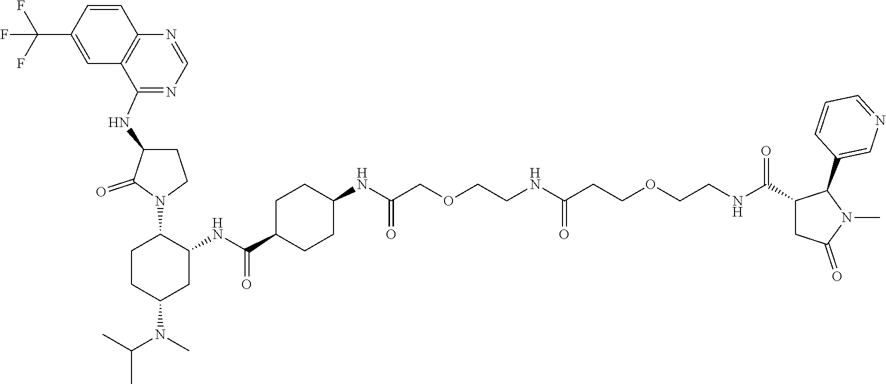 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCNC(=O)CCOCCNC(=O)[C@H]3CC(=O)N(C)[C@@H]3c3cccnc3)CC2)C1] |
| A153 | 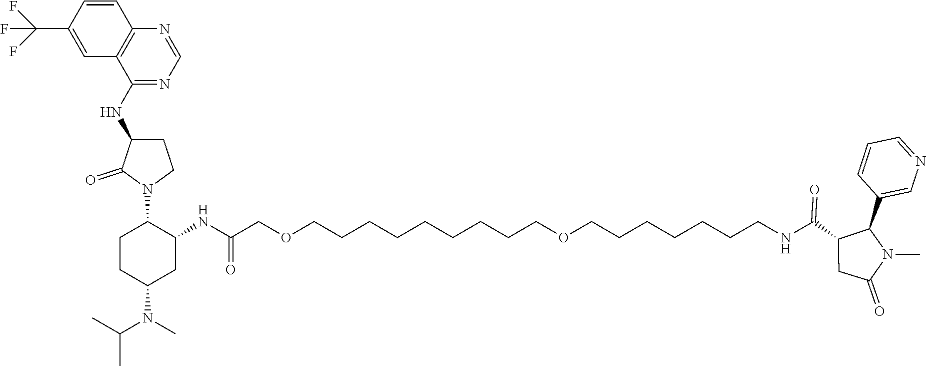 |
| A154 | 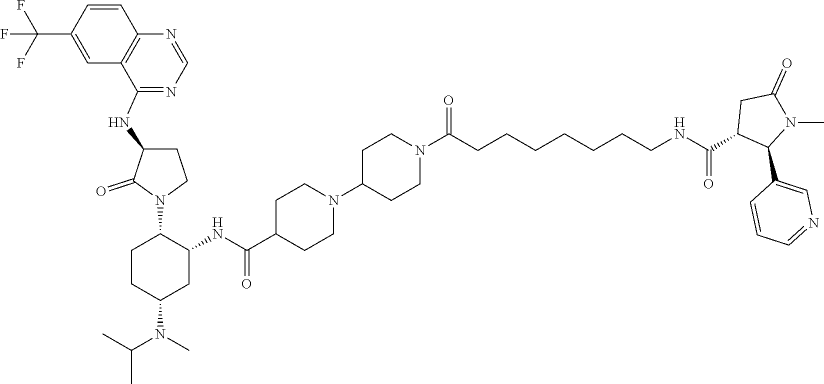 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCCCCCCNC(=O)[C@@H]4CC(=O)N(C)[C@H]4c4cccnc4)CC3)CC2)C1] |
| A155 | 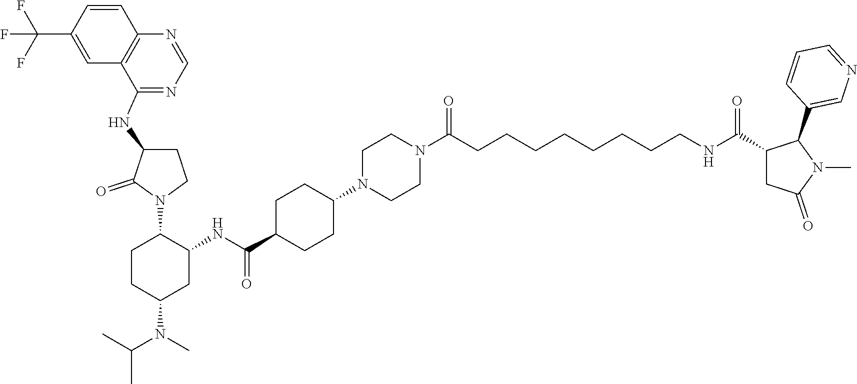 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C(=O)CCCCCCCCNC(=O)[C@H]4CC(=O)N(C)[C@@H]4c4cccnc4)CC3)CC2)C1] |
| A156 | 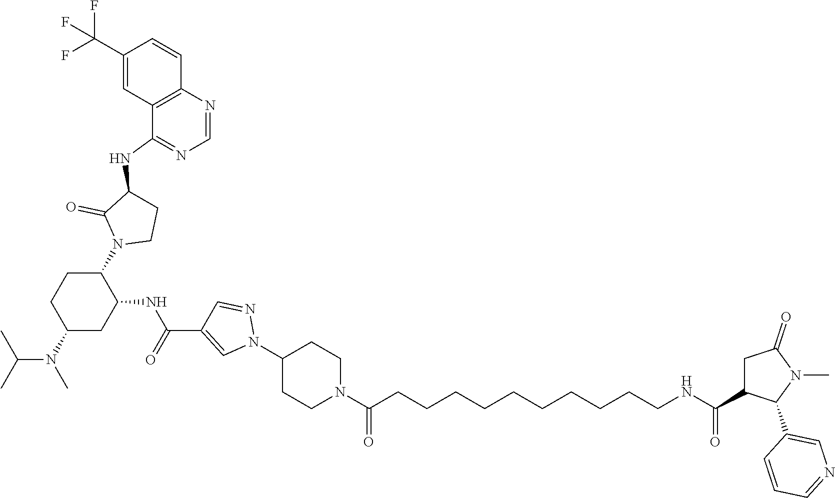 |
| A157 | 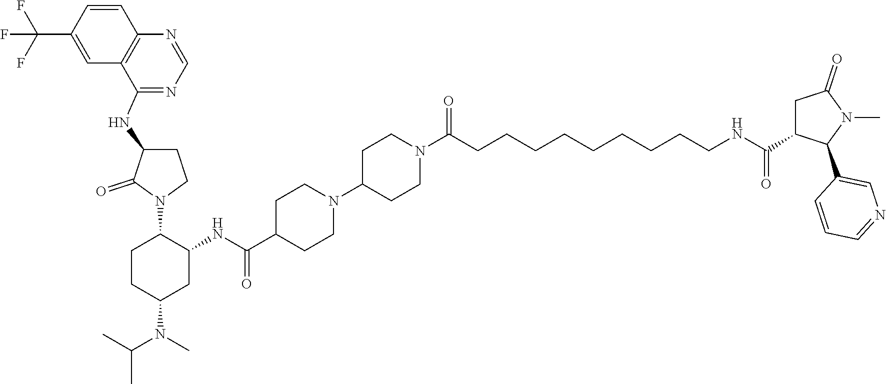 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCCCCCCCCNC(=O)[C@@H]4CC(=O)N(C)[C@H]4c4cccnc4)CC3)CC2)C1] |
| A158 | 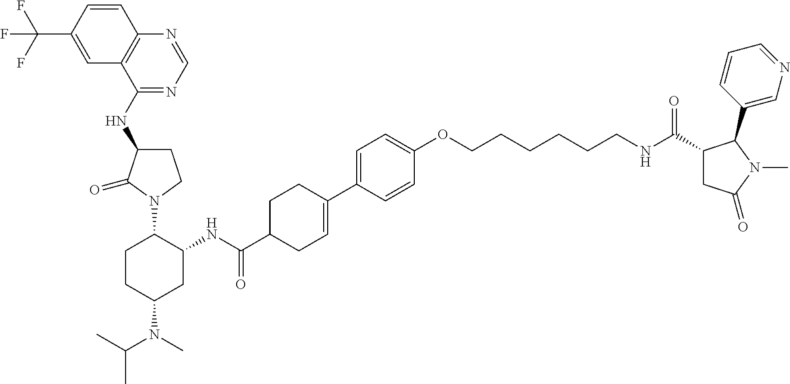 [SMILES: CC(C)N(C)[C@@H]1CC[C@H](N2CC[C@H](Nc3ncnc4ccc(C(F)(F)F)cc34)C2=O)[C@H](NC(=O)C2CC=C(c3ccc(OCCCCCCNC(=O)[C@H]4CC(=O)N(C)[C@@H]4c4cccnc4)cc3)CC2)C1] |
| A159 | 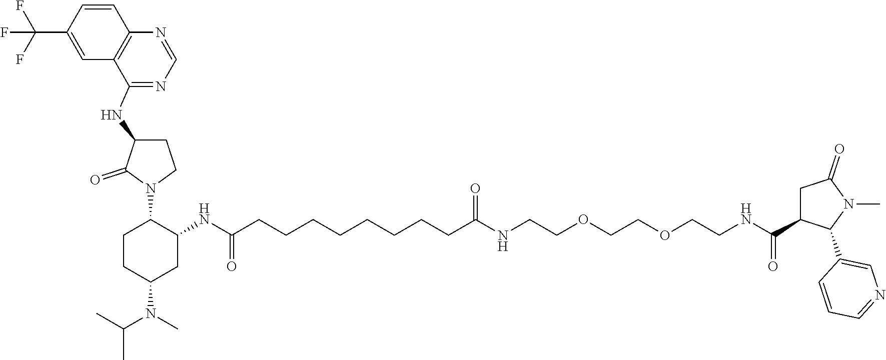 [SMILES: CC(C)N(C)[C@@H]1CC[C@H](N2CC[C@H](Nc3ncnc4ccc(C(F)(F)F)cc34)C2=O)[C@H](NC(=O)CCCCCCCCC(=O)NCCOCCOCCNC(=O)[C@H]2CC(=O)N(C)[C@@H]2c2cccnc2)C1] |
| A160 | 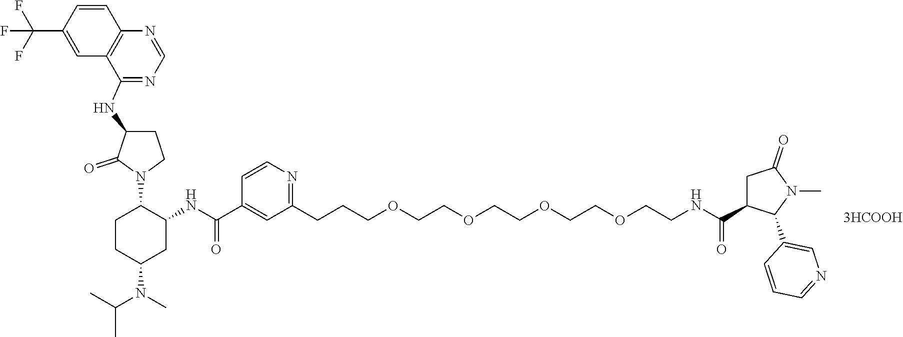 [SMILES: CC(C)N(C)[C@@H]1CC[C@H](N2CC[C@H](Nc3ncnc4ccc(C(F)(F)F)cc34)C2=O)[C@H](NC(=O)c2ccnc(CCCOCCOCCOCCOCCNC(=O)[C@H]3CC(=O)N(C)[C@@H]3c3cccnc3)c2)C1] |
| A161 | 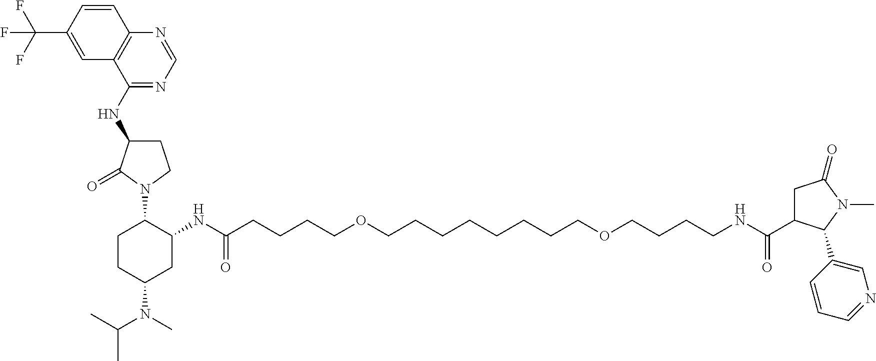 [SMILES: CC(C)N(C)[C@@H]1CC[C@H](N2CC[C@H](Nc3ncnc4ccc(C(F)(F)F)cc34)C2=O)[C@H](NC(=O)CCCCOCCCCCCCCOCCCCNC(=O)C2CC(=O)N(C)[C@@H]2c2cccnc2)C1] |
| A162 | 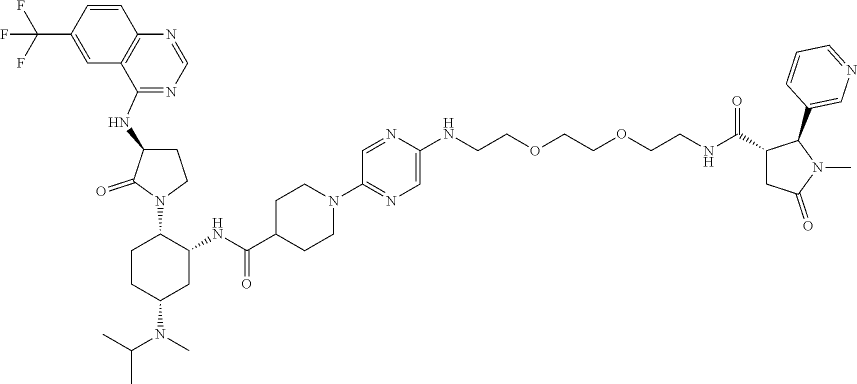 [SMILES: CC(C)N(C)[C@@H]1CC[C@H](N2CC[C@H](Nc3ncnc4ccc(C(F)(F)F)cc34)C2=O)[C@H](NC(=O)C2CCN(c3cnc(NCCOCCOCCNC(=O)[C@H]4CC(=O)N(C)[C@@H]4c4cccnc4)cn3)CC2)C1] |
| A163 | 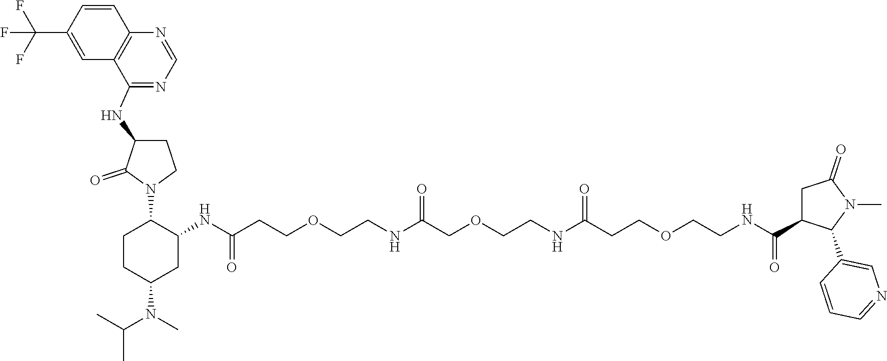 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)COCCNC(=O)CCOCCNC(=O)[C@H]2CC(=O)N(C)[C@@H]2c2cccnc2)C1] |
| A164 | 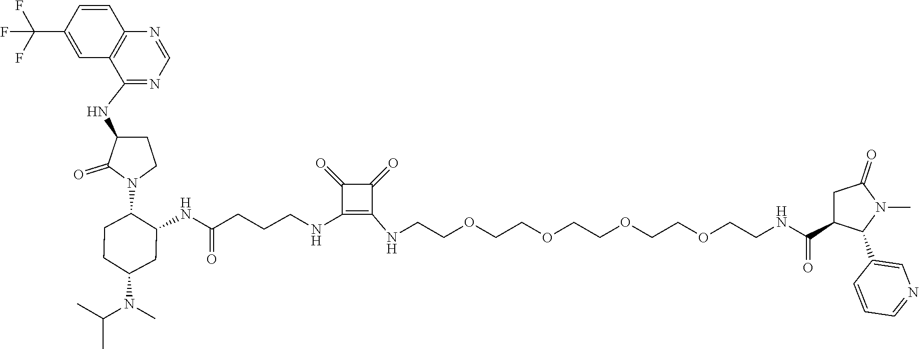 [SMILES: CC(C)N(C)[C@@H]1CC[C@H](N2CC[C@H](Nc3ncnc4ccc(C(F)(F)F)cc34)C2=O)[C@H](NC(=O)CCCNc2c(NCCOCCOCCOCCOCCNC(=O)[C@H]3CC(=O)N(C)[C@@H]3c3cccnc3)c(=O)c2=O)C1] |
| A165 | 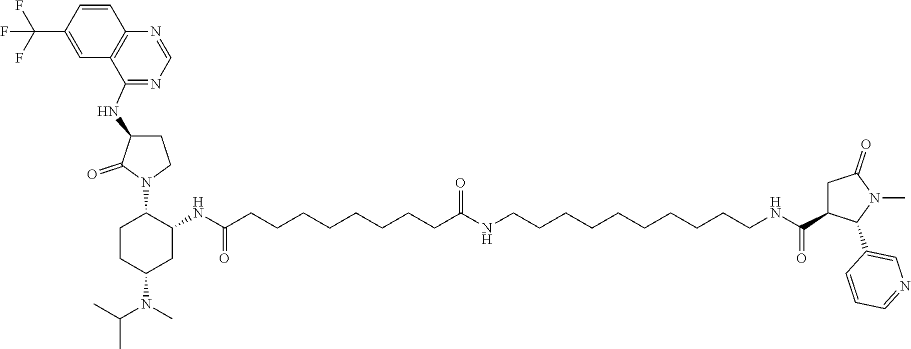 [SMILES: CC(C)N(C)[C@@H]1CC[C@H](N2CC[C@H](Nc3ncnc4ccc(C(F)(F)F)cc34)C2=O)[C@H](NC(=O)CCCCCCCCC(=O)NCCCCCCCCCCNC(=O)[C@H]2CC(=O)N(C)[C@@H]2c2cccnc2)C1] |
| A166 | 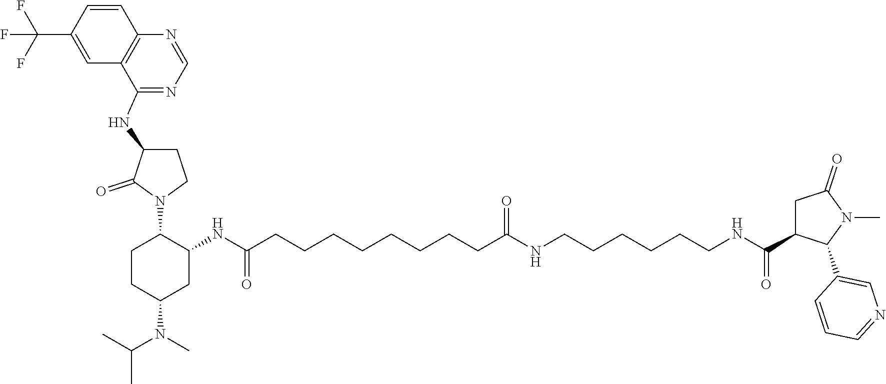 [SMILES: CC(C)N(C)[C@@H]1CC[C@H](N2CC[C@H](Nc3ncnc4ccc(C(F)(F)F)cc34)C2=O)[C@H](NC(=O)CCCCCCCCC(=O)NCCCCCCNC(=O)[C@H]2CC(=O)N(C)[C@@H]2c2cccnc2)C1] |
| A167 | 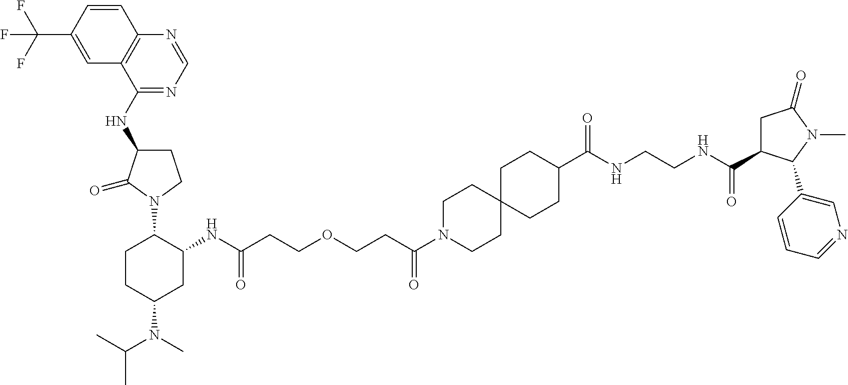 [SMILES: CC(C)N(C)[C@@H]1CC[C@H](N2CC[C@H](Nc3ncnc4ccc(C(F)(F)F)cc34)C2=O)[C@H](NC(=O)CCOCCC(=O)N2CCC3(CCC(C(=O)NCCNC(=O)[C@H]4CC(=O)N(C)[C@@H]4c4cccnc4)CC3)CC2)C1] |
| A168 | 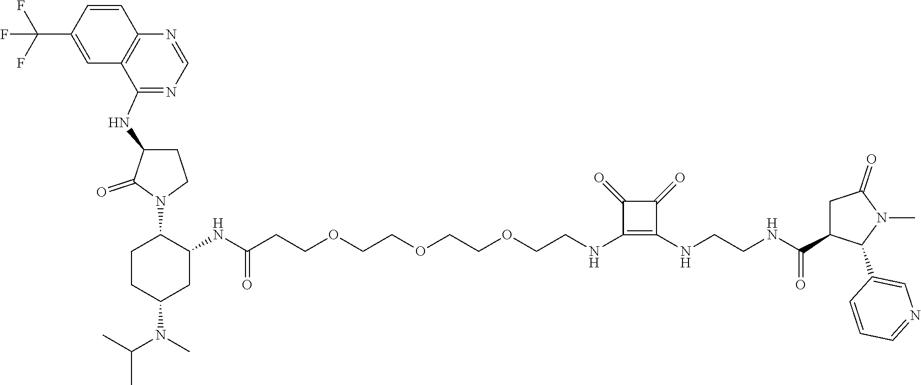 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCNc2c(NCCNC(=O)[C@H]3CC(=O)N(C)[C@@H]3c3cccnc3)c(=O)c2=O)C1] |
| A169 | 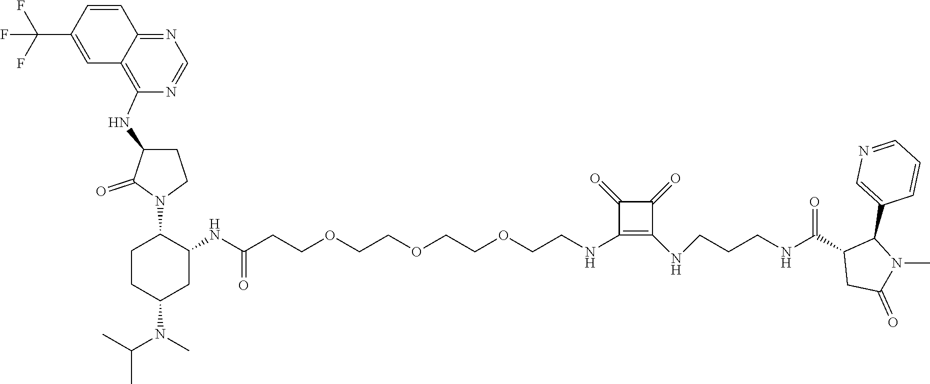 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCNc2c(NCCCNC(=O)[C@H]3CC(=O)N(C)[C@@H]3c3cccnc3)c(=O)c2=O)C1] |
| A170 | 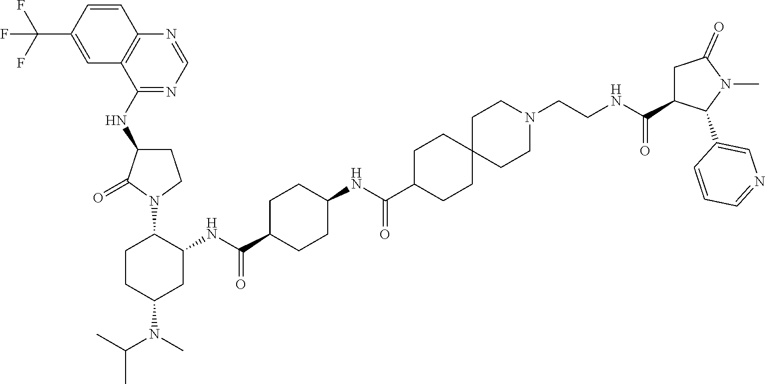 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)C3CCC4(CC3)CCN(CCNC(=O)[C@H]3CC(=O)N(C)[C@@H]3c3cccnc3)CC4)CC2)C1] |
| A171 | 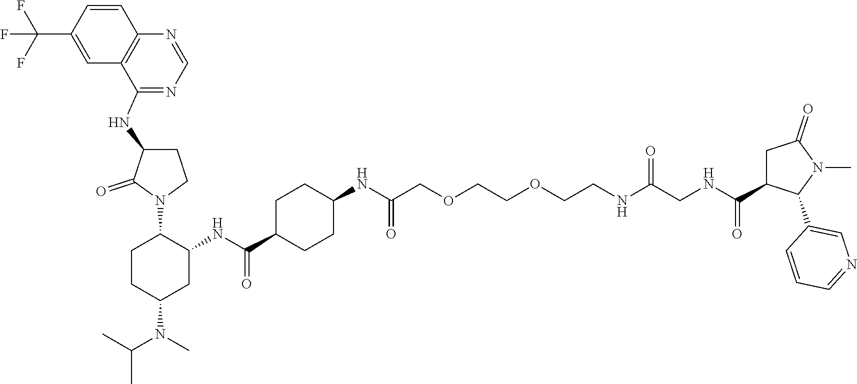 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCOCCNC(=O)CNC(=O)[C@H]3CC(=O)N(C)[C@@H]3c3cccnc3)CC2)C1] |
| A172 | 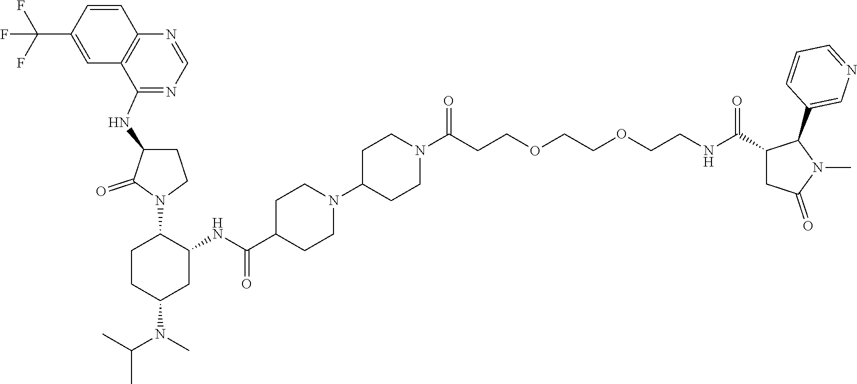 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCOCCOCCNC(=O)[C@H]4CC(=O)N(C)[C@@H]4c4cccnc4)CC3)CC2)C1] |
| A173 | 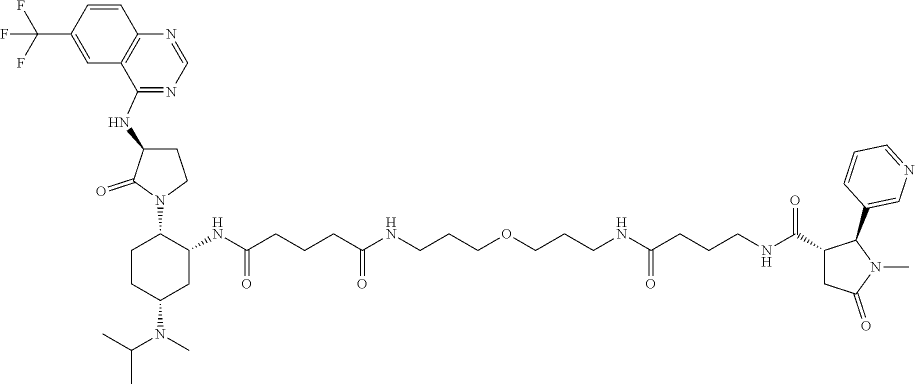 [SMILES: CC(C)N(C)[C@@H]1CC[C@H](N2CC[C@H](Nc3ncnc4ccc(C(F)(F)F)cc34)C2=O)[C@H](NC(=O)CCCC(=O)NCCCOCCCNC(=O)CCCNC(=O)[C@H]2CC(=O)N(C)[C@@H]2c2cccnc2)C1] |
| A175 | 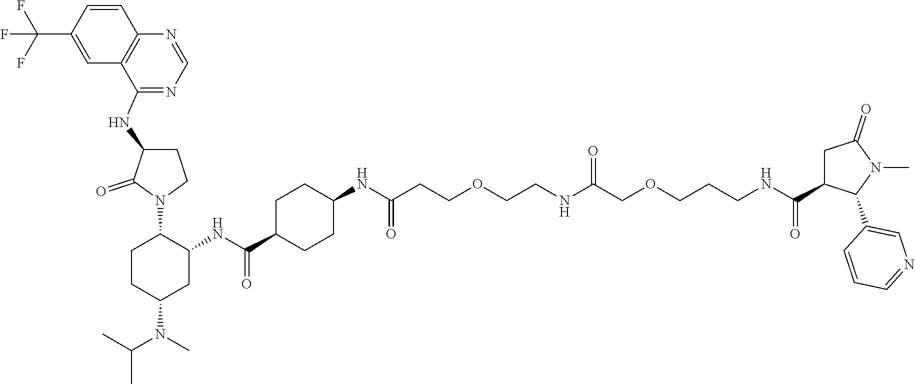 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)COCCCNC(=O)[C@H]3CC(=O)N(C)[C@@H]3c3cccnc3)CC2)C1] |
| A176 | 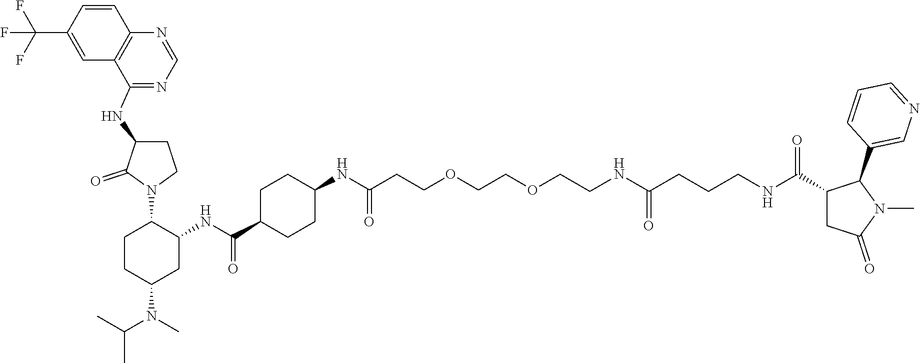 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCNC(=O)CCCNC(=O)[C@H]3CC(=O)N(C)[C@@H]3c3cccnc3)CC2)C1] |
| A177 | 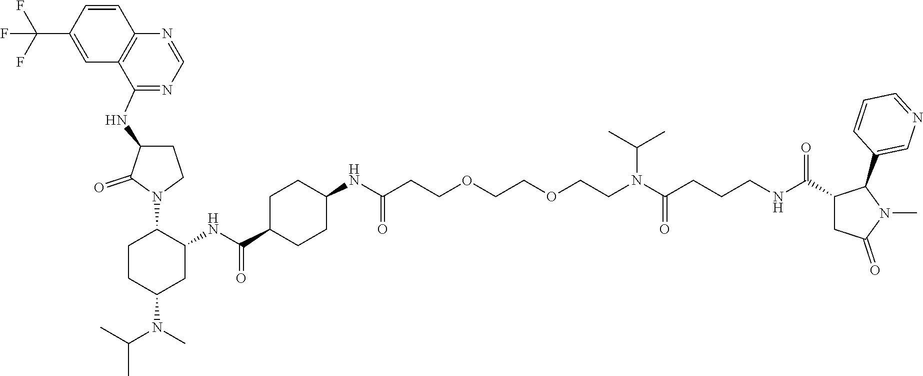 [SMILES: CC(C)N(CCOCCOCCC(=O)N[C@H]1CC[C@@H](C(=O)N[C@@H]2C[C@H](N(C)C(C)C)CC[C@@H]2N2CC[C@H](Nc3ncnc4ccc(C(F)(F)F)cc34)C2=O)CC1)C(=O)CCCNC(=O)[C@H]1CC(=O)N(C)[C@@H]1c1cccnc1] |
| A178 | 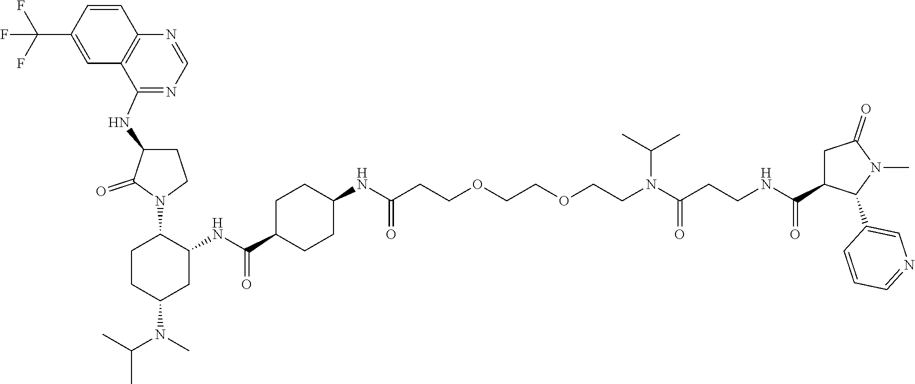 [SMILES: CC(C)N(CCOCCOCCC(=O)N[C@H]1CC[C@@H](C(=O)N[C@@H]2C[C@H](N(C)C(C)C)CC[C@@H]2N2CC[C@H](Nc3ncnc4ccc(C(F)(F)F)cc34)C2=O)CC1)C(=O)CCNC(=O)[C@H]1CC(=O)N(C)[C@@H]1c1cccnc1] |
| A179 | 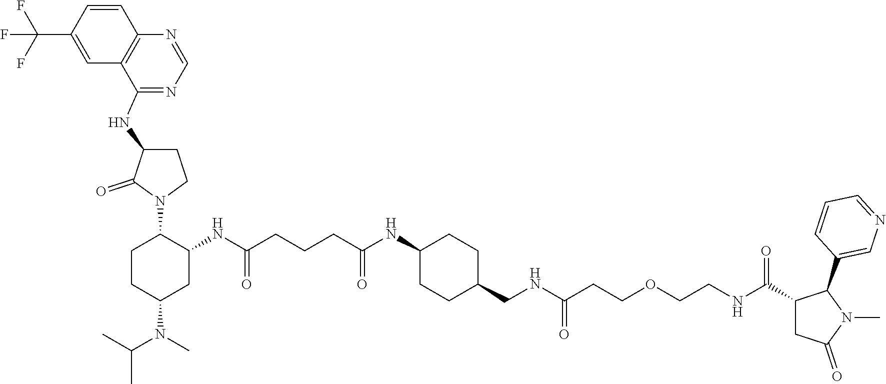 [SMILES: CC(C)N(C)[C@@H]1CC[C@H](N2CC[C@H](Nc3ncnc4ccc(C(F)(F)F)cc34)C2=O)[C@H](NC(=O)CCCC(=O)N[C@H]2CC[C@@H](CNC(=O)CCOCCNC(=O)[C@H]3CC(=O)N(C)[C@@H]3c3cccnc3)CC2)C1] |
| A180 | 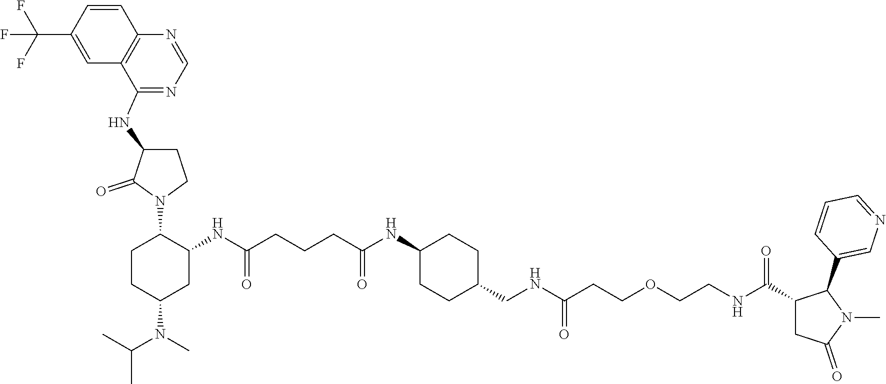 [SMILES: CC(C)N(C)[C@@H]1CC[C@H](N2CC[C@H](Nc3ncnc4ccc(C(F)(F)F)cc34)C2=O)[C@H](NC(=O)CCCC(=O)N[C@H]2CC[C@H](CNC(=O)CCOCCNC(=O)[C@H]3CC(=O)N(C)[C@@H]3c3cccnc3)CC2)C1] |
| A181 | 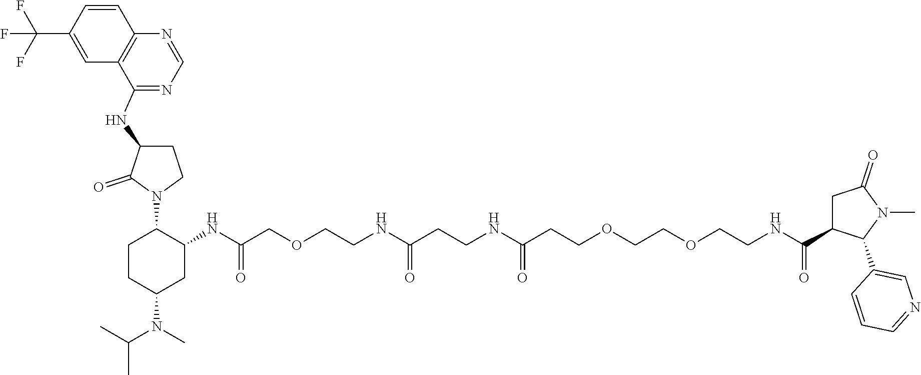 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCNC(=O)CCOCCOCCNC(=O)[C@H]2CC(=O)N(C)[C@@H]2c2cccnc2)C1] |
| A182 | 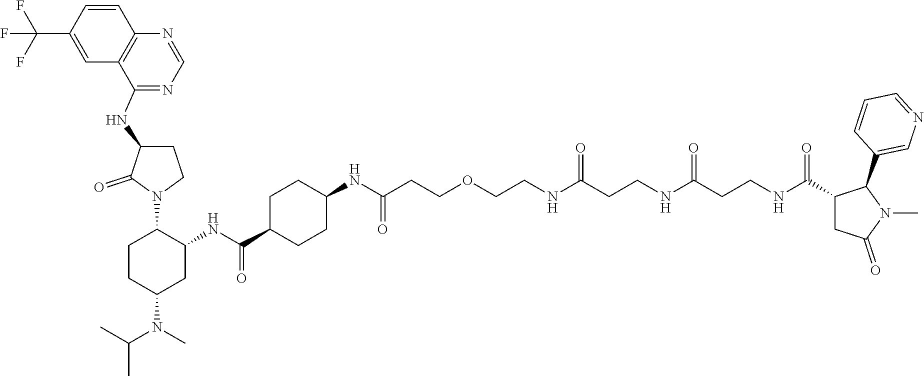 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCNC(=O)CCNC(=O)[C@H]3CC(=O)N(C)[C@@H]3c3cccnc3)CC2)C1] |
| A183 | 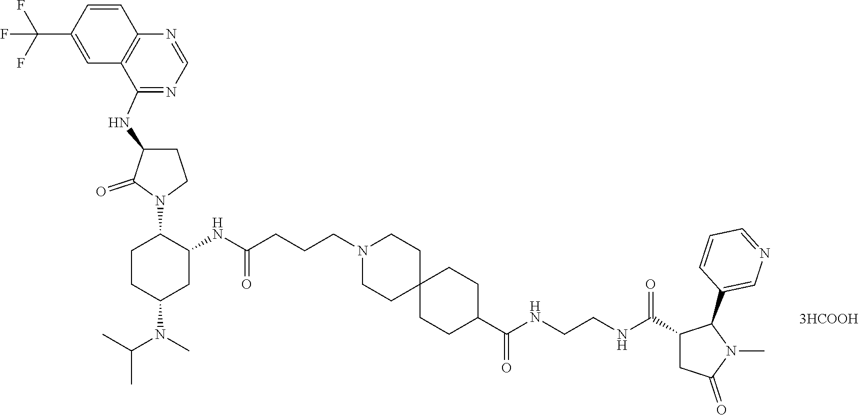 [SMILES: CC(C)N(C)[C@@H]1CC[C@H](N2CC[C@H](Nc3ncnc4ccc(C(F)(F)F)cc34)C2=O)[C@H](NC(=O)CCCN2CCC3(CCC(C(=O)NCCNC(=O)[C@H]4CC(=O)N(C)[C@@H]4c4cccnc4)CC3)CC2)C1] |
| A184 | 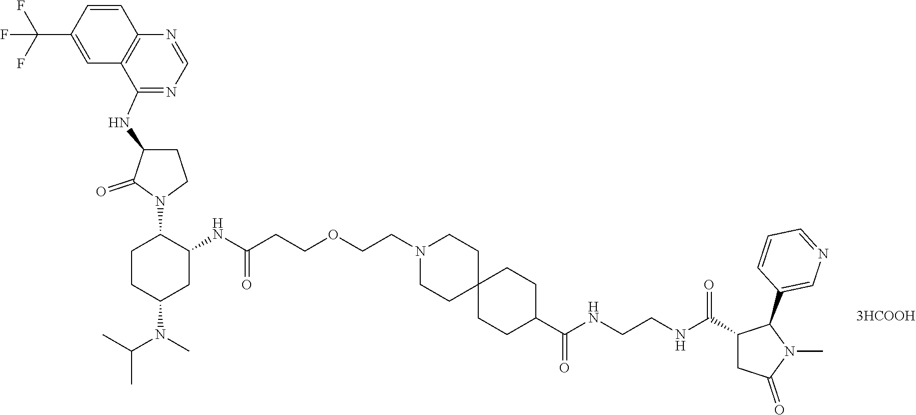 [SMILES: CC(C)N(C)[C@@H]1CC[C@H](N2CC[C@H](Nc3ncnc4ccc(C(F)(F)F)cc34)C2=O)[C@H](NC(=O)CCOCCN2CCC3(CCC(C(=O)NCCNC(=O)[C@H]4CC(=O)N(C)[C@@H]4c4cccnc4)CC3)CC2)C1] |
| A185 | 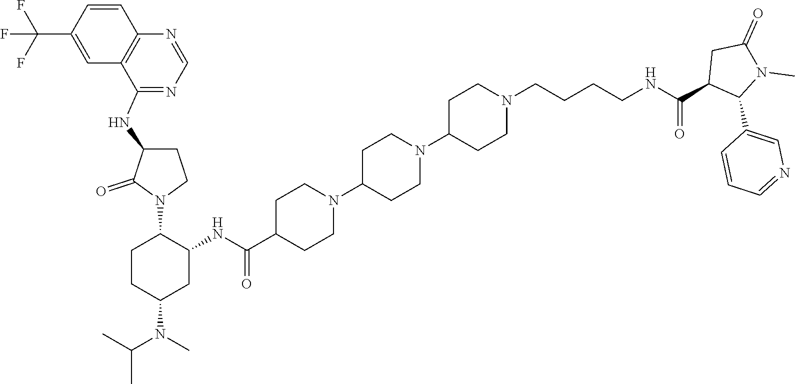 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C4CCN(CCCCNC(=O)[C@H]5CC(=O)N(C)[C@@H]5c5cccnc5)CC4)CC3)CC2)C1] |
| A186 | 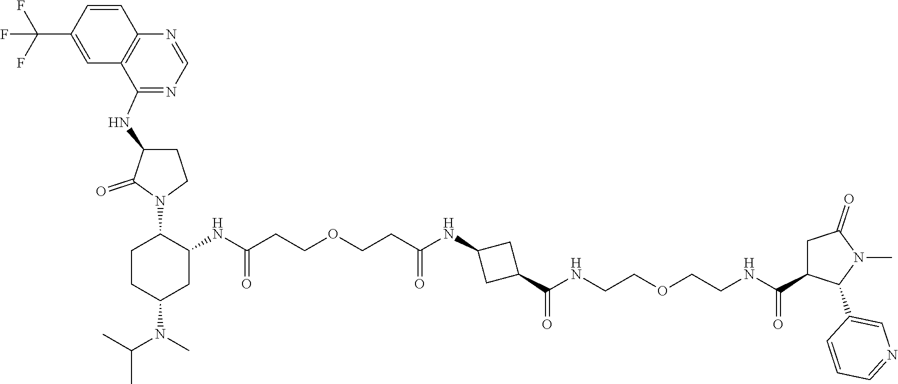 [SMILES: CC(C)N(C)[C@@H]1CC[C@H](N2CC[C@H](Nc3ncnc4ccc(C(F)(F)F)cc34)C2=O)[C@H](NC(=O)CCOCCC(=O)N[C@H]2C[C@@H](C(=O)NCCOCCNC(=O)[C@H]3CC(=O)N(C)[C@@H]3c3cccnc3)C2)C1] |
| A187 | 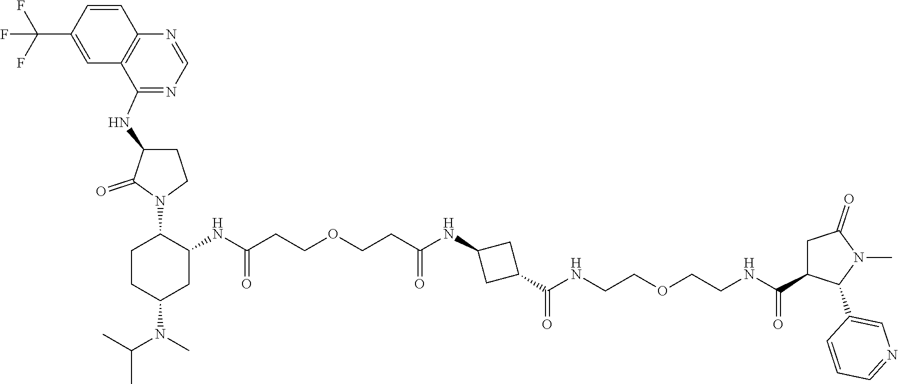 [SMILES: CC(C)N(C)[C@@H]1CC[C@H](N2CC[C@H](Nc3ncnc4ccc(C(F)(F)F)cc34)C2=O)[C@H](NC(=O)CCOCCC(=O)N[C@H]2C[C@H](C(=O)NCCOCCNC(=O)[C@H]3CC(=O)N(C)[C@@H]3c3cccnc3)C2)C1] |
| A188 | 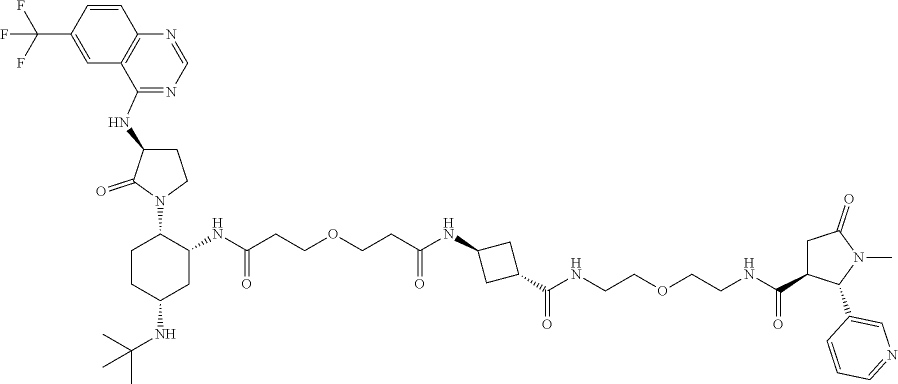 [SMILES: CN1C(=O)C[C@H](C(=O)NCCOCCNC(=O)[C@H]2C[C@H](NC(=O)CCOCCC(=O)N[C@@H]3C[C@H](NC(C)(C)C)CC[C@@H]3N3CC[C@H](Nc4ncnc5ccc(C(F)(F)F)cc45)C3=O)C2)[C@H]1c1cccnc1] |
| A189 | 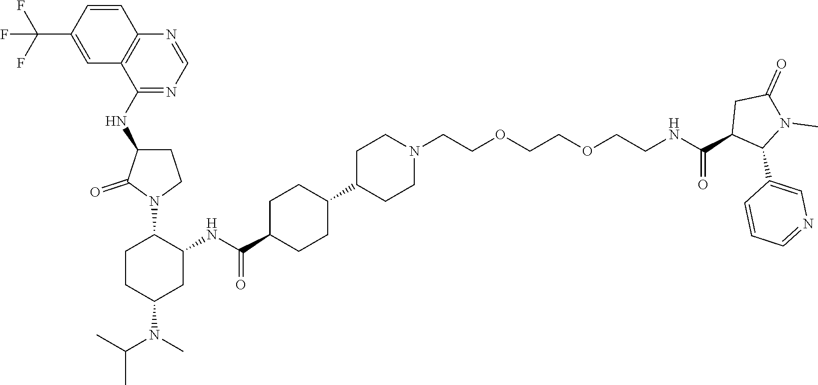 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](C3CCN(CCOCCOCCNC(=O)[C@H]4CC(=O)N(C)[C@@H]4c4cccnc4)CC3)CC2)C1] |
| A190 | 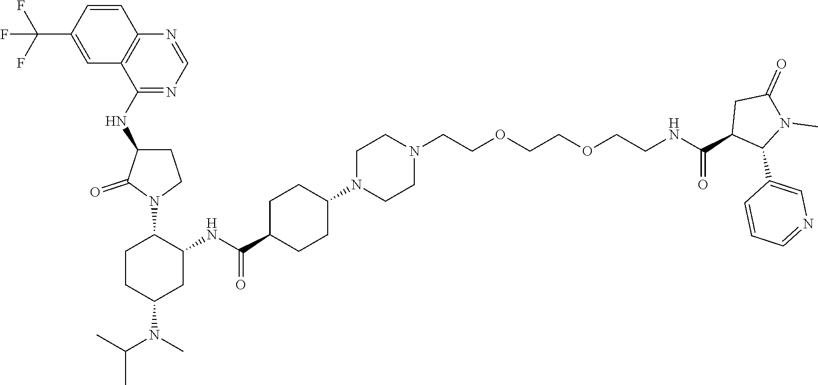 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(CCOCCOCCNC(=O)[C@H]4CC(=O)N(C)[C@@H]4c4cccnc4)CC3)CC2)C1] |
| A191 | 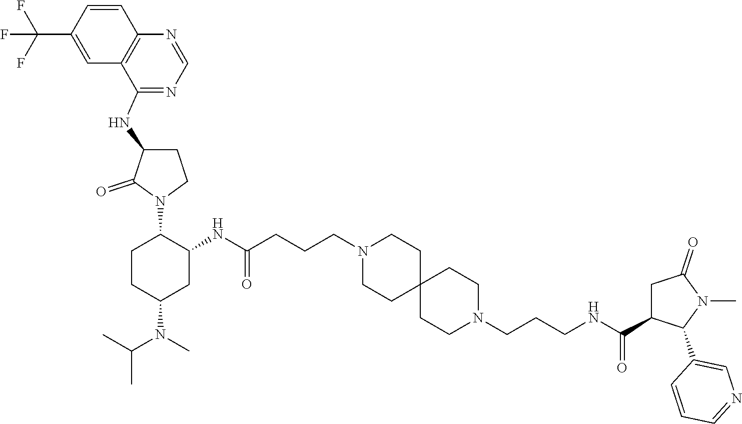 [SMILES: CC(C)N(C)[C@@H]1CC[C@H](N2CC[C@H](Nc3ncnc4ccc(C(F)(F)F)cc34)C2=O)[C@H](NC(=O)CCCN2CCC3(CCN(CCCNC(=O)[C@H]4CC(=O)N(C)[C@@H]4c4cccnc4)CC3)CC2)C1] |
| A192 | 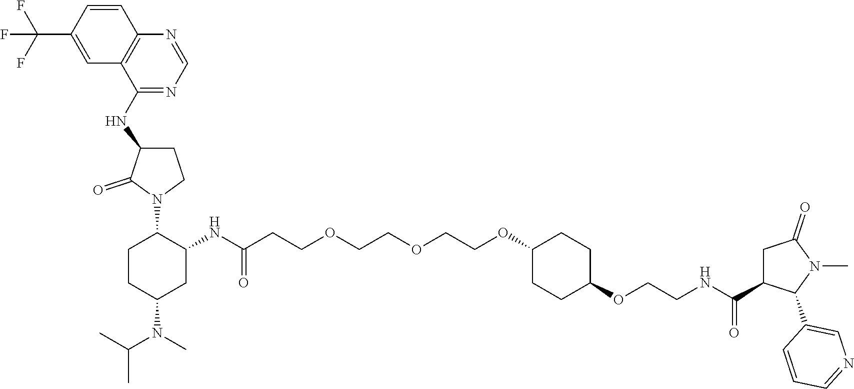 [SMILES: CC(C)N(C)[C@@H]1CC[C@H](N2CC[C@H](Nc3ncnc4ccc(C(F)(F)F)cc34)C2=O)[C@H](NC(=O)CCOCCOCCO[C@H]2CC[C@H](OCCNC(=O)[C@H]3CC(=O)N(C)[C@@H]3c3cccnc3)CC2)C1] |
| A193 | 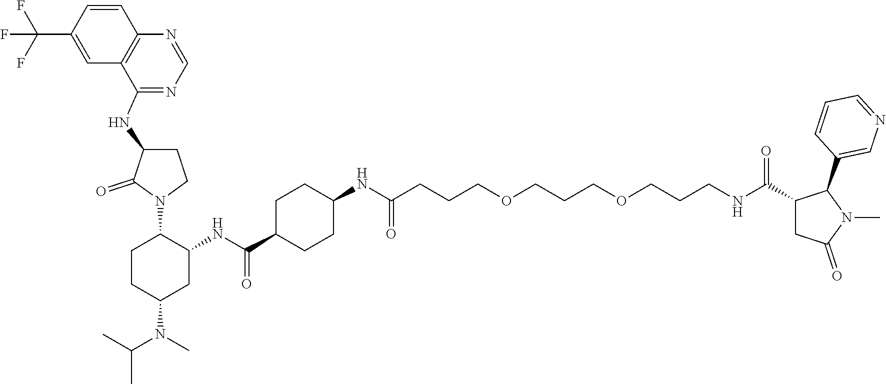 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCOCCCOCCCNC(=O)[C@H]3CC(=O)N(C)[C@@H]3c3cccnc3)CC2)C1] |
| A194 | 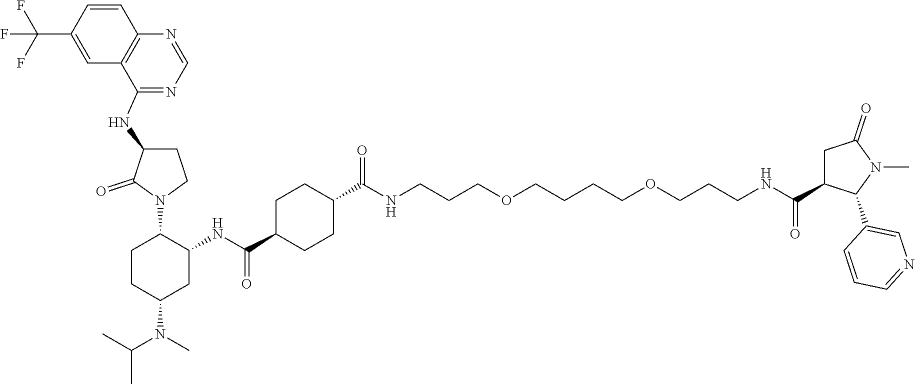 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](C(=O)NCCCOCCCCOCCCNC(=O)[C@H]3CC(=O)N(C)[C@@H]3c3cccnc3)CC2)C1] |
| A195 | 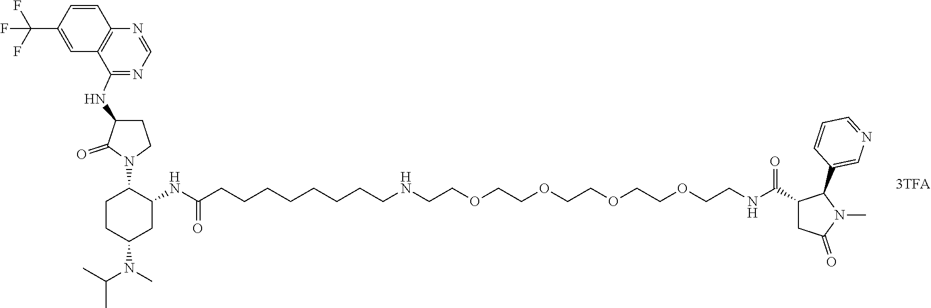 [SMILES: CC(C)N(C)[C@@H]1CC[C@H](N2CC[C@H](Nc3ncnc4ccc(C(F)(F)F)cc34)C2=O)[C@H](NC(=O)CCCCCCCCNCCOCCOCCOCCOCCNC(=O)[C@H]2CC(=O)N(C)[C@@H]2c2cccnc2)C1] |
| A196 | 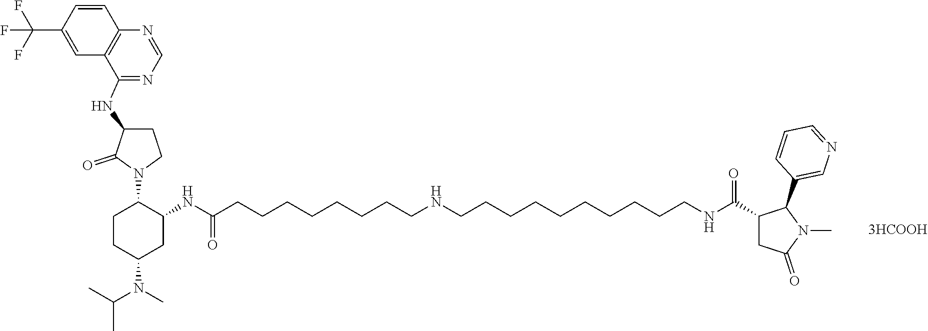 [SMILES: CC(C)N(C)[C@@H]1CC[C@H](N2CC[C@H](Nc3ncnc4ccc(C(F)(F)F)cc34)C2=O)[C@H](NC(=O)CCCCCCCCNCCCCCCCCCCNC(=O)[C@H]2CC(=O)N(C)[C@@H]2c2cccnc2)C1] |
| A197 | 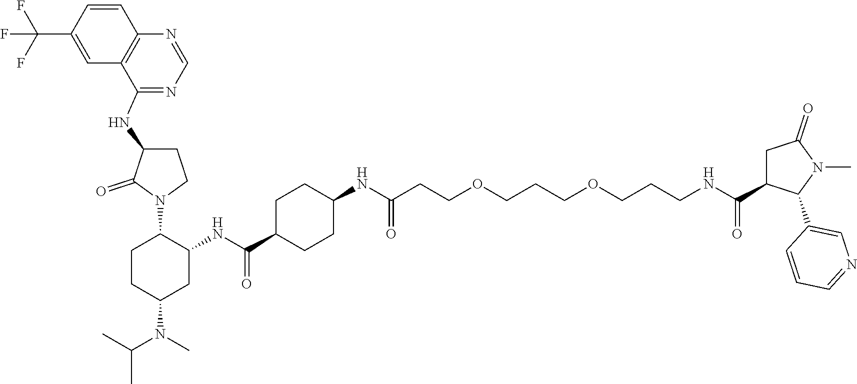 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCCOCCCNC(=O)[C@H]3CC(=O)N(C)[C@@H]3c3cccnc3)CC2)C1] |
| A198 | 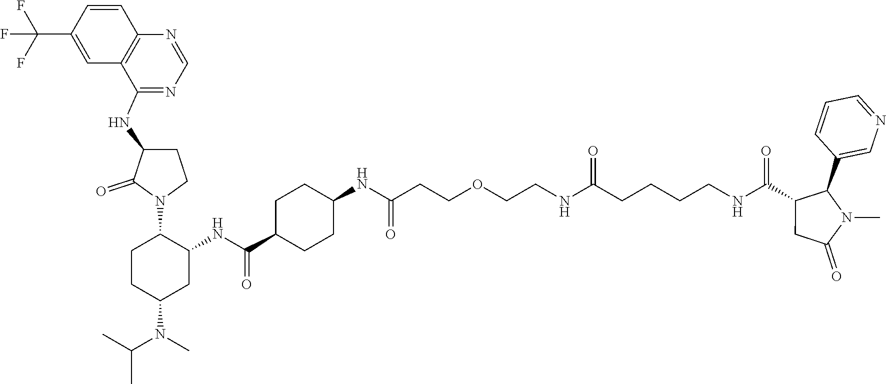 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCCCNC(=O)[C@H]3CC(=O)N(C)[C@@H]3c3cccnc3)CC2)C1] |
| B1 | 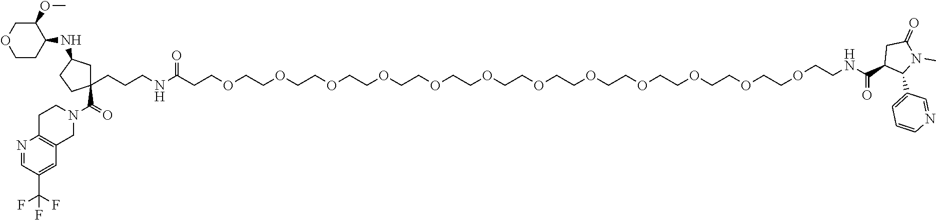 [SMILES: CO[C@@H]1COCC[C@@H]1N[C@@H]1CC[C@](CCCNC(=O)CCOCCOCCOCCOCCOCCOCCOCCOCCOCCOCCOCCOCCNC(=O)[C@H]2CC(=O)N(C)[C@@H]2c2cccnc2)(C(=O)N2CCc3ncc(C(F)(F)F)cc3C2)C1] |
| B2 | 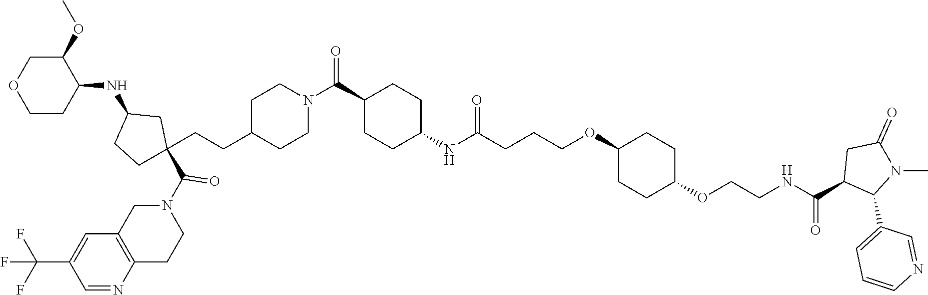 [SMILES: CO[C@@H]1COCC[C@@H]1N[C@@H]1CC[C@](CCC2CCN(C(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)(C(=O)N2CCc3ncc(C(F)(F)F)cc3C2)C1] |
| B3 | 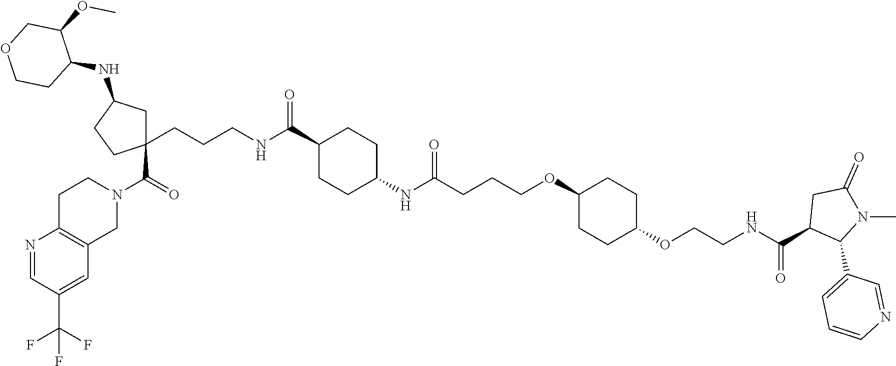 [SMILES: CO[C@@H]1COCC[C@@H]1N[C@@H]1CC[C@](CCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B4 | 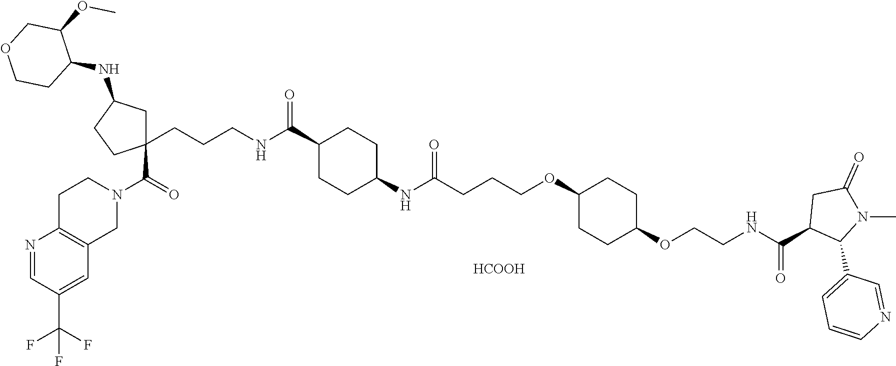 [SMILES: CO[C@@H]1COCC[C@@H]1N[C@@H]1CC[C@](CCCNC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)(C(=O)N2CCc3ncc(C(F)(F)F)cc3C2)C1 ; "HCOOH"] |
| B5 | 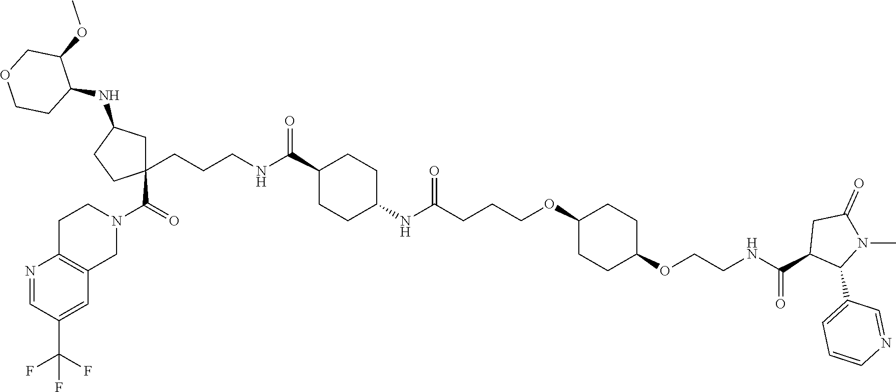 [SMILES: CO[C@@H]1COCC[C@@H]1N[C@@H]1CC[C@](CCCNC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)(C(=O)N2CCc3ncc(C(F)(F)F)cc3C2)C1] |
| B6 | 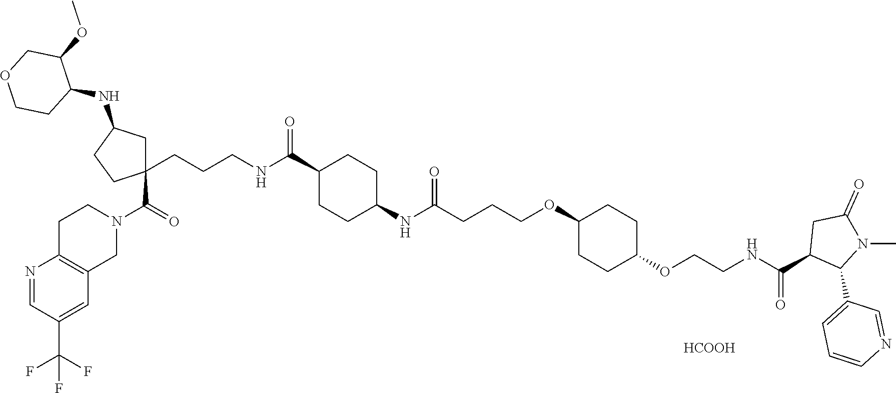 [SMILES: CO[C@@H]1COCC[C@@H]1N[C@@H]1CC[C@](CCCNC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1 ; "HCOOH"] |
| B7 | 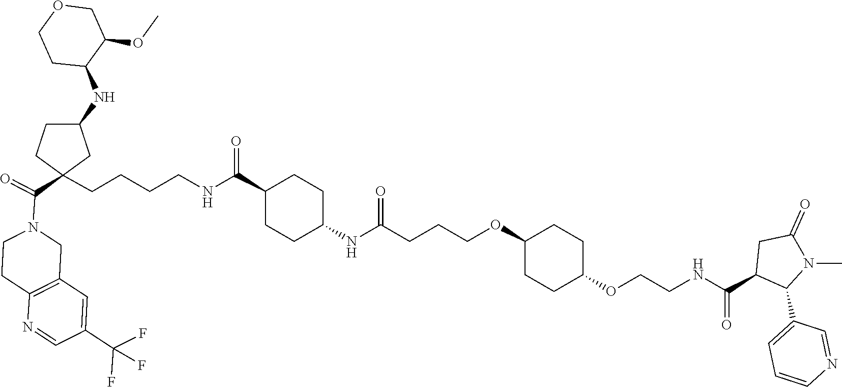 [SMILES: CO[C@@H]1COCC[C@@H]1N[C@@H]1CC[C@](CCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B8 | 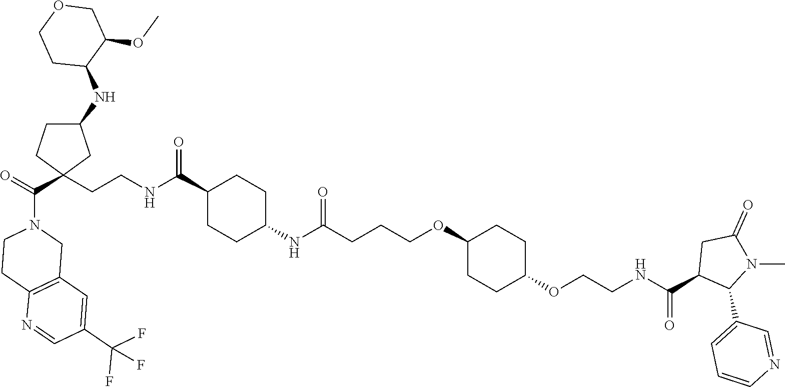 [SMILES: CO[C@@H]1COCC[C@@H]1N[C@@H]1CC[C@](CCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B9 | 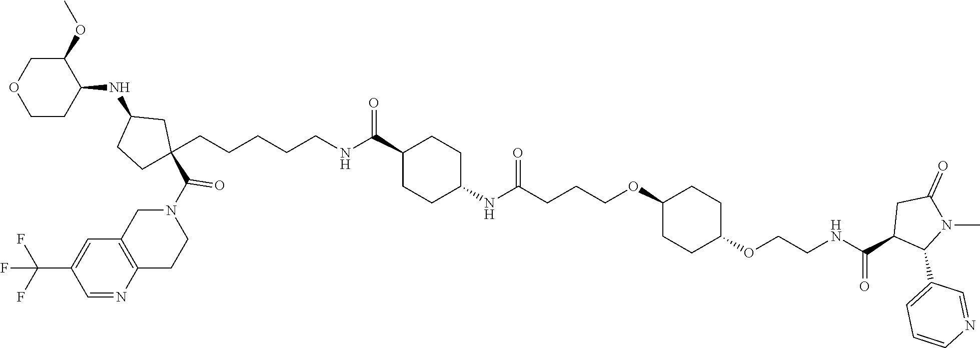 [SMILES: CO[C@@H]1COCC[C@@H]1N[C@@H]1CC[C@](CCCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B10 | 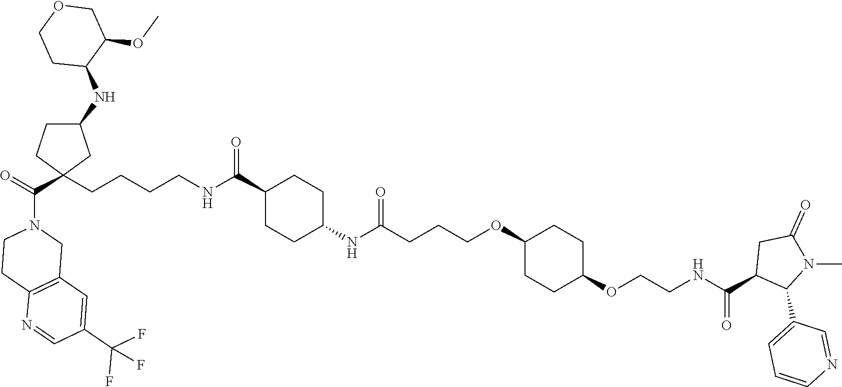 [SMILES: CO[C@@H]1COCC[C@@H]1N[C@@H]1CC[C@](CCCCNC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)(C(=O)N2CCc3ncc(C(F)(F)F)cc3C2)C1] |
| B11 | 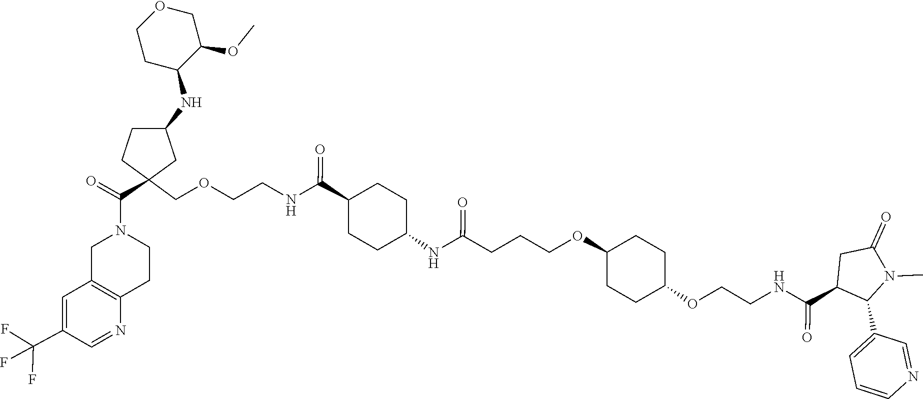 [SMILES: CO[C@@H]1COCC[C@@H]1N[C@@H]1CC[C@](COCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B12 | 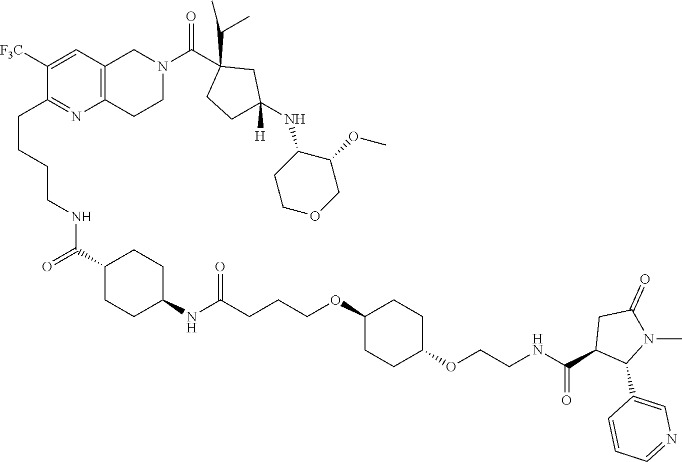 [SMILES: CO[C@@H]1COCC[C@@H]1N[C@@H]1CC[C@@](C(=O)N2CCc3nc(CCCCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)(C(C)C)C1] |
| B13 | 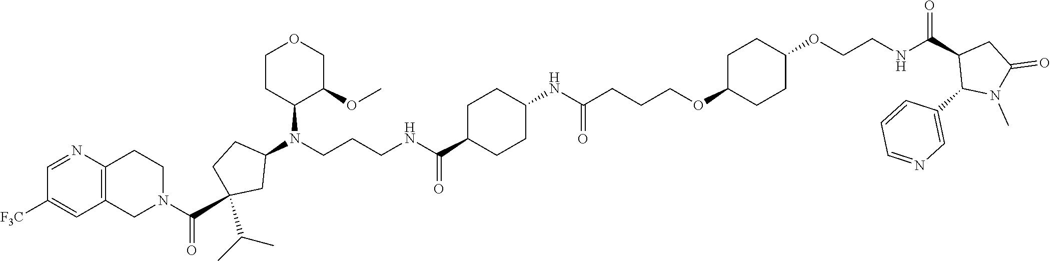 [SMILES: CO[C@@H]1COCC[C@@H]1N(CCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2CCc3ncc(C(F)(F)F)cc3C2)(C(C)C)C1] |
| B14 | 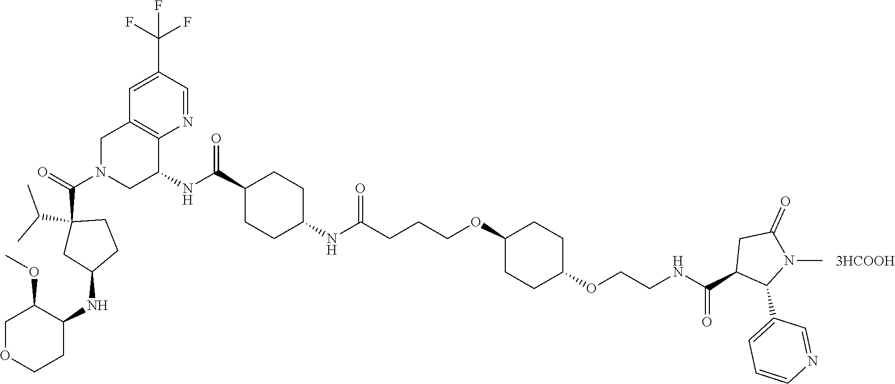 |
| B15 | 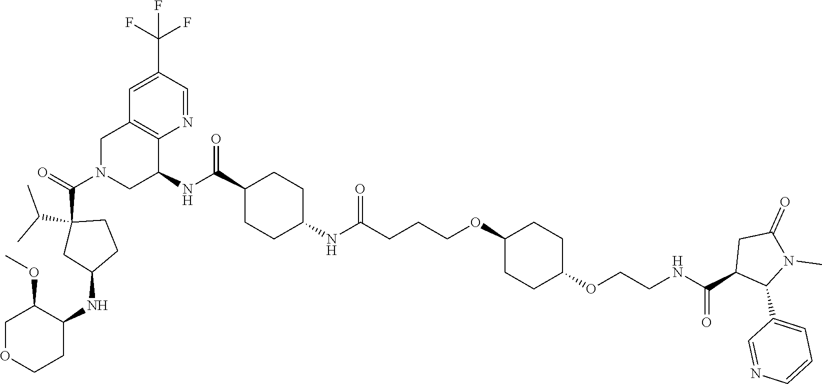 |
| B16 | 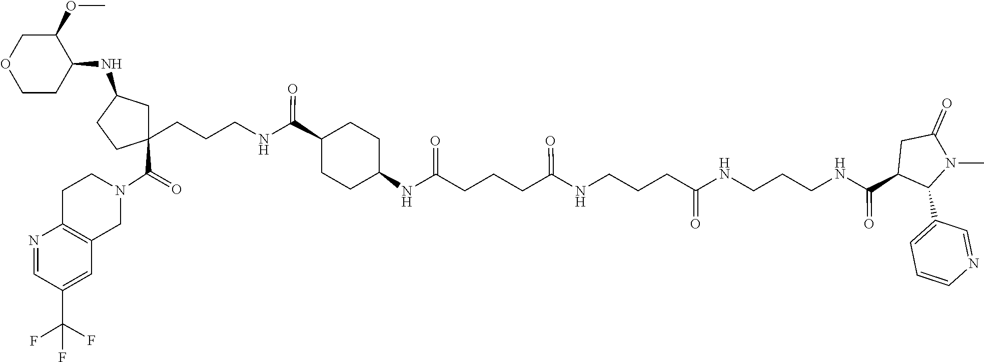 [SMILES: CO[C@@H]1COCC[C@@H]1N[C@@H]1CC[C@](CCCNC(=O)[C@H]2CC[C@@H](NC(=O)CCCC(=O)NCCCC(=O)NCCCNC(=O)[C@H]3CC(=O)N(C)[C@@H]3c3cccnc3)CC2)(C(=O)N2CCc3ncc(C(F)(F)F)cc3C2)C1] |
| B17 | 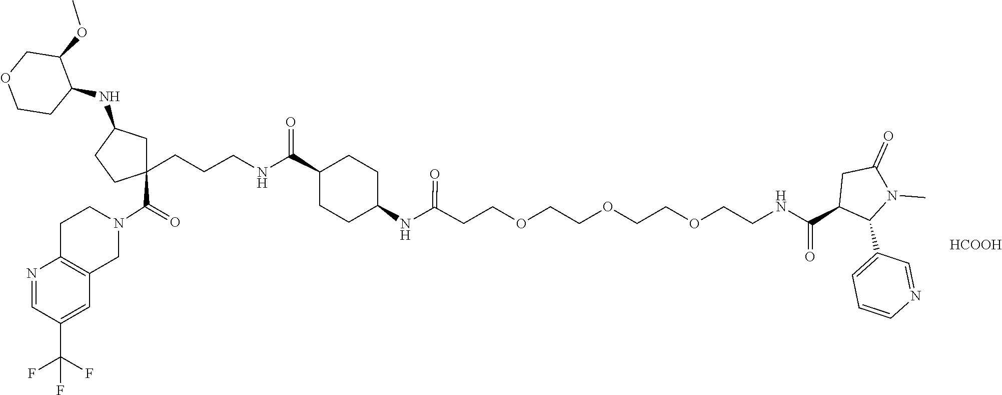 [SMILES: CO[C@@H]1COCC[C@@H]1N[C@@H]1CC[C@](CCCNC(=O)[C@H]2CC[C@@H](NC(=O)CCOCCOCCOCCNC(=O)[C@H]3CC(=O)N(C)[C@@H]3c3cccnc3)CC2)(C(=O)N2CCc3ncc(C(F)(F)F)cc3C2)C1 ; "HCOOH"] |
| B18 | 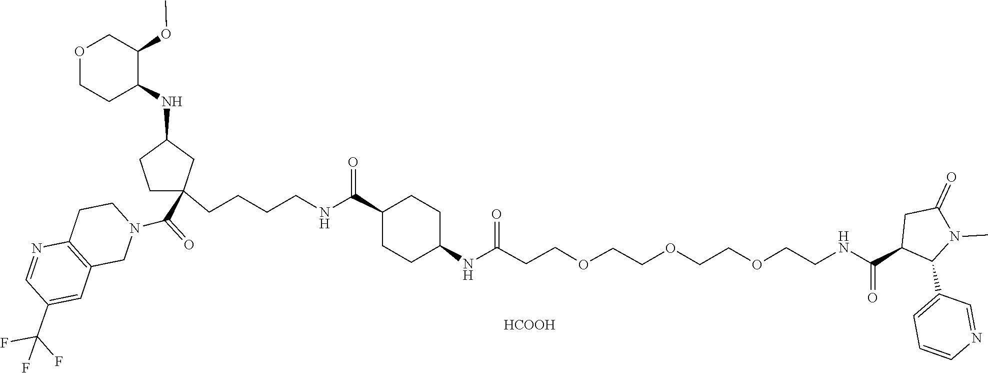 [SMILES: CO[C@@H]1COCC[C@@H]1N[C@@H]1CC[C@](CCCCNC(=O)[C@H]2CC[C@@H](NC(=O)CCOCCOCCOCCNC(=O)[C@H]3CC(=O)N(C)[C@@H]3c3cccnc3)CC2)(C(=O)N2CCc3ncc(C(F)(F)F)cc3C2)C1 ; "HCOOH"] |
| B19 | 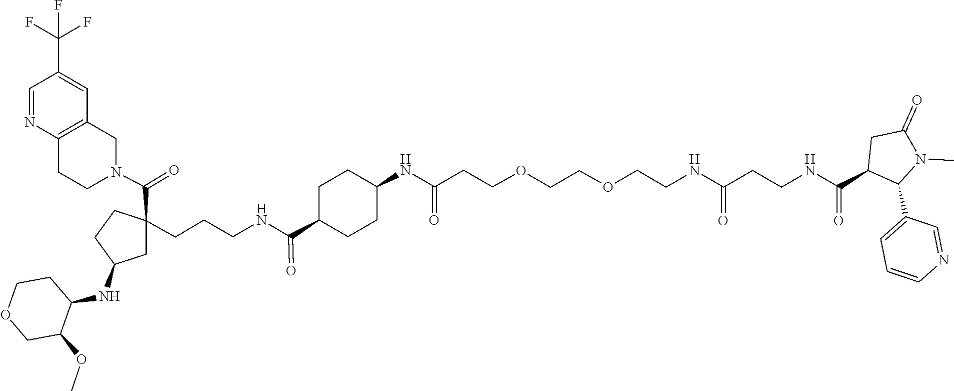 [SMILES: CO[C@H]1COCC[C@H]1N[C@H]1CC[C@@](CCCNC(=O)[C@H]2CC[C@@H](NC(=O)CCOCCOCCNC(=O)CCNC(=O)[C@H]3CC(=O)N(C)[C@@H]3c3cccnc3)CC2)(C(=O)N2CCc3ncc(C(F)(F)F)cc3C2)C1] |
| B20 | 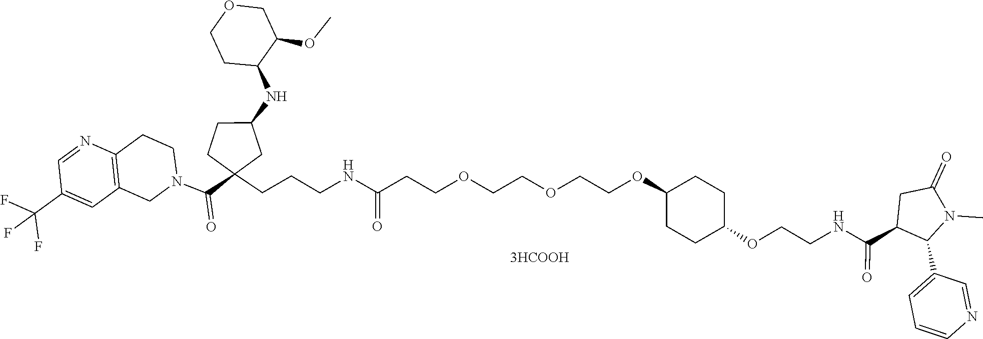 [SMILES: CO[C@@H]1COCC[C@@H]1N[C@@H]1CC[C@](CCCNC(=O)CCOCCOCCO[C@H]2CC[C@H](OCCNC(=O)[C@H]3CC(=O)N(C)[C@@H]3c3cccnc3)CC2)(C(=O)N2CCc3ncc(C(F)(F)F)cc3C2)C1] |
| B21 | 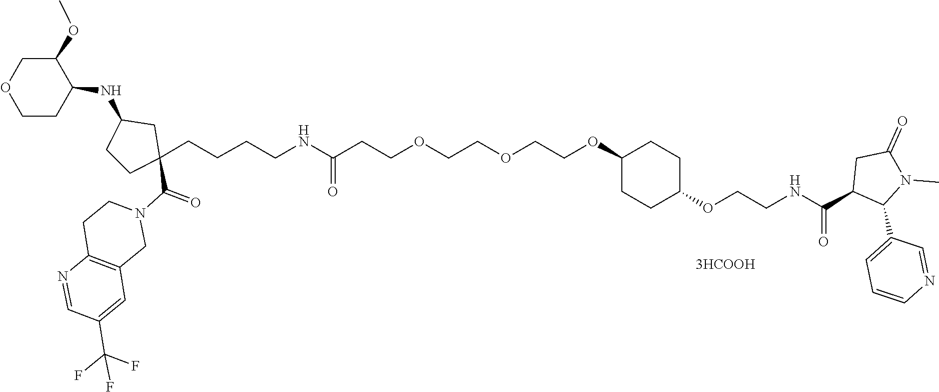 [SMILES: CO[C@@H]1COCC[C@@H]1N[C@@H]1CC[C@](CCCCNC(=O)CCOCCOCCO[C@H]2CC[C@H](OCCNC(=O)[C@H]3CC(=O)N(C)[C@@H]3c3cccnc3)CC2)(C(=O)N2CCc3ncc(C(F)(F)F)cc3C2)C1 ; "3HCOOH"] |
| B22 | 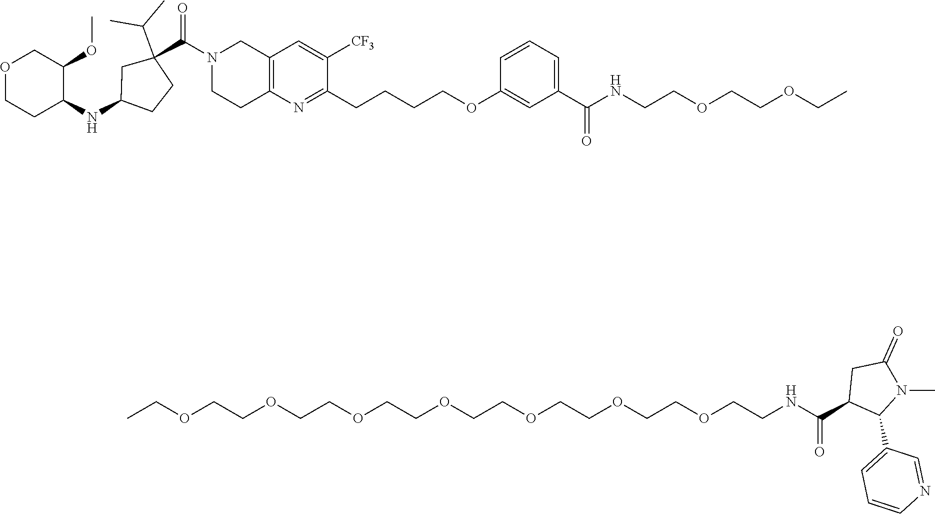 |
| B23 | 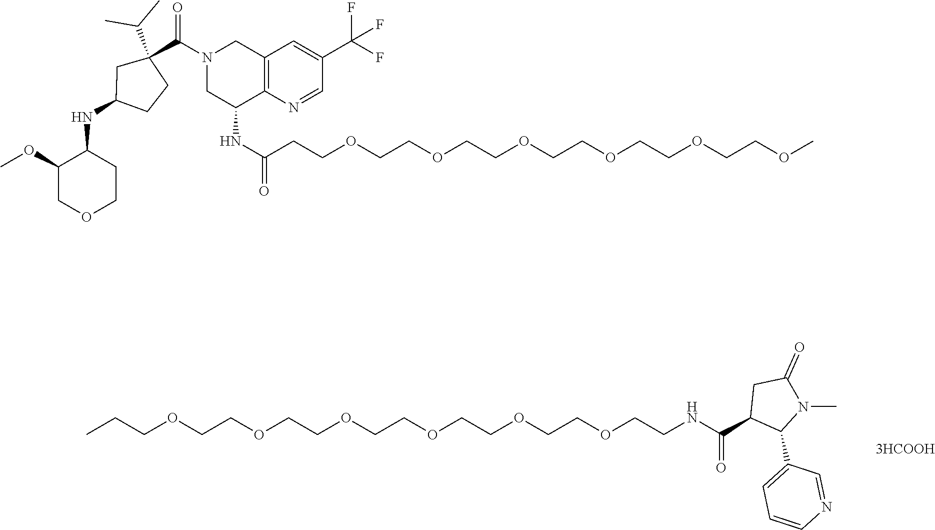 |
| B24 | 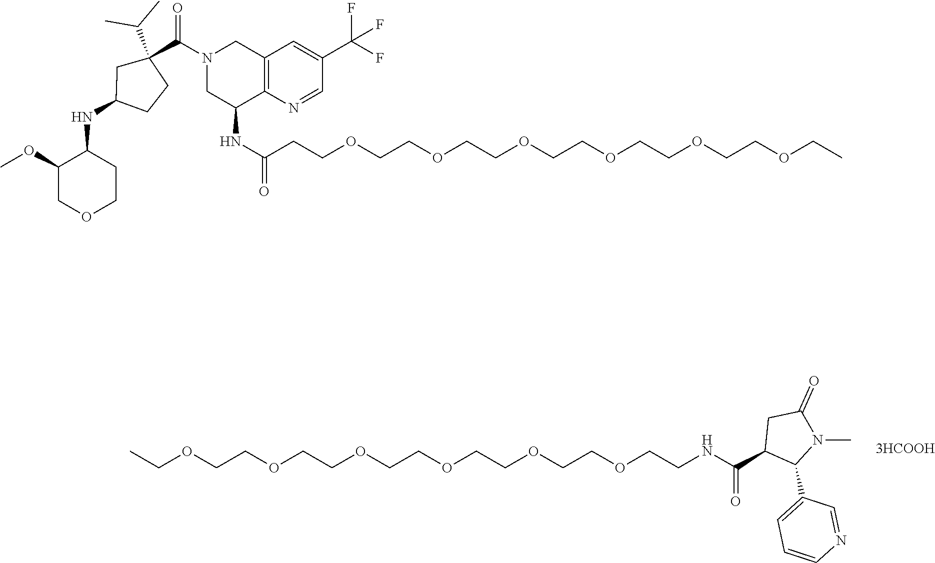 |
| C1 | 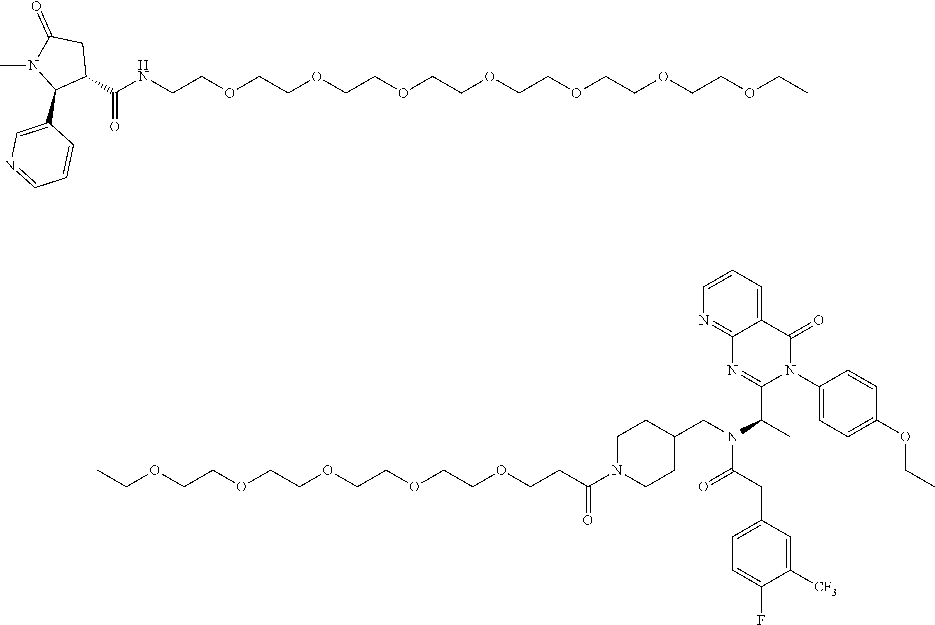 |
| C2 | 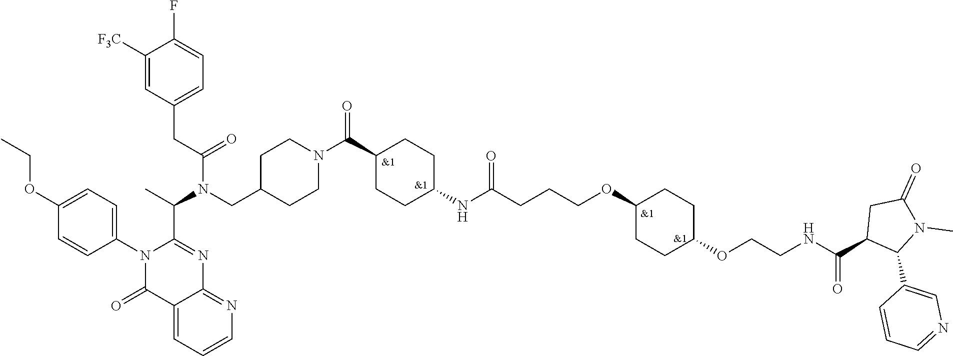 [SMILES: CCOc1ccc(-n2c([C@@H](C)N(CC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)C(=O)Cc3ccc(F)c(C(F)(F)F)c3)nc3ncccc3c2=O)cc1] |
| C3 | 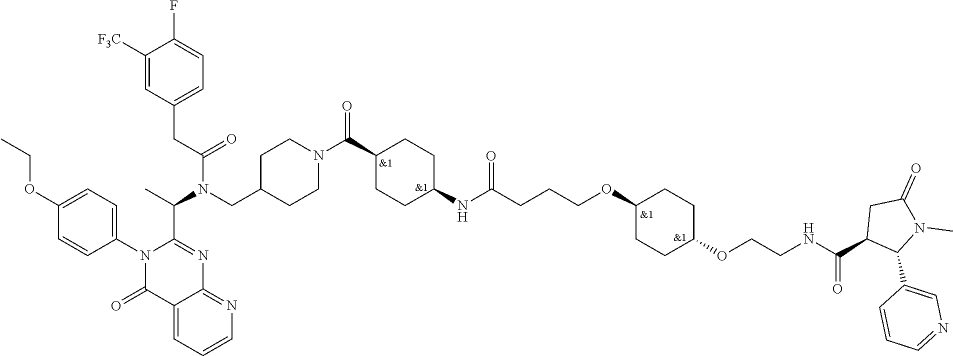 [SMILES: CCOc1ccc(-n2c([C@@H](C)N(CC3CCN(C(=O)[C@H]4CC[C@@H](NC(=O)CCCO[C@H]5CC[C@H](OCCNC(=O)[C@H]6CC(=O)N(C)[C@@H]6c6cccnc6)CC5)CC4)CC3)C(=O)Cc3ccc(F)c(C(F)(F)F)c3)nc3ncccc3c2=O)cc1] |
| D1 | 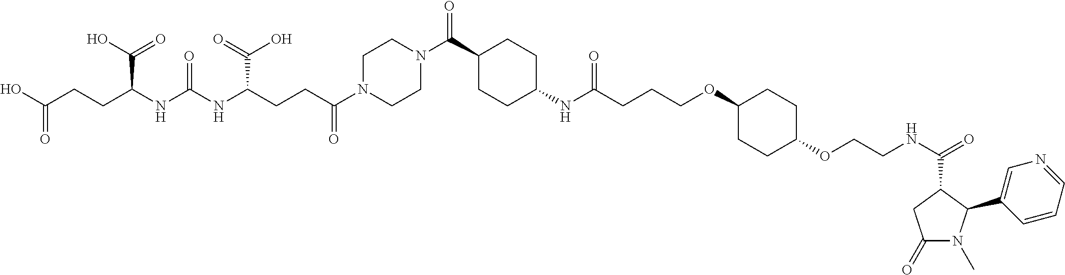 [SMILES: CN1C(=O)C[C@H](C(=O)NCCO[C@H]2CC[C@H](OCCCC(=O)N[C@H]3CC[C@H](C(=O)N4CCN(C(=O)CC[C@H](NC(=O)N[C@@H](CCC(=O)O)C(=O)O)C(=O)O)CC4)CC3)CC2)[C@H]1c1cccnc1] |
| D2 | 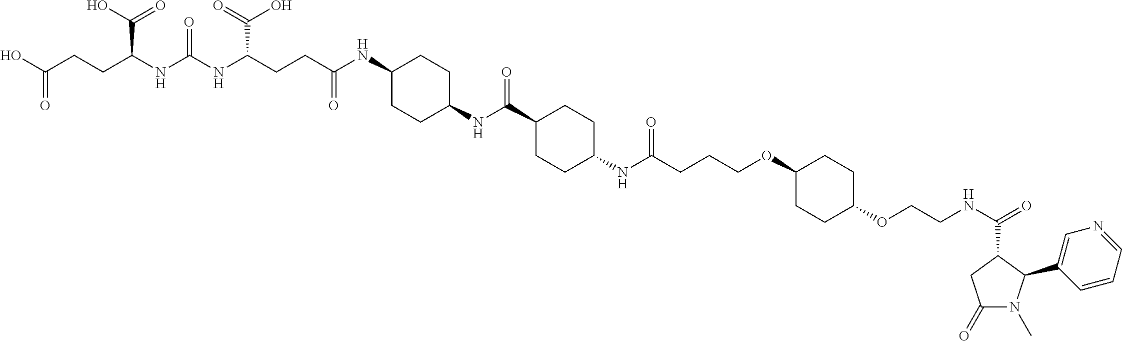 [SMILES: CN1C(=O)C[C@H](C(=O)NCCO[C@H]2CC[C@H](OCCCC(=O)N[C@H]3CC[C@H](C(=O)N[C@H]4CC[C@@H](NC(=O)CC[C@H](NC(=O)N[C@@H](CCC(=O)O)C(=O)O)C(=O)O)CC4)CC3)CC2)[C@H]1c1cccnc1] |
| D3 | 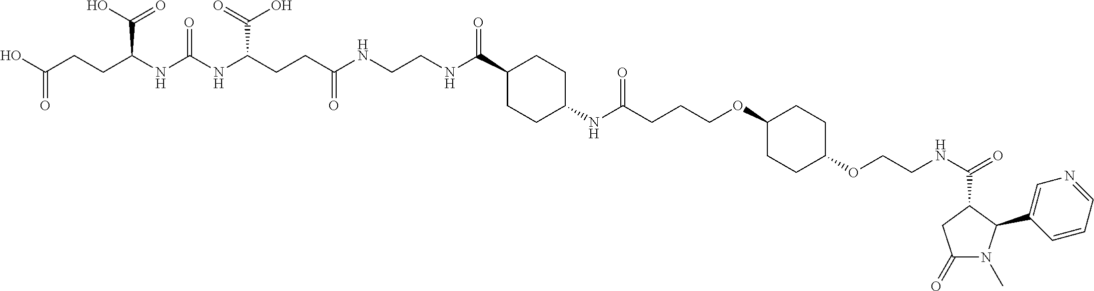 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)N[C@@H](CCC(=O)O)C(=O)O)C(=O)O)CC3)CC2)[C@H]1c1cccnc1] |
| D4 | 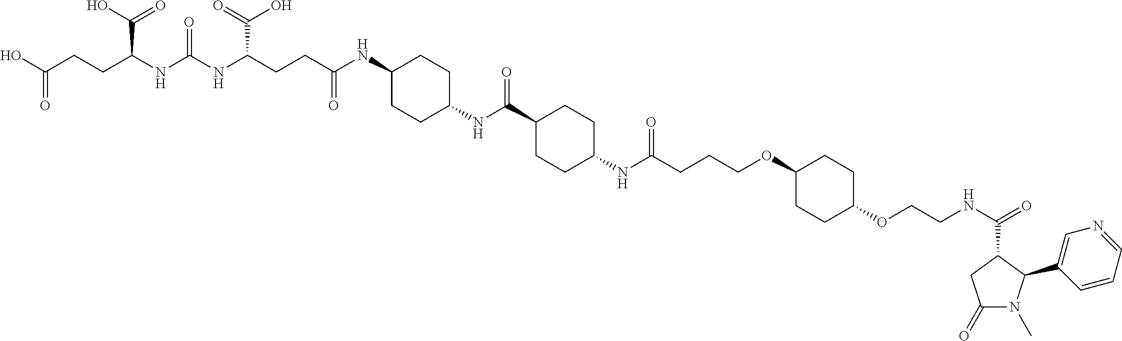 [SMILES: CN1C(=O)C[C@H](C(=O)NCCO[C@H]2CC[C@H](OCCCC(=O)N[C@H]3CC[C@H](C(=O)N[C@H]4CC[C@H](NC(=O)CC[C@H](NC(=O)N[C@@H](CCC(=O)O)C(=O)O)C(=O)O)CC4)CC3)CC2)[C@H]1c1cccnc1] |
| D5 | 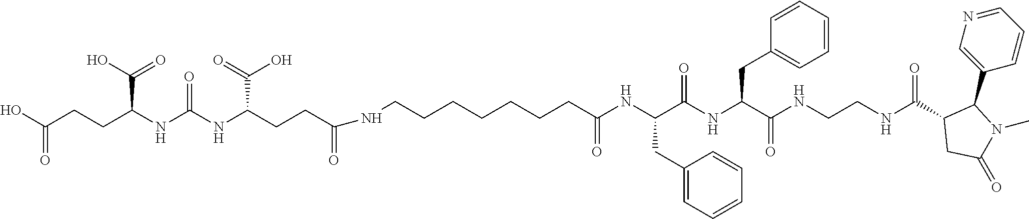 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| D6 | 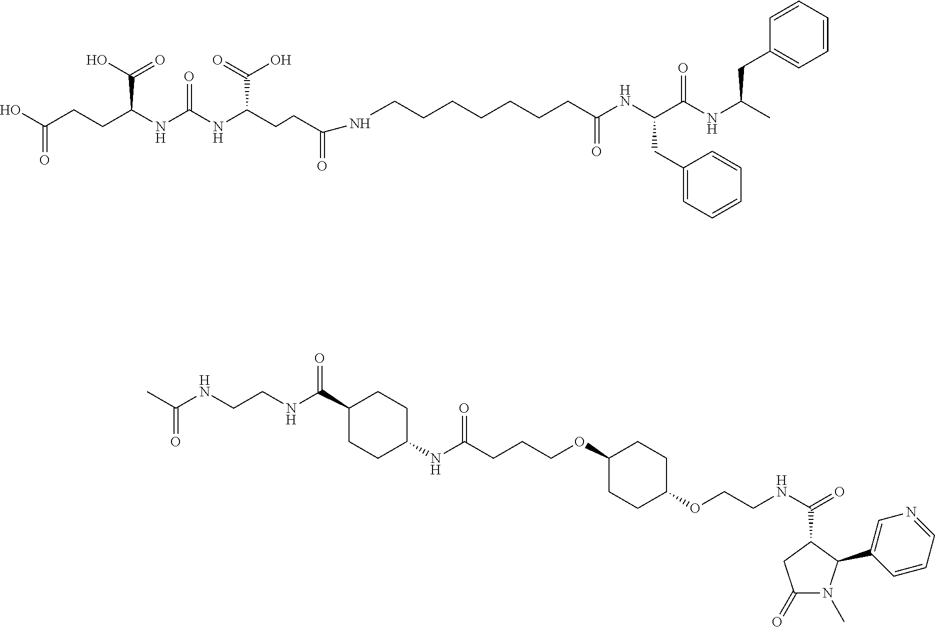 [SMILES: C[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O ; CC(=O)NCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1] |
| D7 | 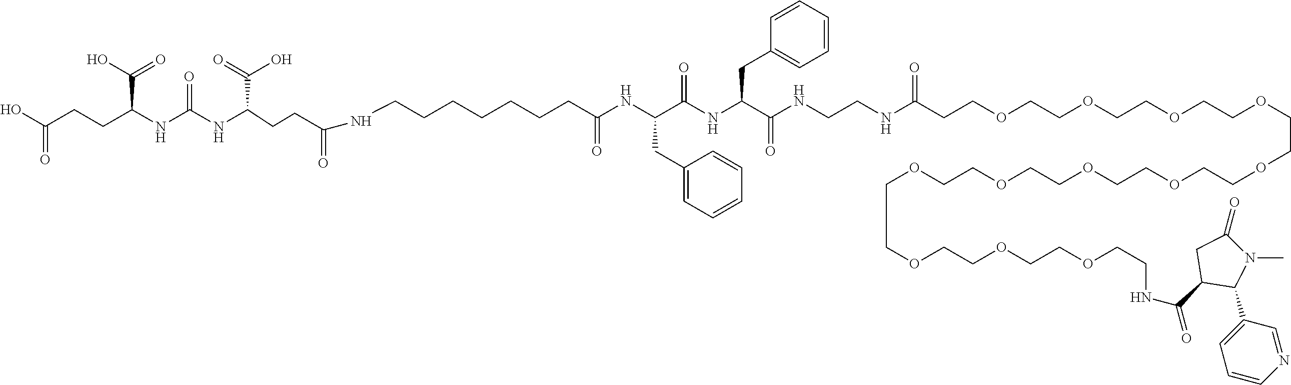 [SMILES: CN1C(=O)C[C@H](C(=O)NCCOCCOCCOCCOCCOCCOCCOCCOCCOCCOCCOCCOCCC(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| D8 | 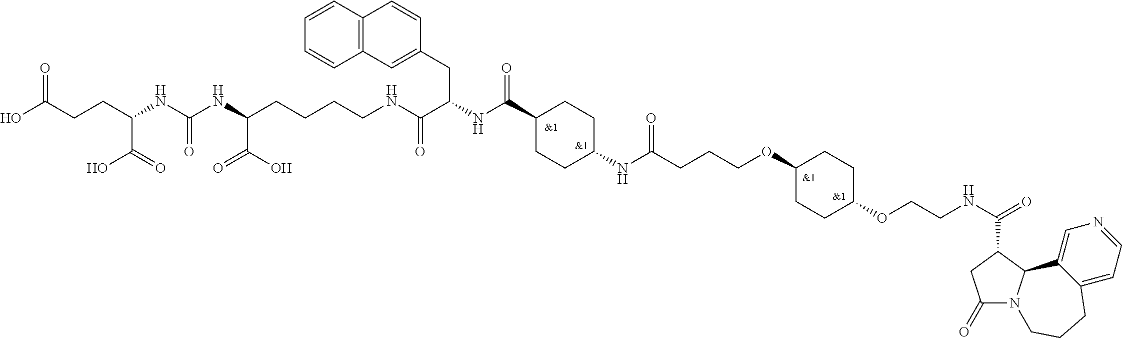 [SMILES: O=C(O)CC[C@H](NC(=O)N[C@@H](CCCCNC(=O)[C@H](Cc1ccc2ccccc2c1)NC(=O)[C@H]1CC[C@H](NC(=O)CCCO[C@H]2CC[C@H](OCCNC(=O)[C@H]3CC(=O)N4CCCc5ccncc5[C@H]34)CC2)CC1)C(=O)O)C(=O)O] |
| D9 | 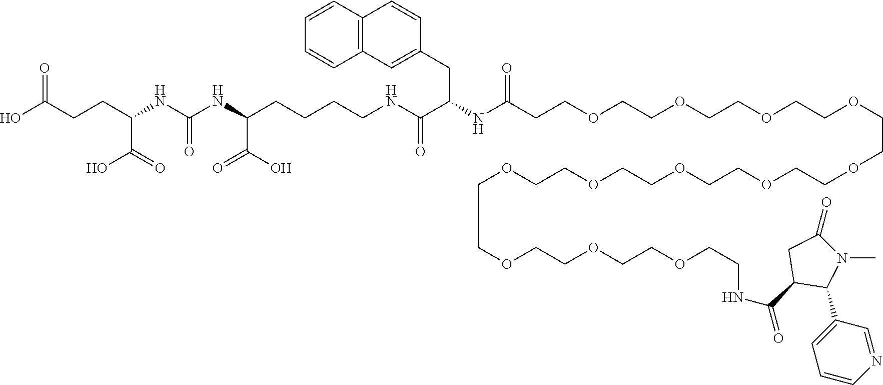 [SMILES: CN1C(=O)C[C@H](C(=O)NCCOCCOCCOCCOCCOCCOCCOCCOCCOCCOCCOCCOCCC(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1] |
| E1 | 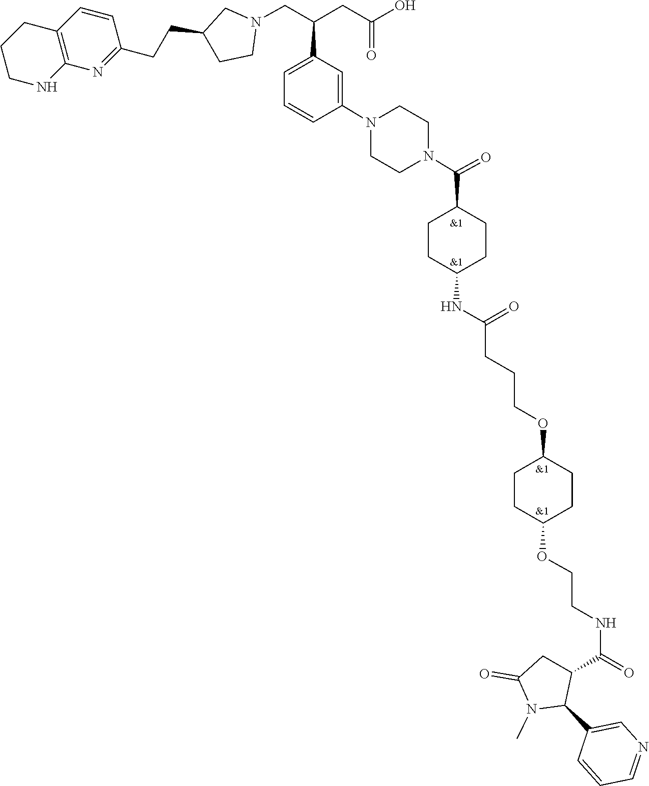 [SMILES: CN1C(=O)C[C@H](C(=O)NCCO[C@H]2CC[C@H](OCCCC(=O)N[C@H]3CC[C@H](C(=O)N4CCN(c5cccc([C@H](CC(=O)O)CN6CC[C@@H](CCc7ccc8c(n7)NCCC8)C6)c5)CC4)CC3)CC2)[C@H]1c1cccnc1] |
| E2 | 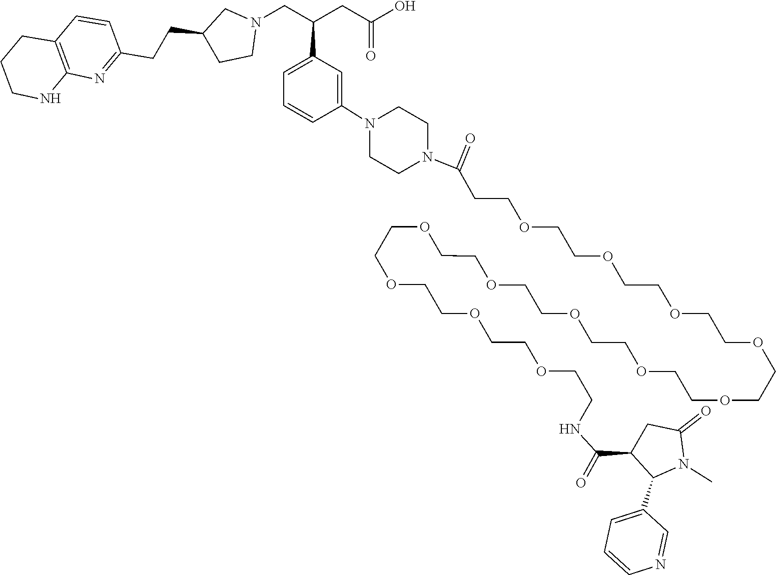 [SMILES: CN1C(=O)C[C@H](C(=O)NCCOCCOCCOCCOCCOCCOCCOCCOCCOCCOCCOCCOCCC(=O)N2CCN(c3cccc([C@H](CC(=O)O)CN4CC[C@@H](CCc5ccc6c(n5)NCCC6)C4)c3)CC2)[C@H]1c1cccnc1] |
| E3 | 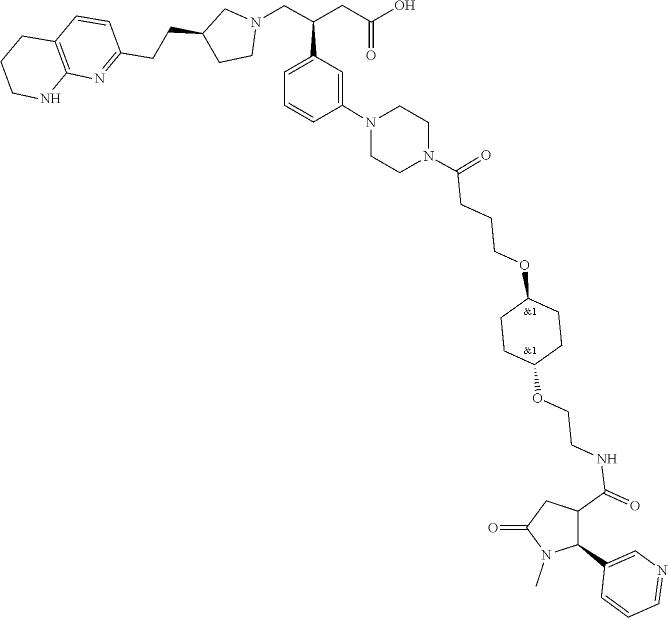 [SMILES: CN1C(=O)CC(C(=O)NCCO[C@H]2CC[C@H](OCCCC(=O)N3CCN(c4cccc([C@H](CC(=O)O)CN5CC[C@@H](CCc6ccc7c(n6)NCCC7)C5)c4)CC3)CC2)[C@H]1c1cccnc1] |
| F2 | 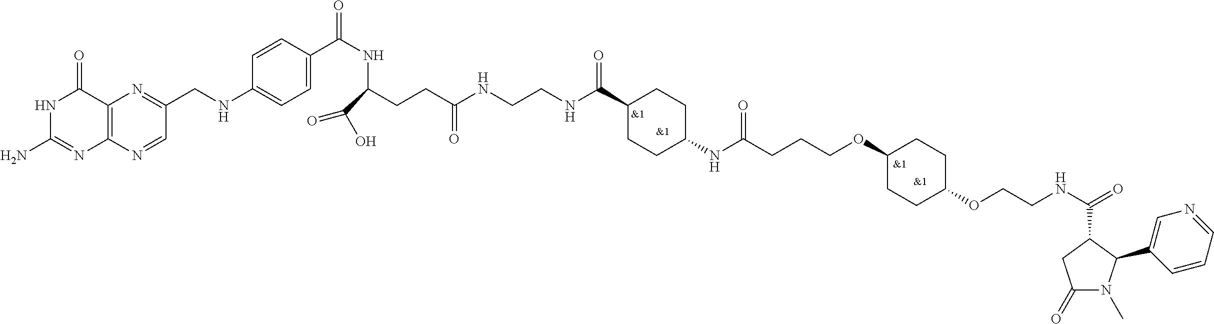 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)c4ccc(NCc5cnc6nc(N)[nH]c(=O)c6n5)cc4)C(=O)O)CC3)CC2)[C@H]1c1cccnc1] |
| F3 | 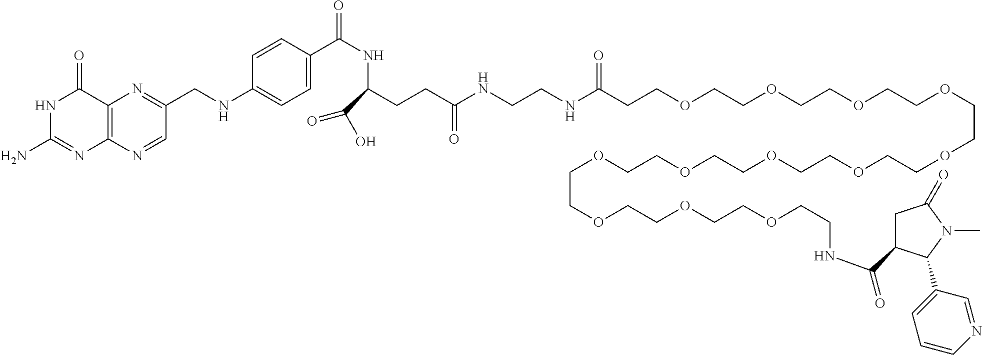 [SMILES: CN1C(=O)C[C@H](C(=O)NCCOCCOCCOCCOCCOCCOCCOCCOCCOCCOCCOCCOCCC(=O)NCCNC(=O)CC[C@H](NC(=O)c2ccc(NCc3cnc4nc(N)[nH]c(=O)c4n3)cc2)C(=O)O)[C@H]1c1cccnc1] |
| G1 | 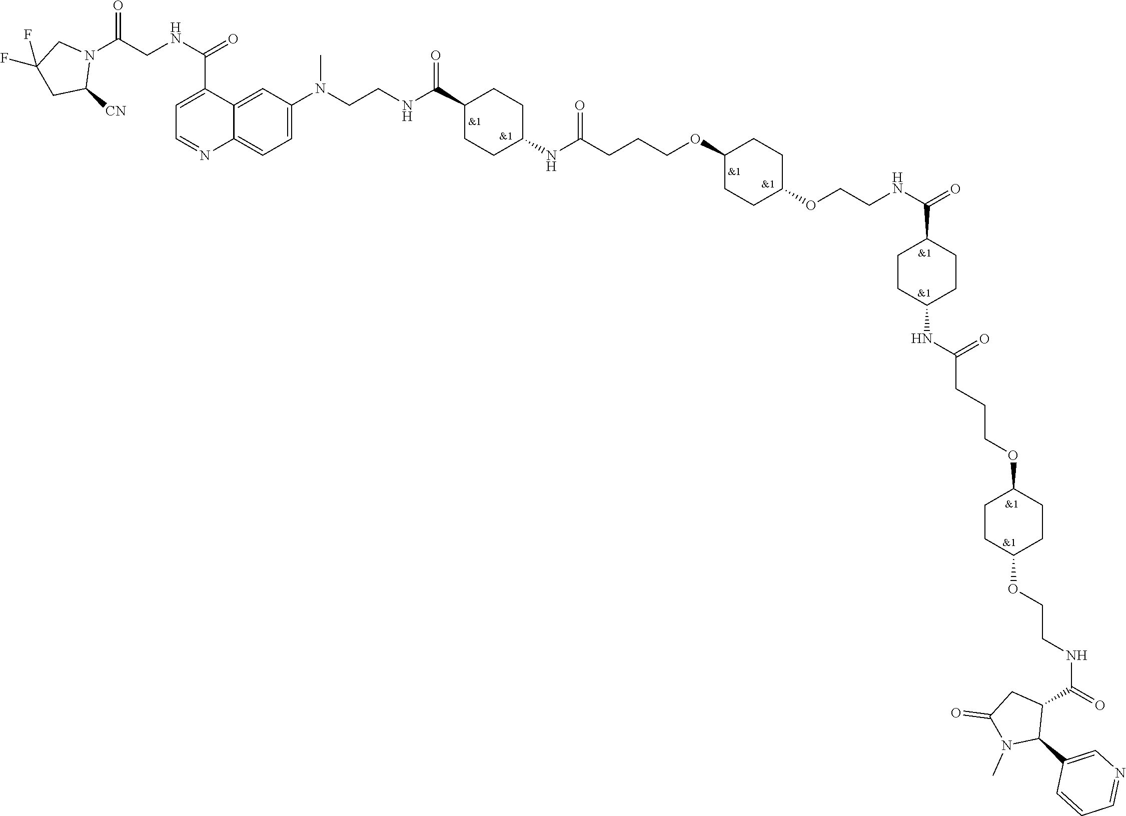 [SMILES: CN(CCNC(=O)[C@H]1CC[C@H](NC(=O)CCCO[C@H]2CC[C@H](OCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)CC1)c1ccc2nccc(C(=O)NCC(=O)N3CC(F)(F)C[C@@H]3C#N)c2c1] |
| G6 | 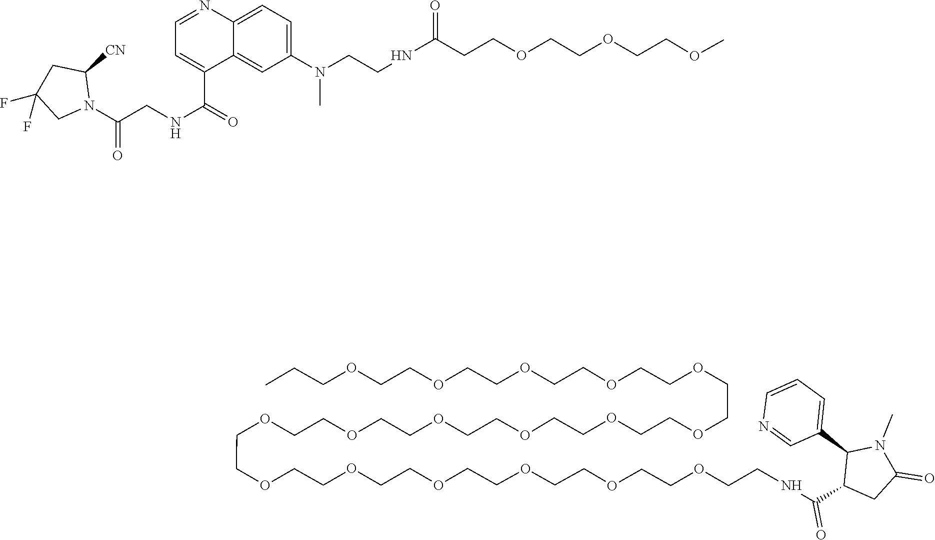 [SMILES: COCCOCCOCCC(=O)NCCN(C)c1ccc2nccc(C(=O)NCC(=O)N3CC(F)(F)C[C@H]3C#N)c2c1 ; CCCOCCOCCOCCOCCOCCOCCOCCOCCOCCOCCOCCOCCOCCOCCOCCOCCOCCNC(=O)[C@H]1CC(=O)N(C)[C@@H]1c1cccnc1] |
| G7 | 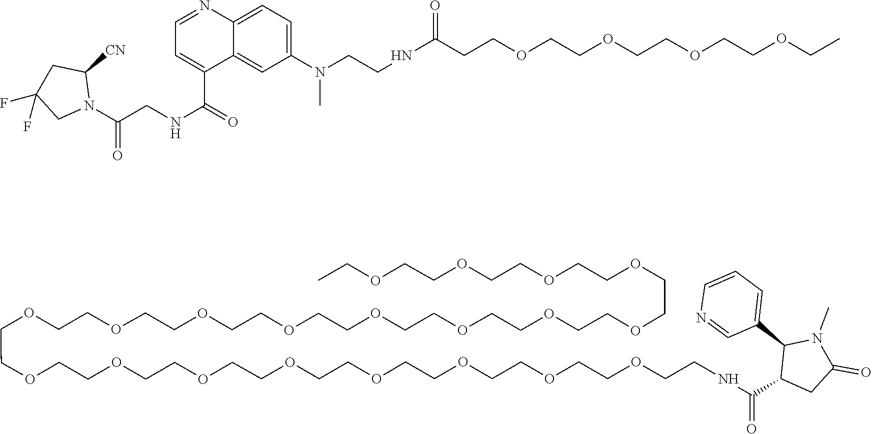 [SMILES: CCOCCOCCOCCOCCC(=O)NCCN(C)c1ccc2nccc(C(=O)NCC(=O)N3CC(F)(F)C[C@H]3C#N)c2c1 ; CCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1] |
| G8 | 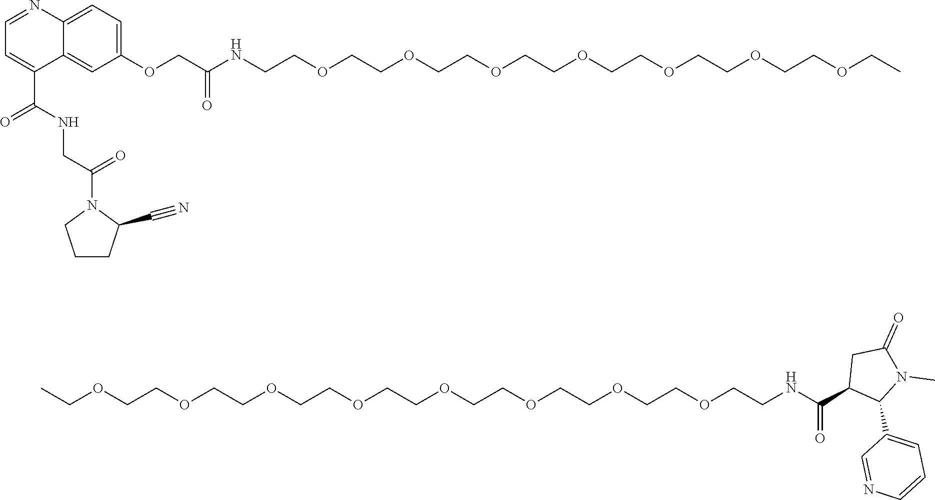 |
| G9 | 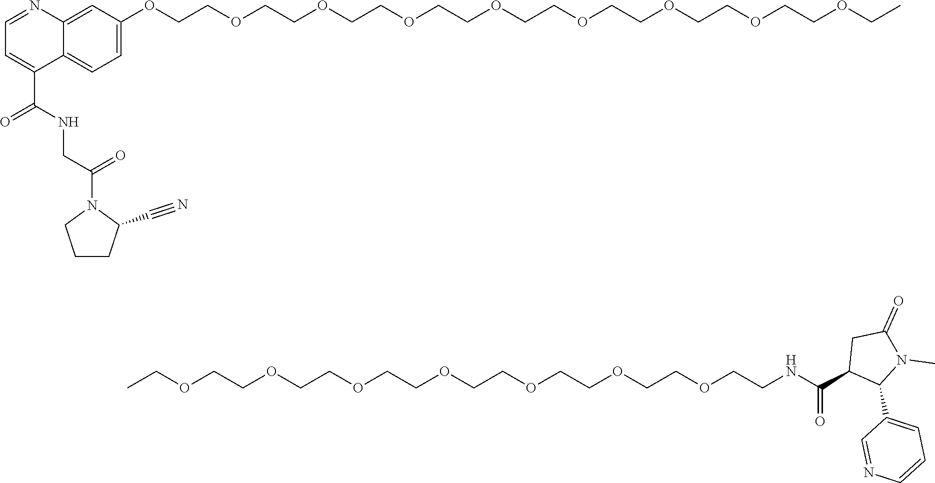 |
| G10 | 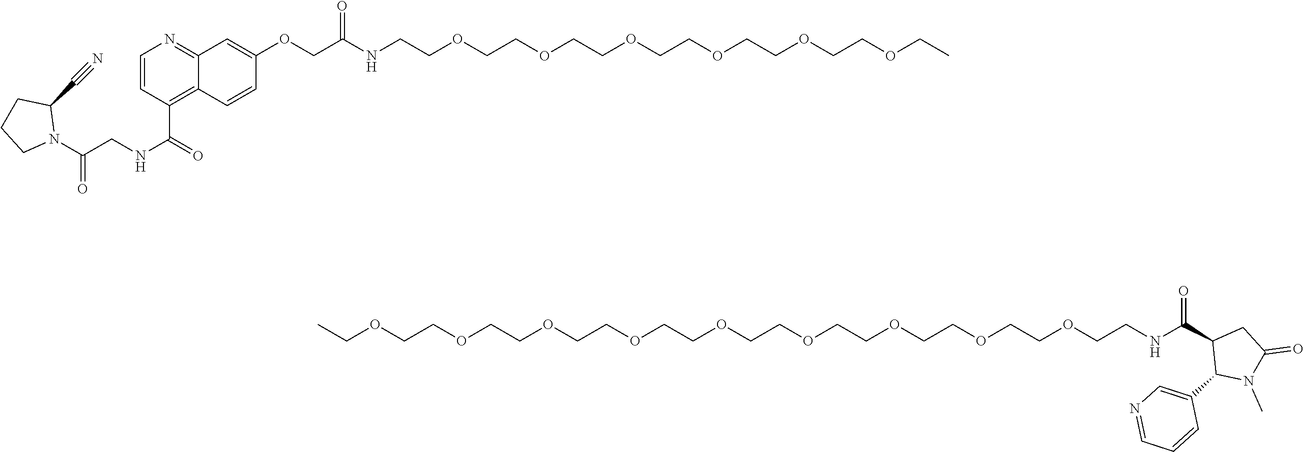 |
| G11 | 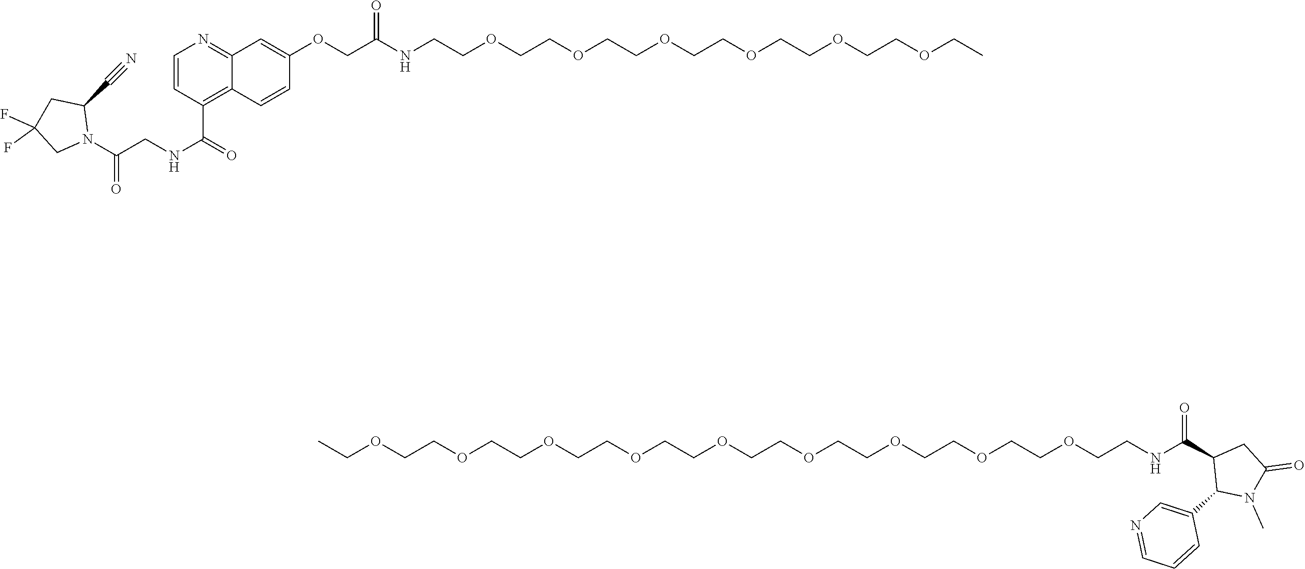 |
| G12 | 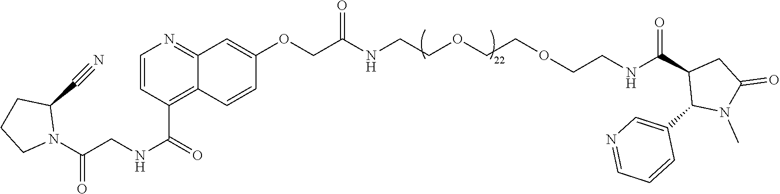 |
| G13 | 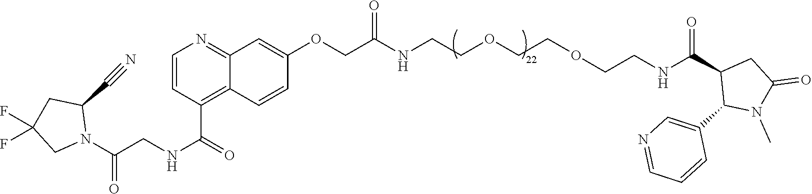 |
| H1 | 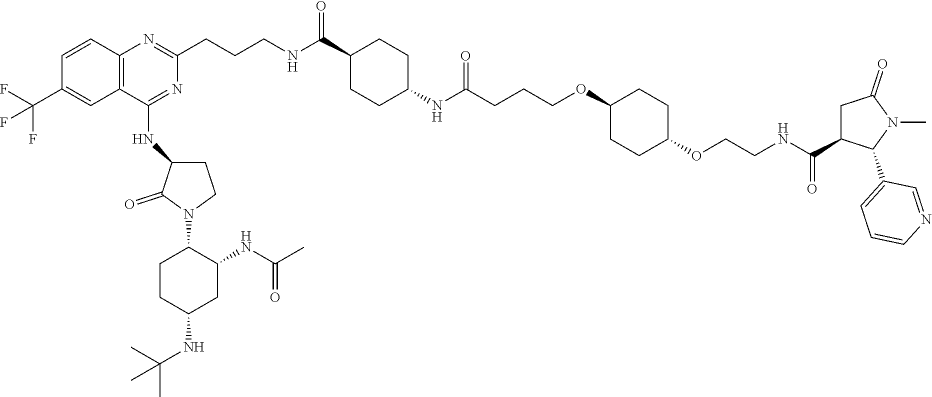 [SMILES: CC(=O)N[C@@H]1C[C@H](NC(C)(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H2 | 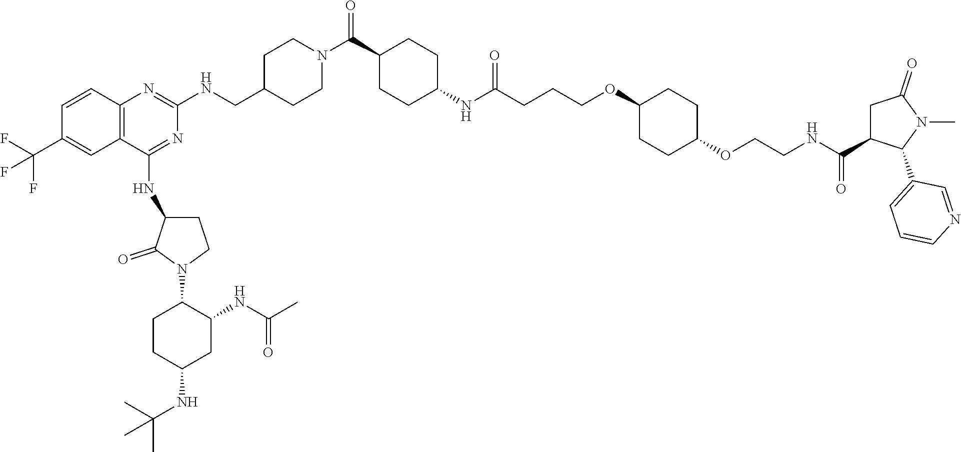 [SMILES: CC(=O)N[C@@H]1C[C@H](NC(C)(C)C)CC[C@@H]1N1CC[C@H](Nc2nc(NCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H3 | 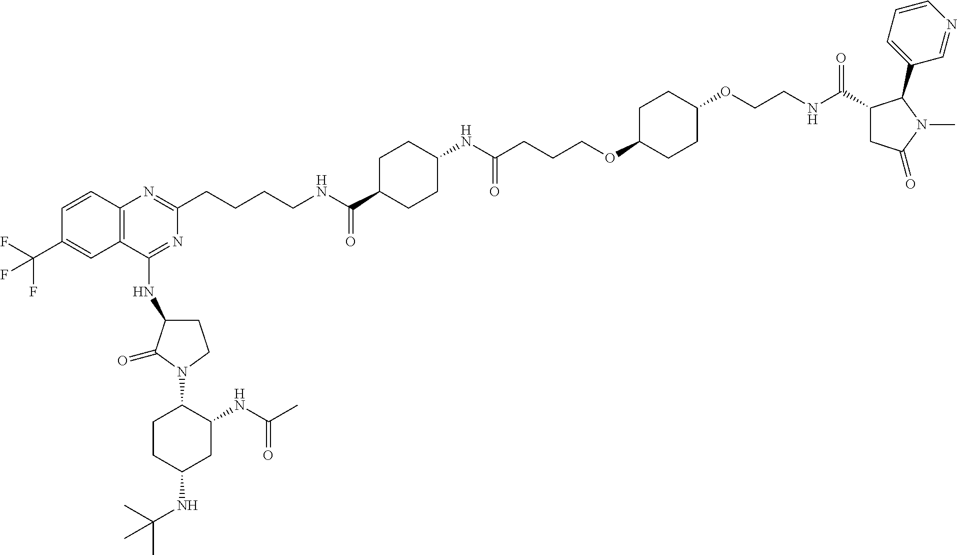 [SMILES: CC(=O)N[C@@H]1C[C@H](NC(C)(C)C)CC[C@@H]1N1CC[C@H](Nc2nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H4 | 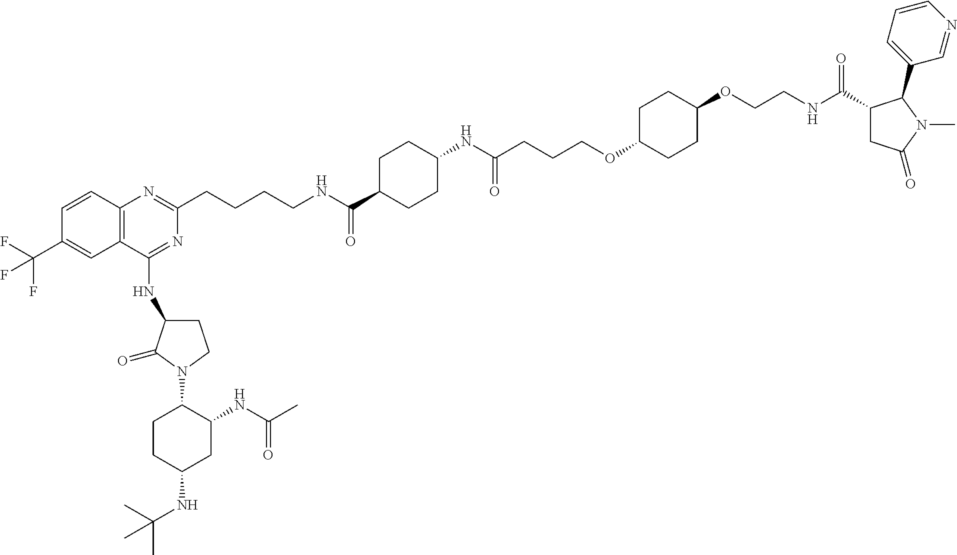 [SMILES: CC(=O)N[C@@H]1C[C@H](NC(C)(C)C)CC[C@@H]1N1CC[C@H](Nc2nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H5 | 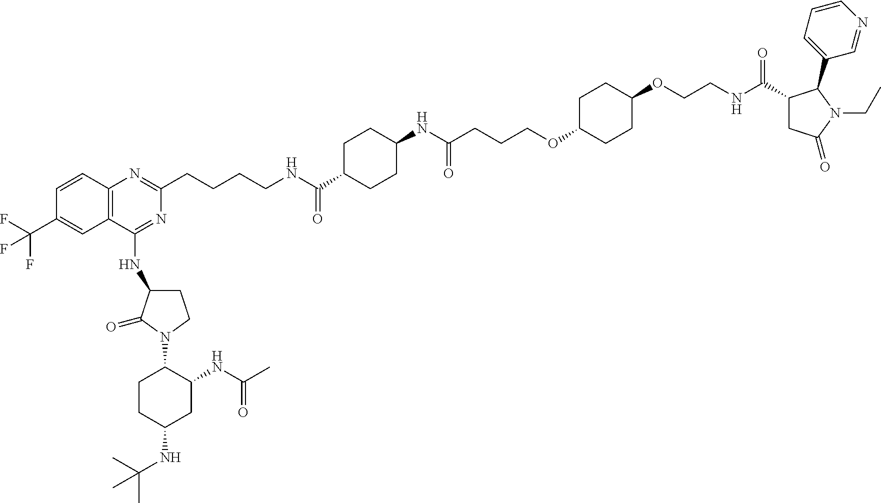 [SMILES: CCN1C(=O)C[C@H](C(=O)NCCO[C@H]2CC[C@H](OCCCC(=O)N[C@H]3CC[C@H](C(=O)NCCCCc4nc(N[C@H]5CCN([C@H]6CC[C@@H](NC(C)(C)C)C[C@H]6NC(C)=O)C5=O)c5cc(C(F)(F)F)ccc5n4)CC3)CC2)[C@H]1c1cccnc1] |
| H6 | 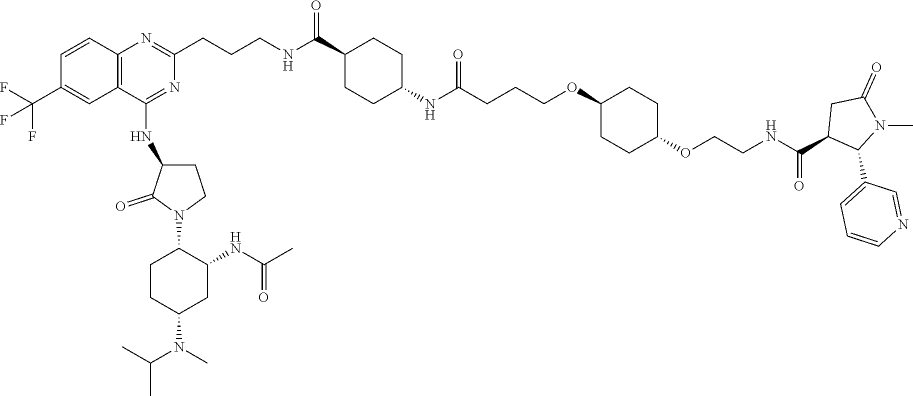 [SMILES: CC(=O)N[C@@H]1C[C@H](N(C)C(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H7 | 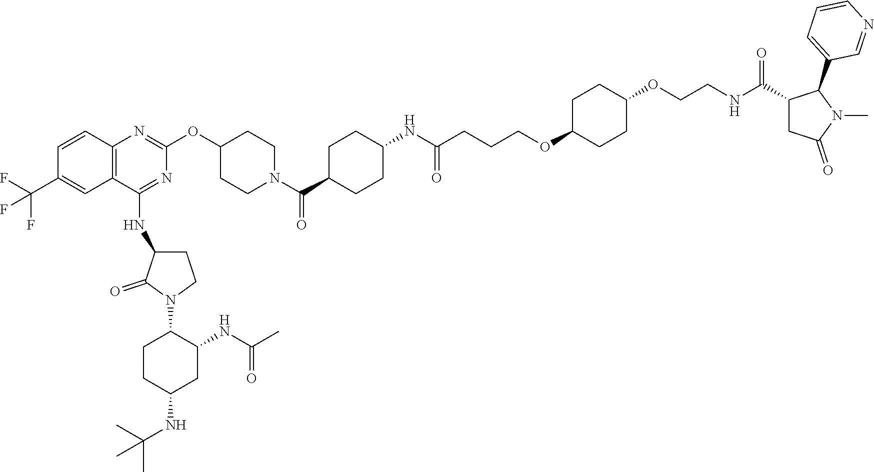 [SMILES: CC(=O)N[C@@H]1C[C@H](NC(C)(C)C)CC[C@@H]1N1CC[C@H](Nc2nc(OC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H8 | 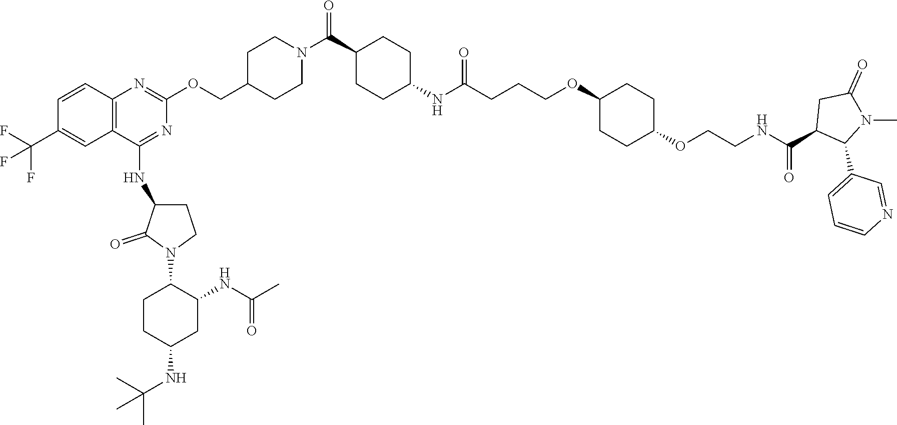 [SMILES: CC(=O)N[C@@H]1C[C@H](NC(C)(C)C)CC[C@@H]1N1CC[C@H](Nc2nc(OCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H9 | 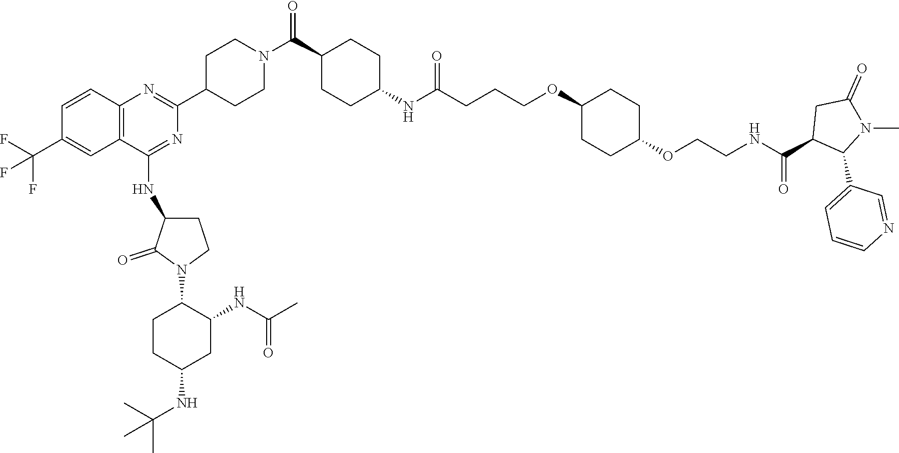 [SMILES: CC(=O)N[C@@H]1C[C@H](NC(C)(C)C)CC[C@@H]1N1CC[C@H](Nc2nc(C3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H10 | 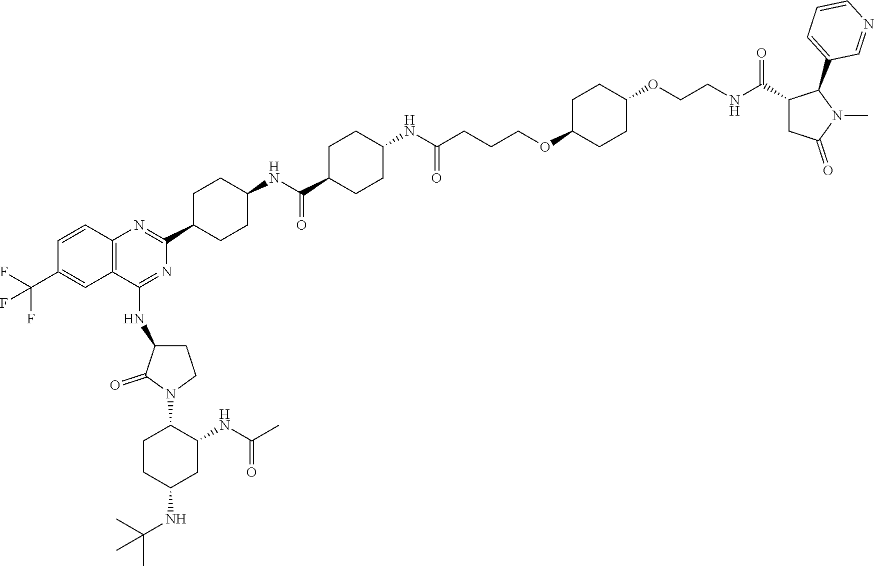 [SMILES: CC(=O)N[C@@H]1C[C@H](NC(C)(C)C)CC[C@@H]1N1CC[C@H](Nc2nc([C@H]3CC[C@@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H11 | 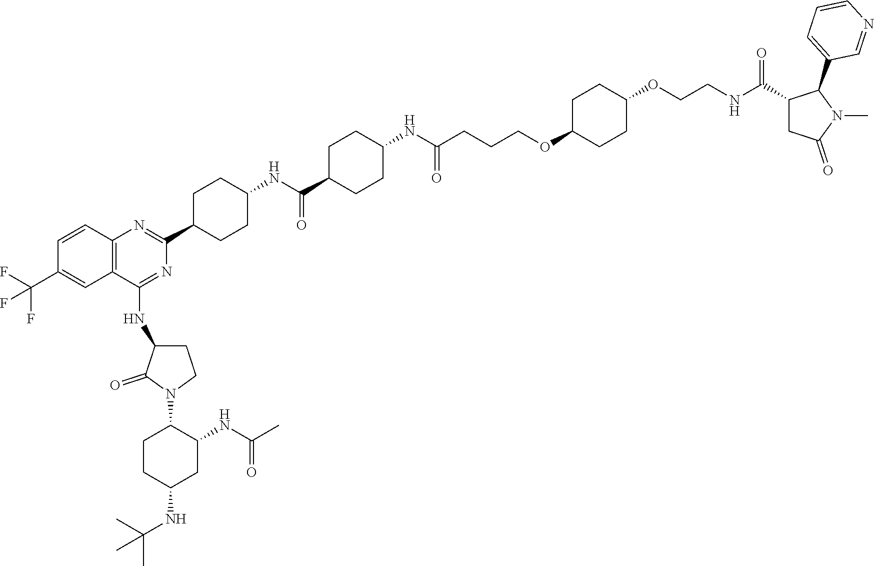 [SMILES: CC(=O)N[C@@H]1C[C@H](NC(C)(C)C)CC[C@@H]1N1CC[C@H](Nc2nc([C@H]3CC[C@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H12 | 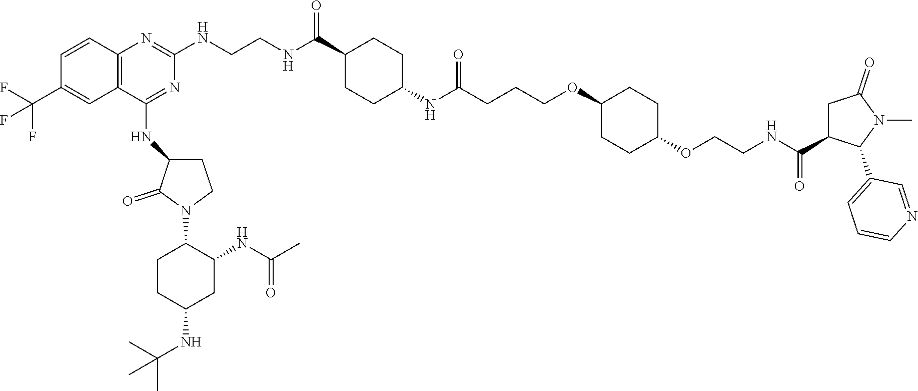 [SMILES: CC(=O)N[C@@H]1C[C@H](NC(C)(C)C)CC[C@@H]1N1CC[C@H](Nc2nc(NCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H13 | 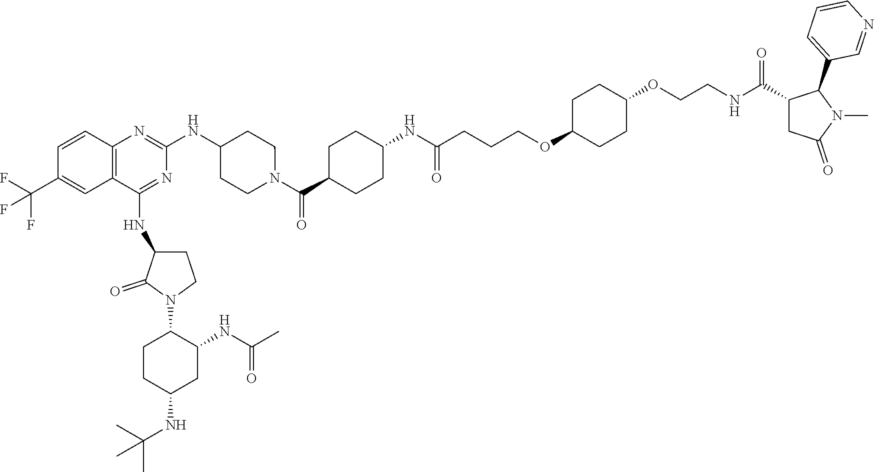 [SMILES: CC(=O)N[C@@H]1C[C@H](NC(C)(C)C)CC[C@@H]1N1CC[C@H](Nc2nc(NC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H14 | 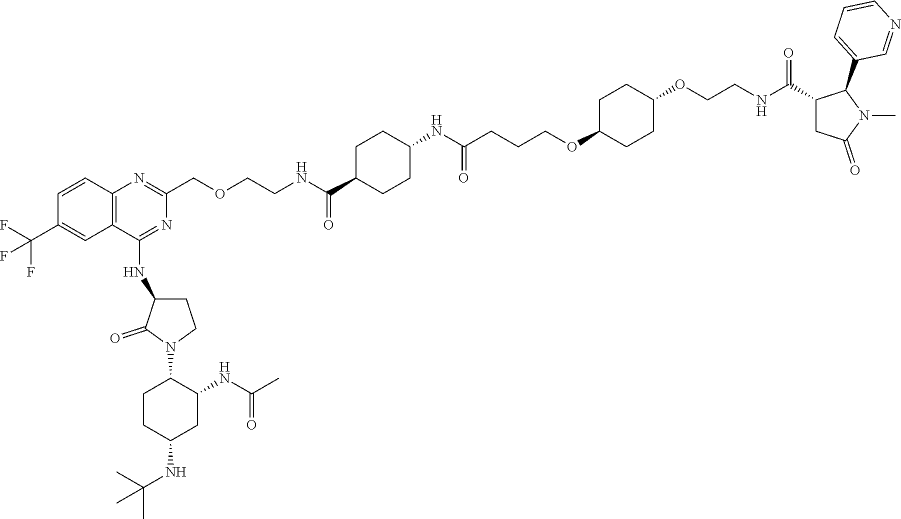 [SMILES: CC(=O)N[C@@H]1C[C@H](NC(C)(C)C)CC[C@@H]1N1CC[C@H](Nc2nc(COCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| J1 | 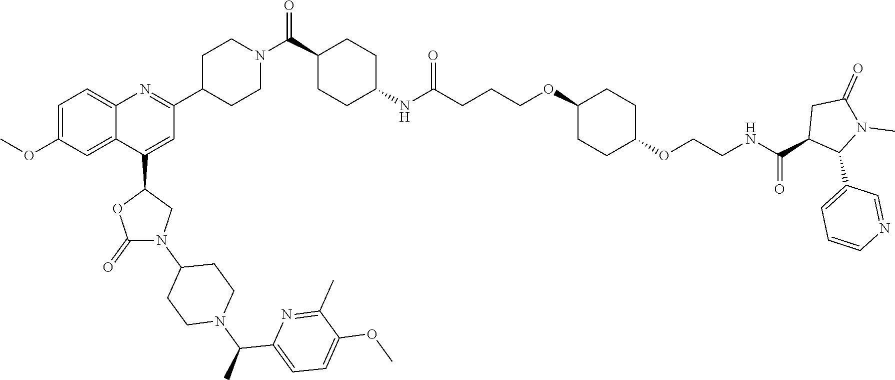 |
| J2 | 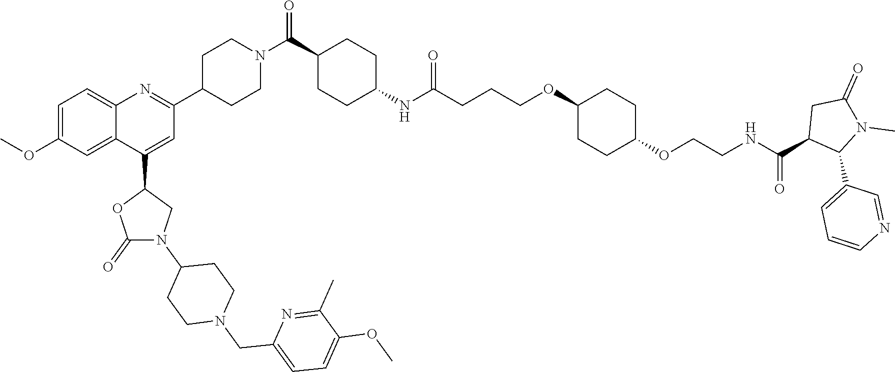 |
| J3 | 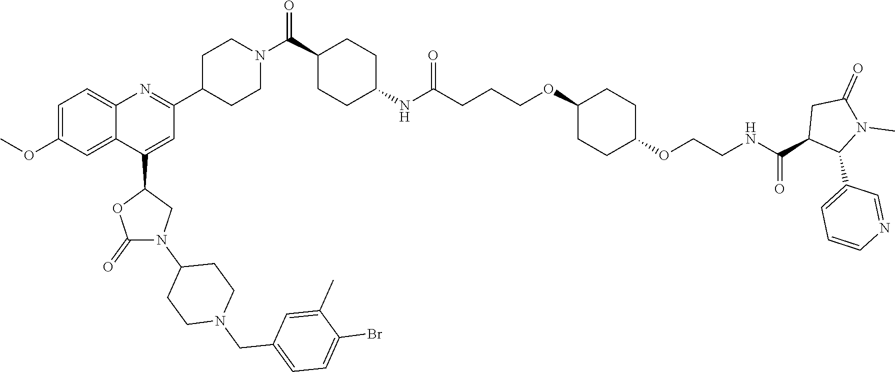 |
| J4 | 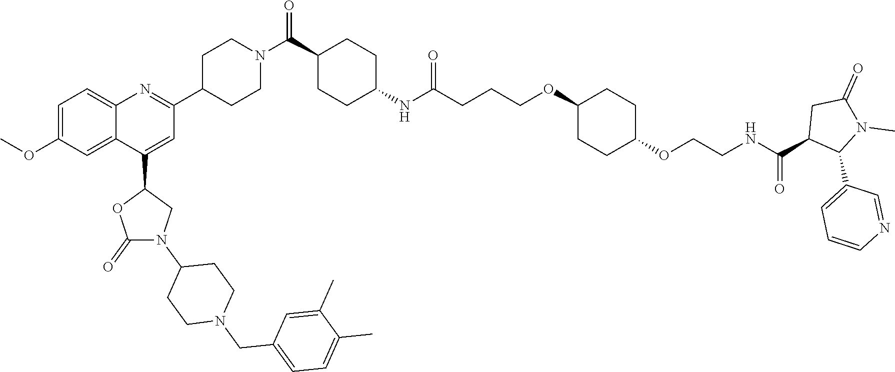 |
| J5 | 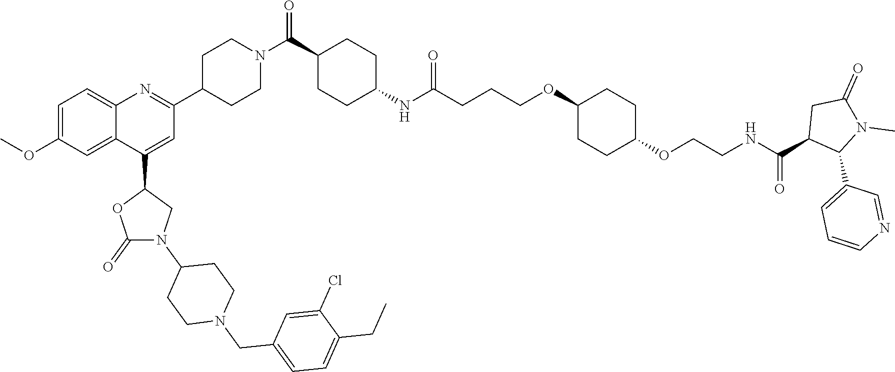 |
| J6 | 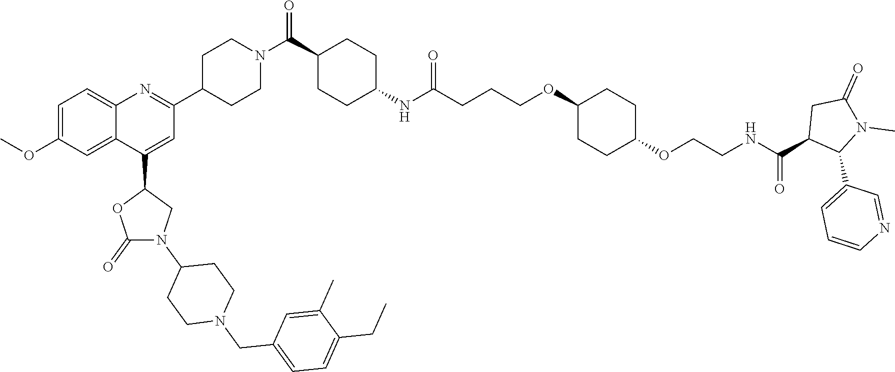 |
| J7 | 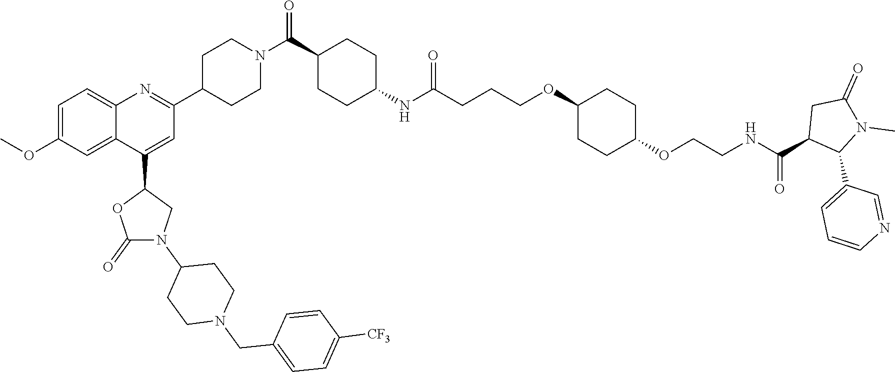 |
| J8 | 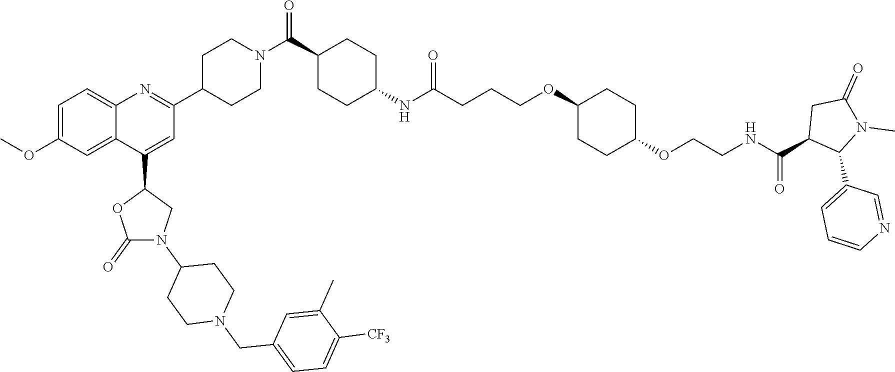 |
| J9 | 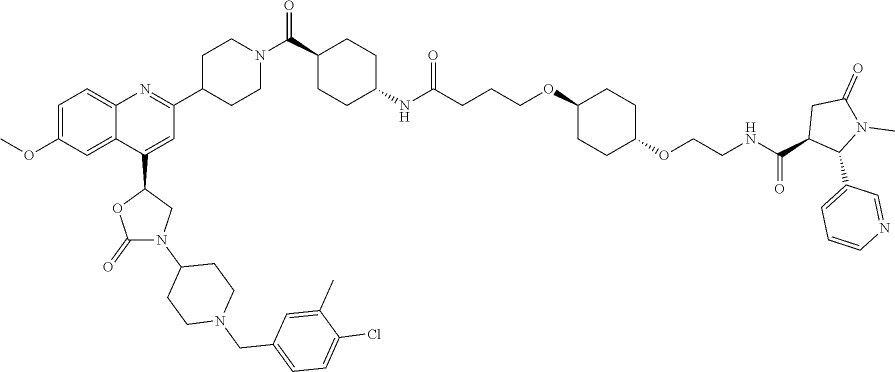 |
| J10 | 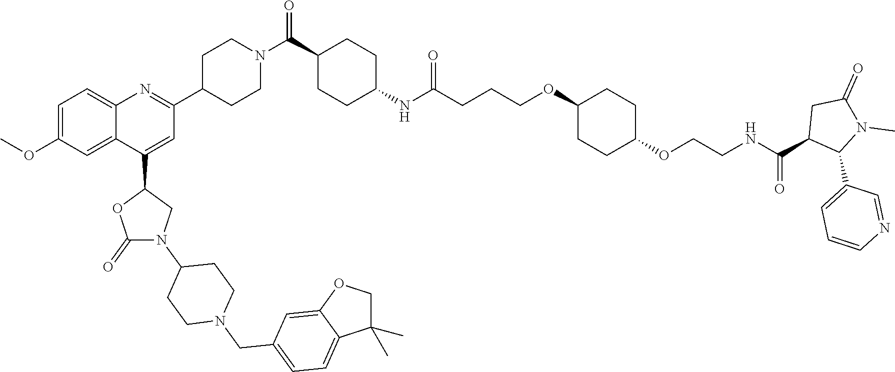 |
| J11 | 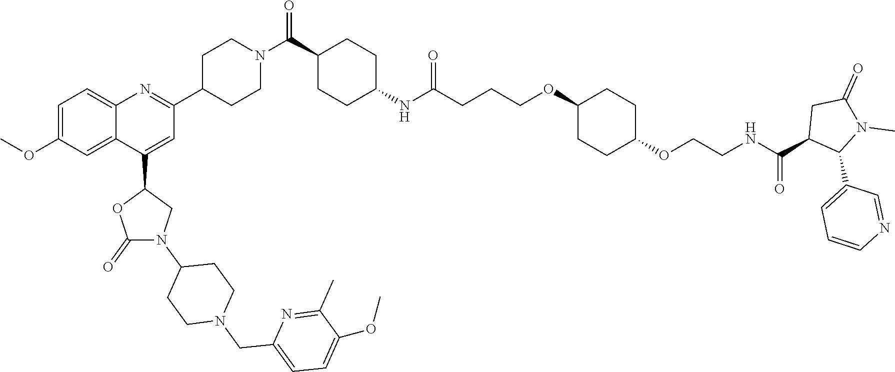 |
| J12 | 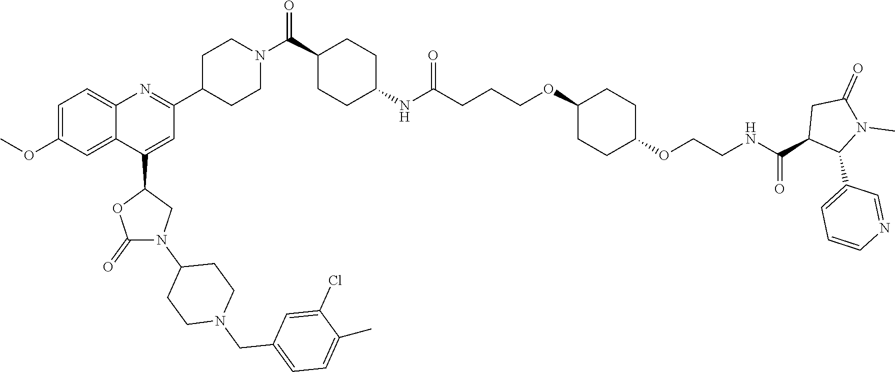 |
| J13 | 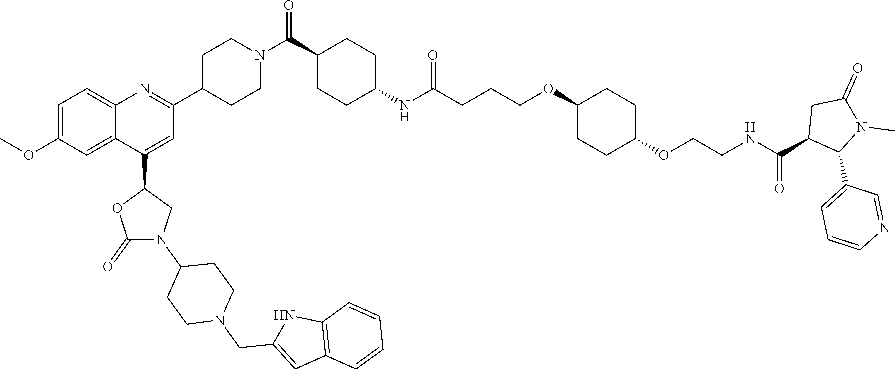 |
| J14 | 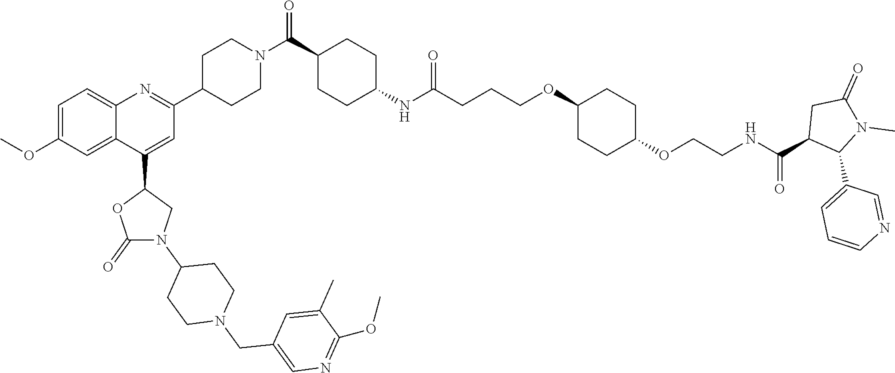 |
| J15 | 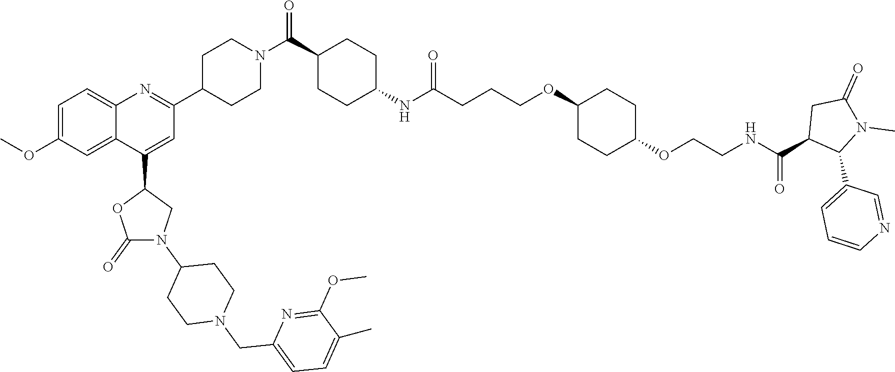 |
| J16 | 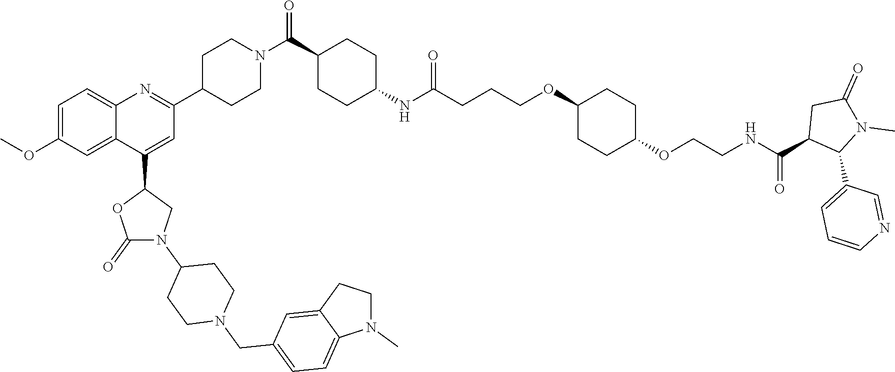 |
| J17 | 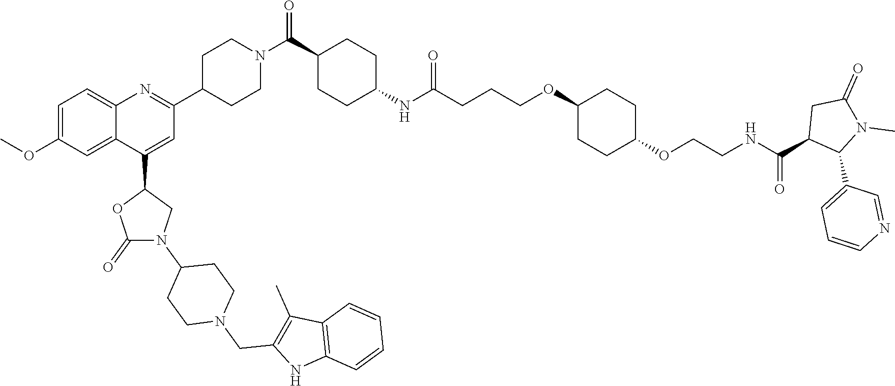 |
| J18 | 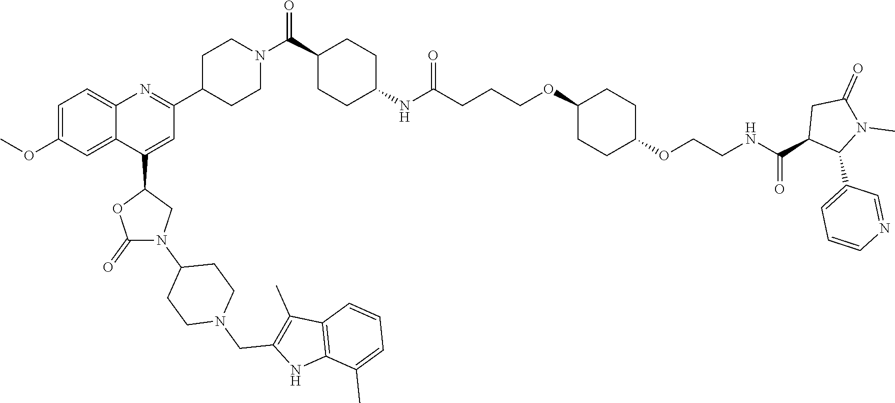 |
| J19 | 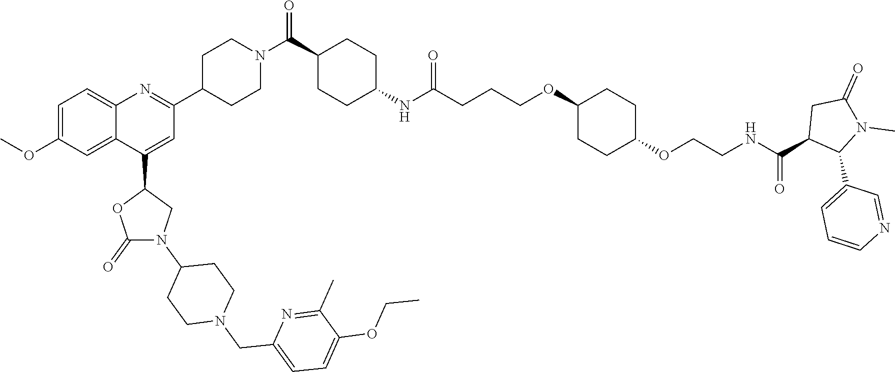 |
| J20 | 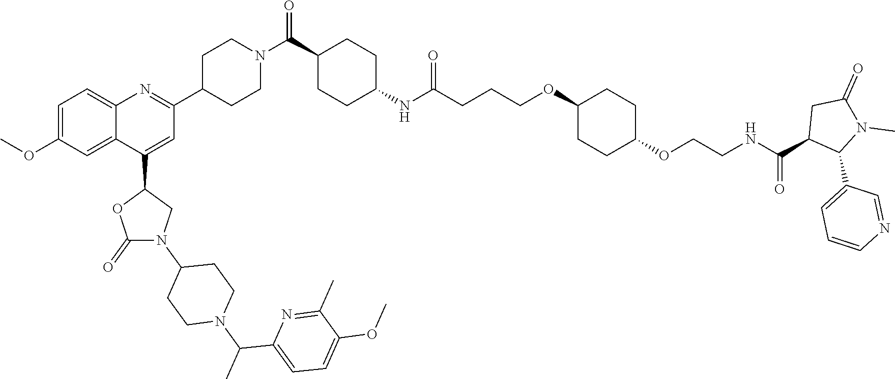 |
| J21 | 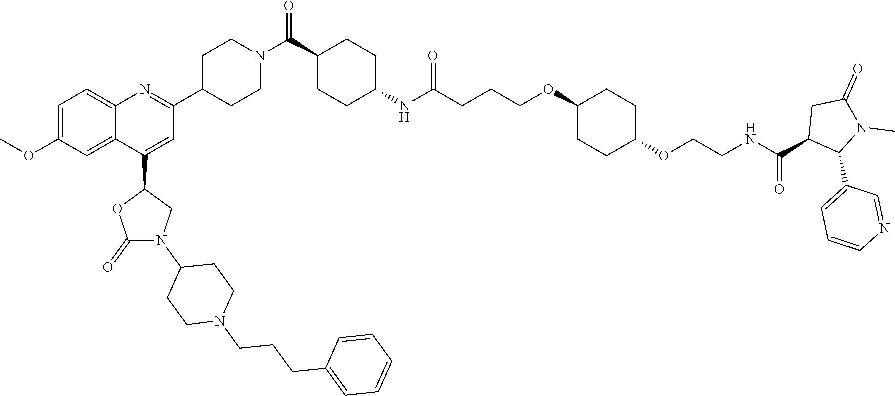 |
| J22 | 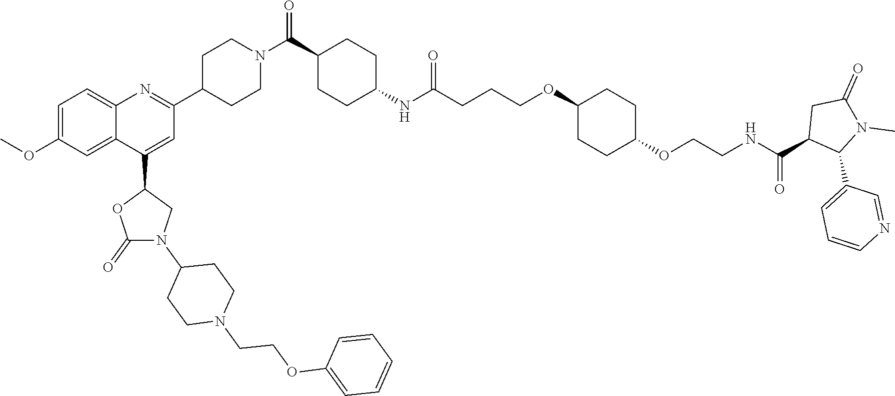 |
| J23 | 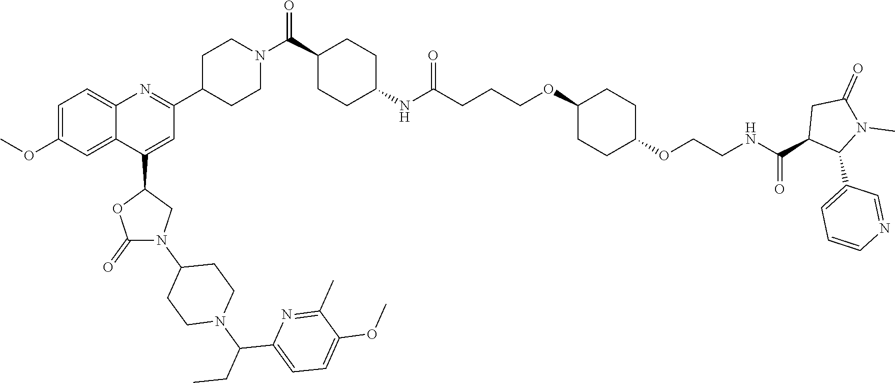 |
| J24 | 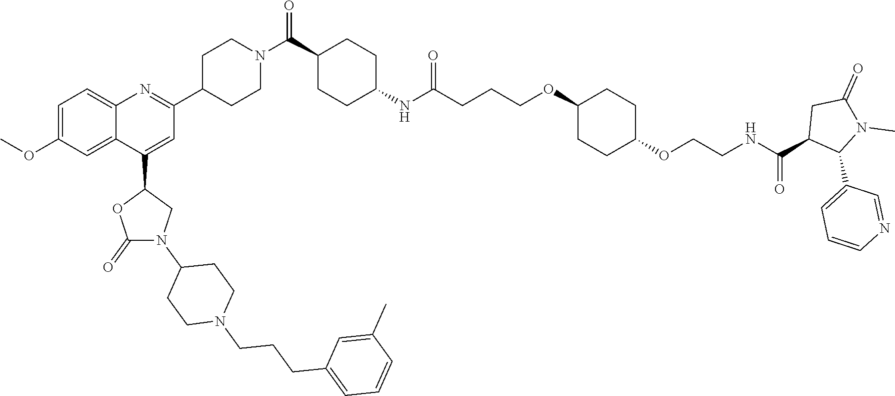 |
| J25 | 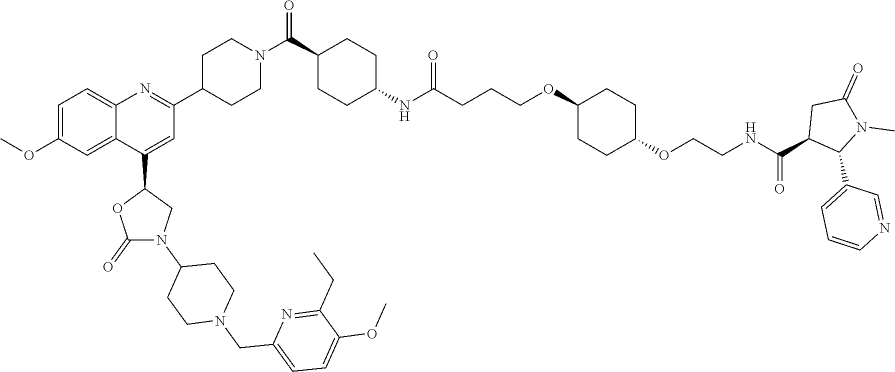 |
| J26 | 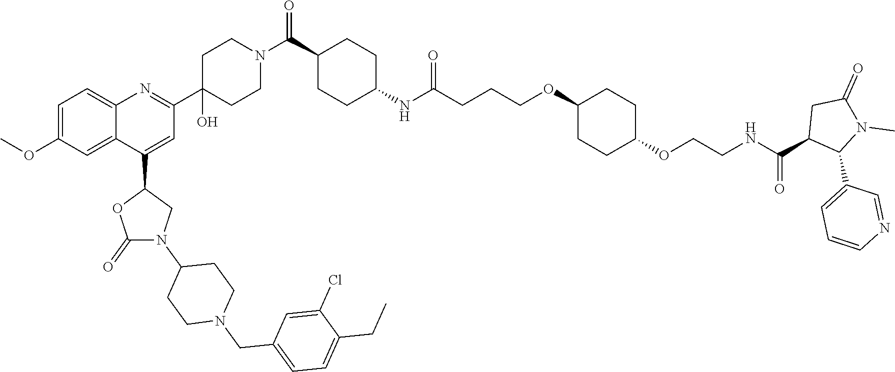 |
| J27 | 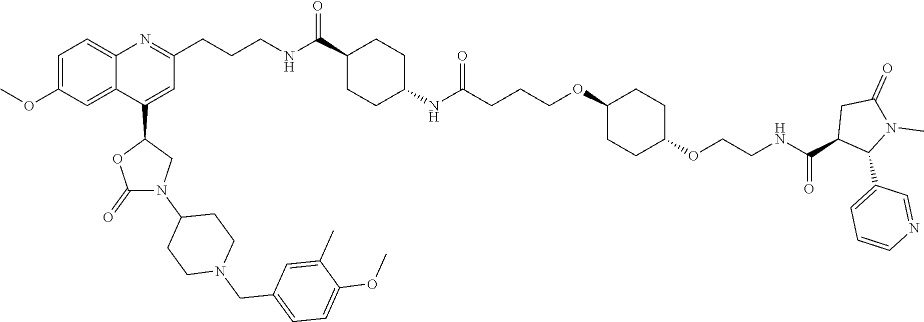 |
| J28 | 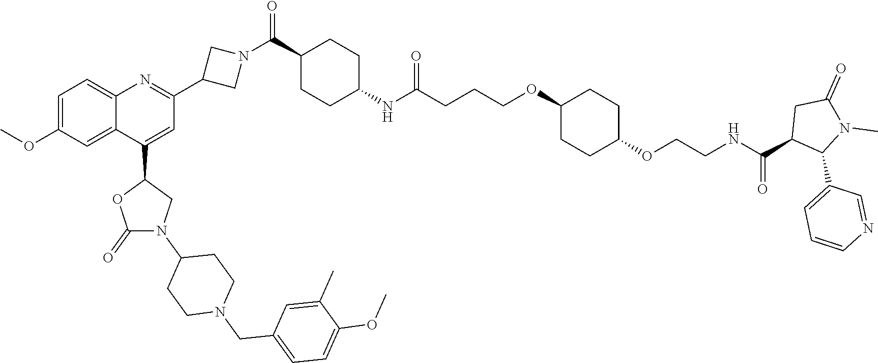 |
| J29 | 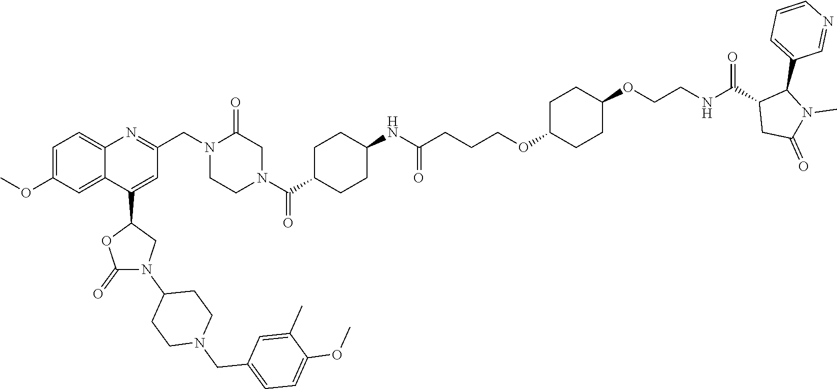 |
| J30 | 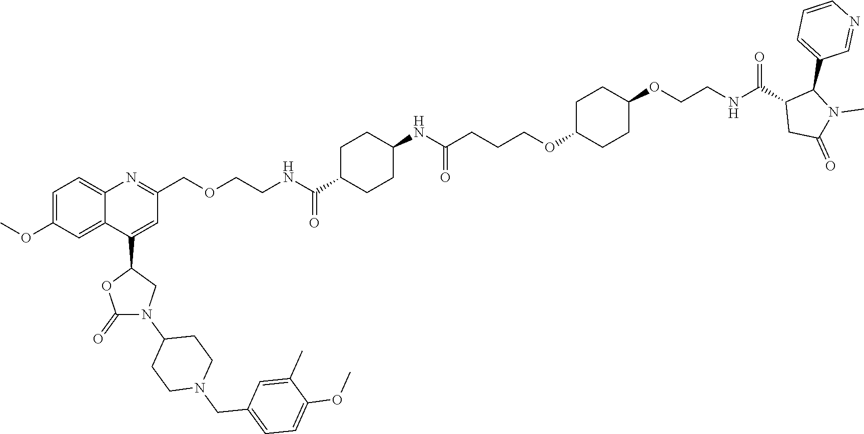 |
| J31 | 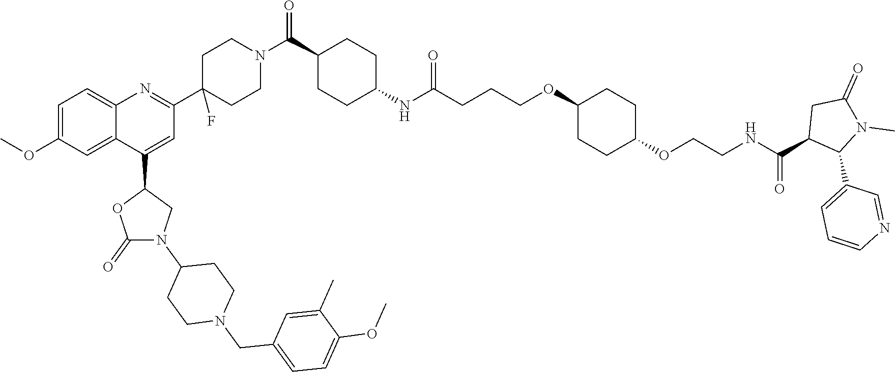 |
| J32 | 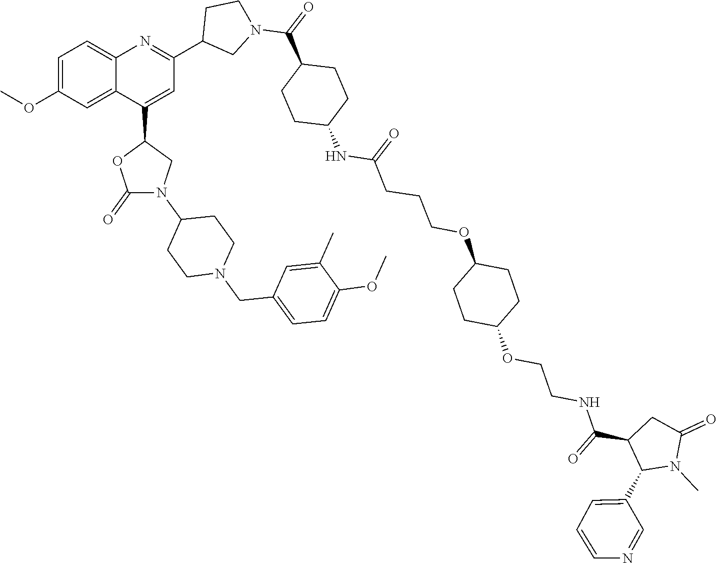 [SMILES: COc1ccc2nc(C3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)C3)cc([C@H]3CN(C4CCN(Cc5ccc(OC)c(C)c5)CC4)C(=O)O3)c2c1] |
| J33 | 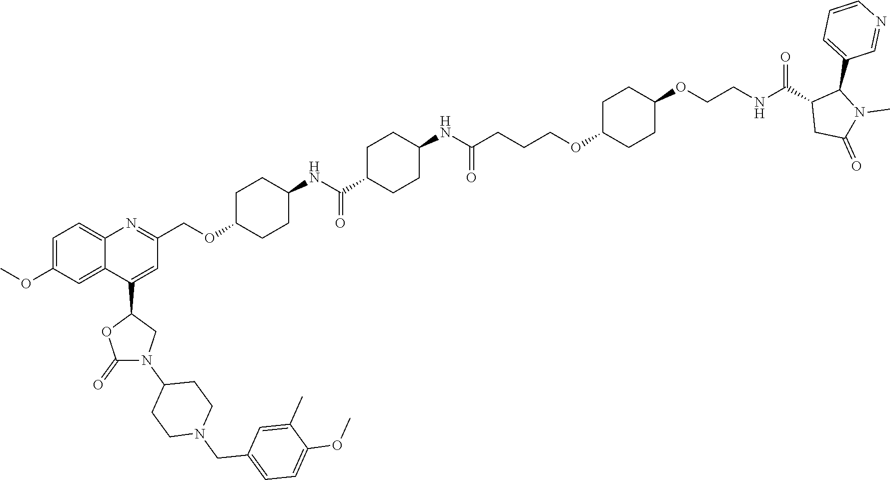 |
| J34 | 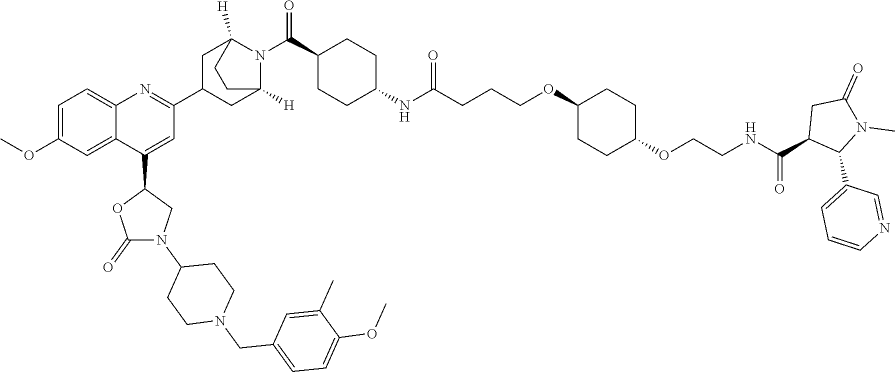 |
| J35 | 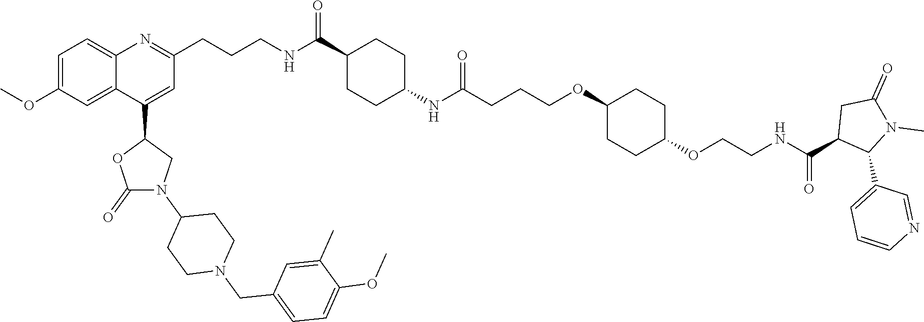 |
| J36 | 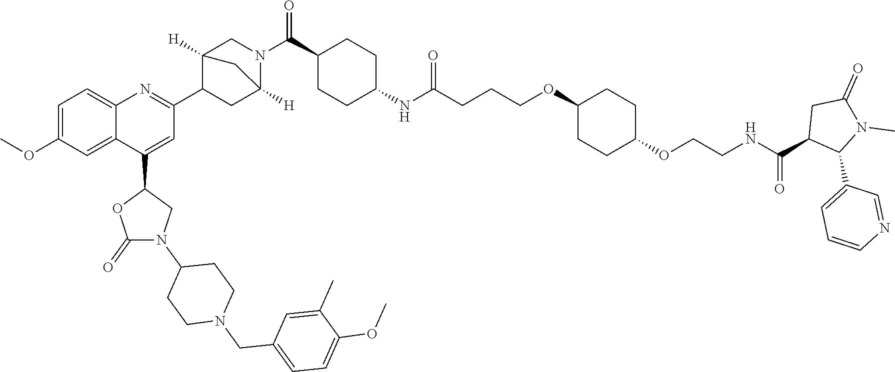 |
| J37 | 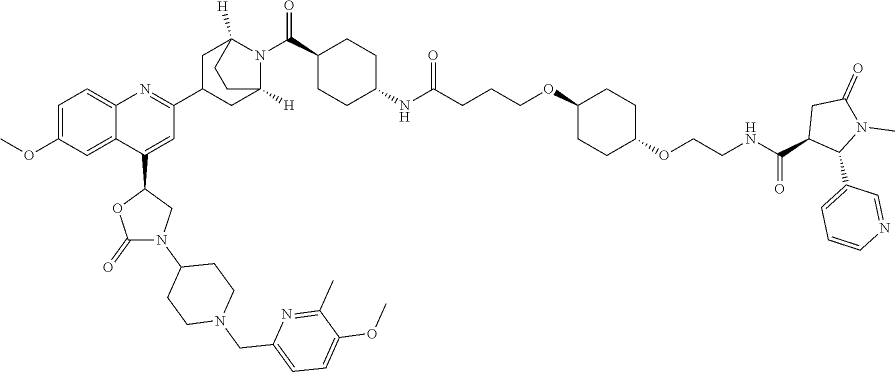 |
| J38 | 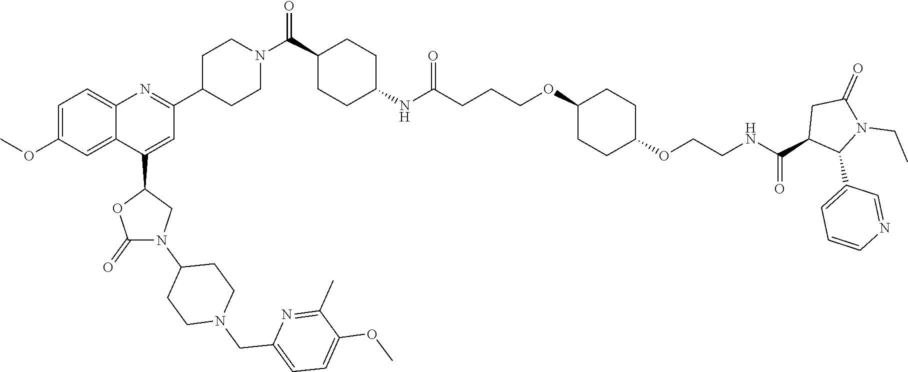 [SMILES: CCN1C(=O)C[C@H](C(=O)NCCO[C@H]2CC[C@H](OCCCC(=O)N[C@H]3CC[C@H](C(=O)N4CCC(c5cc([C@H]6CN(C7CCN(Cc8ccc(OC)c(C)n8)CC7)C(=O)O6)c6cc(OC)ccc6n5)CC4)CC3)CC2)[C@H]1c1cccnc1] |
| J39 | 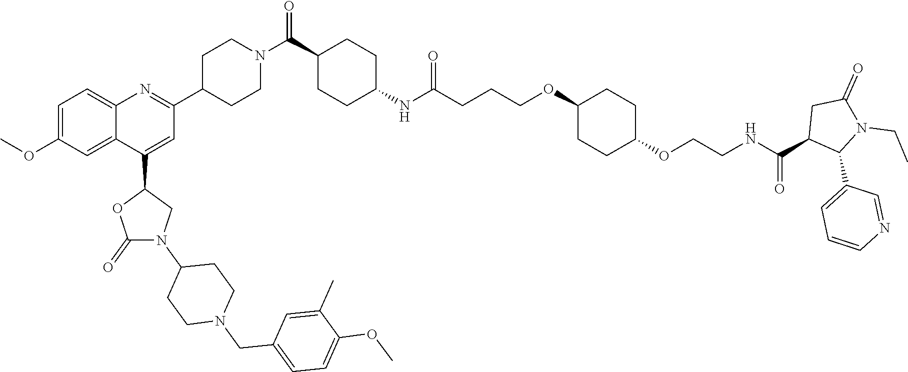 [SMILES: CCN1C(=O)C[C@H](C(=O)NCCO[C@H]2CC[C@H](OCCCC(=O)N[C@H]3CC[C@H](C(=O)N4CCC(c5cc([C@H]6CN(C7CCN(Cc8ccc(OC)c(C)c8)CC7)C(=O)O6)c6cc(OC)ccc6n5)CC4)CC3)CC2)[C@H]1c1cccnc1] |
| J40 | 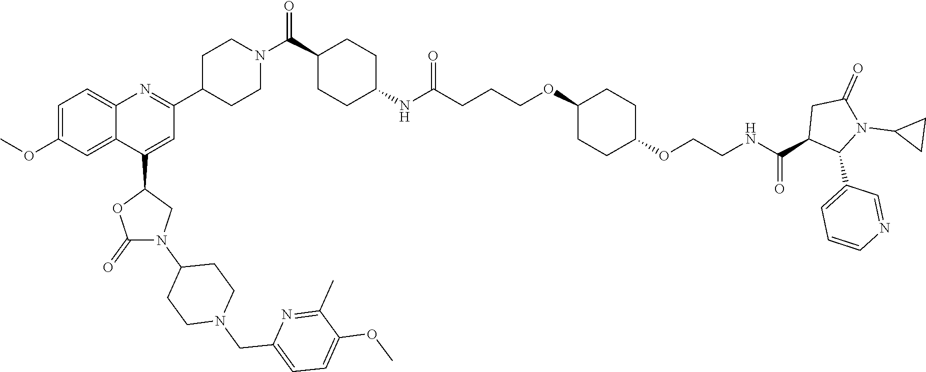 [SMILES: COc1ccc2nc(C3CCN(C(=O)[C@H]4CC[C@H](NC(=O)CCCO[C@H]5CC[C@H](OCCNC(=O)[C@H]6CC(=O)N(C7CC7)[C@@H]6c6cccnc6)CC5)CC4)CC3)cc([C@H]3CN(C4CCN(Cc5ccc(OC)c(C)n5)CC4)C(=O)O3)c2c1] |

### Targets and Target-Binding Moieties

The compounds of Formula (I) as disclosed herein are heterobifunctional synthetic agents designed such that one terminus interacts with a cell surface target, while the other terminus binds a specific antibody, including specific antibodies that form a portion of an antibody-drug conjugate or a bispecific antibody or bispecific antigen binding fragment thereof, including a bispecific T-cell engager. More specifically, the ARM simultaneously binds the cell surface target as well as the specific antibody. This ternary complex directs immune surveillance to target expressing tissue/cells and unites the mechanisms of antibody function with the dose-control of small molecules. This mechanism may include increased cell killing of targeting-expressing cells, depletion of target-expressing cells, antibody dependent cellular cytotoxicity (ADCC), antibody dependent cellular phagocytosis (ADCP), or complement dependant cytotoxicity (CDC). The same Fc receptor expressing immune cells that initiate destruction of the ARM/antibody tagged cells also participate in presentation of endogenous antigens for the potential for long term cellular immunity.

The compounds of Formula (I) as disclosed herein include a target-binding moiety that is capable of binding a target protein (e.g., a receptor) present on the surface of a cell. A person skilled in the art can select molecules known to bind the target protein for use as the target-binding moiety in the ARM.

In one embodiment, the target of the target binding moiety is a cell surface protein. In a further embodiment, the target of the target binding moiety is a target protein expressed on a pathogenic cell.

In a further embodiment, the pathogenic cell is a pathogenic immune cell, a tumor cell or cancer cell, or a stromal cell (including stromal cells present in a tumor microenvironment).

In a further embodiment, the target of the target binding moiety is present on the surface of a pathogenic agent selected from a virus or a bacterial cell. Examples of a virus expressing cell surface targets include, but are not limited to, influenza. Examples of cell surface targets on influenza virus include, but are not limited to, neuraminidase.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or yδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), or exhausted T cells.

In a further embodiment, the tumor cells or cancer cells are solid tumor cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, brain cancer cells, endocrine cancer cells, testicular cancer cells, bladder cancer cells, bone cancer cells, esophogeal cancer cells, gastric cancer cells, renal cell cancer cells, melanoma cancer cells, thyroid cancer cells, or breast cancer cells, preferably cells selected from mCRPC cells, breast cancer cells, lung cancer cells, colorectal cancer cells, or renal cell cancer cells.

In a further embodiment, the stromal cells are cancer associated fibroblasts (CAFs).

In one embodiment, the target of the target binding moiety is selected from a G protein-coupled receptor (GPCR), an enzyme (such as a dehydrogenase, an esterase, a phosphodiesterase, a hydrolase, a lipase, a phosphatase, a kinase, a reductase, or a transferase), a transporter (e.g., an ion channel), a protease, or a receptor. In a further embodiment, the target of the target binding moiety is selected from a GPCR, an enzyme (such as a dehydrogenase, an esterase, a phosphodiesterase, a hydrolase, a lipase, a phosphatase, a kinase, a reductase, or a transferase), a transporter (e . . . g, an ion channel), a protease, or a receptor, wherein the target is associated with and/or expressed on immune cells (including pathogenic immune cells), tumor cells or cancer cells, or stromal cells (including stromal cells present in a tumor microenvironment).

In a further embodiment, the target of the target binding moiety is selected from 15-hydroxyprostaglandin dehydrogenases, 5-hydroxytryptamine receptors, activated leukocyte cell adhesion molecules, ADAM metallopeptidases, adenosine receptors, adenosine deaminases, adrenoceptor beta, advanced glycosylation end-product specific receptors, membrane alanyl aminopeptidases, alkaline phosphatases, calcium voltage-gated channels, cannabinoid receptors, carcinoembryonic antigen related cell adhesion molecules, C-C motif chemokine receptors, CD14, CD19, CD200 receptors, CD22, CD274, CD276, CD33, CD37, CD38, CD3e, CD4, CD44, CD48, CD70, CD74, CD80, CD99, muscarinic cholinergic receptors, nicotinic cholinergic receptors, coagulation factor II thrombin receptors, colony stimulating factor 2 receptors, complement C5a receptors, C-type lectin domains, C-X-C motif chemokine receptors, cysteinyl leukotriene receptors, cytotoxic T-lymphocyte associated proteins, delta like canonical Notch ligands, dipeptidyl peptidases, ectonucleoside triphosphate diphosphohydrolases, erythropoietin receptors, F11 receptors, formyl peptide receptors, FXYD domain containing ion transport regulators, G protein-coupled bile acid receptors, G protein-coupled receptors, gamma-aminobutyric acid type A receptors, gastric inhibitory polypeptide receptors, glutamate metabotropic receptor, platelet glycoproteins, hepatitis A virus cellular receptors, histamine receptors, hydroxycarboxylic acid receptors, integrins, intercellular adhesion molecules, interleukin receptor accessory proteins, interleukin receptors, killer cell lectin like receptors, KISS1 receptors, leukotriene receptors, lymphocyte activating gene proteins, lymphocyte antigens, mannose receptors, membrane metalloendopeptidases, membrane spanning 4-domains, platelet activating factor receptors, potassium calcium-activated channels, potassium voltage-gated channels, programmed cell death proteins, prostaglandin receptors, prostaglandin synthases, protein tyrosine phosphatases, purinergic receptors, pyrimidinergic receptors, scavenger receptors, selectins, signaling lymphocytic activation molecule (SLAM) proteins, sodium voltage-gated channels, somatostatin receptors, sphingosine-1-phosphate receptors, suppression of tumorigenicity proteins, T cell immunoreceptors, thromboxane receptors, TNF receptors, toll like receptors, transient receptor potential cation channels, triggering receptors expressed on myeloid cells, or V-set immunoregulatory receptors.

In a further embodiment, the target of the target binding moiety is a target as listed in Table 4:

TABLE 4

| | | | | | Immune |
|---|---|---|---|---|---|
| | | | | | Class (based |
| Entrez | | | | | on GTEx, |
| Gene | | | Topology_{—} | TargetClass_{—} | GeneLogic, |
| ID | Symbol | Target | Type | Specific | Blueprint) |
| 1233 | CCR4 | C-C motif chemokine receptor 4 | MultiTM | GPCR | Immune |
| 1234 | CCR5 | C-C motif chemokine receptor 5 | MultiTM | GPCR | Immune |
| | | (gene/pseudogene) | | | |
| 1268 | CNR1 | cannabinoid receptor 1 | MultiTM | GPCR | Likely Immune |
| 7852 | CXCR4 | C-X-C motif chemokine receptor 4 | MultiTM | GPCR | Immune |
| 728 | C5AR1 | complement C5a receptor 1 | MultiTM | GPCR | Immune |
| 1909 | EDNRA | endothelin receptor type A | MultiTM | GPCR | Possible |
| | | | | | Immune |
| 3577 | CXCR1 | C-X-C motif chemokine receptor 1 | MultiTM | GPCR | Immune |
| 3579 | CXCR2 | C-X-C motif chemokine receptor 2 | MultiTM | GPCR | Immune |
| 8549 | LGR5 | leucine rich repeat containing G | MultiTM | GPCR | Likely Immune |
| | | protein-coupled receptor 5 | | | |
| 1232 | CCR3 | C-C motif chemokine receptor 3 | MultiTM | GPCR | Immune |
| 134391 | GPR151 | G protein-coupled receptor 151 | MultiTM | GPCR | Undetermined |
| 729230 | CCR2 | C-C motif chemokine receptor 2 | MultiTM | GPCR | Immune |
| 2843 | GPR20 | G protein-coupled receptor 20 | MultiTM | GPCR | Likely Immune |
| 4987 | OPRL1 | opioid related nociceptin receptor 1 | MultiTM | GPCR | Likely Immune |
| 5031 | P2RY6 | pyrimidinergic receptor P2Y6 | MultiTM | GPCR | Immune |
| 5732 | PTGER2 | prostaglandin E receptor 2 | MultiTM | GPCR | Immune |
| 5734 | PTGER4 | prostaglandin E receptor 4 | MultiTM | GPCR | Likely Immune |
| 10803 | CCR9 | C-C motif chemokine receptor 9 | MultiTM | GPCR | Immune |
| 1910 | EDNRB | endothelin receptor type B | MultiTM | GPCR | Non-Immune |
| 3356 | HTR2A | 5-hydroxytryptamine receptor 2A | MultiTM | GPCR | Possible |
| | | | | | Immune |
| 146 | ADRA1D | adrenoceptor alpha 1D | MultiTM | GPCR | Non-Immune |
| 887 | CCKBR | cholecystokinin B receptor | MultiTM | GPCR | Possible |
| | | | | | Immune |
| 2149 | F2R | coagulation factor II thrombin | MultiTM | GPCR | Immune |
| | | receptor | | | |
| 2492 | FSHR | follicle stimulating hormone | MultiTM | GPCR | Undetermined |
| | | receptor | | | |
| 2847 | MCHR1 | melanin concentrating hormone | MultiTM | GPCR | Possible |
| | | receptor 1 | | | Immune |
| 2862 | MLNR | motilin receptor | MultiTM | GPCR | Likely Immune |
| 3363 | HTR7 | 5-hydroxytryptamine receptor 7 | MultiTM | GPCR | Immune |
| 5733 | PTGER3 | prostaglandin E receptor 3 | MultiTM | GPCR | Possible |
| | | | | | Immune |
| 10886 | NPFFR2 | neuropeptide FF receptor 2 | MultiTM | GPCR | Likely Immune |
| 57105 | CYSLTR2 | cysteinyl leukotriene receptor 2 | MultiTM | GPCR | Immune |
| 350383 | GPR142 | G protein-coupled receptor 142 | MultiTM | GPCR | Immune |
| 4142 | MAS1 | MAS1 proto-oncogene, G protein- | MultiTM | GPCR | Non-Immune |
| | | coupled receptor | | | |
| 6752 | SSTR2 | somatostatin receptor 2 | MultiTM | GPCR | Immune |
| 134 | ADORA1 | adenosine A1 receptor | MultiTM | GPCR | Non-Immune |
| 150 | ADRA2A | adrenoceptor alpha 2A | MultiTM | GPCR | Likely Immune |
| 1230 | CCR1 | C-C motif chemokine receptor 1 | MultiTM | GPCR | Immune |
| 1235 | CCR6 | C-C motif chemokine receptor 6 | MultiTM | GPCR | Immune |
| 1813 | DRD2 | dopamine receptor D2 | MultiTM | GPCR | Non-Immune |
| 1814 | DRD3 | dopamine receptor D3 | MultiTM | GPCR | Undetermined |
| 2358 | FPR2 | formyl peptide receptor 2 | MultiTM | GPCR | Immune |
| 2925 | GRPR | gastrin releasing peptide receptor | MultiTM | GPCR | Likely Immune |
| 3269 | HRH1 | histamine receptor H1 | MultiTM | GPCR | Immune |
| 5724 | PTAFR | platelet activating factor receptor | MultiTM | GPCR | Immune |
| 9290 | GPR55 | G protein-coupled receptor 55 | MultiTM | GPCR | Immune |
| 151306 | GPBAR1 | G protein-coupled bile acid | MultiTM | GPCR | Immune |
| | | receptor 1 | | | |
| 338442 | HCAR2 | hydroxycarboxylic acid receptor 2 | MultiTM | GPCR | Immune |
| 136 | ADORA2B | adenosine A2b receptor | MultiTM | GPCR | Immune |
| 140 | ADORA3 | adenosine A3 receptor | MultiTM | GPCR | Undetermined |
| 151 | ADRA2B | adrenoceptor alpha 2B | MultiTM | GPCR | Likely Immune |
| 153 | ADRB1 | adrenoceptor beta 1 | MultiTM | GPCR | Likely Immune |
| 154 | ADRB2 | adrenoceptor beta 2 | MultiTM | GPCR | Immune |
| 155 | ADRB3 | adrenoceptor beta 3 | MultiTM | GPCR | Possible |
| | | | | | Immune |
| 185 | AGTR1 | angiotensin II receptor type 1 | MultiTM | GPCR | Likely Immune |
| 886 | CCKAR | cholecystokinin A receptor | MultiTM | GPCR | Possible |
| | | | | | Immune |
| 1128 | CHRM1 | cholinergic receptor muscarinic 1 | MultiTM | GPCR | Non-Immune |
| 1129 | CHRM2 | cholinergic receptor muscarinic 2 | MultiTM | GPCR | Non-Immune |
| 1131 | CHRM3 | cholinergic receptor muscarinic 3 | MultiTM | GPCR | Likely Immune |
| 1132 | CHRM4 | cholinergic receptor muscarinic 4 | MultiTM | GPCR | Immune |
| 1133 | CHRM5 | cholinergic receptor muscarinic 5 | MultiTM | GPCR | Possible |
| | | | | | Immune |
| 1237 | CCR8 | C-C motif chemokine receptor 8 | MultiTM | GPCR | Immune |
| 1241 | LTB4R | leukotriene B4 receptor | MultiTM | GPCR | Immune |
| 1269 | CNR2 | cannabinoid receptor 2 | MultiTM | GPCR | Immune |
| 1815 | DRD4 | dopamine receptor D4 | MultiTM | GPCR | Undetermined |
| 1816 | DRD5 | dopamine receptor D5 | MultiTM | GPCR | Non-Immune |
| 1901 | S1PR1 | sphingosine-1-phosphate receptor | MultiTM | GPCR | Likely Immune |
| | | 1 | | | |
| 1902 | LPAR1 | lysophosphatidic acid receptor 1 | MultiTM | GPCR | Possible |
| | | | | | Immune |
| 2693 | GHSR | growth hormone secretagogue | MultiTM | GPCR | Possible |
| | | receptor | | | Immune |
| 2798 | GNRHR | gonadotropin releasing hormone | MultiTM | GPCR | Likely Immune |
| | | receptor | | | |
| 2834 | PRLHR | prolactin releasing hormone | MultiTM | GPCR | Non-Immune |
| | | receptor | | | |
| 2864 | FFAR1 | free fatty acid receptor 1 | MultiTM | GPCR | Likely Immune |
| 3061 | HCRTR1 | hypocretin receptor 1 | MultiTM | GPCR | Possible |
| | | | | | Immune |
| 3062 | HCRTR2 | hypocretin receptor 2 | MultiTM | GPCR | Possible |
| | | | | | Immune |
| 3274 | HRH2 | histamine receptor H2 | MultiTM | GPCR | Likely Immune |
| 3350 | HTR1A | 5-hydroxytryptamine receptor 1A | MultiTM | GPCR | Non-Immune |
| 3351 | HTR1B | 5-hydroxytryptamine receptor 1B | MultiTM | GPCR | Possible |
| | | | | | Immune |
| 3352 | HTR1D | 5-hydroxytryptamine receptor 1D | MultiTM | GPCR | Possible |
| | | | | | Immune |
| 3354 | HTR1E | 5-hydroxytryptamine receptor 1E | MultiTM | GPCR | Non-Immune |
| 3355 | HTR1F | 5-hydroxytryptamine receptor 1F | MultiTM | GPCR | Likely Immune |
| 3357 | HTR2B | 5-hydroxytryptamine receptor 2B | MultiTM | GPCR | Possible |
| | | | | | Immune |
| 3358 | HTR2C | 5-hydroxytryptamine receptor 2C | MultiTM | GPCR | Possible |
| | | | | | Immune |
| 3361 | HTR5A | 5-hydroxytryptamine receptor 5A | MultiTM | GPCR | Possible |
| | | | | | Immune |
| 3362 | HTR6 | 5-hydroxytryptamine receptor 6 | MultiTM | GPCR | Possible |
| | | | | | Immune |
| 4157 | MC1R | melanocortin 1 receptor | MultiTM | GPCR | Non-Immune |
| 4543 | MTNR1A | melatonin receptor 1A | MultiTM | GPCR | Possible |
| | | | | | Immune |
| 4544 | MTNR1B | melatonin receptor 1B | MultiTM | GPCR | Undetermined |
| 4889 | NPY5R | neuropeptide Y receptor Y5 | MultiTM | GPCR | Likely Immune |
| 4985 | OPRD1 | opioid receptor delta 1 | MultiTM | GPCR | Possible |
| | | | | | Immune |
| 4986 | OPRK1 | opioid receptor kappa 1 | MultiTM | GPCR | Non-Immune |
| 5021 | OXTR | oxytocin receptor | MultiTM | GPCR | Likely Immune |
| 5029 | P2RY2 | purinergic receptor P2Y2 | MultiTM | GPCR | Immune |
| 5729 | PTGDR | prostaglandin D2 receptor | MultiTM | GPCR | Immune |
| 5731 | PTGER1 | prostaglandin E receptor 1 | MultiTM | GPCR | Likely Immune |
| 5737 | PTGFR | prostaglandin F receptor | MultiTM | GPCR | Likely Immune |
| 5739 | PTGIR | prostaglandin I2 receptor | MultiTM | GPCR | Immune |
| 6754 | SSTR4 | somatostatin receptor 4 | MultiTM | GPCR | Possible |
| | | | | | Immune |
| 6755 | SSTR5 | somatostatin receptor 5 | MultiTM | GPCR | Non-Immune |
| 6865 | TACR2 | tachykinin receptor 2 | MultiTM | GPCR | Non-Immune |
| 6869 | TACR1 | tachykinin receptor 1 | MultiTM | GPCR | Non-Immune |
| 6915 | TBXA2R | thromboxane A2 receptor | MultiTM | GPCR | Immune |
| 7201 | TRHR | thyrotropin releasing hormone | MultiTM | GPCR | Likely Immune |
| | | receptor | | | |
| 8477 | GPR65 | G protein-coupled receptor 65 | MultiTM | GPCR | Immune |
| 8698 | S1PR4 | sphingosine-1-phosphate receptor | MultiTM | GPCR | Immune |
| | | 4 | | | |
| 8811 | GALR2 | galanin receptor 2 | MultiTM | GPCR | Likely Immune |
| 8843 | HCAR3 | hydroxycarboxylic acid receptor 3 | MultiTM | GPCR | Immune |
| 9002 | F2RL3 | F2R like thrombin or trypsin | MultiTM | GPCR | Likely Immune |
| | | receptor 3 | | | |
| 10800 | CYSLTR1 | cysteinyl leukotriene receptor 1 | MultiTM | GPCR | Immune |
| 11251 | PTGDR2 | prostaglandin D2 receptor 2 | MultiTM | GPCR | Likely Immune |
| 11255 | HRH3 | histamine receptor H3 | MultiTM | GPCR | Possible |
| | | | | | Immune |
| 23432 | GPR161 | G protein-coupled receptor 161 | MultiTM | GPCR | Possible |
| | | | | | Immune |
| 29933 | GPR132 | G protein-coupled receptor 132 | MultiTM | GPCR | Immune |
| 53637 | S1PR5 | sphingosine-1-phosphate receptor | MultiTM | GPCR | Immune |
| | | 5 | | | |
| 53831 | GPR84 | G protein-coupled receptor 84 | MultiTM | GPCR | Immune |
| 59340 | HRH4 | histamine receptor H4 | MultiTM | GPCR | Likely Immune |
| 64805 | P2RY12 | purinergic receptor P2Y12 | MultiTM | GPCR | Immune |
| 84539 | MCHR2 | melanin concentrating hormone | MultiTM | GPCR | Possible |
| | | receptor 2 | | | Immune |
| 84634 | KISS1R | KISS1 receptor | MultiTM | GPCR | Immune |
| 84636 | GPR174 | G protein-coupled receptor 174 | MultiTM | GPCR | Immune |
| 124274 | GPR139 | G protein-coupled receptor 139 | MultiTM | GPCR | Undetermined |
| 139760 | GPR119 | G protein-coupled receptor 119 | MultiTM | GPCR | Undetermined |
| 338557 | FFAR4 | free fatty acid receptor 4 | MultiTM | GPCR | Likely Immune |
| 6870 | TACR3 | tachykinin receptor 3 | MultiTM | GPCR | Non-Immune |
| 10203 | CALCRL | calcitonin receptor like receptor | MultiTM | GPCR | Likely Immune |
| 2740 | GLP1R | glucagon like peptide 1 receptor | MultiTM | GPCR | Possible |
| | | | | | Immune |
| 2696 | GIPR | gastric inhibitory polypeptide | MultiTM | GPCR | Immune |
| | | receptor | | | |
| 5745 | PTH1R | parathyroid hormone 1 receptor | MultiTM | GPCR | Possible |
| | | | | | Immune |
| 1395 | CRHR2 | corticotropin releasing hormone | MultiTM | GPCR | Non-Immune |
| | | receptor 2 | | | |
| 2550 | GABBR1 | gamma-aminobutyric acid type B | MultiTM | GPCR | Non-Immune |
| | | receptor subunit 1 | | | |
| 2913 | GRM3 | glutamate metabotropic receptor 3 | MultiTM | GPCR | Likely Immune |
| 6608 | SMO | smoothened, frizzled class | MultiTM | GPCR | Non-Immune |
| | | receptor | | | |
| 799 | CALCR | calcitonin receptor | MultiTM | GPCR | Possible |
| | | | | | Immune |
| 846 | CASR | calcium sensing receptor | MultiTM | GPCR | Likely Immune |
| 1394 | CRHR1 | corticotropin releasing hormone | MultiTM | GPCR | Non-Immune |
| | | receptor 1 | | | |
| 2692 | GHRHR | growth hormone releasing | MultiTM | GPCR | Non-Immune |
| | | hormone receptor | | | |
| 2912 | GRM2 | glutamate metabotropic receptor 2 | MultiTM | GPCR | Immune |
| 2914 | GRM4 | glutamate metabotropic receptor 4 | MultiTM | GPCR | Non-Immune |
| 2915 | GRM5 | glutamate metabotropic receptor 5 | MultiTM | GPCR | Possible |
| | | | | | Immune |
| 2917 | GRM7 | glutamate metabotropic receptor 7 | MultiTM | GPCR | Undetermined |
| 9340 | GLP2R | glucagon like peptide 2 receptor | MultiTM | GPCR | Non-Immune |
| 3248 | HPGD | 15-hydroxyprostaglandin | Membrane | Enzyme_{—} | Immune |
| | | dehydrogenase | | Dehydrohgenase | |
| 43 | ACHE | acetylcholinesterase (Cartwright | Membrane | Enzyme_Esterase_{—} | Likely Immune |
| | | blood group) | | Other | |
| 5139 | PDE3A | phosphodiesterase 3A | MultiTM | Enzyme_Esterase_{—} | Possible |
| | | | | Phosphodiesterase | Immune |
| 953 | ENTPD1 | ectonucleoside triphosphate | MultiTM | Enzyme_Hydrolase | Immune |
| | | diphosphohydrolase 1 | | | |
| 4023 | LPL | lipoprotein lipase | Membrane | Enzyme_Lipase | Likely Immune |
| 4907 | NT5E | 5′-nucleotidase ecto | Membrane | Enzyme_other | Likely Immune |
| 2984 | GUCY2C | guanylate cyclase 2C | SingleTM | Enzyme_other | Possible |
| | | | | | Immune |
| 760 | CA2 | carbonic anhydrase 2 | Membrane | Enzyme_other | Likely Immune |
| 7173 | TPO | thyroid peroxidase | SingleTM | Enzyme_other | Likely Immune |
| 50507 | NOX4 | NADPH oxidase 4 | MultiTM | Enzyme_other | Non-Immune |
| 3309 | HSPA5 | heat shock protein family A | Membrane | Enzyme_other | Likely Immune |
| | | (Hsp70) member 5 | | | |
| 100 | ADA | adenosine deaminase | Membrane | Enzyme_other | Immune |
| 2280 | FKBP1A | FK506 binding protein 1A | Membrane | Enzyme_other | Possible |
| | | | | | Immune |
| 9536 | PTGES | prostaglandin E synthase | MultiTM | Enzyme_other | Immune |
| 130013 | ACMSD | aminocarboxymuconate | SingleTM | Enzyme_other | Non-Immune |
| | | semialdehyde decarboxylase | | | |
| 5169 | ENPP3 | ectonucleotide | SingleTM | Enzyme_Phosphatase | Possible |
| | | pyrophosphatase/ | | | Immune |
| | | phosphodiesterase 3 | | | |
| 6647 | SOD1 | superoxide dismutase 1 | Membrane | Enzyme_Phosphatase | Possible |
| | | | | | Immune |
| 5167 | ENPP1 | ectonucleotide | SingleTM | Enzyme_Phosphatase | Non-Immune |
| | | pyrophosphatase/ | | | |
| | | phosphodiesterase 1 | | | |
| 249 | ALPL | alkaline phosphatase, | Membrane | Enzyme_Phosphatase | Immune |
| | | biomineralization associated | | | |
| 5788 | PTPRC | protein tyrosine phosphatase, | SingleTM | Enzyme_Phosphatase | Immune |
| | | receptor type C | | | |
| 8639 | AOC3 | amine oxidase, copper containing | SingleTM | Enzyme_Reductase | Non-Immune |
| | | 3 | | | |
| 6121 | RPE65 | RPE65, retinoid isomerohydrolase | Membrane | Enzyme_Reductase | Non-Immune |
| 952 | CD38 | CD38 molecule | SingleTM | Enzyme_Transerface | Immune |
| 7124 | TNF | tumor necrosis factor | SingleTM | Extracellular | Immune |
| | | | | MB_Bound | |
| | | | | Ligand | |
| 1435 | CSF1 | colony stimulating factor 1 | SingleTM | Extracellular | Immune |
| | | | | MB_Bound | |
| | | | | Ligand | |
| 6376 | CX3CL1 | C-X3-C motif chemokine ligand 1 | SingleTM | Extracellular | Non-Immune |
| | | | | MB_Bound | |
| | | | | Ligand | |
| 8743 | TNFSF10 | TNF superfamily member 10 | SingleTM | Extracellular | Likely Immune |
| | | | | MB_Bound | |
| | | | | Ligand | |
| 5027 | P2RX7 | purinergic receptor P2X 7 | MultiTM | Ion Channel | Likely Immune |
| 3738 | KCNA3 | potassium voltage-gated channel | MultiTM | Ion Channel | Immune |
| | | subfamily A member 3 | | | |
| 6335 | SCN9A | sodium voltage-gated channel | MultiTM | Ion Channel | Immune |
| | | alpha subunit 9 | | | |
| 6336 | SCN10A | sodium voltage-gated channel | MultiTM | Ion Channel | Non-Immune |
| | | alpha subunit 10 | | | |
| 7442 | TRPV1 | transient receptor potential cation | MultiTM | Ion Channel | Undetermined |
| | | channel subfamily V member 1 | | | |
| 8989 | TRPA1 | transient receptor potential cation | MultiTM | Ion Channel | Likely Immune |
| | | channel subfamily A member 1 | | | |
| 3783 | KCNN4 | potassium calcium-activated | MultiTM | Ion Channel | Immune |
| | | channel subfamily N member 4 | | | |
| 53827 | FXYD5 | FXYD domain containing ion | SingleTM | Ion Channel | Immune |
| | | transport regulator 5 | | | |
| 2554 | GABRA1 | gamma-aminobutyric acid type A | MultiTM | Ion Channel | Likely Immune |
| | | receptor alpha1 subunit | | | |
| 2890 | GRIA1 | glutamate ionotropic receptor | MultiTM | Ion Channel | Possible |
| | | AMPA type subunit 1 | | | Immune |
| 2891 | GRIA2 | glutamate ionotropic receptor | MultiTM | Ion Channel | Non-Immune |
| | | AMPA type subunit 2 | | | |
| 8913 | CACNA1G | calcium voltage-gated channel | MultiTM | Ion Channel | Non-Immune |
| | | subunit alpha1 G | | | |
| 22953 | P2RX2 | purinergic receptor P2X 2 | MultiTM | Ion Channel | Likely Immune |
| 774 | CACNA1B | calcium voltage-gated channel | MultiTM | Ion Channel | Possible |
| | | subunit alpha1 B | | | Immune |
| 778 | CACNA1F | calcium voltage-gated channel | MultiTM | Ion Channel | Immune |
| | | subunit alpha1 F | | | |
| 1136 | CHRNA3 | cholinergic receptor nicotinic alpha | MultiTM | Ion Channel | Likely Immune |
| | | 3 subunit | | | |
| 1139 | CHRNA7 | cholinergic receptor nicotinic alpha | MultiTM | Ion Channel | Undetermined |
| | | 7 subunit | | | |
| 1141 | CHRNB2 | cholinergic receptor nicotinic beta | MultiTM | Ion Channel | Possible |
| | | 2 subunit | | | Immune |
| 1143 | CHRNB4 | cholinergic receptor nicotinic beta | MultiTM | Ion Channel | Possible |
| | | 4 subunit | | | Immune |
| 2555 | GABRA2 | gamma-aminobutyric acid type A | MultiTM | Ion Channel | Non-Immune |
| | | receptor alpha2 subunit | | | |
| 2556 | GABRA3 | gamma-aminobutyric acid type A | MultiTM | Ion Channel | Possible |
| | | receptor alpha3 subunit | | | Immune |
| 2558 | GABRA5 | gamma-aminobutyric acid type A | MultiTM | Ion Channel | Possible |
| | | receptor alpha5 subunit | | | Immune |
| 2561 | GABRB2 | gamma-aminobutyric acid type A | MultiTM | Ion Channel | Immune |
| | | receptor beta2 subunit | | | |
| 2902 | GRIN1 | glutamate ionotropic receptor | MultiTM | Ion Channel | Possible |
| | | NMDA type subunit 1 | | | Immune |
| 2904 | GRIN2B | glutamate ionotropic receptor | MultiTM | Ion Channel | Possible |
| | | NMDA type subunit 2B | | | Immune |
| 3359 | HTR3A | 5-hydroxytryptamine receptor 3A | MultiTM | Ion Channel | Immune |
| 5025 | P2RX4 | purinergic receptor P2X 4 | MultiTM | Ion Channel | Likely Immune |
| 6337 | SCNN1A | sodium channel epithelial 1 alpha | MultiTM | Ion Channel | Possible |
| | | subunit | | | Immune |
| 7223 | TRPC4 | transient receptor potential cation | MultiTM | Ion Channel | Non-Immune |
| | | channel subfamily C member 4 | | | |
| 7225 | TRPC6 | transient receptor potential cation | MultiTM | Ion Channel | Likely Immune |
| | | channel subfamily C member 6 | | | |
| 8001 | GLRA3 | glycine receptor alpha 3 | MultiTM | Ion Channel | Possible |
| | | | | | Immune |
| 8973 | CHRNA6 | cholinergic receptor nicotinic alpha | MultiTM | Ion Channel | Immune |
| | | 6 subunit | | | |
| 54822 | TRPM7 | transient receptor potential cation | MultiTM | Ion Channel | Possible |
| | | channel subfamily M member 7 | | | Immune |
| 79054 | TRPM8 | transient receptor potential cation | MultiTM | Ion Channel | Possible |
| | | channel subfamily M member 8 | | | Immune |
| 84876 | ORAI1 | ORAI calcium release-activated | MultiTM | Ion Channel | Likely Immune |
| | | calcium modulator 1 | | | |
| 361 | AQP4 | aquaporin 4 | MultiTM | Ion Channel | Likely Immune |
| 3758 | KCNJ1 | potassium voltage-gated channel | MultiTM | Ion Channel | Likely Immune |
| | | subfamily J member 1 | | | |
| 3786 | KCNQ3 | potassium voltage-gated channel | MultiTM | Ion Channel | Likely Immune |
| | | subfamily Q member 3 | | | |
| 6323 | SCN1A | sodium voltage-gated channel | MultiTM | Ion Channel | Possible |
| | | alpha subunit 1 | | | Immune |
| 6326 | SCN2A | sodium voltage-gated channel | MultiTM | Ion Channel | Likely Immune |
| | | alpha subunit 2 | | | |
| 6328 | SCN3A | sodium voltage-gated channel | MultiTM | Ion Channel | Likely Immune |
| | | alpha subunit 3 | | | |
| 6334 | SCN8A | sodium voltage-gated channel | MultiTM | Ion Channel | Immune |
| | | alpha subunit 8 | | | |
| 57657 | HCN3 | hyperpolarization activated cyclic | MultiTM | Ion Channel | Non-Immune |
| | | nucleotide gated potassium | | | |
| | | channel 3 | | | |
| 59341 | TRPV4 | transient receptor potential cation | MultiTM | Ion Channel | Immune |
| | | channel subfamily V member 4 | | | |
| 359 | AQP2 | aquaporin 2 | MultiTM | Ion Channel | Possible |
| | | | | | Immune |
| 610 | HCN2 | hyperpolarization activated cyclic | MultiTM | Ion Channel | Possible |
| | | nucleotide gated potassium and | | | Immune |
| | | sodium channel 2 | | | |
| 775 | CACNA1C | calcium voltage-gated channel | MultiTM | Ion Channel | Non-Immune |
| | | subunit alpha1 C | | | |
| 779 | CACNA1S | calcium voltage-gated channel | MultiTM | Ion Channel | Non-Immune |
| | | subunit alpha1 S | | | |
| 1180 | CLCN1 | chloride voltage-gated channel 1 | MultiTM | Ion Channel | Possible |
| | | | | | Immune |
| 3751 | KCND2 | potassium voltage-gated channel | MultiTM | Ion Channel | Likely Immune |
| | | subfamily D member 2 | | | |
| 3757 | KCNH2 | potassium voltage-gated channel | MultiTM | Ion Channel | Likely Immune |
| | | subfamily H member 2 | | | |
| 3778 | KCNMA1 | potassium calcium-activated | MultiTM | Ion Channel | Possible |
| | | channel subfamily M alpha 1 | | | Immune |
| 3784 | KCNQ1 | potassium voltage-gated channel | MultiTM | Ion Channel | Immune |
| | | subfamily Q member 1 | | | |
| 3785 | KCNQ2 | potassium voltage-gated channel | MultiTM | Ion Channel | Likely Immune |
| | | subfamily Q member 2 | | | |
| 6329 | SCN4A | sodium voltage-gated channel | MultiTM | Ion Channel | Likely Immune |
| | | alpha subunit 4 | | | |
| 6331 | SCN5A | sodium voltage-gated channel | MultiTM | Ion Channel | Possible |
| | | alpha subunit 5 | | | Immune |
| 10021 | HCN4 | hyperpolarization activated cyclic | MultiTM | Ion Channel | Non-Immune |
| | | nucleotide gated potassium | | | |
| | | channel 4 | | | |
| 11280 | SCN11A | sodium voltage-gated channel | MultiTM | Ion Channel | Immune |
| | | alpha subunit 11 | | | |
| 55503 | TRPV6 | transient receptor potential cation | MultiTM | Ion Channel | Possible |
| | | channel subfamily V member 6 | | | Immune |
| 348980 | HCN1 | hyperpolarization activated cyclic | MultiTM | Ion Channel | Non-Immune |
| | | nucleotide gated potassium | | | |
| | | channel 1 | | | |
| 3760 | KCNJ3 | potassium voltage-gated channel | MultiTM | Ion Channel | Non-Immune |
| | | subfamily J member 3 | | | |
| 3762 | KCNJ5 | potassium voltage-gated channel | MultiTM | Ion Channel | Immune |
| | | subfamily J member 5 | | | |
| 23416 | KCNH3 | potassium voltage-gated channel | MultiTM | Ion Channel | Likely Immune |
| | | subfamily H member 3 | | | |
| 1137 | CHRNA4 | cholinergic receptor nicotinic alpha | MultiTM | Ion Channel | Likely Immune |
| | | 4 subunit | | | |
| 2563 | GABRD | gamma-aminobutyric acid type A | MultiTM | Ion Channel | Non-Immune |
| | | receptor delta subunit | | | |
| 2892 | GRIA3 | glutamate ionotropic receptor | MultiTM | Ion Channel | Possible |
| | | AMPA type subunit 3 | | | Immune |
| 2893 | GRIA4 | glutamate ionotropic receptor | MultiTM | Ion Channel | Non-Immune |
| | | AMPA type subunit 4 | | | |
| 776 | CACNA1D | calcium voltage-gated channel | MultiTM | Ion Channel | Likely Immune |
| | | subunit alpha1 D | | | |
| 1181 | CLCN2 | chloride voltage-gated channel 2 | MultiTM | Ion Channel | Non-Immune |
| 3746 | KCNC1 | potassium voltage-gated channel | MultiTM | Ion Channel | Non-Immune |
| | | subfamily C member 1 | | | |
| 3747 | KCNC2 | potassium voltage-gated channel | MultiTM | Ion Channel | Possible |
| | | subfamily C member 2 | | | Immune |
| 3776 | KCNK2 | potassium two pore domain | MultiTM | Ion Channel | Possible |
| | | channel subfamily K member 2 | | | Immune |
| 1956 | EGFR | epidermal growth factor receptor | SingleTM | Kinase_Protein | Possible |
| | | | | | Immune |
| 2064 | ERBB2 | erb-b2 receptor tyrosine kinase 2 | SingleTM | Kinase_Protein | Non-Immune |
| 3791 | KDR | kinase insert domain receptor | SingleTM | Kinase_Protein | Non-Immune |
| 94 | ACVRL1 | activin A receptor like type 1 | SingleTM | Kinase_Protein | Possible |
| | | | | | Immune |
| 1436 | CSF1R | colony stimulating factor 1 | SingleTM | Kinase_Protein | Immune |
| | | receptor | | | |
| 2065 | ERBB3 | erb-b2 receptor tyrosine kinase 3 | SingleTM | Kinase_Protein | Possible |
| | | | | | Immune |
| 2261 | FGFR3 | fibroblast growth factor receptor 3 | SingleTM | Kinase_Protein | Possible |
| | | | | | Immune |
| 3480 | IGF1R | insulin like growth factor 1 | SingleTM | Kinase_Protein | Possible |
| | | receptor | | | Immune |
| 4233 | MET | MET proto-oncogene, receptor | SingleTM | Kinase_Protein | Possible |
| | | tyrosine kinase | | | Immune |
| 90 | ACVR1 | activin A receptor type 1 | SingleTM | Kinase_Protein | Possible |
| | | | | | Immune |
| 558 | AXL | AXL receptor tyrosine kinase | SingleTM | Kinase_Protein | Likely Immune |
| 2263 | FGFR2 | fibroblast growth factor receptor 2 | SingleTM | Kinase_Protein | Possible |
| | | | | | Immune |
| 2322 | FLT3 | fms related tyrosine kinase 3 | SingleTM | Kinase_Protein | Immune |
| 4919 | ROR1 | receptor tyrosine kinase like | SingleTM | Kinase_Protein | Immune |
| | | orphan receptor 1 | | | |
| 2260 | FGFR1 | fibroblast growth factor receptor 1 | SingleTM | Kinase_Protein | Possible |
| | | | | | Immune |
| 3815 | KIT | KIT proto-oncogene receptor | SingleTM | Kinase_Protein | Likely Immune |
| | | tyrosine kinase | | | |
| 4914 | NTRK1 | neurotrophic receptor tyrosine | SingleTM | Kinase_Protein | Likely Immune |
| | | kinase 1 | | | |
| 2321 | FLT1 | fms related tyrosine kinase 1 | SingleTM | Kinase_Protein | Likely Immune |
| 2324 | FLT4 | fms related tyrosine kinase 4 | SingleTM | Kinase_Protein | Likely Immune |
| 7010 | TEK | TEK receptor tyrosine kinase | SingleTM | Kinase_Protein | Likely Immune |
| 238 | ALK | ALK receptor tyrosine kinase | SingleTM | Kinase_Protein | Possible |
| | | | | | Immune |
| 7046 | TGFBR1 | transforming growth factor beta | SingleTM | Kinase_Protein | Likely Immune |
| | | receptor 1 | | | |
| 10461 | MERTK | MER proto-oncogene, tyrosine | SingleTM | Kinase_Protein | Likely Immune |
| | | kinase | | | |
| 7301 | TYRO3 | TYRO3 protein tyrosine kinase | SingleTM | Kinase_Protein | Possible |
| | | | | | Immune |
| 2050 | EPHB4 | EPH receptor B4 | SingleTM | Kinase_Protein | Possible |
| | | | | | Immune |
| 1969 | EPHA2 | EPH receptor A2 | SingleTM | Kinase_Protein | Possible |
| | | | | | Immune |
| 2066 | ERBB4 | erb-b2 receptor tyrosine kinase 4 | SingleTM | Kinase_Protein | Non-Immune |
| 2264 | FGFR4 | fibroblast growth factor receptor 4 | SingleTM | Kinase_Protein | Likely Immune |
| 4915 | NTRK2 | neurotrophic receptor tyrosine | SingleTM | Kinase_Protein | Non-Immune |
| | | kinase 2 | | | |
| 5159 | PDGFRB | platelet derived growth factor | SingleTM | Kinase_Protein | Possible |
| | | receptor beta | | | Immune |
| 657 | BMPR1A | bone morphogenetic protein | SingleTM | Kinase_Protein | Non-Immune |
| | | receptor type 1A | | | |
| 780 | DDR1 | discoidin domain receptor tyrosine | SingleTM | Kinase_Protein | Undetermined |
| | | kinase 1 | | | |
| 2043 | EPHA4 | EPH receptor A4 | SingleTM | Kinase_Protein | Possible |
| | | | | | Immune |
| 4486 | MST1R | macrophage stimulating 1 receptor | SingleTM | Kinase_Protein | Non-Immune |
| 4916 | NTRK3 | neurotrophic receptor tyrosine | SingleTM | Kinase_Protein | Non-Immune |
| | | kinase 3 | | | |
| 4921 | DDR2 | discoidin domain receptor tyrosine | SingleTM | Kinase_Protein | Possible |
| | | kinase 2 | | | Immune |
| 5156 | PDGFRA | platelet derived growth factor | SingleTM | Kinase_Protein | Likely Immune |
| | | receptor alpha | | | |
| 5979 | RET | ret proto-oncogene | SingleTM | Kinase_Protein | Likely Immune |
| 7048 | TGFBR2 | transforming growth factor beta | SingleTM | Kinase_Protein | Immune |
| | | receptor 2 | | | |
| 6098 | ROS1 | ROS proto-oncogene 1, receptor | SingleTM | Kinase_Protein | Possible |
| | | tyrosine kinase | | | Immune |
| 2268 | FGR | FGR proto-oncogene, Src family | Membrane | Kinase_Protein | Immune |
| | | tyrosine kinase | | | |
| 5747 | PTK2 | protein tyrosine kinase 2 | Membrane | Kinase_Protein | Possible |
| | | | | | Immune |
| 23043 | TNIK | TRAF2 and NCK interacting | SingleTM | Kinase_Protein | Immune |
| | | kinase | | | |
| 4070 | TACSTD2 | tumor associated calcium signal | SingleTM | Other | Likely Immune |
| | | transducer 2 | | | |
| 4072 | EPCAM | epithelial cell adhesion molecule | SingleTM | Other | Likely Immune |
| 10232 | MSLN | mesothelin | Membrane | Other | Likely Immune |
| 94025 | MUC16 | mucin 16, cell surface associated | SingleTM | Other | Likely Immune |
| 3977 | LIFR | LIF receptor alpha | SingleTM | Other | Possible |
| | | | | | Immune |
| 4071 | TM4SF1 | transmembrane 4 L six family | MultiTM | Other | Possible |
| | | member 1 | | | Immune |
| 4851 | NOTCH1 | notch 1 | SingleTM | Other | Likely Immune |
| 7037 | TFRC | transferrin receptor | SingleTM | Other | Immune |
| 8788 | DLK1 | delta like non-canonical Notch | SingleTM | Other | Likely Immune |
| | | ligand 1 | | | |
| 29881 | NPC1L1 | NPC1 like intracellular cholesterol | MultiTM | Other | Non-Immune |
| | | transporter 1 | | | |
| 55107 | ANO1 | anoctamin 1 | MultiTM | Other | Non-Immune |
| 4853 | NOTCH2 | notch 2 | SingleTM | Other | Likely Immune |
| 10267 | RAMP1 | receptor activity modifying protein | SingleTM | Other | Possible |
| | | 1 | | | Immune |
| 781 | CACNA2D1 | calcium voltage-gated channel | SingleTM | Other | Possible |
| | | auxiliary subunit alpha2delta 1 | | | Immune |
| 9365 | KL | klotho | SingleTM | Other | Likely Immune |
| 59283 | CACNG8 | calcium voltage-gated channel | MultiTM | Other | Likely Immune |
| | | auxiliary subunit gamma 8 | | | |
| 3958 | LGALS3 | galectin 3 | Membrane | Other | Possible |
| | | | | | Immune |
| 1191 | CLU | clusterin | Membrane | Other | Likely Immune |
| 6280 | S100A9 | S100 calcium binding protein A9 | Membrane | Other | Immune |
| 10519 | CIB1 | calcium and integrin binding 1 | Membrane | Other | Non-Immune |
| 1803 | DPP4 | dipeptidyl peptidase 4 | SingleTM | Protease | Immune |
| 2346 | FOLH1 | folate hydrolase 1, prostate | SingleTM | Protease | Undetermined |
| | | specific membrane antigen, PSMA | | | |
| 8754 | ADAM9 | ADAM metallopeptidase domain 9 | SingleTM | Protease | Immune |
| 2028 | ENPEP | glutamyl aminopeptidase | SingleTM | Protease | Likely Immune |
| 23621 | BACE1 | beta-secretase 1 | SingleTM | Protease | Non-Immune |
| 2191 | FAP | fibroblast activation protein alpha | SingleTM | Protease | Likely Immune |
| 8751 | ADAM15 | ADAM metallopeptidase domain | SingleTM | Protease | Non-Immune |
| | | 15 | | | |
| 290 | ANPEP | alanyl aminopeptidase, membrane | SingleTM | Protease | Immune |
| 1636 | ACE | angiotensin I converting enzyme | SingleTM | Protease | Likely Immune |
| 4311 | MME | membrane metalloendopeptidase | SingleTM | Protease | Immune |
| 5651 | TMPRSS15 | transmembrane serine protease 15 | SingleTM | Protease | Possible |
| | | | | | Immune |
| 9097 | USP14 | ubiquitin specific peptidase 14 | Membrane | Protease | Possible |
| | | | | | Immune |
| 6768 | ST14 | suppression of tumorigenicity 14 | SingleTM | Protease | Immune |
| 931 | MS4A1 | membrane spanning 4-domains | MultiTM | Receptor_all_{—} | Immune |
| | | A1 | | others | |
| 916 | CD3E | CD3e molecule | SingleTM | Receptor_all_{—} | Immune |
| | | | | others | |
| 920 | CD4 | CD4 molecule | SingleTM | Receptor_all_{—} | Immune |
| | | | | others | |
| 930 | CD19 | CD19 molecule | SingleTM | Receptor_all_{—} | Immune |
| | | | | others | |
| 1493 | CTLA4 | cytotoxic T-lymphocyte associated | SingleTM | Receptor_all_{—} | Immune |
| | | protein 4 | | others | |
| 3559 | IL2RA | interleukin 2 receptor subunit | SingleTM | Receptor_all_{—} | Immune |
| | | alpha | | others | |
| 3570 | IL6R | interleukin 6 receptor | SingleTM | Receptor_all_{—} | Immune |
| | | | | others | |
| 3674 | ITGA2B | integrin subunit alpha 2b | SingleTM | Receptor_all_{—} | Immune |
| | | | | others | |
| 3676 | ITGA4 | integrin subunit alpha 4 | SingleTM | Receptor_all_{—} | Immune |
| | | | | others | |
| 3685 | ITGAV | integrin subunit alpha V | SingleTM | Receptor_all_{—} | Possible |
| | | | | others | Immune |
| 3688 | ITGB1 | integrin subunit beta 1 | SingleTM | Receptor_all_{—} | Likely Immune |
| | | | | others | |
| 3689 | ITGB2 | integrin subunit beta 2 | SingleTM | Receptor_all_{—} | Immune |
| | | | | others | |
| 3690 | ITGB3 | integrin subunit beta 3 | SingleTM | Receptor_all_{—} | Immune |
| | | | | others | |
| 3695 | ITGB7 | integrin subunit beta 7 | SingleTM | Receptor_all_{—} | Immune |
| | | | | others | |
| 5133 | PDCD1 | programmed cell death 1 | SingleTM | Receptor_all_{—} | Immune |
| | | | | others | |
| 23765 | IL17RA | interleukin 17 receptor A | SingleTM | Receptor_all_{—} | Immune |
| | | | | others | |
| 29126 | CD274 | CD274 molecule | SingleTM | Receptor_all_{—} | Immune |
| | | | | others | |
| 57823 | SLAMF7 | SLAM family member 7 | SingleTM | Receptor_all_{—} | Immune |
| | | | | others | |
| 933 | CD22 | CD22 molecule | SingleTM | Receptor_all_{—} | Immune |
| | | | | others | |
| 941 | CD80 | CD80 molecule | SingleTM | Receptor_all_{—} | Immune |
| | | | | others | |
| 951 | CD37 | CD37 molecule | MultiTM | Receptor_all_{—} | Immune |
| | | | | others | |
| 351 | APP | amyloid beta precursor protein | SingleTM | Receptor_all_{—} | Non-Immune |
| | | | | others | |
| 970 | CD70 | CD70 molecule | SingleTM | Receptor_all_{—} | Immune |
| | | | | others | |
| 972 | CD74 | CD74 molecule | SingleTM | Receptor_all_{—} | Immune |
| | | | | others | |
| 1438 | CSF2RA | colony stimulating factor 2 | SingleTM | Receptor_all_{—} | Immune |
| | | receptor alpha subunit | | others | |
| 2152 | F3 | coagulation factor III, tissue factor | SingleTM | Receptor_all_{—} | Non-Immune |
| | | | | others | |
| 2348 | FOLR1 | folate receptor 1 | Membrane | Receptor_all_{—} | Possible |
| | | | | others | Immune |
| 2350 | FOLR2 | Folate receptor beta | Membrane | Receptor_all_{—} | Immune |
| | | | | others | |
| 3604 | TNFRSF9 | TNF receptor superfamily member | SingleTM | Receptor_all_{—} | Immune |
| | | 9 | | others | |
| 3693 | ITGB5 | integrin subunit beta 5 | SingleTM | Receptor_all_{—} | Likely Immune |
| | | | | others | |
| 3694 | ITGB6 | integrin subunit beta 6 | SingleTM | Receptor_all_{—} | Likely Immune |
| | | | | others | |
| 3821 | KLRC1 | killer cell lectin like receptor C1 | SingleTM | Receptor_all_{—} | Immune |
| | | | | others | |
| 3902 | LAG3 | lymphocyte activating 3 | SingleTM | Receptor_all_{—} | Immune |
| | | | | others | |
| 4345 | CD200 | CD200 molecule | SingleTM | Receptor_all_{—} | Likely Immune |
| | | | | others | |
| 4582 | MUC1 | mucin 1, cell surface associated | SingleTM | Receptor_all_{—} | Possible |
| | | | | others | Immune |
| 6403 | SELP | selectin P | SingleTM | Receptor_all_{—} | Possible |
| | | | | others | Immune |
| 7097 | TLR2 | toll like receptor 2 | SingleTM | Receptor_all_{—} | Immune |
| | | | | others | |
| 7293 | TNFRSF4 | TNF receptor superfamily member | SingleTM | Receptor_all_{—} | Immune |
| | | 4 | | others | |
| 8784 | TNFRSF18 | TNF receptor superfamily member | SingleTM | Receptor_all_{—} | Immune |
| | | 18 | | others | |
| 80381 | CD276 | CD276 molecule | SingleTM | Receptor_all_{—} | Immune |
| | | | | others | |
| 84868 | HAVCR2 | hepatitis A virus cellular receptor 2 | SingleTM | Receptor_all_{—} | Immune |
| | | | | others | |
| 201633 | TIGIT | T cell immunoreceptor with Ig and | SingleTM | Receptor_all_{—} | Immune |
| | | ITIM domains | | others | |
| 3556 | IL1RAP | interleukin 1 receptor accessory | SingleTM | Receptor_all_{—} | Immune |
| | | protein | | others | |
| 4680 | CEACAM6 | carcinoembryonic antigen related | Membrane | Receptor_all_{—} | Immune |
| | | cell adhesion molecule 6 | | others | |
| 7099 | TLR4 | toll like receptor 4 | SingleTM | Receptor_all_{—} | Immune |
| | | | | others | |
| 51206 | GP6 | glycoprotein VI platelet | SingleTM | Receptor_all_{—} | Immune |
| | | | | others | |
| 7162 | TPBG | trophoblast glycoprotein | SingleTM | Receptor_all_{—} | Possible |
| | | | | others | Immune |
| 81607 | NECTIN4 | nectin cell adhesion molecule 4 | SingleTM | Receptor_all_{—} | Likely Immune |
| | | | | others | |
| 943 | TNFRSF8 | TNF receptor superfamily member | SingleTM | Receptor_all_{—} | Immune |
| | | 8 | | others | |
| 945 | CD33 | CD33 molecule | SingleTM | Receptor_all_{—} | Immune |
| | | | | others | |
| 1048 | CEACAM5 | carcinoembryonic antigen related | Membrane | Receptor_all_{—} | Likely Immune |
| | | cell adhesion molecule 5 | | others | |
| 10683 | DLL3 | delta like canonical Notch ligand 3 | SingleTM | Receptor_all_{—} | Immune |
| | | | | others | |
| 3563 | IL3RA | interleukin 3 receptor subunit | SingleTM | Receptor_all_{—} | Immune |
| | | alpha | | others | |
| 160364 | CLEC12A | C-type lectin domain family 12 | SingleTM | Receptor_all_{—} | Immune |
| | | member A | | others | |
| 929 | CD14 | CD14 molecule | Membrane | Receptor_all_{—} | Immune |
| | | | | others | |
| 58985 | IL22RA1 | interleukin 22 receptor subunit | SingleTM | Receptor_all_{—} | Non-Immune |
| | | alpha 1 | | others | |
| 389400 | GFRAL | GDNF family receptor alpha like | SingleTM | Receptor_all_{—} | Undetermined |
| | | | | others | |
| 4179 | CD46 | CD46 molecule | SingleTM | Receptor_all_{—} | Possible |
| | | | | others | Immune |
| 131450 | CD200R1 | CD200 receptor 1 | SingleTM | Receptor_all_{—} | Immune |
| | | | | others | |
| 962 | CD48 | CD48 molecule | Membrane | Receptor_all_{—} | Immune |
| | | | | others | |
| 4065 | LY75 | lymphocyte antigen 75 | SingleTM | Receptor_all_{—} | Immune |
| | | | | others | |
| 131578 | LRRC15 | leucine rich repeat containing 15 | SingleTM | Receptor_all_{—} | Possible |
| | | | | others | Immune |
| 8718 | TNFRSF25 | TNF receptor superfamily member | SingleTM | Receptor_all_{—} | Immune |
| | | 25 | | others | |
| 64115 | VSIR | V-set immunoregulatory receptor | SingleTM | Receptor_all_{—} | Immune |
| | | | | others | |
| 2057 | EPOR | erythropoietin receptor | SingleTM | Receptor_all_{—} | Immune |
| | | | | others | |
| 3594 | IL12RB1 | interleukin 12 receptor subunit | SingleTM | Receptor_all_{—} | Immune |
| | | beta 1 | | others | |
| 3595 | IL12RB2 | interleukin 12 receptor subunit | SingleTM | Receptor_all_{—} | Immune |
| | | beta 2 | | others | |
| 3824 | KLRD1 | killer cell lectin like receptor D1 | SingleTM | Receptor_all_{—} | Immune |
| | | | | others | |
| 949 | SCARB1 | scavenger receptor class B | MultiTM | Receptor_all_{—} | Immune |
| | | member 1 | | others | |
| 3554 | IL1R1 | interleukin 1 receptor type 1 | SingleTM | Receptor_all_{—} | Immune |
| | | | | others | |
| 4352 | MPL | MPL proto-oncogene, | SingleTM | Receptor_all_{—} | Likely Immune |
| | | thrombopoietin receptor | | others | |
| 7850 | IL1R2 | interleukin 1 receptor type 2 | SingleTM | Receptor_all_{—} | Immune |
| | | | | others | |
| 177 | AGER | advanced glycosylation end- | SingleTM | Receptor_all_{—} | Immune |
| | | product specific receptor | | others | |
| 214 | ALCAM | activated leukocyte cell adhesion | SingleTM | Receptor_all_{—} | Immune |
| | | molecule | | others | |
| 3560 | IL2RB | interleukin 2 receptor subunit beta | SingleTM | Receptor_all_{—} | Immune |
| | | | | others | |
| 3683 | ITGAL | integrin subunit alpha L | SingleTM | Receptor_all_{—} | Immune |
| | | | | others | |
| 3696 | ITGB8 | integrin subunit beta 8 | SingleTM | Receptor_all_{—} | Likely Immune |
| | | | | others | |
| 4360 | MRC1 | mannose receptor C-type 1 | SingleTM | Receptor_all_{—} | Immune |
| | | | | others | |
| 6330 | SCN4B | sodium voltage-gated channel | SingleTM | Receptor_all_{—} | Possible |
| | | beta subunit 4 | | others | Immune |
| 7100 | TLR5 | toll like receptor 5 | SingleTM | Receptor_all_{—} | Immune |
| | | | | others | |
| 8764 | TNFRSF14 | TNF receptor superfamily member | SingleTM | Receptor_all_{—} | Likely Immune |
| | | 14 | | others | |
| 8792 | TNFRSF11A | TNF receptor superfamily member | SingleTM | Receptor_all_{—} | Immune |
| | | 11a | | others | |
| 50848 | F11R | F11 receptor | SingleTM | Receptor_all_{—} | Immune |
| | | | | others | |
| 8842 | PROM1 | prominin 1 | MultiTM | Receptor_all_{—} | Likely Immune |
| | | | | others | |
| 3678 | ITGA5 | integrin subunit alpha 5 | SingleTM | Receptor_all_{—} | Likely Immune |
| | | | | others | |
| 3105 | HLA-A | major histocompatibility complex, | SingleTM | Receptor_all_{—} | Likely Immune |
| | | class I, A | | others | |
| 3383 | ICAM1 | intercellular adhesion molecule 1 | SingleTM | Receptor_all_{—} | Immune |
| | | | | others | |
| 4804 | NGFR | nerve growth factor receptor | SingleTM | Receptor_all_{—} | Possible |
| | | | | others | Immune |
| 960 | CD44 | CD44 molecule (Indian blood | SingleTM | Receptor_all_{—} | Immune |
| | | group) | | others | |
| 1000 | CDH2 | cadherin 2 | SingleTM | Receptor_all_{—} | Possible |
| | | | | others | Immune |
| 1003 | CDH5 | cadherin 5 | SingleTM | Receptor_all_{—} | Possible |
| | | | | others | Immune |
| 4267 | CD99 | CD99 molecule (Xg blood group) | SingleTM | Receptor_all_{—} | Immune |
| | | | | others | |
| 6401 | SELE | selectin E | SingleTM | Receptor_all_{—} | Possible |
| | | | | others | Immune |
| 6402 | SELL | selectin L | SingleTM | Receptor_all_{—} | Immune |
| | | | | others | |
| 54106 | TLR9 | toll like receptor 9 | SingleTM | Receptor_all_{—} | Immune |
| | | | | others | |
| 54210 | TREM1 | triggering receptor expressed on | SingleTM | Receptor_all_{—} | Immune |
| | | myeloid cells 1 | | others | |
| 64581 | CLEC7A | C-type lectin domain containing | SingleTM | Receptor_all_{—} | Immune |
| | | 7A | | others | |
| 90952 | ESAM | endothelial cell adhesion molecule | SingleTM | Receptor_all_{—} | Likely Immune |
| | | | | others | |
| 3916 | LAMP1 | lysosomal associated membrane | SingleTM | Receptor_all_{—} | Possible |
| | | protein 1 | | others | Immune |
| 4155 | MBP | myelin basic protein | Membrane | Receptor_all_{—} | Likely Immune |
| | | | | others | |
| 476 | ATP1A1 | ATPase Na+/K+ transporting | MultiTM | Transporter | Non-Immune |
| | | subunit alpha 1 | | | |
| 6510 | SLC1A5 | solute carrier family 1 member 5 | MultiTM | Transporter | Immune |
| 10568 | SLC34A2 | solute carrier family 34 member 2 | MultiTM | Transporter | Possible |
| | | | | | Immune |
| 6517 | SLC2A4 | solute carrier family 2 member 4 | MultiTM | Transporter | Possible |
| | | | | | Immune |
| 116085 | SLC22A12 | solute carrier family 22 member 12 | MultiTM | Transporter | Non-Immune |
| 284111 | SLC13A5 | solute carrier family 13 member 5 | MultiTM | Transporter | Likely Immune |
| 8140 | SLC7A5 | solute carrier family 7 member 5 | MultiTM | Transporter | Immune |
| 19 | ABCA1 | ATP binding cassette subfamily A | MultiTM | Transporter | Likely Immune |
| | | member 1 | | | |
| 6531 | SLC6A3 | solute carrier family 6 member 3 | MultiTM | Transporter | Non-Immune |
| 6558 | SLC12A2 | solute carrier family 12 member 2 | MultiTM | Transporter | Non-Immune |
| 9152 | SLC6A5 | solute carrier family 6 member 5 | MultiTM | Transporter | Non-Immune |
| 9900 | SV2A | synaptic vesicle glycoprotein 2A | MultiTM | Transporter | Possible |
| | | | | | Immune |
| 30061 | SLC40A1 | solute carrier family 40 member 1 | MultiTM | Transporter | Immune |
| 496 | ATP4B | ATPase H+/K+ transporting | SingleTM | Transporter | Possible |
| | | subunit beta | | | Immune |
| 6554 | SLC10A1 | solute carrier family 10 member 1 | MultiTM | Transporter | Possible |
| | | | | | Immune |
| 6574 | SLC20A1 | solute carrier family 20 member 1 | MultiTM | Transporter | Likely Immune |
| 1080 | CFTR | cystic fibrosis transmembrane | MultiTM | Transporter | Non-Immune |
| | | conductance regulator | | | |
| 6530 | SLC6A2 | solute carrier family 6 member 2 | MultiTM | Transporter | Non-Immune |
| 6575 | SLC20A2 | solute carrier family 20 member 2 | MultiTM | Transporter | Non-Immune |
| 6833 | ABCC8 | ATP binding cassette subfamily C | MultiTM | Transporter | Non-Immune |
| | | member 8 | | | |
| 5243 | ABCB1 | ATP binding cassette subfamily B | MultiTM | Transporter | Likely Immune |
| | | member 1 | | | |
| 6532 | SLC6A4 | solute carrier family 6 member 4 | MultiTM | Transporter | Immune |
| 6546 | SLC8A1 | solute carrier family 8 member A1 | MultiTM | Transporter | Possible |
| | | | | | Immune |
| 6548 | SLC9A1 | solute carrier family 9 member A1 | MultiTM | Transporter | Possible |
| | | | | | Immune |
| 6566 | SLC16A1 | solute carrier family 16 member 1 | MultiTM | Transporter | Immune |
| 6573 | SLC19A1 | solute carrier family 19 member 1 | MultiTM | Transporter | Immune |
| 6535 | SLC6A8 | solute carrier family 6 member 8 | MultiTM | Transporter | Likely Immune |
| 6550 | SLC9A3 | solute carrier family 9 member A3 | MultiTM | Transporter | Undetermined |
| 6555 | SLC10A2 | solute carrier family 10 member 2 | MultiTM | Transporter | Non-Immune |
| 10060 | ABCC9 | ATP binding cassette subfamily C | MultiTM | Transporter | Likely Immune |
| | | member 9 | | | |
| 6529 | SLC6A1 | solute carrier family 6 member 1 | MultiTM | Transporter | Possible |
| | | | | | Immune |
| 6523 | SLC5A1 | solute carrier family 5 member 1 | MultiTM | Transporter | Possible |
| | | | | | Immune |
| 6524 | SLC5A2 | solute carrier family 5 member 2 | MultiTM | Transporter | Non-Immune |
| 6534 | SLC6A7 | solute carrier family 6 member 7 | MultiTM | Transporter | Undetermined |
| 6536 | SLC6A9 | solute carrier family 6 member 9 | MultiTM | Transporter | Likely Immune |
| 221074 | SLC39A12 | solute carrier family 39 member 12 | MultiTM | Transporter | Possible |
| | | | | | Immune |
| 6559 | SLC12A3 | solute carrier family 12 member 3 | MultiTM | Transporter | Likely Immune |
| 495 | ATP4A | ATPase H+/K+ transporting | MultiTM | Transporter | Likely Immune |
| | | subunit alpha | | | |
| 6557 | SLC12A1 | solute carrier family 12 member 1 | MultiTM | Transporter | Possible |
| | | | | | Immune |
| 1236 | CCR7 | C-C motif chemokine receptor 7 | MultiTM | GPCR | Immune |
| 2826 | CCR10 | C-C motif chemokine receptor 10 | MultiTM | GPCR | Immune |
| 2833 | CXCR3 | C-X-C motif chemokine receptor 3 | MultiTM | GPCR | Immune |
| 643 | CXCR5 | C-X-C motif chemokine receptor 5 | MultiTM | GPCR | Immune |
| 10663 | CXCR6 | C-X-C motif chemokine receptor 6 | MultiTM | GPCR | Immune |
| 57007 | ACKR3 | atypical chemokine receptor 3 | MultiTM | GPCR | Immune |
| 719 | C3AR1 | complement C3a receptor 1 | MultiTM | GPCR | Immune |
| 1880 | GPR183 | G-protein coupled receptor 183 | MultiTM | GPCR | Immune |
| 2859 | GPR35 | G protein-coupled receptor 35 | MultiTM | GPCR | Immune |
| 2866 | GPR42 | G protein-coupled receptor 42 | MultiTM | GPCR | Immune |
| 1524 | CX3CR1 | C-X3-C motif chemokine receptor | MultiTM | GPCR | Immune |
| | | 1 | | | |
| 4973 | OLR1 | oxidized low density lipoprotein | SingleTM | Receptor_all_{—} | Immune |
| | | receptor 1 | | others | |
| 5329 | PLAUR | plasminogen activator, urokinase | Membrane | Receptor_all_{—} | Immune |
| | | receptor | | others | |
| 768 | CA9 | Carbonic anhydrase 9 | SingleTM | Enzyme_other | Possible |
| | | | | | Immune |
| 771 | CA12 | carbonic anhydrase 12 | SingleTM | Enzyme_other | Possible |
| | | | | | Immune |
| 117194 | MRGPRX2 | Mas-related G-protein coupled | MultiTM | Exposed | Possible |
| | | receptor member X2 | | | Immune |
| 3320 | HSP90AA1 | heat shock protein 90 alpha family | Unattached | Enzyme_other | Possible |
| | | class A member 1 | | | Immune |
| 1803 | DPP4 | dipeptidyl peptidase 4 | SingleTM | Protease | Immune |
| 2358 | FPR2 | formyl peptide receptor 2 | MultiTM | GPCR | Immune |
| 56670 | SUCNR1 | succinate receptor 1 | MultiTM | GPCR | Immune |
| 6332 | SCN7A | sodium voltage-gated channel | MultiTM | Ion Channel | Possible |
| | | alpha subunit 7 | | | Immune |

In a further embodiment, the target of the target binding moiety is a chemokine receptor (CCR). In a further embodiment, the target of the target binding moiety is selected from CCR1, CCR2, CCR3, or CCR5.

In a further embodiment, the target of the target binding moiety is selected from C-C motif chemokine receptor (CCR)₂ (CCR2), CCR1, CCR3, CCR4, CCR5, CCR6, CCR7, CCR8, CCR9, CCR10, C-X-C motif chemokine receptor 1 (CXCR1), C-X-C motif chemokine receptor 2 (CXCR2), C-X-C motif chemokine receptor 3 (CXCR3), C-X-C motif chemokine receptor 4 (CXCR4), C-X-C motif chemokine receptor 5 (CXCR5), C-X-C motif chemokine receptor 6 (CXCR6), atypical chemokine receptor 3 (ACKR3), integrin avβ6, fibroblast activation protein-alpha (FAPa), prostate specific membrane antigen (PSMA), folate receptor (folate receptor 1 or folate receptor beta), complement C₃a receptor 1 (C₃AR1), complement C5a receptor 1 (C5AR1), G protein-coupled receptor (GPR)₆₅ (GPR65), GRP132, GPR84, GPR183, GPR35, GPR42, cholecystokinin A receptor (CCKAR), leukotriene B4 receptor (LTB4R), somatostatin receptor 2 (SSTR2), free fatty acid receptor 1 (FFAR1), purinergic receptor P2Y² (P2RY2), prostaglandin D2 receptor (PTGDR), calcitonin receptor (CALCR), CD38, purinergic receptor P2X 7 (P2RX7), integrin subunit alpha V (ITGAV), integrin subunit alpha 5 (ITGA5), integrin subunit beta 1 (ITGB1), integrin subunit beta 6 (ITGB6), integrin subunit beta 3 (ITGB3) prostaglandin D2 receptor 2 (PTGDR2), gastrin releasing peptide receptor (GRPR), MER proto-oncogene tyrosine kinase (MERTK), C-X3-C motif chemokine receptor 1 (CX3CR1), oxidized low density lipoprotein receptor 1 (OLR1), plasminogen activator urokinase receptor (PLAUR), carbonic anhydrase 9 (CA9), carbonic anhydrase 12 (CA 12), mas -related G-protein coupled receptor member X² (MRGPRX2), heat shock protein 90 alpha family class A member 1 (HSP90AA1), dipeptidyl peptidase 4 (DPP4), formyl peptide receptor 2 (FPR2), and succinate receptor 1 (SUCNR1).

In a further embodiment, the target-binding moiety T is a small molecule that binds a target as listed in Table 4. A person skilled in the art can select small molecules known to bind the target protein for use as the target-binding moiety in the ARM. In one embodiment, the target-binding small molecule is modified to include a functional group such as —NH₂ or -COOH to facilitate covalent coupling of the target-binding small molecule to the divalent linker moiety by amide bond formation.

The present disclosure also provides a pharmaceutical composition comprising a compound of Formula (I) as disclosed herein, and a pharmaceutically acceptable excipient, carrier, or diluent.

### Statement of Use of Antibody-Drug Conjugates

The compounds of Formula (I) and pharmaceutically acceptable salts thereof are capable of simultaneously binding a cell surface-expressed target and an antibody-drug conjugate comprising an anti-cotinine antibody, or antigen binding fragment thereof to form a ternary complex for the treatment and/or prevention of diseases or disorders associated with target-expressing cells.

In one embodiment, the present disclosure provides a method of treating and/or preventing a disease or disorder in a patient in need thereof comprising administering to the patient a therapeutically effective amount of a combination comprising an antibody-drug conjugate as disclosed herein and the compound of Formula (I), or a pharmaceutically acceptable salt thereof, wherein the disease or disorder is selected from a cancer, an inflammatory disease, an autoimmune disease, a viral infection, or a bacterial infection.

In a further embodiment, the compound and the antibody-drug conjugate are administered simultaneously. In a further embodiment, the compound and the antibody-drug conjugate are administered simultaneously from a single composition, including as a fixed-dose composition or by pre-mixing the compound and the antibody-drug conjugate prior to administration. For example, the compound and the antibody-drug conjugate can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the antibody-drug conjugate are administered simultaneously from two separate compositions.

In a further embodiment, the compound and the antibody-drug conjugate are administered sequentially.

In certain embodiments, the compound and the antibody-drug conjugate whether administered simultaneously or sequentially, may be administered by the same route or may be administered by different routes. In one embodiment, the compound and the antibody-drug conjugate are both administered intravenously or subcutaneously, in the same composition or in separate compositions. In another embodiment, the compound is administered orally and the antibody-drug conjugate is administered intravenously or subcutaneously.

In a further embodiment, the compound and the antibody-drug conjugate are administered in a molar ratio of compound to antibody-drug conjugate of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody-drug conjugate are present as a combination in a molar ratio of compound to antibody-drug conjugate of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody-drug conjugate are administered at a dosage of compound of 0.0001 mg/kg to 1 mg/kg and antibody-drug conjugate of 0.01 mg/kg to 100 mg/kg. For example, in a further embodiment, the compound is administered at a dosage of about 0.0001 mg/kg to about 0.0002 mg/kg, about 0.0002 mg/kg to about 0.0003 mg/kg, about 0.0003 mg/kg to about 0.0004 mg/kg, about 0.0004 mg/kg to about 0.0005 mg/kg, about 0.0005 mg/kg to about 0.001 mg/kg, about 0.001 mg/kg to about 0.002 mg/kg, about 0.002 mg/kg to about 0.003 mg/kg, about 0.003 mg/kg to about 0.004 mg/kg, about 0.004 mg/kg to about 0.005 mg/kg, about 0.005 mg/kg to about 0.01 mg/kg, about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, and/or about 0.5 mg/kg to about 1 mg/kg, and the antibody-drug conjugate is administered at a dosage of about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, and/or about 90 mg/kg to about 100 mg/kg.

In a further embodiment, the compound and the antibody-drug conjugate are administered at a dosage of compound of 0.007 mg to 70 mg and antibody-drug conjugate of 0.7 mg to 7000 mg. For example, in a further embodiment, the compound is administered at a dosage of about 0.007 mg to about 0.01 mg, about 0.01 mg to about 0.02 mg, about 0.02 mg to about 0.03 mg, about 0.03 mg to about 0.04 mg, about 0.04 mg to about 0.05 mg, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, and/or about 60 mg to about 70 mg, and the antibody-drug conjugate is administered at a dosage of about 0.7 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 100 mg, about 100 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, and/or about 6500 mg to about 7000 mg.

In a further embodiment, the compound and the antibody-drug conjugate are administered in a molar ratio and/or dosage as described herein once every week, once every two weeks, once every three weeks, once every four weeks, once every five weeks, or once every six weeks for a period of one week to one year, such as a period of one week, one month, two months, three months, four months, five months, six months, seven months, eight months, nine months, ten months, eleven months, or twelve months.

In a further embodiment, the present disclosure provides a therapeutically effective amount of the compound of Formula (I), or a pharmaceutically acceptable salt thereof, and the antibody-drug conjugate for use in therapy. The compound of Formula (I), or a pharmaceutically acceptable salt thereof, and the antibody-drug conjugate can be used in treating or preventing a disease or disorder selected from a cancer, an inflammatory disease, an autoimmune disease, a viral infection, or a bacterial infection.

In a further embodiment, the present disclosure provides a therapeutically effective amount of the compound of Formula (I), or a pharmaceutically acceptable salt thereof, and the antibody-drug conjugate for the manufacture of a medicament. The medicament can be used in treating or preventing a disease or disorder selected from a cancer, an inflammatory disease, an autoimmune disease, a viral infection, or a bacterial infection.

In a further embodiment, the disease or disorder is mediated by chemokine receptor 2 (CCR2) and/or is associated with CCR2-positive pathogenic cells. In a further embodiment, CCR2-positive cell types are identified by testing for expression of CCR2 such as by immunohistochemistry or flow cytometry.

In a further embodiment, the disease or disorder is mediated by C-X-C motif chemokine receptor 3 (CXCR3) and/or is associated with CXCR3-positive pathogenic cells. In a further embodiment, CXCR3-positive cell types are identified by testing for expression of CXCR3 such as by immunohistochemistry or flow cytometry.

In a further embodiment, the disease or disorder is mediated by PSMA and/or is associated with PSMA-positive pathogenic cells. In a further embodiment, PSMA-positive cell types are identified by testing for expression of PSMA such as by immunohistochemistry or flow cytometry.

In a further embodiment, the disease or disorder is mediated by integrin αVβ6 and/or is associated with integrin αVβ6-positive pathogenic cells. In a further embodiment, integrin αVβ6-positive cell types are identified by testing for expression of integrin αVβ6 such as by immunohistochemistry or flow cytometry.

In a further embodiment, the disease or disorder is mediated by folate receptor a (FRα) and/or folate receptor β (FRβ) and/or is associated with FRα- and/or FRβ-positive pathogenic cells. In a further embodiment, FRα- and/or FRβ-positive cell types are identified by testing for expression of FRα and/or FRβ such as by immunohistochemistry or flow cytometry.

In a further embodiment, the disease or disorder is mediated by fibroblast activation protein (FAP) and/or is associated with FAP-positive pathogenic cells. In a further embodiment, FAP-positive cell types are identified by testing for expression of FAP such as by immunohistochemistry or flow cytometry.

In a further embodiment, the disease or disorder is mediated by chemokine receptor 8 (CCR8) and/or is associated with CCR8-positive pathogenic cells. In a further embodiment, CCR8-positive cell types are identified by testing for expression of CCR8 such as by immunohistochemistry or flow cytometry.

In a further embodiment, the disease or disorder is a cancer selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer, preferably a cancer selected from mCRPC, breast cancer, lung cancer, colorectal cancer, or renal cell cancer.

In a further embodiment, the disease or disorder is a solid tumor. In a further embodiment, the disease or disorder is a solid tumor selected from lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer, preferably a solid tumor selected from mCRPC, breast cancer, lung cancer, colorectal cancer, or renal cell cancer.

In a further embodiment, the disease or disorder is a PD-1 relapsed or refractory cancer, such as a PD-1 relapsed or refractory lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer, preferably a PD-1 relapsed or refractory breast cancer, lung cancer, head and neck cancer, or cervical cancer.

In a further embodiment, the disease or disorder is a non-solid cancer. In a further embodiment, the disease or disorder is a leukemia, a lymphoma, or a myeloma.

In a further embodiment, the disease or disorder is a viral infection. In a further embodiment, the viral infection is caused by an influenza virus, a coronavirus (e.g., COVID-19), or a hepatitis B virus.

In a further embodiment, the disease or disorder is a bacterial infection. In a further embodiment, the bacterial infection is a chronic bacterial infection.

In a further embodiment, the disease is an autoimmune or inflammatory disease selected from vitiligo and type I diabetes.

In one embodiment, the present disclosure provides a method of increasing antibody-dependent cell cytotoxicity (ADCC) of target-expressing cells comprising contacting the cells with an effective amount of the compound of Formula (I), or pharmaceutically acceptable salt thereof, and an antibody-drug conjugate as disclosed herein, wherein the target-binding moiety of the compound binds the target expressed on the cells.

In one embodiment, the present disclosure provides a method of increasing antibody dependent cellular phagocytosis (ADCP) of target-expressing cells comprising contacting the cells with an effective amount of the compound of Formula (I), or pharmaceutically acceptable salt thereof, and an antibody-drug conjugate as disclosed herein, wherein the target-binding moiety of the compound binds the target expressed on the cells.

In one embodiment, the present disclosure provides a method of increasing complement dependant cytotoxicity (CDC) of target-expressing cells comprising contacting the cells with an effective amount of the compound of Formula (I), or pharmaceutically acceptable salt thereof, and an antibody-drug conjugate as disclosed herein, wherein the target-binding moiety of the compound binds the target expressed on the cells.

A compound of Formula (I), or pharmaceutically acceptable salt thereof, and an antibody-drug conjugate as disclosed herein may be administered as a conditioning therapy or combination therapy to improve efficacy in treatment of solid tumor cancers. In other embodiments, a compound of Formula (I), or pharmaceutically acceptable salt thereof, and an antibody-drug conjugate as disclosed herein may be administered as a neoadjuvant treatment for other therapies, including but not limited to immunotherapy, surgical resection, radiation, and/or chemotherapy.

In one embodiment, the present disclosure provides method of increasing cell killing of target-expressing cells comprising: contacting the cells with an effective amount of a combination comprising an antibody-drug conjugate as disclosed herein and a heterobifunctional molecule as disclosed herein, wherein the target-binding moiety of the compound binds the target expressed on the cells.

In one embodiment, the present disclosure provides a method of depleting target-expressing cells comprising: contacting the cells with an effective amount of a combination comprising an antibody-drug conjugate as disclosed herein and a heterobifunctional molecule as disclosed herein, wherein the target-binding moiety of the compound binds the target expressed on the cells.

In a further embodiment, the target-expressing cells are CCR2-expressing cells. In a further embodiment, the CCR2-expressing cells are myeloid-derived suppressor cells (MDSCs), T regulatory cells (Tregs), neutrophils, macrophages, B regulatory cells (Bregs), CD8 regulatory cells, (CD8regs), exhausted T cells, or cancer-associated fibroblasts (CAFs).

In a further embodiment, the target-expressing cells are CXCR3-expressing cells. In a further embodiment, the CXCR3-expressing cells are activated T cells, autoreactive T cells, T regulatory cells (Tregs), CD4 regulatory T cells (CD4regs), CD8 regulatory T cells, (CD8regs), T helper (Th) T cells, Th1 T cells, natural killer T (NKT) cells, natural killer (NK) cells, dendritic cells, B cells, yδT cells, or tumor cells.

In a further embodiment, the target-expressing cells are PSMA-expressing cells. In a further embodiment, the PSMA-expressing cells are tumor cells.

In a further embodiment, the target-expressing cells are integrin αVβ6-expressing cells. In a further embodiment, the integrin αVβ6-expressing cells are tumor cells.

In a further embodiment, the target-expressing cells are FRα- and/or FRβ-expressing cells. In a further embodiment, the FRα- and/or FRβ-expressing cells are myeloid derived suppressor cells (MDSCs), macrophages, B cells, or tumor cells.

In a further embodiment, the target-expressing cells are FAP-expressing cells. In a further embodiment, the FAP-expressing cells are cancer-associated fibroblasts (CAFs), macrophages, or tumor cells.

In a further embodiment, the target-expressing cells are CCR8-expressing cells. In a further embodiment, the CCR8-expressing cells are T regulatory cells (Tregs) or tumor cells.

In a further embodiment, the target-expressing cells are pathogenic cells.

In a further embodiment, the pathogenic cell is a pathogenic immune cell, a tumor cell or cancer cell, or a stromal cell.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or yδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), exhausted T cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, brain cancer cells, endocrine cancer cells, testicular cancer cells, bladder cancer cells, bone cancer cells, esophogeal cancer cells, gastric cancer cells, renal cell cancer cells, melanoma cancer cells, thyroid cancer cells, or breast cancer cells, preferably cells selected from mCRPC cells, breast cancer cells, lung cancer cells, colorectal cancer cells, or renal cell cancer cells.

In a further embodiment, the stromal cells are cancer associated fibroblasts (CAFs).

### Statement of Use of Bispecific Antibodies or Bispecific Antigen Binding Fragments Thereof

The compounds of Formula (I) and pharmaceutically acceptable salts thereof are capable of simultaneously binding a cell surface-expressed target and a bispecific antibody or bispecific antigen binding fragment thereof comprising a cotinine binding domain to form a ternary complex for the treatment and/or prevention of diseases or disorders associated with target-expressing cells. In an embodiment, the compounds of Formula (I) and pharmaceutically acceptable salts thereof are capable of simultaneously binding a cell surface-expressed target and a bispecific T-cell engager comprising a scFv that binds a cotinine moiety to form a ternary complex for the treatment and/or prevention of diseases or disorders associated with target-expressing cells.

In one embodiment, the present disclosure provides a method of treating and/or preventing a disease or disorder in a patient in need thereof comprising administering to the patient a therapeutically effective amount of a combination comprising a bispecific antibody or bispecific antigen binding fragment thereof (e.g., a bispecific T-cell engager) as disclosed herein and the compound of Formula (I), or a pharmaceutically acceptable salt thereof, wherein the disease or disorder is selected from a cancer, an inflammatory disease, an autoimmune disease, a viral infection, or a bacterial infection.

In a further embodiment, the compound and the bispecific antibody or bispecific antigen binding fragment thereof (e.g., a bispecific T-cell engager) are administered simultaneously. In a further embodiment, the compound and the bispecific antibody or bispecific antigen binding fragment thereof (e.g., a bispecific T-cell engager) are administered simultaneously from a single composition, including as a fixed-dose composition or by pre-mixing the compound and the bispecific antibody or bispecific antigen binding fragment thereof (e.g., a bispecific T-cell engager) prior to administration. For example, the compound and the bispecific antibody or bispecific antigen binding fragment thereof (e.g., a bispecific T-cell engager) can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the bispecific antibody or bispecific antigen binding fragment thereof (e.g., a bispecific T-cell engager) are administered simultaneously from two separate compositions.

In a further embodiment, the compound and the bispecific antibody or bispecific antigen binding fragment thereof (e.g., a bispecific T-cell engager) are administered sequentially.

In certain embodiments, the compound and the bispecific antibody or bispecific antigen binding fragment thereof (e.g., a bispecific T-cell engager) whether administered simultaneously or sequentially, may be administered by the same route or may be administered by different routes. In one embodiment, the compound and the bispecific antibody or bispecific antigen binding fragment thereof (e.g., a bispecific T-cell engager) are both administered intravenously or subcutaneously, in the same composition or in separate compositions. In another embodiment, the compound is administered orally and the bispecific antibody or bispecific antigen binding fragment thereof (e.g., a bispecific T-cell engager) is administered intravenously or subcutaneously.

In a further embodiment, the compound and the bispecific antibody or bispecific antigen binding fragment thereof (e.g., a bispecific T-cell engager) are administered in a molar ratio of compound to bispecific antibody or bispecific antigen binding fragment thereof (e.g., a bispecific T-cell engager) of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the bispecific antibody or bispecific antigen binding fragment thereof (e.g., a bispecific T-cell engager) are present as a combination in a molar ratio of compound to bispecific antibody or bispecific antigen binding fragment thereof (e.g., a bispecific T-cell engager) of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the bispecific antibody or bispecific antigen binding fragment thereof (e.g., a bispecific T-cell engager) are administered at a dosage of compound of 0.0001 mg/kg to 1 mg/kg and bispecific antibody or bispecific antigen binding fragment thereof (e.g., a bispecific T-cell engager) of 0.01 mg/kg to 100 mg/kg. For example, in a further embodiment, the compound is administered at a dosage of about 0.0001 mg/kg to about 0.0002 mg/kg, about 0.0002 mg/kg to about 0.0003 mg/kg, about 0.0003 mg/kg to about 0.0004 mg/kg, about 0.0004 mg/kg to about 0.0005 mg/kg, about 0.0005 mg/kg to about 0.001 mg/kg, about 0.001 mg/kg to about 0.002 mg/kg, about 0.002 mg/kg to about 0.003 mg/kg, about 0.003 mg/kg to about 0.004 mg/kg, about 0.004 mg/kg to about 0.005 mg/kg, about 0.005 mg/kg to about 0.01 mg/kg, about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, and/or about 0.5 mg/kg to about 1 mg/kg, and the bispecific antibody or bispecific antigen binding fragment thereof (e.g., a bispecific T-cell engager) is administered at a dosage of about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, and/or about 90 mg/kg to about 100 mg/kg.

In a further embodiment, the compound and the bispecific antibody or bispecific antigen binding fragment thereof (e.g., a bispecific T-cell engager) are administered at a dosage of compound of 0.007 mg to 70 mg and bispecific antibody or bispecific antigen binding fragment thereof (e.g., a bispecific T-cell engager) of 0.7 mg to 7000 mg. For example, in a further embodiment, the compound is administered at a dosage of about 0.007 mg to about 0.01 mg, about 0.01 mg to about 0.02 mg, about 0.02 mg to about 0.03 mg, about 0.03 mg to about 0.04 mg, about 0.04 mg to about 0.05 mg, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, and/or about 60 mg to about 70 mg, and the bispecific antibody or bispecific antigen binding fragment thereof (e.g., a bispecific T-cell engager) is administered at a dosage of about 0.7 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 100 mg, about 100 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, and/or about 6500 mg to about 7000 mg.

In a further embodiment, the compound and the bispecific antibody or bispecific antigen binding fragment thereof (e.g., a bispecific T-cell engager) are administered in a molar ratio and/or dosage as described herein once every week, once every two weeks, once every three weeks, once every four weeks, once every five weeks, or once every six weeks for a period of one week to one year, such as a period of one week, one month, two months, three months, four months, five months, six months, seven months, eight months, nine months, ten months, eleven months, or twelve months.

In a further embodiment, the present disclosure provides a therapeutically effective amount of the compound of Formula (I), or a pharmaceutically acceptable salt thereof, and the bispecific antibody or bispecific antigen binding fragment thereof (e.g., a bispecific T-cell engager) for use in therapy. The compound of Formula (I), or a pharmaceutically acceptable salt thereof, and the bispecific antibody or bispecific antigen binding fragment thereof (e.g., a bispecific T-cell engager) can be used in treating or preventing a disease or disorder selected from a cancer, an inflammatory disease, an autoimmune disease, a viral infection, or a bacterial infection.

In a further embodiment, the present disclosure provides a therapeutically effective amount of the compound of Formula (I), or a pharmaceutically acceptable salt thereof, and the bispecific antibody or bispecific antigen binding fragment thereof (e.g., a bispecific T-cell engager) for the manufacture of a medicament. The medicament can be used in treating or preventing a disease or disorder selected from a cancer, an inflammatory disease, an autoimmune disease, a viral infection, or a bacterial infection.

In a further embodiment, the disease or disorder is mediated by chemokine receptor 2 (CCR2) and/or is associated with CCR2-positive pathogenic cells. In a further embodiment, CCR2-positive cell types are identified by testing for expression of CCR2 such as by immunohistochemistry or flow cytometry.

In a further embodiment, the disease or disorder is mediated by C-X-C motif chemokine receptor 3 (CXCR3) and/or is associated with CXCR3-positive pathogenic cells. In a further embodiment, CXCR3-positive cell types are identified by testing for expression of CXCR3 such as by immunohistochemistry or flow cytometry.

In a further embodiment, the disease or disorder is mediated by PSMA and/or is associated with PSMA-positive pathogenic cells. In a further embodiment, PSMA-positive cell types are identified by testing for expression of PSMA such as by immunohistochemistry or flow cytometry.

In a further embodiment, the disease or disorder is mediated by integrin αVβ6 and/or is associated with integrin αVβ6-positive pathogenic cells. In a further embodiment, integrin αVβ6-positive cell types are identified by testing for expression of integrin αVβ6 such as by immunohistochemistry or flow cytometry.

In a further embodiment, the disease or disorder is mediated by folate receptor a (FRα) and/or folate receptor β (FRβ) and/or is associated with FRα- and/or FRβ-positive pathogenic cells. In a further embodiment, FRα- and/or FRβ-positive cell types are identified by testing for expression of FRα and/or FRβ such as by immunohistochemistry or flow cytometry.

In a further embodiment, the disease or disorder is mediated by fibroblast activation protein (FAP) and/or is associated with FAP-positive pathogenic cells. In a further embodiment, FAP-positive cell types are identified by testing for expression of FAP such as by immunohistochemistry or flow cytometry.

In a further embodiment, the disease or disorder is mediated by chemokine receptor 8 (CCR8) and/or is associated with CCR8-positive pathogenic cells. In a further embodiment, CCR8-positive cell types are identified by testing for expression of CCR8 such as by immunohistochemistry or flow cytometry.

In a further embodiment, the disease or disorder is a cancer selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer, preferably a cancer selected from mCRPC, breast cancer, lung cancer, colorectal cancer, or renal cell cancer.

In a further embodiment, the disease or disorder is a solid tumor. In a further embodiment, the disease or disorder is a solid tumor selected from lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer, preferably a solid tumor selected from mCRPC, breast cancer, lung cancer, colorectal cancer, or renal cell cancer.

In a further embodiment, the disease or disorder is a PD-1 relapsed or refractory cancer, such as a PD-1 relapsed or refractory lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer, preferably a PD-1 relapsed or refractory breast cancer, lung cancer, head and neck cancer, or cervical cancer.

In a further embodiment, the disease or disorder is a non-solid cancer. In a further embodiment, the disease or disorder is a leukemia, a lymphoma, or a myeloma.

In a further embodiment, the disease or disorder is a viral infection. In a further embodiment, the viral infection is caused by an influenza virus, a coronavirus (e.g., COVID-19), or a hepatitis B virus.

In a further embodiment, the disease or disorder is a bacterial infection. In a further embodiment, the bacterial infection is a chronic bacterial infection.

In a further embodiment, the disease is an autoimmune or inflammatory disease selected from vitiligo and type I diabetes.

In one embodiment, the present disclosure provides a method of increasing antibody-dependent cell cytotoxicity (ADCC) of target-expressing cells comprising contacting the cells with an effective amount of the compound of Formula (I), or pharmaceutically acceptable salt thereof, and a bispecific antibody or bispecific antigen binding fragment thereof (e.g., a bispecific T-cell engager) as disclosed herein, wherein the target-binding moiety of the compound binds the target expressed on the cells.

In one embodiment, the present disclosure provides a method of increasing antibody dependent cellular phagocytosis (ADCP) of target-expressing cells comprising contacting the cells with an effective amount of the compound of Formula (I), or pharmaceutically acceptable salt thereof, and a bispecific antibody or bispecific antigen binding fragment thereof (e.g., a bispecific T-cell engager) as disclosed herein, wherein the target-binding moiety of the compound binds the target expressed on the cells.

In one embodiment, the present disclosure provides a method of increasing complement dependant cytotoxicity (CDC) of target-expressing cells comprising contacting the cells with an effective amount of the compound of Formula (I), or pharmaceutically acceptable salt thereof, and a bispecific antibody or bispecific antigen binding fragment thereof (e.g., a bispecific T-cell engager) as disclosed herein, wherein the target-binding moiety of the compound binds the target expressed on the cells.

A compound of Formula (I), or pharmaceutically acceptable salt thereof, and a bispecific antibody or bispecific antigen binding fragment thereof (e.g., a bispecific T-cell engager) as disclosed herein may be administered as a conditioning therapy or combination therapy to improve efficacy in treatment of solid tumor cancers. In other embodiments, a compound of Formula (I), or pharmaceutically acceptable salt thereof, and a bispecific antibody or bispecific antigen binding fragment thereof (e.g., a bispecific T-cell engager) as disclosed herein may be administered as a neoadjuvant treatment for other therapies, including but not limited to immunotherapy, surgical resection, radiation, and/or chemotherapy.

In one embodiment, the present disclosure provides method of increasing cell killing of target-expressing cells comprising: contacting the cells with an effective amount of a combination comprising a bispecific antibody or bispecific antigen binding fragment thereof (e.g., a bispecific T-cell engager) as disclosed herein and a heterobifunctional molecule as disclosed herein, wherein the target-binding moiety of the compound binds the target expressed on the cells.

In one embodiment, the present disclosure provides a method of depleting target-expressing cells comprising: contacting the cells with an effective amount of a combination comprising a bispecific antibody or bispecific antigen binding fragment thereof (e.g., a bispecific T-cell engager) as disclosed herein and a heterobifunctional molecule as disclosed herein, wherein the target-binding moiety of the compound binds the target expressed on the cells.

In a further embodiment, the target-expressing cells are CCR2-expressing cells. In a further embodiment, the CCR2-expressing cells are myeloid-derived suppressor cells (MDSCs), T regulatory cells (Tregs), neutrophils, macrophages, B regulatory cells (Bregs), CD8 regulatory cells, (CD8regs), exhausted T cells, or cancer-associated fibroblasts (CAFs).

In a further embodiment, the target-expressing cells are CXCR3-expressing cells. In a further embodiment, the CXCR3-expressing cells are activated T cells, autoreactive T cells, T regulatory cells (Tregs), CD4 regulatory T cells (CD4regs), CD8 regulatory T cells, (CD8regs), T helper (Th) T cells, Th1 T cells, natural killer T (NKT) cells, natural killer (NK) cells, dendritic cells, B cells, yδT cells, or tumor cells.

In a further embodiment, the target-expressing cells are PSMA-expressing cells. In a further embodiment, the PSMA-expressing cells are tumor cells.

In a further embodiment, the target-expressing cells are integrin αVβ6-expressing cells. In a further embodiment, the integrin αVβ6-expressing cells are tumor cells.

In a further embodiment, the target-expressing cells are FRα- and/or FRβ-expressing cells. In a further embodiment, the FRα- and/or FRβ-expressing cells are myeloid derived suppressor cells (MDSCs), macrophages, B cells, or tumor cells.

In a further embodiment, the target-expressing cells are FAP-expressing cells. In a further embodiment, the FAP-expressing cells are cancer-associated fibroblasts (CAFs), macrophages, or tumor cells.

In a further embodiment, the target-expressing cells are CCR8-expressing cells. In a further embodiment, the CCR8-expressing cells are T regulatory cells (Tregs) or tumor cells. In a further embodiment, the target-expressing cells are pathogenic cells.

In a further embodiment, the pathogenic cell is a pathogenic immune cell, a tumor cell or cancer cell, or a stromal cell.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or yδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), exhausted T cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, brain cancer cells, endocrine cancer cells, testicular cancer cells, bladder cancer cells, bone cancer cells, esophogeal cancer cells, gastric cancer cells, renal cell cancer cells, melanoma cancer cells, thyroid cancer cells, or breast cancer cells, preferably cells selected from mCRPC cells, breast cancer cells, lung cancer cells, colorectal cancer cells, or renal cell cancer cells.

In a further embodiment, the stromal cells are cancer associated fibroblasts (CAFs).

### Combination Therapies

The compounds of the invention may be employed alone or in combination with other therapeutic agents. Combination therapies according to the present invention thus comprise the administration of at least one compound of Formula (I) or a pharmaceutically acceptable salt thereof, and the use of at least one other pharmaceutically active agent. The compounds of the invention and the other pharmaceutically active agents may be administered together in a single pharmaceutical composition or separately and, when administered separately this may occur simultaneously or sequentially in any order. The amounts of the compounds of the invention and the other pharmaceutically active agents and the relative timings of administration will be selected in order to achieve the desired combined therapeutic effect.

It will be appreciated that when the compound of the present invention is administered in combination with one or more other therapeutically active agents normally administered by the inhaled, intravenous, oral, intranasal, ocular topical or other route, that the resultant pharmaceutical composition may be administered by the same route. Alternatively, the individual components of the composition may be administered by different routes.

In one embodiment, the compounds and pharmaceutical composition disclosed herein are used in combination with, or include, one or more additional therapeutic agents. In a further embodiment, the additional therapeutic agent is a checkpoint inhibitor or an immune modulator.

In a further embodiment, the checkpoint inhibitor is selected from a PD-1 inhibitor (e.g., an anti-PD-1 antibody including, but not limited to, pembrolizumab, nivolumab, cemiplimab, or dostarlimab), a PD-L¹ inhibitor (e.g., an anti-PD-L¹ antibody including, but not limited to, atezolizumab, avelumab, or durvalumab), or a CTLA-4 inhibitor (e.g., an anti-CTLA-4 antibody including, but not limited to, ipilimumab or tremilumumab).

In a further embodiment, the checkpoint inhibitor is selected from a CD226 axis inhibitor, including but not limited to a TIGIT inhibitor (e.g., an anti-TIGIT antibody), a CD96 inhibitor (e.g., an anti-CD96 antibody), and/or a PVRIG inhibitor (e.g., an anti-PVRIG antibody).

In a further embodiment, the immune modulator is an ICOS agonist (e.g., an anti-ICOS antibody including, but not limited to feladilimab), a PARP inhibitor (e.g., niraparib, olaparib), or a STING agonist.

### Pharmaceutical Compositions, Dosages, and Dosage Forms

For the purposes of administration, in certain embodiments, the ARMs described herein are administered as a raw chemical or are formulated as pharmaceutical compositions.

Pharmaceutical compositions disclosed herein include an ARM and one or more of: a pharmaceutically acceptable carrier, diluent or excipient. An ARM is present in the composition in an amount which is effective to treat a particular disease, disorder or condition of interest. The activity of the ARM can be determined by one skilled in the art, for example, as described in the biological assays described below. Appropriate concentrations and dosages can be readily determined by one skilled in the art. In certain embodiments, the ARM is present in the pharmaceutical composition in an amount from about 25 mg to about 500 mg. In certain embodiments, the ARM is present in the pharmaceutical composition in an amount of about 0.01 mg to about 300 mg. In certain embodiments, the ARM is present in the pharmaceutical composition in an amount of about 0.01 mg, 0.1 mg, 1 mg, 5 mg, 10 mg, 25 mg, 50 mg, 100 mg, 200 mg, 300 mg, 400 mg or about 500 mg.

Administration of the compounds of the invention, or their pharmaceutically acceptable salts, in pure form or in an appropriate pharmaceutical composition, is carried out via any of the accepted modes of administration of agents for serving similar utilities. The pharmaceutical compositions of the invention are prepared by combining a compound of the invention with an appropriate pharmaceutically acceptable carrier, diluent or excipient, and in specific embodiments are formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral (e.g., intramuscular, subcutaneous, intravenous, or intradermal), sublingual, buccal, rectal, vaginal, and intranasal. Pharmaceutical compositions of the invention are formulated so as to allow the active ingredients contained therein to be bioavailable upon administration of the composition to a patient.

Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia. College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings described herein.

The pharmaceutical compositions disclosed herein are prepared by methodologies well known in the pharmaceutical art. For example, in certain embodiments, a pharmaceutical composition intended to be administered by injection is prepared by combining a compound of the invention with sterile, distilled water so as to form a solution. In some embodiments, a surfactant is added to facilitate the formation of a homogeneous solution or suspension. Surfactants are compounds that non-covalently interact with the compound of the invention so as to facilitate dissolution or homogeneous suspension of the compound in the aqueous delivery system.

Traditional antibody therapeutics have several disadvantages that are addressed by the ARMs approach described herein including difficulties in managing adverse events via adjusting dose and dose frequency of administration, challenges in generating antibodies to certain classes of drug targets (e.g., GPCRs, ion channels, and enzymes), and a new cell line for development is required for each new antibody which can be slow and costly. In contrast, the ARMs approach provides the following advantages: uniting the pharmacology of antibodies with the dose-control of small molecules, dose controlled PK/PD allowing temporal cell depletion, simpler multimerization, and rapid reversal of cell depletion through dosing of the antibody-binding component (e.g., cotinine hapten) which can uncouple therapeutic effects from potential adverse events. Further, the ARMs approach disclosed herein provides a route to both small molecule-activated antibody-drug conjugates and small molecule-activated bispecific antibodies or bispecific antigen binding fragments thereof (e.g., bispecific T-cell engagers).

### Examples

The following examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made.

### Compound Synthesis

The compounds according to Formula (I) were prepared using conventional organic synthetic methods as described, for example, in International Patent Application No. PCT/IB2022/057562, which is hereby incorporated by reference in its entirety.

### Biological Assays

Compounds 1-2 which are compounds of Formula (I) having a PSMA- or CCR2-binding moiety were tested in various biological assays as described in more detail below.

### EXAMPLE 1: Preparation and Analysis of Cotinine Targeting Antibody-Drug Conjugate (ADC)

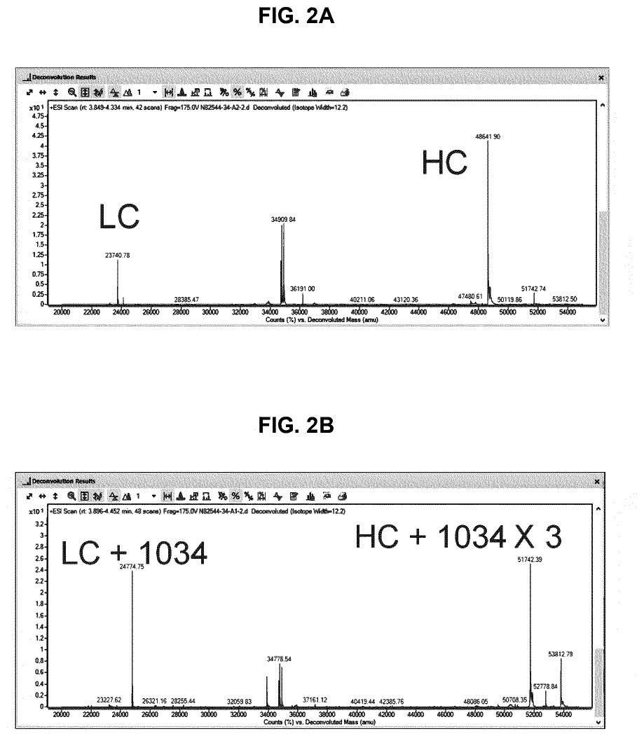

Anti-cotinine antibody having a heavy chain sequence of SEQ ID NO: 9 and a light chain sequence of SEQ ID NO: 10 was diluted from the original storage solution (122.94 mg/mL) into 3.86 mg/mL (by UV 280 nm) reaction medium with commercial PBS pH 7.4 (1X). Then, 500 μL of the anti-cotinine antibody was placed in a 1 mL vial and warmed up to 37° C. on a thermal mixer. 13 μL of 10 mM Tris -(2-carboxyethyl) phosphine hydrochloride (TCEP) solution made using de-ionized water was added. The resultant medium was incubated on a thermal mixer at 500 rpm at 37° C. for 90 min. The reaction vial was cooled to 23° C. Then, 13 μL of 10 mM deruxtecan (molecular weight 1034.07, commercially sourced) in DMSO solution was added. The vial was incubated for 2 hours at 500 rpm. The conjugation medium was ultra-filtered by a pre-conditioned Amicro spin filter (MWCO 10k, 0.5 mL, Millipore) into PBS (50 μL, 6.5 mg/mL at UV 280 nm). The resultant anti-cotinine ADC was analyzed by analytical LCMS as described below and shown in FIG. 2.

Analytical LCMS analysis: 1 μL of the conjugate was treated with N-glycosidase F (PNGase F) (9 μL, 10X diluted from commercial stock solution, New England Biolabs) at room temperature overnight and 2 μL was injected on Agilent TOF LCMS 6224A. The data was processed using MassHunter Acquisition & Analysis Software v B. 10. The molecular weight of deruxtecan is 1034. Mass of unconjugated light chain (LC) of anti-cotinine antibody: 23740.78; mass of light chain of the conjugated product was 24774.75 (LC+1034); mass of unconjugated heavy chain (HC) of anti-cotinine antibody: 48641.90; mass of heavy chain of the conjugated product was 51742.39 (HC+1034×3). The drug-antibody ratio was determined to be 8.0. Hydrophobic interaction HPLC analysis (AdvanceBio, HIC 4.6×100 mm, PN685975-908) displayed a single peak chromatograph.

### EXAMPLE 2: Antibody-Drug Conjugate Cell Killing Assay

An antibody-drug conjugate (ADC) cell killing assay was developed to screen cotinine targeted ADCs in a 384-well plate format. This assay utilized (i) an anti-cotinine antibody having a heavy chain sequence of SEQ ID NO: 9 and a light chain sequence of SEQ ID NO: conjugated with Deruxtecan and (ii) a Prostate Specific Membrane Antigen (PSMA) Cytotoxicity Targeting Chimera (CyTaC) to target PSMA expressing cells for death. The structures of the ADC and PSMA targeted CyTaC used in this experiment are shown below:

### ADC Structure:

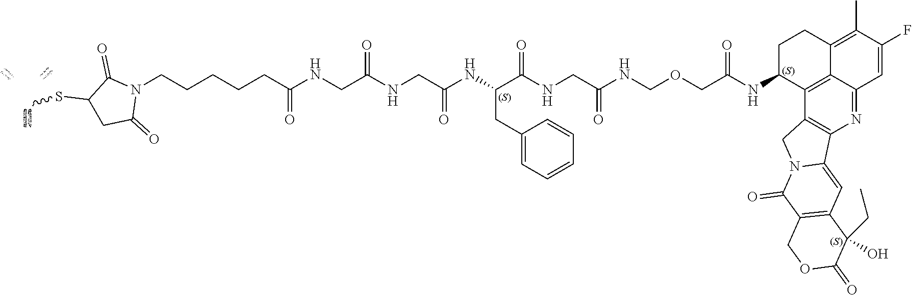

### PSMA Cytac Structure:

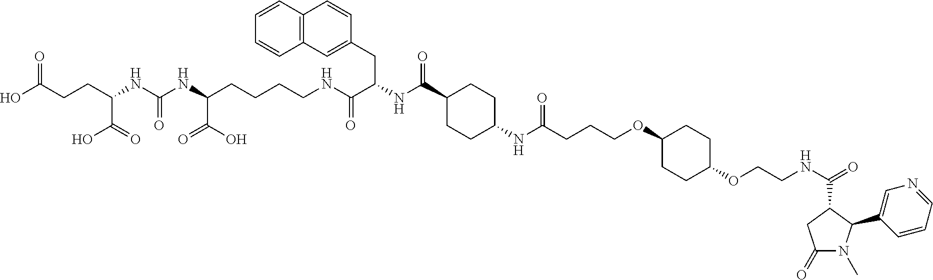 [SMILES: CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1]

To screen ADCs, 10,000 PSMA-expressing cells (LNCAP) or 10,000 non-expressing cells (CHO) in media (RPMI 1640 (Gibco) containing 10% fetal bovine serum (Gibco)) were transferred to a 384-well plate (Greiner Bio-One). To each well containing cells, 100 nM ADC was added. Next, serial diluted PSMA targeted CyTaC (maximum concentration of 20 μM) was added to appropriate wells. The plates were then incubated for 48 hours at 37° C. with 5% CO₂. Following the 48 hour incubation, an equal volume of CellTiter Glo (Promega) was added to each well and the plates were read on a PheraStar Plate Reader (BMG) to measure cell viability via Luminescence signal. Signal: background was calculated by dividing the signal of a test well by the signal obtained when no CyTaC was added. The % cell death was calculated by: (1-(test well signal/no CyTaC signal))*100. All other calculations were performed using Graphpad Prism Software.

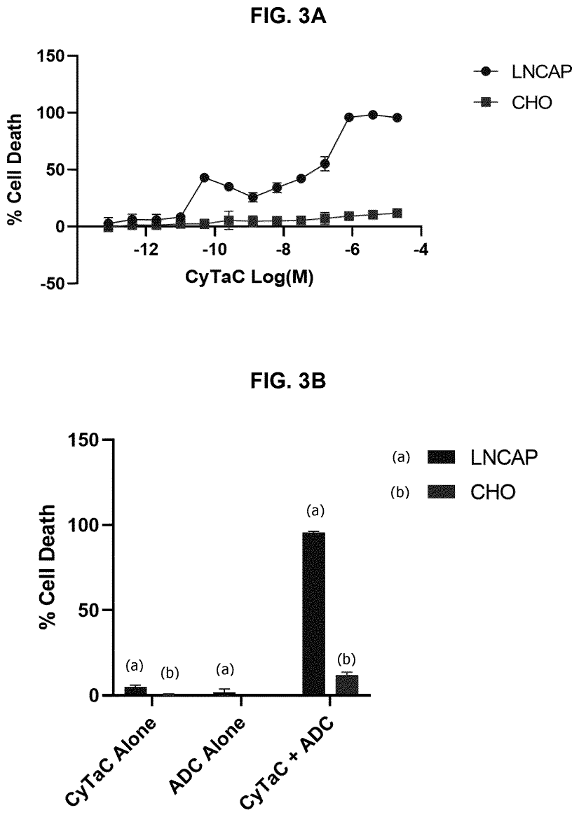

The results are shown in FIG. 3. The results demonstrate ADC mediated cell killing of PSMA expressing cells in the presence of PSMA targeted CyTaC molecule.

### EXAMPLE 3: Bispecific Antibody T-Cell Activation Assay

A T-cell Activation reporter assay was conducted using the following four assay components: (i) ARM compound of Formula (I) targeting CCR2 (concentrations ranging from 1 μM to 10 μM) (ii) a cotinine and CD3 bispecific antibody comprising a cotinine binding domain comprising a heavy chain of SEQ ID NO: 28 and a light chain of SEQ ID NO: 10; and a CD3 binding domain comprising a heavy chain of SEQ ID NO: 26 and a light chain of SEQ ID NO: 27 (concentrations ranging from 0.01 μg/mL to 200 μg/mL); (iii) target cells: CHOK1 cells engineered to overexpress either human CCR2 (typically 1000-20,000 cells per well) and (iv) reporter cells: Jurkat cells engineered to express CD3 with the reporter gene luciferase under the control of the NFAT promoter (typically 3000-75,000 cells per well). The structure of the CCR2 targeted CyTaC used in this experiment is shown below:

### CCR2 Cytac Structure:

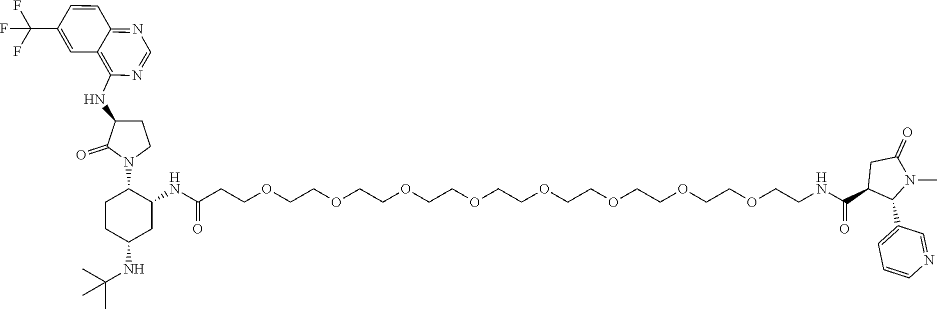 [SMILES: CN1C(=O)C[C@H](C(=O)NCCOCCOCCOCCOCCOCCOCCOCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1]

Reagents were combined in a final volume of 20 μL in a 384-well tissue culture treated plate. All four assay components were incubated together for about 12-18 hours. Thereafter, BioGlo Detection reagent (Promega) was added to the wells to lyse the cells and provide a substrate for the luciferase reporter protein. Luminescence signal was measured on a microplate reader and signal: background was calculated by dividing the signal of a test well by the signal obtained when no heterobivalent compound of Formula (I) was added. EC50 calculations were done using Graphpad Prism Software, specifically a nonlinear regression curve fit (Y=Bottom+(Top−Bottom)/(1+10 \hat{ }((Log EC50−X)*HillSlope))).

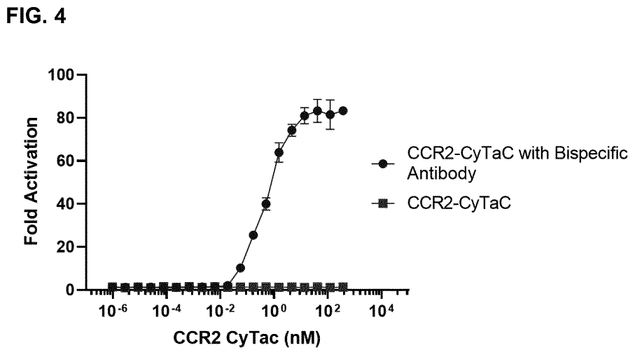

The results are shown in FIG. 4.

SEQUENCE LISTINGS

Heavy chain CDR1 amino acid sequence (cotinine)
SEQ ID NO: 1
NYWMS
Heavy chain CDR2 amino acid sequence (cotinine)
SEQ ID NO: 2
DIHGNRGFNYHASWAKG
Heavy chain CDR3 amino acid sequence (cotinine)
SEQ ID NO: 3
ADDSGSHDI
Light chain CDR1 amino acid sequence (cotinine)
SEQ ID NO: 4
QSSQSVYSAKLS
Light chain CDR2 amino acid sequence (cotinine)
SEQ ID NO: 5
YGSTLAS
Light chain CDR3 amino acid sequence (cotinine)
SEQ ID NO: 6
QGTFYGPDWYFA
Variable heavy chain amino acid sequence (cotinine)
SEQ ID NO: 7
EVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIW
GQGTLVTVSS
Variable light chain amino acid sequence (cotinine)
SEQ ID NO: 8
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIK
Heavy chain amino acid sequence (cotinine)
SEQ ID NO: 9
EVQLVESGGGLVQPGGSLRLSCAASGFTFSNYWMSWVRQAPGKGLEWVGDIHGNRGFN
YHASWAKGRFTVSRSKNTLYLQMNSLRAEDTAVYYCAKADDSGSHDIWGQGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPDVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPEEKTISKAKGQPREPQVYTLPPSRDELTKNQ
VSLTCLVKGFYPSDIAVEWESNGQPENNYKTTPPVLDSDGSFFLYSKLTVDKSRWQQGNV
FSCSVMHEALHNHYTQKSLSLSPGK
Light chain amino acid sequence (cotinine)
SEQ ID NO: 10
DIQMTQSPSSLSASVGDRVTITCQSSQSVYSAKLSWYQQKPGKAPKLLIYYGST
LASGVPSRFSGSGSGTQFTLTISSLQPEDFATYYCQGTFYGPDWYFAFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
Heavy chain amino acid sequence
SEQ ID NO: 11
QQQLVESGGR LVTPGGSLTL TCTASGFSLN NYWMSWVRQA PGKGLEWIGD
IHGNRGFNYH ASWAKGRFTV SRTSTTVDLR MTSLTTEDTA IYFCARADDS
GSHDIWGPGT LVTVSSASTK GPSVFPLAPS SKSTSGGTAA LGCLVKDYFP
EPVTVSWNSG ALTSGVHTFP AVLQSSGLYS LSSVVTVPSS SLGTQTYICN
VNHKPSNTKV DKKVEPKSCD KTHTCPPCPA PELLGGPDVF LFPPKPKDTL
MISRTPEVTC VVVDVSHEDP EVKFNWYVDG VEVHNAKTKP REEQYNSTYR
VVSVLTVLHQ DWLNGKEYKC KVSNKALPAP EEKTISKAKG QPREPQVYTL
PPSRDELTKN QVSLTCLVKG FYPSDIAVEW ESNGQPENNY KTTPPVLDSD
GSFFLYSKLT VDKSRWQQGN VFSCSVMHEA LHNHYTQKSL SLSPGK
Light chain amino acid sequence
SEQ ID NO: 12
ELDLTQTPSPVSAAVGDTVTINCQSSQSVYSAKLSWYQQKPGQPPKLLIYYGSTLASGVPS
RFKGSGSGTQFSLTISDVQCADAATYYCQGTYYGPDWYFAFGGGTEVVVKRTVAAPSVFI
FPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Heavy chain amino acid sequence
SEQ ID NO: 13
QQQLVESGGRLVTPGGSLTLTCTASGFSLNNYWMSWVRQAPGKGLEWIGDIHGNRGFNY
HASWAKGRFTVSRTSTTVDLRMTSLTTEDTAIYFCARADDSGSHDIWGPGTLVTVSSAKTT
APSVYPLAPVCGDTTGSSVTLGCLVKGYFPEPVTLTWNSGSLSSGVHTFPAVLQSDLYTLS
SSVTVTSSTWPSQSITCNVAHPASSTKVDKKIEPRGPTIKPCPPCKCPAPNLLGGPSVFIFPP
KIKDVLMISLSPMVTCVVVDVSEDDPDVQISWFVNNVEVLTAQTQTHREDYNSTLRVVSALP
IQHQDWMSGKEFKCKVNNKALPAPIERTISKPKGSVRAPQVYVLPPPEEEMTKKQVTLTCM
VTDFMPEDIYVEWTNNGKTELNYKNTEPVLDSDGSYFMYSKLRVEKKNWVERNSYSCSVV
HEGLHNHHTTKSFSRTPGK
Light chain amino acid sequence
SEQ ID NO: 14
ELDLTQTPSPVSAAVGDTVTINCQSSQSVYSAKLSWYQQKPGQPPKLLIYYGSTLASGVPS
RFKGSGSGTQFSLTISDVQCADAATYYCQGTYYGPDWYFAFGGGTEVVVKRADAAPTVSI
FPPSSEQLTSGGASVVCFLNNFYPKDINVKWKIDGSERQNGVLNSWTDQDSKDSTYSMSS
TLTLTKDEYERHNSYTCEATHKTSTSPIVKSFNRNEC
Anti-cotinine scFv
SEQ ID NO: 15
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSEVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIWGQGTLV
TVSS
Linker; CDR; VH/VL
Heavy chain CDR1 amino acid sequence (CD3)
SEQ ID NO: 16
GYTFTNYYIH
Heavy chain CDR2 amino acid sequence (CD3)
SEQ ID NO: 17
GWIYPGDGNTKYNEKFKG
Heavy chain CDR3 amino acid sequence (CD3)
SEQ ID NO: 18
DSYSNYYFDY
Light chain CDR1 amino acid sequence (CD3)
SEQ ID NO: 19
KSSQSLLNSRTRKNYLA
Light chain CDR2 amino acid sequence (CD3)
SEQ ID NO: 20
WASTRES
Light chain CDR3 amino acid sequence (CD3)
SEQ ID NO: 21
TQSFILRT
Variable heavy chain amino acid sequence (CD3)
SEQ ID NO: 22
EVQLVQSGAEVKKPGASVKVSCKASGYTFTNYYIHWVRQAPGQGLEWIGWIYPGDGNTKY
NEKFKGRATLTADTSTSTAYLELSSLRSEDTAVYYCARDSYSNYYFDYWGQGTLVTVSS
Variable light chain amino acid sequence (CD3)
SEQ ID NO: 23
DIVMTQSPDSLAVSLGERATINCKSSQSLLNSRTRKNYLAWYQQKPGQPPKLLIYWASTRE
SGVPDRFSGSGSGTDFTLTISSLQAEDVAVYYCTQSFILRTFGQGTKVEIK
Anti-CD3 scFv
SEQ ID NO: 24
EVQLVESGGGLVQPGGSLKLSCAASGFTFNKYAMNWVRQAPGKGLEWVARIRSKYNNYA
TYYADSVKDRFTISRDDSKNTAYLQMNNLKTEDTAVYYCVRHGNFGNSYISYWAYWGQGT
LVTVSSGGGGSGGGGSGGGGSQTVVTQEPSLTVSPGGTVTLTCGSSTGAVTSGNYPNW
VQQKPGQAPRGLIGGTKFLAPGTPARFSGSLLGGKAALTLSGVQPEDEAEYYCVLWYSNR
WVFGGGTKLTVL
Linker; CDR; VH/VL
Bispecific T-cell engager
SEQ ID NO: 25
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSEVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIWGQGTLV
TVSSSGGGGSEVQLVESGGGLVQPGGSLKLSCAASGFTFNKYAMNWVRQAPGKGLEWV
ARIRSKYNNYATYYADSVKDRFTISRDDSKNTAYLQMNNLKTEDTAVYYCVRHGNFGNSYI
SYWAYWGQGTLVTVSSGGGGSGGGGSGGGGSQTVVTQEPSLTVSPGGTVTLTCGSSTG
AVTSGNYPNWVQQKPGQAPRGLIGGTKFLAPGTPARFSGSLLGGKAALTLSGVQPEDEAE
YYCVLWYSNRWVFGGGTKLTVLHHHHHH
Linker; CDR; VH/VL; 6x His tag
Heavy chain amino acid sequence (CD3)
SEQ ID NO: 26
EVQLVQSGAEVKKPGASVKVSCKASGYTFTNYYIHWVRQAPGQGLEWIGWIYPGDGNTKY
NEKFKGRATLTADTSTSTAYLELSSLRSEDTAVYYCARDSYSNYYFDYWGQGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPDVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPEEKTISKAKGQPREPQVCTLPPSRDELTKNQ
VSLSCAVKGFYPSDIAVEWESNGQPENNYKTTPPVLDSDGSFFLVSKLTVDKSRWQQGNV
FSCSVMHEALHNHYTQKSLSLSPGK
Light chain amino acid sequence (CD3)
SEQ ID NO: 27
DIVMTQSPDSLAVSLGERATINCKSSQSLLNSRTRKNYLAWYQQKPGQPPKLLIYWASTRE
SGVPDRFSGSGSGTDFTLTISSLQAEDVAVYYCTQSFILRTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Heavy chain amino acid sequence (cotinine)
SEQ ID NO: 28
EVQLVESGGGLVQPGGSLRLSCAASGFTFSNYWMSWVRQAPGKGLEWVGDIHGNRGFN
YHASWAKGRFTVSRSKNTLYLQMNSLRAEDTAVYYCAKADDSGSHDIWGQGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPDVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPEEKTISKAKGQPREPQVYTLPPCRDELTKNQ
VSLWCLVKGFYPSDIAVEWESNGQPENNYKTTPPVLDSDGSFFLYSKLTVDKSRWQQGNV
FSCSVMHEALHNHYTQKSLSLSPGK
Bispecific T-cell engager (excluding 6x HIS tag)
SEQ ID NO: 29
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSEVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIWGQGTLV
TVSSSGGGGSEVQLVESGGGLVQPGGSLKLSCAASGFTFNKYAMNWVRQAPGKGLEWV
ARIRSKYNNYATYYADSVKDRFTISRDDSKNTAYLQMNNLKTEDTAVYYCVRHGNFGNSYI
SYWAYWGQGTLVTVSSGGGGSGGGGSGGGGSQTVVTQEPSLTVSPGGTVTLTCGSSTG
AVTSGNYPNWVQQKPGQAPRGLIGGTKFLAPGTPARFSGSLLGGKAALTLSGVQPEDEAE
YYCVLWYSNRWVFGGGTKLTVL
Linker; CDR; VH/VL
Heavy chain CDR1 amino acid sequence (CD3)
SEQ ID NO: 30
KYAMN
Heavy chain CDR2 amino acid sequence (CD3)
SEQ ID NO: 31
RIRSKYNNYATYYADSVKD
Heavy chain CDR3 amino acid sequence (CD3)
SEQ ID NO: 32
HGNFGNSYISYWAY
Light chain CDR1 amino acid sequence (CD3)
SEQ ID NO: 33
GSSTGAVTSGNYPN
Light chain CDR2 amino acid sequence (CD3)
SEQ ID NO: 34
GTKFLAP
Light chain CDR3 amino acid sequence (CD3)
SEQ ID NO: 35
VLWYSNRWV
Variable heavy chain amino acid sequence (CD3)
SEQ ID NO: 36
EVQLVESGGGLVQPGGSLKLSCAASGFTFNKYAMNWRQAPGKGLEWVARIRSKYNNYA
TYYADSVKDRFTISRDDSKNTAYLQMNNLKTEDTAVYYCVRHGNFGNSYISYWAYWGQGT
LVTVSS
Variable light chain amino acid sequence (CD3)
SEQ ID NO: 37
QTVVTQEPSLTVSPGGTVTLTCGSSTGAVTSGNYPNWVQQKPGQAPRGLIGGTKFLAPGT
PARFSGSLLGGKAALTLSGVQPEDEAEYYCVLWYSNRWVFGGGTKLTVL

1. An antibody-drug conjugate comprising: an anti-cotinine antibody or antigen-binding fragment thereof covalently bound to a cytotoxic agent, wherein the anti-cotinine antibody or antigen-binding fragment thereof comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6.

2. The antibody-drug conjugate of claim 1, wherein the cytotoxic agent is bound to the antibody or antigen-binding fragment thereof via a linker.

3. The antibody-drug conjugate of claim 1 or 2, wherein the cytotoxic agent is Dxd.

4. The antibody-drug conjugate of any one of the preceding claims, wherein the ratio of cytotoxic agent to antibody or antigen-binding fragment thereof is in a range of about 1:1 to about 10:1.

5. The antibody-drug conjugate of any one of the preceding claims, wherein the antibody or antigen-binding fragment thereof comprises a heavy chain variable region (VH) as set forth in SEQ ID NO: 7 and a light chain variable region (VL) as set forth in SEQ ID NO: 8.

6. The antibody-drug conjugate of any one of the preceding claims, wherein the antibody comprises a heavy chain as set forth in SEQ ID NO: 9 and a light chain as set forth in SEQ ID NO: 10.

7. A combination comprising the antibody-drug conjugate of any one of claims 1 to 6 and a heterobifunctional molecule comprising a moiety that binds a target cell-surface protein covalently linked to a cotinine moiety.

8. A bispecific antibody or bispecific antigen binding fragment thereof comprising a cotinine binding domain and a CD3 binding domain.

9. The bispecific antibody or bispecific antigen binding fragment thereof of claim 8, wherein the cotinine binding domain comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6.

10. The bispecific antibody or bispecific antigen binding fragment thereof of claim 8 or 9, wherein the CD3 binding domain comprises: (i) a heavy chain CDR1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21; or (ii) a heavy chain CDR1 having SEQ ID NO: 30, a heavy chain CDR2 having SEQ ID NO: 31, a heavy chain CDR3 having SEQ ID NO: 32, a light chain CDR 1 having SEQ ID NO: 33, a light chain CDR2 having SEQ ID NO: 34, and a light chain CDR3 having SEQ ID NO: 35.

11. The bispecific antibody or bispecific antigen binding fragment thereof of any one of claims 8 to 10, wherein the cotinine binding domain comprises a first single chain variable fragment (scFv) comprising a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6, and the CD3 binding domain comprises a second scFv that binds CD3.

12. The bispecific antibody or bispecific antigen binding fragment thereof of claim 11, wherein the first scFv comprises a heavy chain variable region (VH) and a light chain variable region (VL) joined by a first polypeptide linker.

13. The bispecific antibody or bispecific antigen binding fragment thereof of claim 11 or 12, wherein the first scFv comprises a VH as set forth in SEQ ID NO: 7 and a VL as set forth in SEQ ID NO: 8.

14. The bispecific antibody or bispecific antigen binding fragment thereof of claim 11, wherein the first scFv is as set forth in SEQ ID NO: 15.

15. The bispecific antibody or bispecific antigen binding fragment thereof of any one of claims 11 to 14, wherein the second scFv comprises (i) a heavy chain CDR1 having SEQ ID NO: 16, a heavy chain CDR2 having SEQ ID NO: 17, a heavy chain CDR3 having SEQ ID NO: 18, a light chain CDR1 having SEQ ID NO: 19, a light chain CDR2 having SEQ ID NO: 20, and a light chain CDR3 having SEQ ID NO: 21; or (ii) a heavy chain CDR1 having SEQ ID NO: 30, a heavy chain CDR2 having SEQ ID NO: 31, a heavy chain CDR3 having SEQ ID NO: 32, a light chain CDR 1 having SEQ ID NO: 33, a light chain CDR2 having SEQ ID NO: 34, and a light chain CDR3 having SEQ ID NO: 35.

16. The bispecific antibody or bispecific antigen binding fragment thereof of claim 15, wherein the second scFv comprises a VH and a VL joined by a second polypeptide linker.

17. The bispecific antibody or bispecific antigen binding fragment thereof of claim 15 or 16, wherein the second scFv comprises (i) a VH as set forth in SEQ ID NO: 22 and VL as set forth in SEQ ID NO: 23; or (ii) a VH as set forth in SEQ ID NO: 36 and a VL as set forth in SEQ ID NO: 37.

18. The bispecific antibody or bispecific antigen binding fragment thereof of claim 15,
wherein the second scFv is as set forth in SEQ ID NO: 24.

19. The bispecific antibody or bispecific antigen binding fragment thereof of any one of claims 11 to 18, wherein the first scFv and the second scFv are joined by a third polypeptide linker.

20. The bispecific antibody or bispecific antigen binding fragment thereof of any one of claims 11 to 19, wherein the bispecific antibody or bispecific antigen binding fragment thereof is a bispecific T cell engager is as set forth in SEQ ID NO: 25 or SEQ ID NO: 29.

21. The bispecific antibody or antigen binding fragment thereof of any one of claims 8 to 10, being a bispecific antibody.

22. The bispecific antibody or bispecific antigen binding fragment thereof of claim 21, wherein the bispecific antibody comprises: a cotinine binding domain comprising a heavy chain comprising a heavy chain CDR1 having SEQ ID NO: 1, a heavy chain CDR2 having SEQ ID NO: 2, and a heavy chain CDR3 having SEQ ID NO: 3, and a light chain comprising a light chain CDR1 having SEQ ID NO: 4, a light chain CDR2 having SEQ ID NO: 5, and a light chain CDR3 having SEQ ID NO: 6; and a CD3 binding domain comprising (i) a heavy chain comprising a heavy chain CDR1 having SEQ ID NO: 16, a heavy chain CDR2 having SEQ ID NO: 17, and a heavy chain CDR3 having SEQ ID NO: 18, and a light chain comprising a light chain CDR1 having SEQ ID NO: 19, a light chain CDR2 having SEQ ID NO: 20, and a light chain CDR3 having SEQ ID NO: 21, or (ii) a heavy chain comprising a heavy chain CDR1 having SEQ ID NO: 30, a heavy chain CDR2 having SEQ ID NO: 31, and a heavy chain CDR3 having SEQ ID NO: 32, and a light chain comprising a light chain CDR1 having SEQ ID NO: 33, a light chain CDR2 having SEQ ID NO: 34, and a light chain CDR3 having SEQ ID NO: 35.

23. The bispecific antibody or bispecific antigen binding fragment thereof of claim 21 or 22, wherein the cotinine binding domain comprises a heavy chain comprising a heavy chain variable region (VH) as set forth in SEQ ID NO: 7 and a light chain comprising a light chain variable region (VL) as set forth in SEQ ID NO: 8.

24. The bispecific antibody or bispecific antigen binding fragment thereof of any one of claims 21 to 23, wherein the CD3 binding domain comprises (i) a heavy chain comprising a heavy chain variable region (VH) as set forth in SEQ ID NO: 22 and a light chain comprising a light chain variable region (VL) as set forth in SEQ ID NO: 23; or (ii) a heavy chain comprising a heavy chain variable region (VH) as set forth in SEQ ID NO: 36 and a light chain comprising a light chain variable region (VL) as set forth in SEQ ID NO: 37.

25. The bispecific antibody or bispecific antigen binding fragment thereof of any one of claims 21 to 24, wherein the cotinine binding domain comprises a heavy chain as set forth in SEQ ID NO: 28 and a light chain as set forth in SEQ ID NO: 10; and the CD3 binding domain comprises a heavy chain as set forth in SEQ ID NO: 26 and a light chain as set forth in SEQ ID NO: 27.

26. A polynucleotide encoding the bispecific antibody or bispecific antigen binding fragment thereof of any one of claims 8 to 25.

27. An expression vector comprising the polynucleotide of claim 26.

28. A cell comprising the polynucleotide of claim 26 or expression vector of claim 27.

29. A combination comprising the bispecific antibody or bispecific antigen binding fragment thereof of any one of claims 8 to 25 and a heterobifunctional molecule comprising a moiety that binds a target cell-surface protein covalently linked to a cotinine moiety.

30. The combination of claim 7 or 29, wherein the heterobifunctional molecule is a compound of Formula (I): 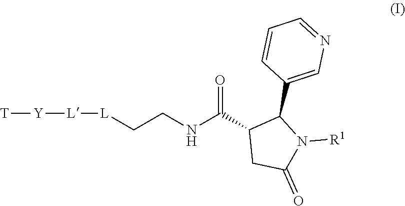
or a pharmaceutically acceptable salt thereof,
wherein:
T is a target binding moiety;
R¹ is C₁₋₄ alkyl or C₃₋₆ cycloalkyl;
L′ is a bond, 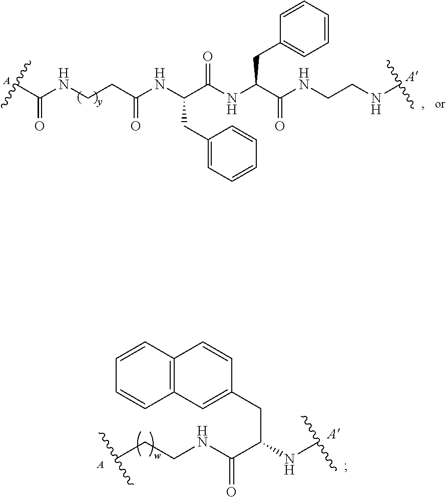
y is an integer of 1 to 9;
w is an integer of 0 to 5;
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), (L-n-iv), (L-p), (L-q), (L-r), or (L-s): 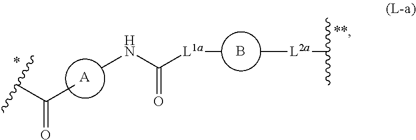
or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O- or —NR^{a}-;
each R^{a} is independently hydrogen or C₁₋₃ alkyl; and
L^{2a} is —O—, —NHC(O)—, or —CH₂—O—; 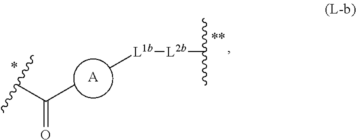
or a stereoisomer thereof,
wherein:
Ring A is C₄₋₆ cycloalkylene or C₇₋₉ bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}-, —C(O)NR^{1b}-, or —NR^{1b}C(O)—; or
L^{2b} is 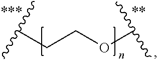
wherein n is 1, 2, 3, or 4, and 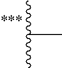
represents a covalent bond to L^{1b}; and
each R^{1b} is independently hydrogen or C₁₋₃ alkyl; 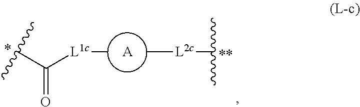
or a stereoisomer thereof,
wherein:
L¹c is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇₋₉ bridged bicyclic cycloalkylene; and
L²c is —O- or a saturated C₂₋₁₀ linear alkylene, wherein 1, 2, or 3 methylene units are replaced with —O—, —NH—, —NHC(O)—, or —C(O)NH—; 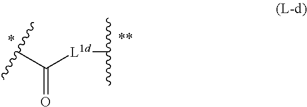
wherein:
L¹d is C₁₂₋₂₂ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—; 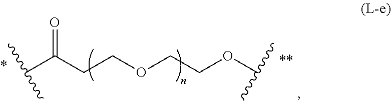
wherein n is an integer of 3 to 50; 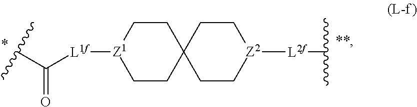
or a stereoisomer thereof,
wherein:
L¹f is a bond; C₁₋₆ linear alkylene, wherein 0, 1, or 2 methylene units are replaced with —O—, —NH—, or —C(O)—; or -(C₃₋₆ cycloalkylene)—NHC(O)—;
L²f is a bond, —NHC(O)—, —C(O)NH—, or a C₁₋₆ linear alkylene,
wherein 0, 1, or 2 methylene units are replaced with —O—; and
each of Z¹ and Z² is independently N or CH; 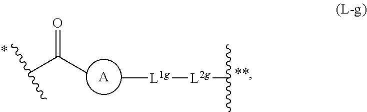
wherein:
Ring A is a 5 to 6 membered heteroarylene having 1 or 2 nitrogen ring atoms;
L¹g is a bond, —CH₂—, —NH—, or —O—; and
L²g is 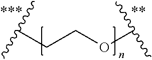
wherein n is 1, 2, 3, 4, or 5, and 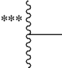
represents a covalent bond to L¹g; 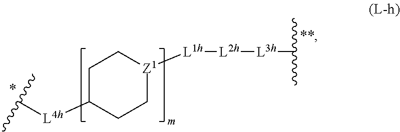
or a stereoisomer thereof,
wherein:
each Z¹ is independently N or CH;
L¹ h is a bond, —C(O)—, —C(O)—NH—, or —NHC(O)—;
L² h is C₂₋₁₀ linear alkylene or 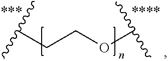
wherein n is 1, 2, 3, or 4, and 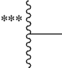
represents a covalent bond to L¹ h and 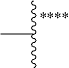
represents a covalent bond to L³h;
L³ h is a bond, —C(O) CH₂—, —O—(C₃₋₆ cycloalkylene)—O—, or —C(O)NH(CH₂)₃OCH₂-;
L⁴ h is a bond, —C(O)—, —CH₂C(O)—, or —C(O) CH₂—; and
m is 1, 2, or 3; 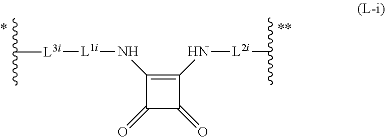
wherein:
L¹ⁱ is a bond, C₁₋₁₂ linear alkylene, or 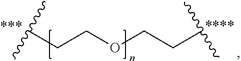
wherein n is 1, 2, 3, 4, or 5, and 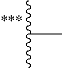
represents a covalent bond to L³ⁱ and 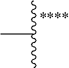
represents a covalent bond to NH;
L²ⁱ is a bond, C₁₋₁₂ linear alkylene, or 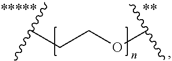
wherein
n is 1, 2, 3, 4, or 5, and 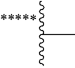
represents a covalent bond to HN; and
L³ⁱ is a bond or —C(O)—; 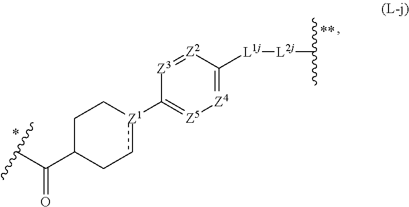
or a stereoisomer thereof,
wherein:
Z¹ is C, CH, or N;
each of Z², Z³, Z⁴ and Z⁵ is independently CH or N, provided that no more than two of Z², Z³, Z⁴ and Z⁵ are N;
L¹j is —NH—, —C(O)NH—, —NHC(O)—, or —O—;
L²j is C₁₋₆ linear alkylene or 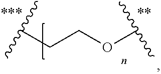
wherein n is 1 or 2,
and 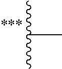
represents a covalent bond to L¹j; and [symbol]
represents a single bond or a double bond; 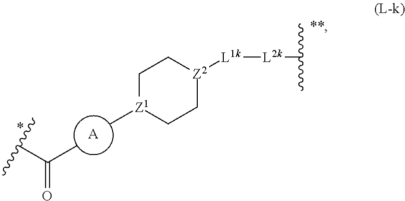
or a stereoisomer thereof,
wherein:
Ring A is phenyl or a 5 or 6 membered heteroarylene having 1 or 2 nitrogen ring atoms;
each of Z¹ and Z² is independently CH or N;
L¹k is a bond, —C(O)—, —C(O)NH- or —NHC(O)—; and
L²k is a C₃₋₈ straight chain alkylene or 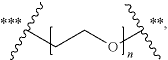
wherein
n is 1, 2, or 3, and 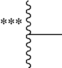
represents a covalent bond to L^{1k}; 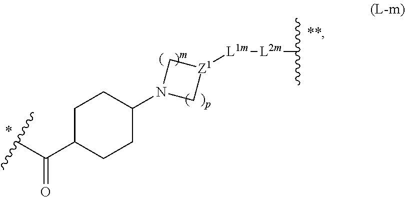
or a stereoisomer thereof,
wherein:
Z¹ is CH or N;
m is 1 or 2;
p is 1 or 2;
0, 1, or 2 hydrogen atoms of 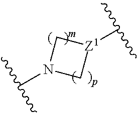
are replaced with F;
L^{1m} is a bond, —C(O)—, —C(O)NH—, —NHC(O)—, -S(O)₂NH- or —NHS(O)₂—; and
L²m is C₃₋₆ linear alkylene, C₃₋₆ cycloalkylene, or 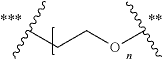
wherein n is 1 or 2, and 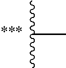
represents a covalent bond to L¹m; 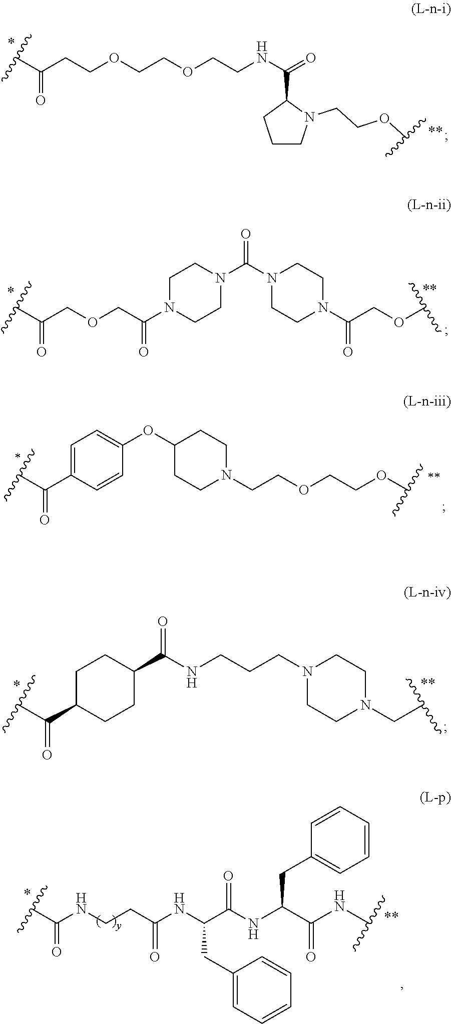
or a stereoisomer thereof, wherein y is an integer of 1 to 9; 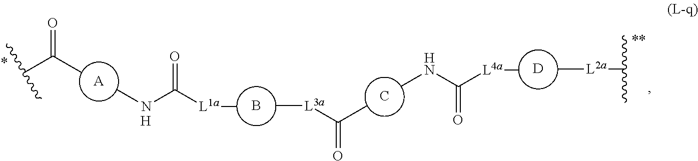
or a stereoisomer thereof,
wherein:
Ring A, Ring B, Ring C, and Ring D are each independently C₄₋₆ cycloalkylene;
L^{1a}, L³a, and L⁴a are each independently C₃₋₅ linear alkylene,
wherein 1 or 2 methylene units are replaced with —O- or —NR^{a}-;
each R^{a} is independently hydrogen or C₁₋₃ alkyl; and
L^{2a} is —O—, —NHC(O)—, or —CH₂—O—; 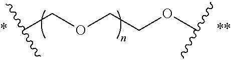
wherein n is an integer of 10 to 30; or 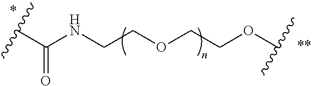
wherein n is an integer of 10 to 30,
wherein each 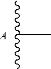
represents a covalent bond to the Y group of Formula (I), or when Y is a bond, a covalent bond to the T group of Formula (I), and each 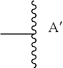
represents a covalent bond to the L group of Formula (I); and
wherein each 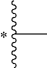
represents a covalent bond to the L′ group of Formula (I), or when L′ is a bond, a covalent bond to the Y group of Formula (I), or when both L′ and Y are a bond, a covalent bond to the T group of Formula (I), and each 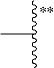
represents a covalent bond to the methylene group of Formula (I); and
Y is a bond or a divalent spacer moiety of one to twelve atoms in length.

31. The combination of claim 30, wherein R¹ is —CH₃.

32. The combination of claim 30 or 31, wherein L′ is a bond.

33. The combination of any one of claims 30 to 32, wherein L is a divalent linker of Formula (L-a-i): 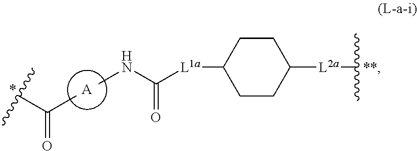
or a stereoisomer thereof,
wherein Ring A, L^{1a}, L^{2a}, 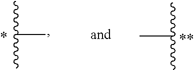
are as defined for Formula (L-a).

34. The combination of any one of claims 30 to 33, wherein L is a divalent linker of Formula (L-a-ii): 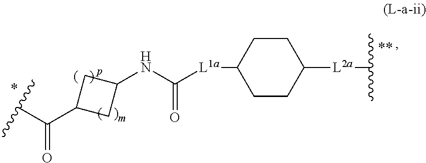
or a stereoisomer thereof,
wherein L^{1a}, L^{2a}, 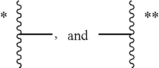
are as defined for Formula (L-a); p is 1 or 2; and m is 1 or 2.

35. The combination of any one of claims 30 to 34, wherein L is a divalent linker of Formula (L-a-iii): 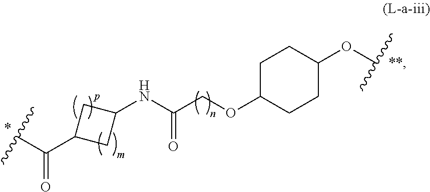
or a stereoisomer thereof,
wherein p is 1 or 2; m is 1 or 2; n is 1, 2, or 3; and 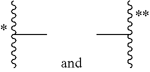
are as defined for Formula (L-a).

36. The combination of any one of claims 30 to 34, wherein L is a divalent linker of Formula (L-a) selected from the group consisting of:

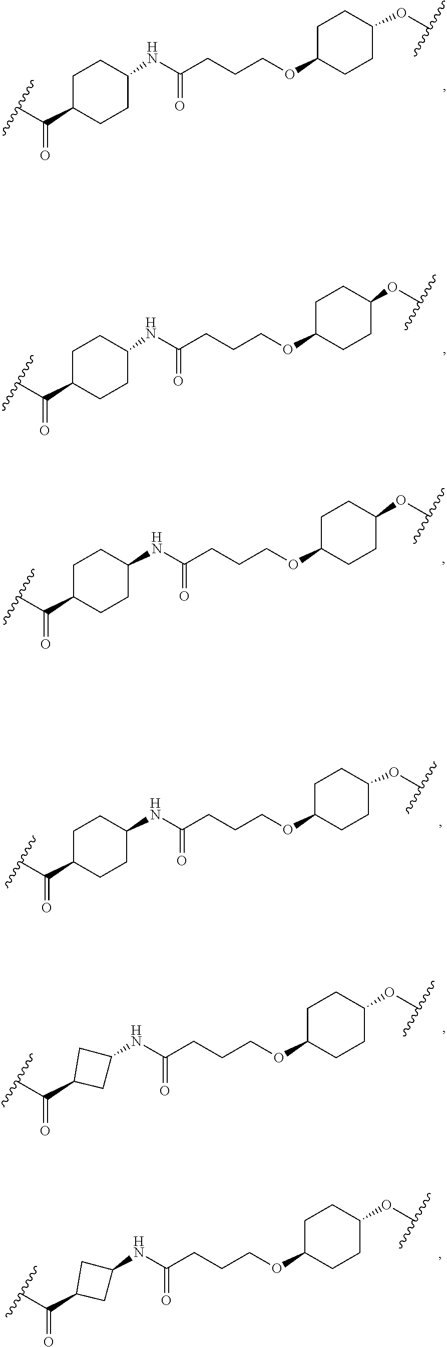 [SMILES: *O[C@H]1CC[C@H](OCCCC(=O)N[C@H]2CC[C@H](C(*)=O)CC2)CC1 ; *O[C@H]1CC[C@@H](OCCCC(=O)N[C@H]2CC[C@H](C(*)=O)CC2)CC1 ; *O[C@H]1CC[C@@H](OCCCC(=O)N[C@H]2CC[C@@H](C(*)=O)CC2)CC1 ; *O[C@H]1CC[C@H](OCCCC(=O)N[C@H]2CC[C@@H](C(*)=O)CC2)CC1 ; *O[C@H]1CC[C@H](OCCCC(=O)N[C@H]2C[C@H](C(*)=O)C2)CC1 ; *O[C@H]1CC[C@H](OCCCC(=O)N[C@H]2C[C@@H](C(*)=O)C2)CC1]

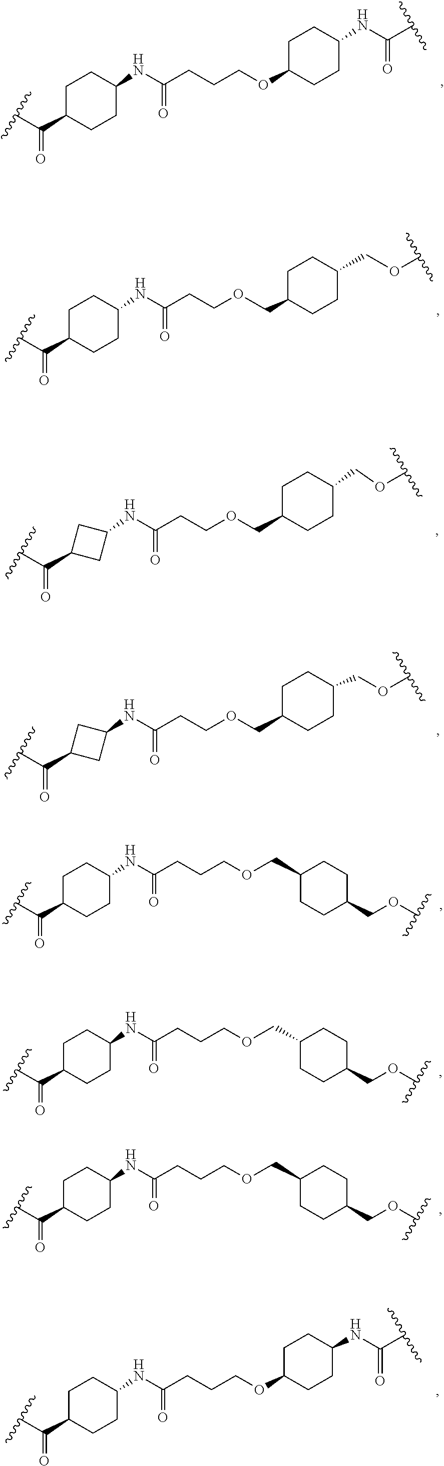

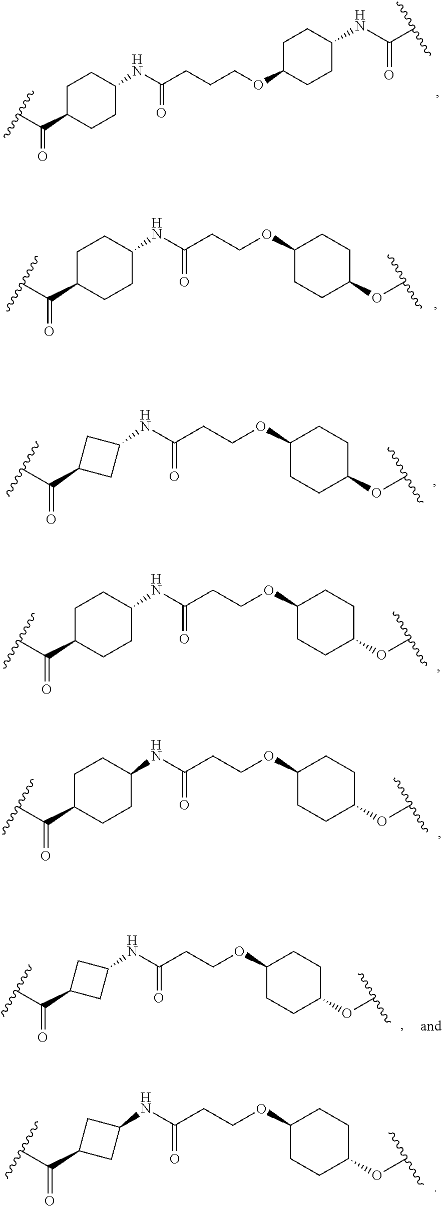

37. The combination of any one of claims 30 to 32, wherein L is a divalent linker of Formula (L-b-i): 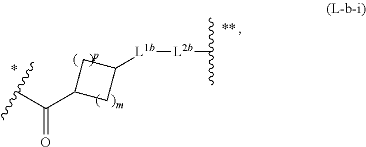
or a stereoisomer thereof,
wherein L^{1b}, L^{2b}, 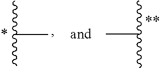
are as defined for Formula (L-b); p is 1 or 2; and m is 1 or 2.

38. The combination of any one of claim 30, 31, 32, or 37, wherein L is a divalent linker of Formula (L-b) selected from the group consisting of:

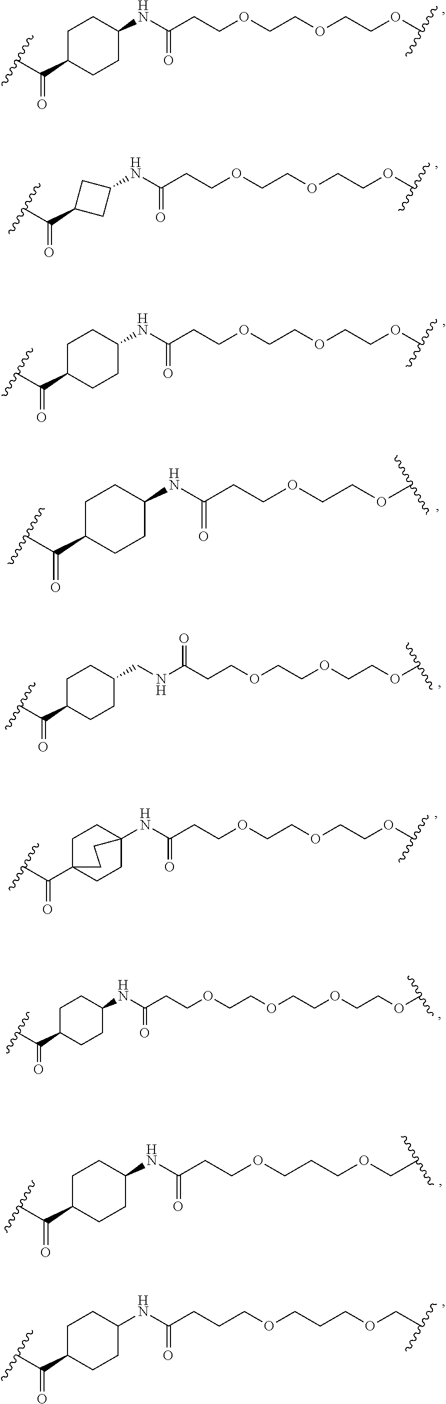

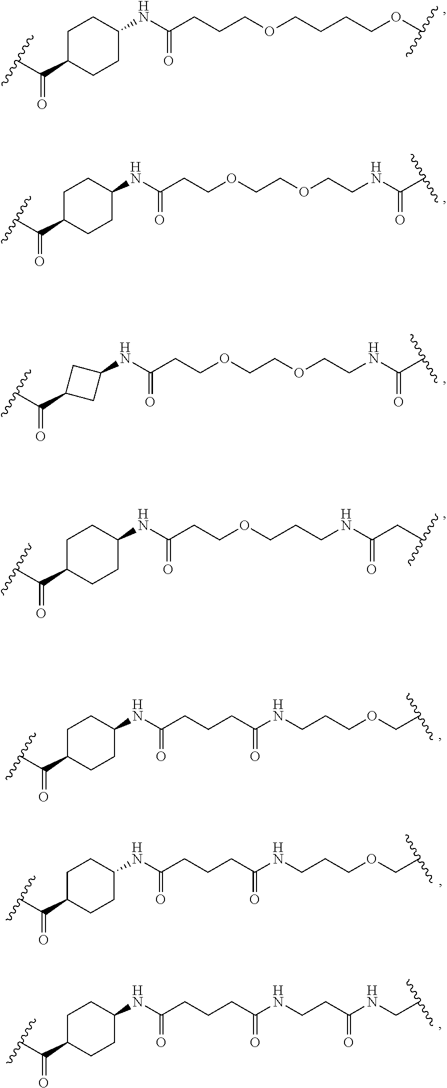

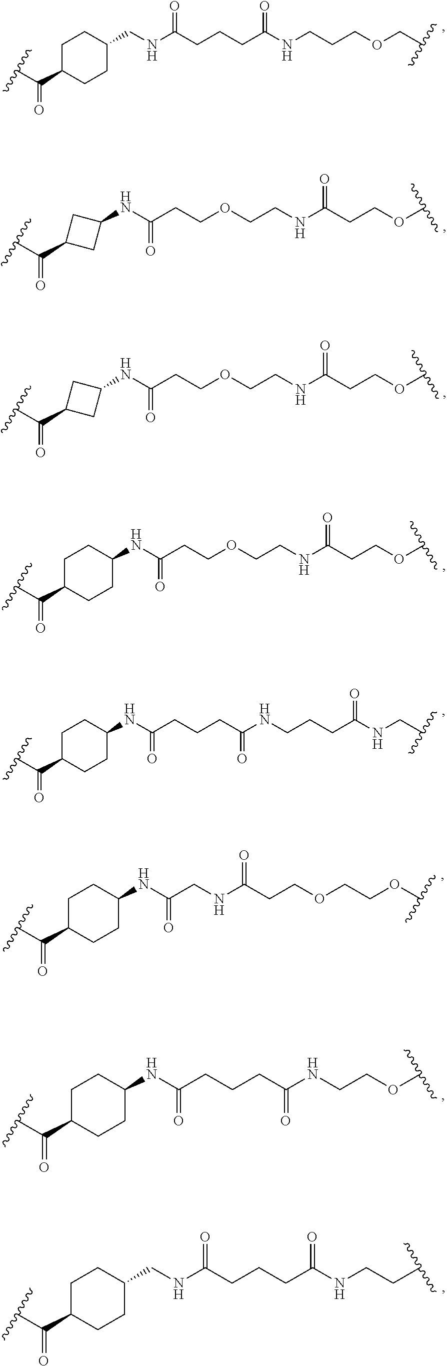

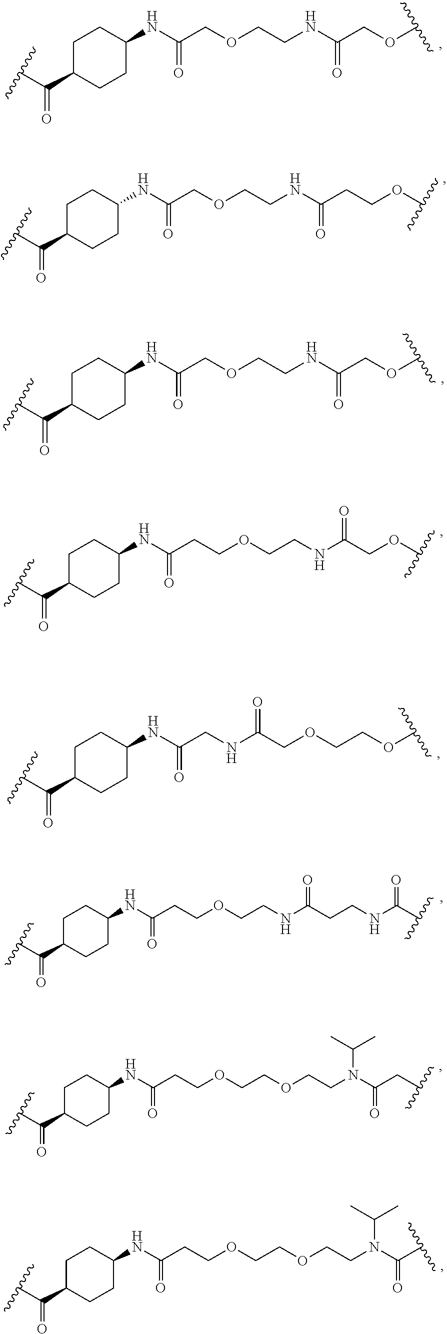

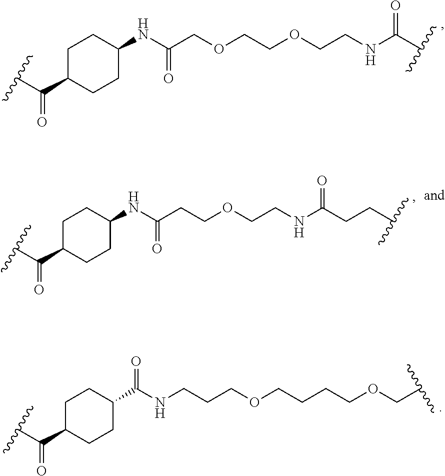 [SMILES: *C(=O)NCCOCCOCC(=O)N[C@H]1CC[C@@H](C(*)=O)CC1 ; *CCC(=O)NCCOCCC(=O)N[C@H]1CC[C@@H](C(*)=O)CC1 ; *COCCCCOCCCNC(=O)[C@H]1CC[C@H](C(*)=O)CC1]

39. The combination of any one of claim 30, 31, or 32, wherein L is a divalent linker of Formula (L-c-i): 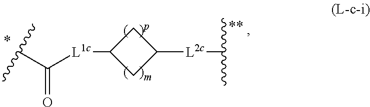
or a stereoisomer thereof,
wherein L¹, L^{2c}, 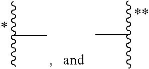
are as defined for Formula (L-c); p is 1 or 2; and m is 1 or 2.

40. The combination of any one of claim 30, 31, or 32, wherein L is a divalent linker of Formula (L-c) selected from the group consisting of:

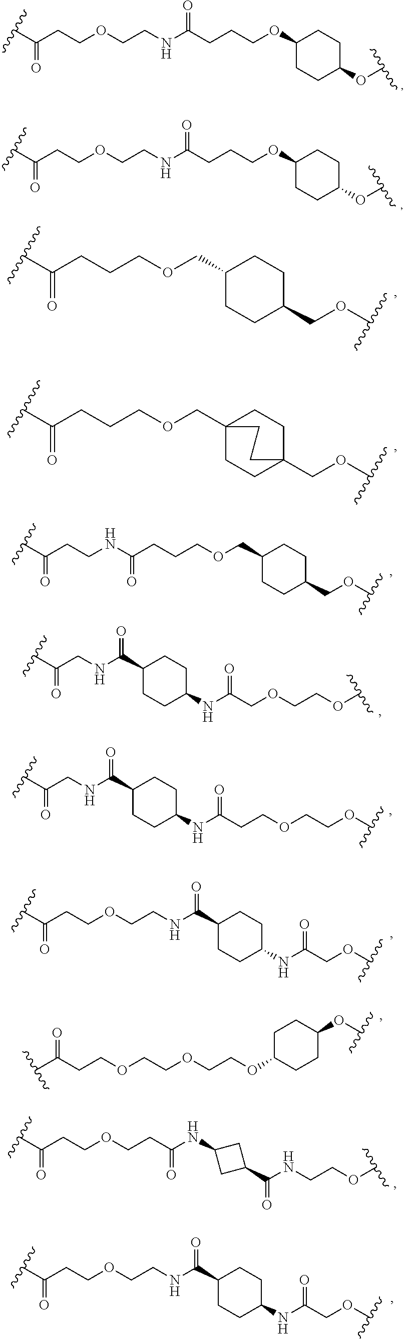

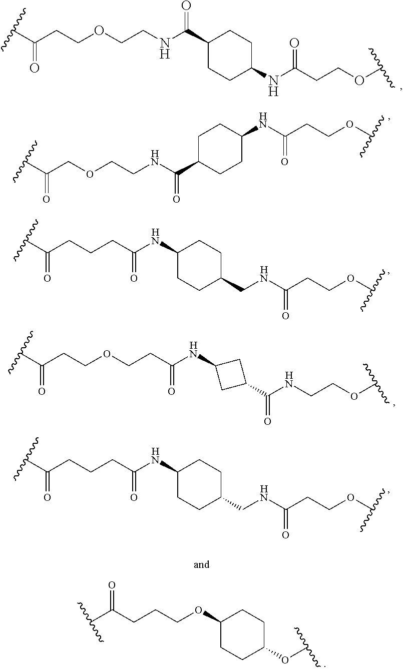 [SMILES: *OCCC(=O)N[C@H]1CC[C@@H](C(=O)NCCOCCC(*)=O)CC1 ; *OCCC(=O)N[C@H]1CC[C@@H](C(=O)NCCOCC(*)=O)CC1 ; *OCCC(=O)NC[C@H]1CC[C@@H](NC(=O)CCCC(*)=O)CC1 ; *OCCNC(=O)[C@H]1C[C@H](NC(=O)CCOCCC(*)=O)C1 ; *OCCC(=O)NC[C@H]1CC[C@H](NC(=O)CCCC(*)=O)CC1 ; *O[C@H]1CC[C@H](OCCCC(*)=O)CC1]

41. The combination of any one of claim 30, 31, or 32, wherein L is a divalent linker of Formula (L-d) selected from the group consisting of:

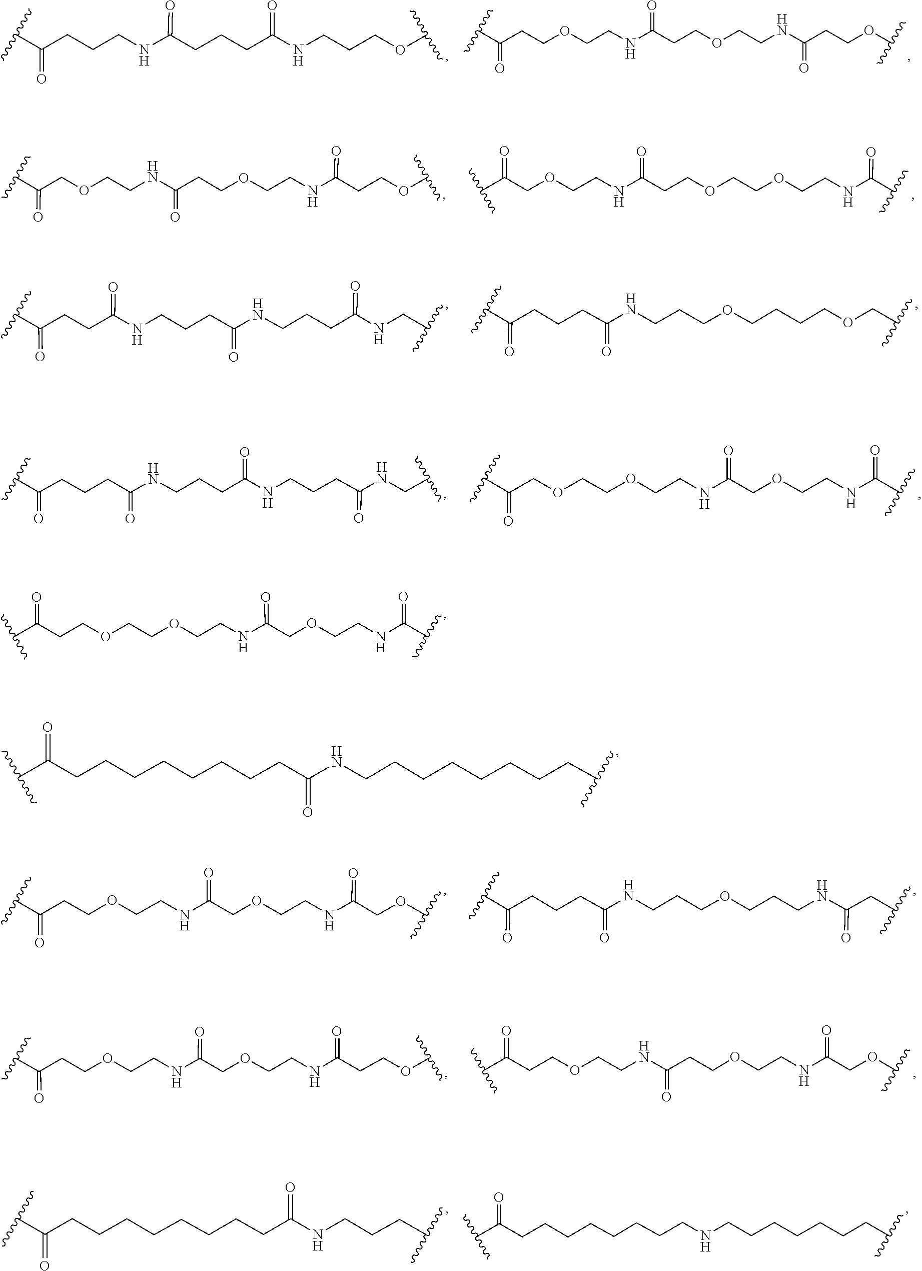

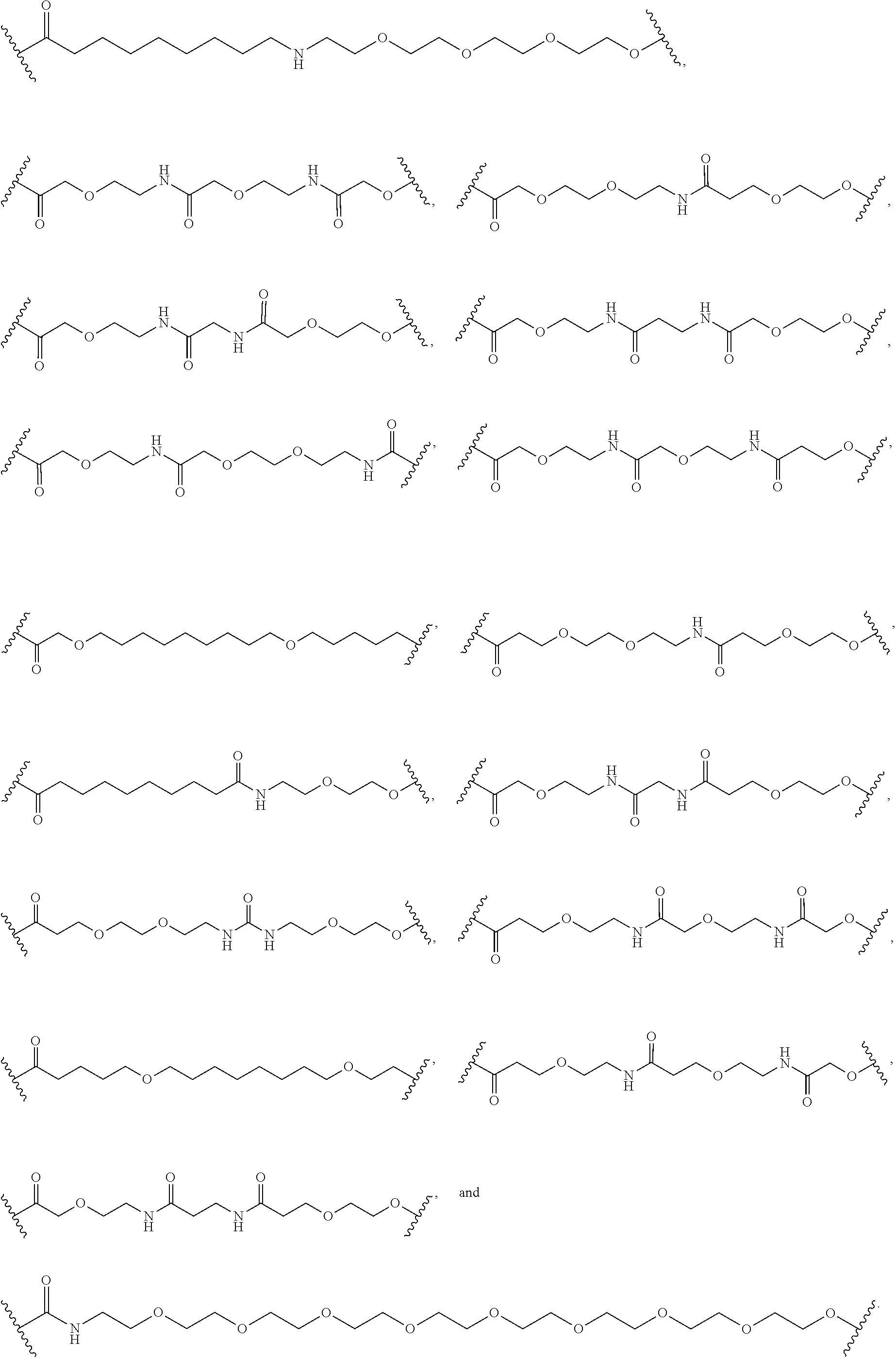

42. The combination of any one of claim 30, 31, or 32, wherein L is a divalent linker of Formula (L-f) selected from the group consisting of:

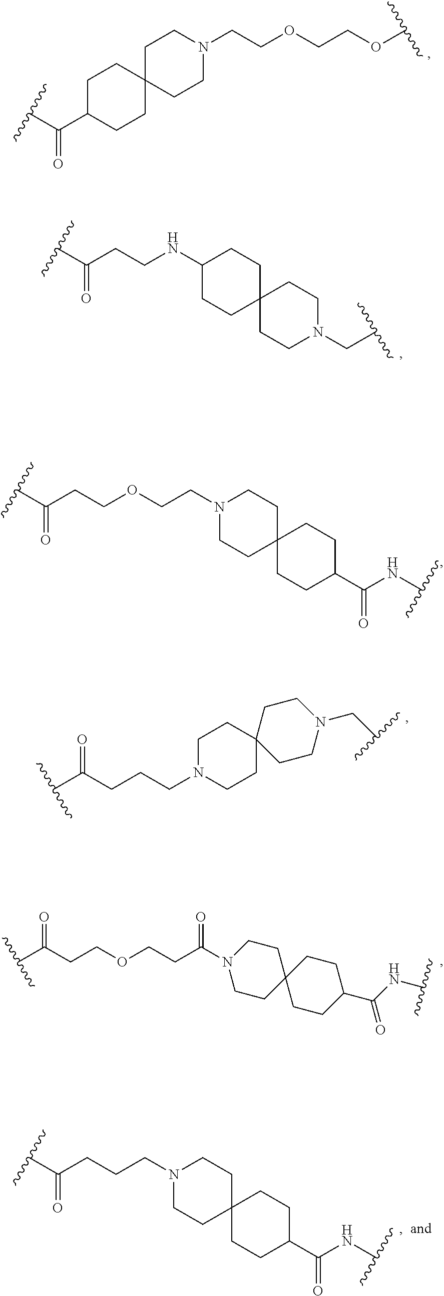 [SMILES: *OCCOCCN1CCC2(CCC(C(*)=O)CC2)CC1 ; *CN1CCC2(CCC(NCCC(*)=O)CC2)CC1 ; *NC(=O)C1CCC2(CC1)CCN(CCOCCC(*)=O)CC2 ; *CN1CCC2(CC1)CCN(CCCC(*)=O)CC2 ; *NC(=O)C1CCC2(CC1)CCN(C(=O)CCOCCC(*)=O)CC2 ; *NC(=O)C1CCC2(CC1)CCN(CCCC(*)=O)CC2]

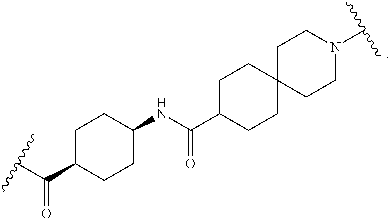 [SMILES: *C(=O)[C@H]1CC[C@@H](NC(=O)C2CCC3(CC2)CCN(*)CC3)CC1]

43. The combination of any one of claim 30, 31, or 32, wherein L is a divalent linker of Formula (L-g-i): 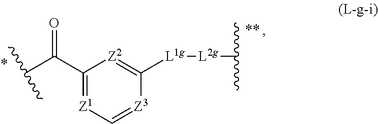
wherein L^{1g}, L^{2g}, 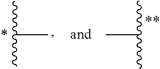
are as defined for Formula (L-g); Z¹, Z², and Z³ are each independently selected from N or CH, provided that one or two of Z¹, Z², and Z³ is N.

44. The combination of any one of claim 30, 31, 32, or 43, wherein L is a divalent linker of Formula (L-g) selected from the group consisting of:

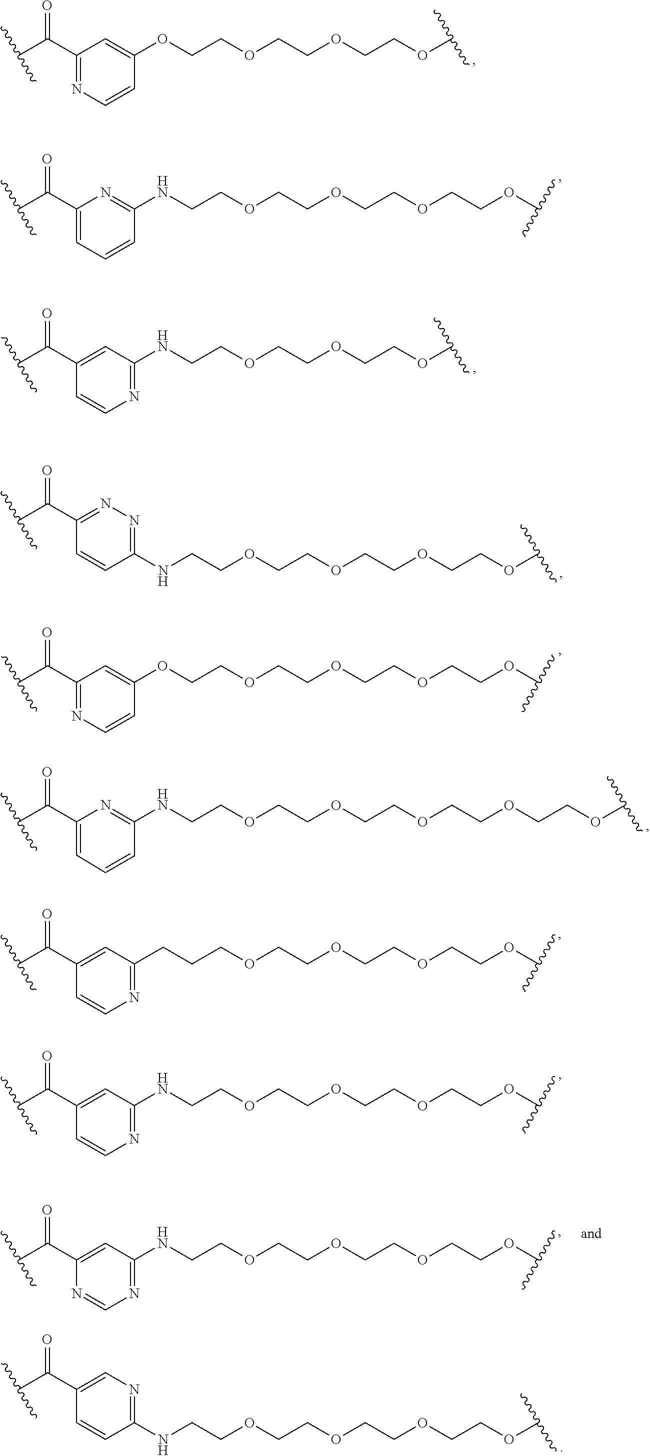

45. The combination of any one of claim 30, 31, or 32, wherein L is a divalent linker of Formula (L-h) selected from the group consisting of:

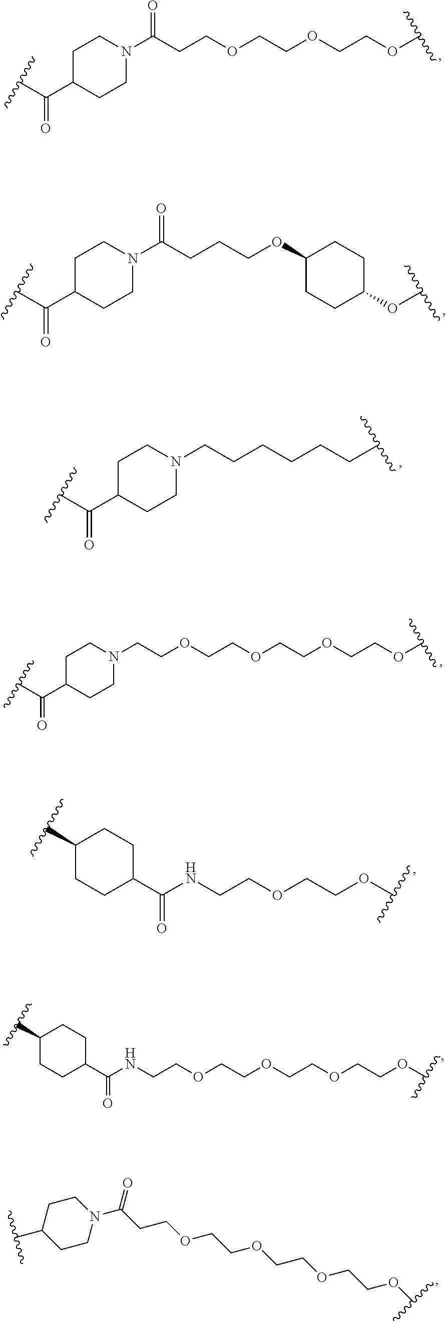

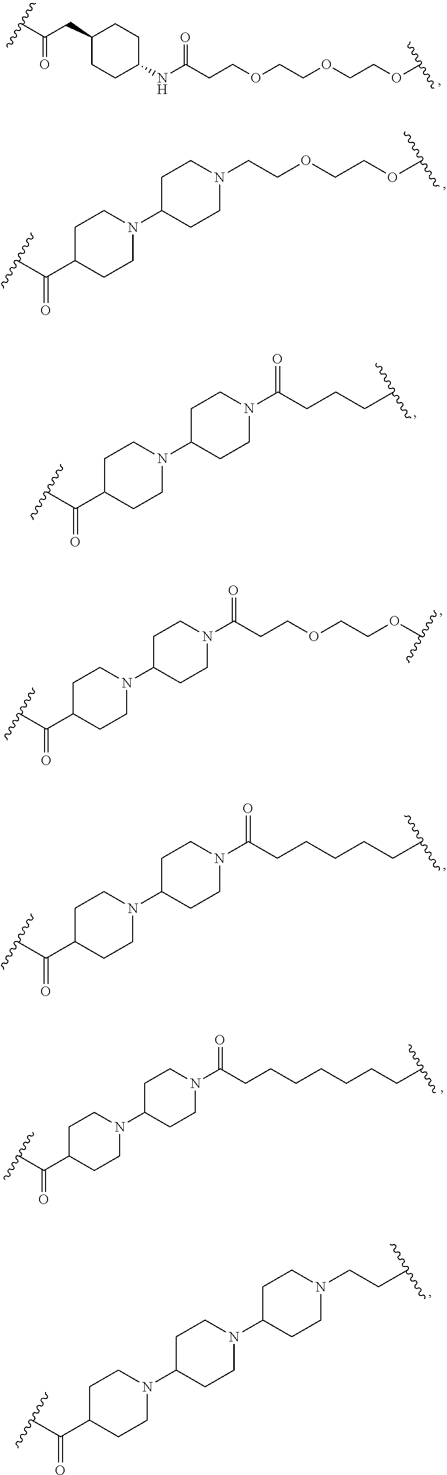

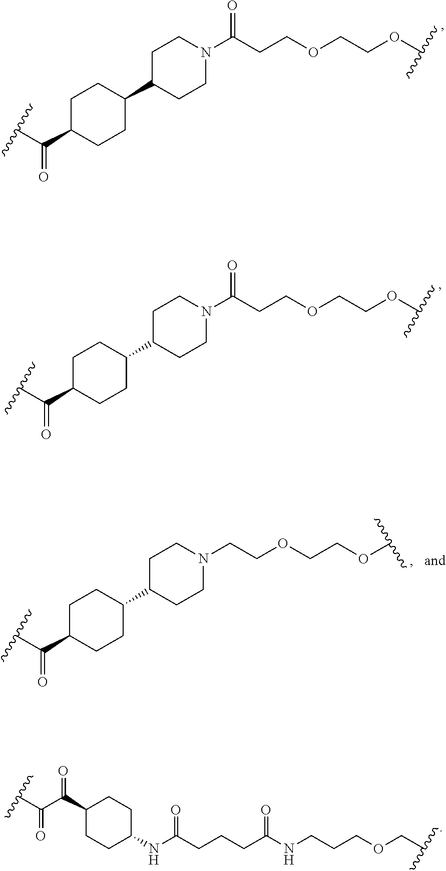 [SMILES: *OCCOCCC(=O)N1CCC([C@H]2CC[C@@H](C(*)=O)CC2)CC1 ; *OCCOCCC(=O)N1CCC([C@H]2CC[C@H](C(*)=O)CC2)CC1 ; *OCCOCCN1CCC([C@H]2CC[C@H](C(*)=O)CC2)CC1 ; *COCCCNC(=O)CCCC(=O)N[C@H]1CC[C@H](C(=O)C(*)=O)CC1]

46. The combination of any one of claim 30, 31, or 32, wherein L is a divalent linker of Formula (L-i) selected from the group consisting of:

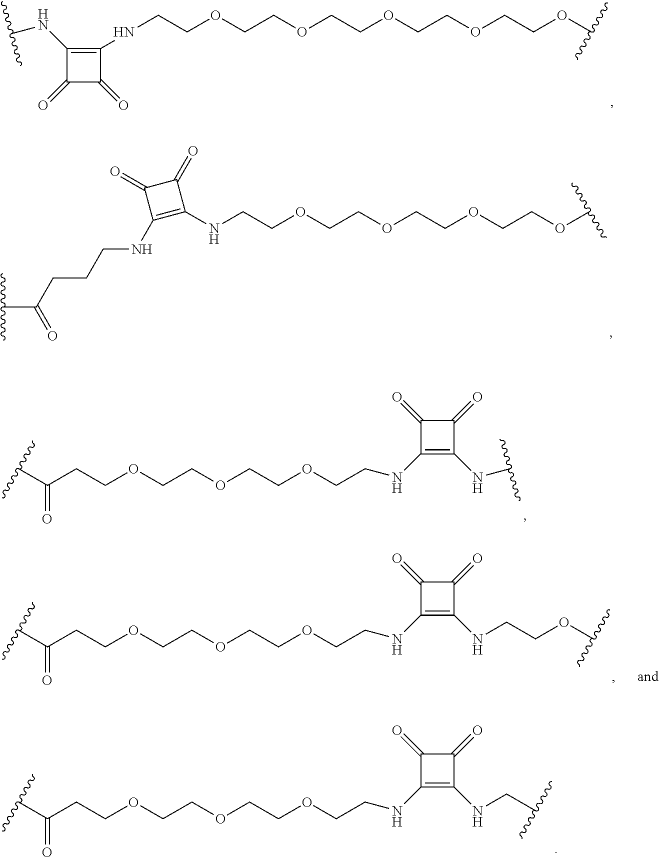 [SMILES: *Nc1c(NCCOCCOCCOCCOCCO*)c(=O)c1=O ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

47. The combination of any one of claim 30, 31, or 32, wherein L is a divalent linker of Formula (L-j) selected from the group consisting of:

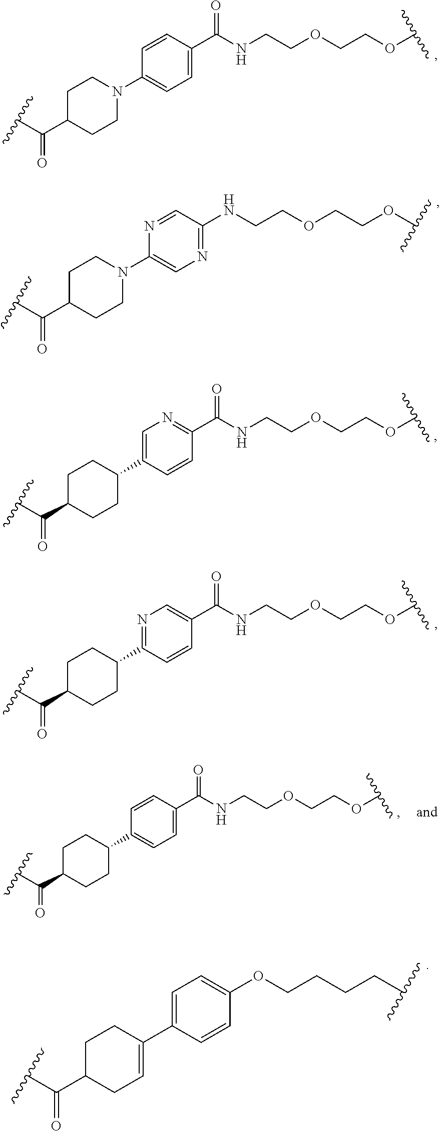 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

48. The combination of any one of claim 30, 31, or 32, wherein L is a divalent linker of Formula (L-k) selected from the group consisting of:

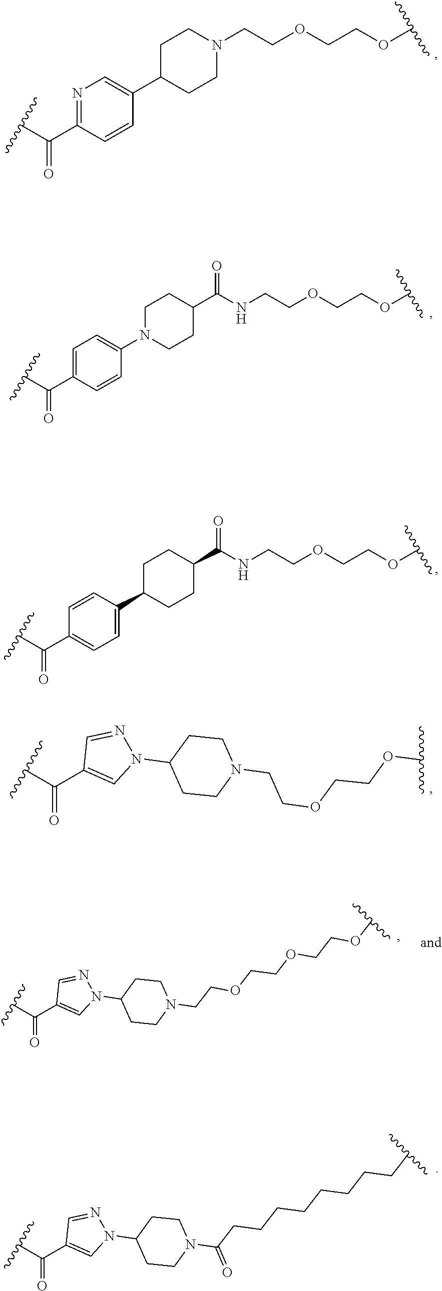 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

49. The combination of any one of claim 30, 31, or 32, wherein L is a divalent linker of Formula (L-m) selected from the group consisting of:

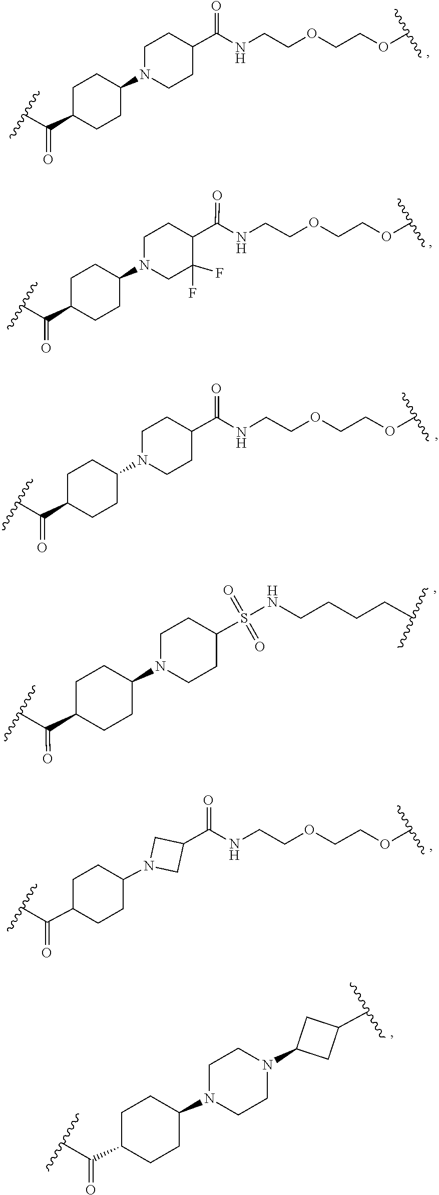

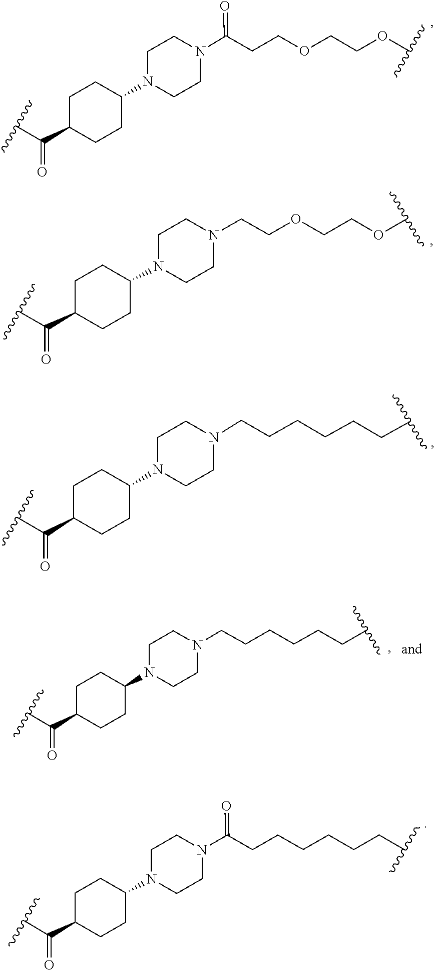 [SMILES: *OCCOCCC(=O)N1CCN([C@H]2CC[C@H](C(*)=O)CC2)CC1 ; *OCCOCCN1CCN([C@H]2CC[C@H](C(*)=O)CC2)CC1 ; *CCCCCCN1CCN([C@H]2CC[C@H](C(*)=O)CC2)CC1 ; *CCCCCCN1CCN([C@H]2CC[C@@H](C(*)=O)CC2)CC1 ; *CCCCCCC(=O)N1CCN([C@H]2CC[C@H](C(*)=O)CC2)CC1]

50. The combination of any one of claim 30, 31, or 32, wherein L is a divalent linker of Formula (L-q-i): 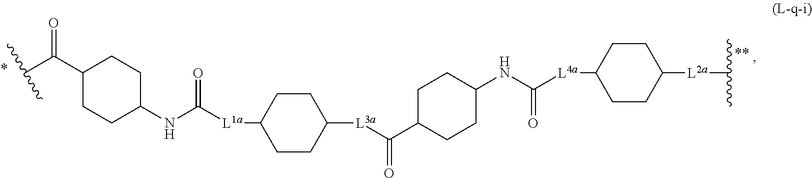
or a stereoisomer thereof,
wherein L^{1a}, L³a, L⁴a, L²a, 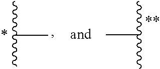
are as defined for Formula (L-q).

51. The combination of any one of claim 30, 31, 32, or 50, wherein L is a divalent linker of Formula (L-q-ii): 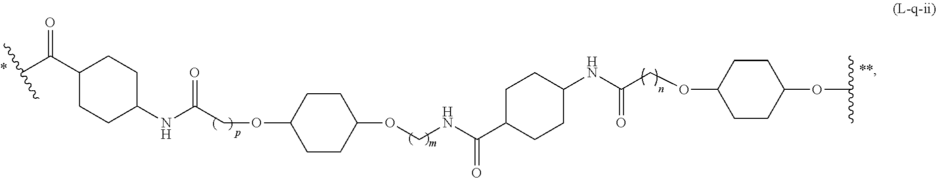
or a stereoisomer thereof,
wherein p is 1, 2, or 3; m is 1, 2, or 3; n is 1, 2, or 3; and 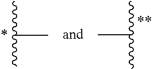
are as
defined for Formula (L-q).

52. The combination of any one of claims 30 to 51, wherein Y is selected from a bond; —NH—; -(C₁₋₁₂ alkylene)-, wherein 1, 2, or 3 methylene units are replaced with —O—, —NH—, N(CH₃)—, —C(O)—, NHC(O)—, —C(O)NH—, -(C₃₋₆ cycloalkylene)-, -(C₃₋₆ cycloalkenylene)-, 3- to 1O-membered heterocycloalkylene, arylene, or heteroarylene; or -(C₂₋₁₂ alkenylene)-, wherein 1, 2, or 3 methylene units are replaced with —O—, —NH—, N(CH₃)—, —C(O)—, NHC(O)—, —C(O)NH—, -(C₃₋₆ cycloalkylene)-, -(C₃₋₆ cycloalkenylene)-, 3- to 1O-membered heterocycloalkylene, arylene, or heteroarylene.

53. The combination of any one of claims 30 to 52, wherein Y is selected from a bond; —NH—; -(C₁₋₆ alkylene)—O—; —O—(C₁-6 alkylene)-; -(C₂₋₆ alkenylene)—O—; -(C₁₋₆ alkylene)—C(O)—; -(C₂₋₆ alkenylene)—C(O)—; phenylene; piperidinylene; hydroxypiperidinylene; fluoropiperidinylene; azetidinylene; —C(O)-piperazinylene-; -(C₁₋₆ alkylene)-oxopiperazinylene-; pyrrolidinylene; 7- to 9-membered bridged bicyclic heterocycloalkylene; -(C₁₋₆ alkylene)—O-phenylene-; -(C₂₋₆ alkenylene)—O-piperidinylene; -(C₁₋₅ alkylene)—NH—, wherein 0, 1, or 2 methylene units are replaced with —O—; —NH-(C₁₋₅ alkylene)—NH—; —N(CH₃)-(C₁₋₅ alkylene)—NH—; NH-(C₁₋₅ alkylene)—N(CH₃)-; —N(CH₃)-(C₁₋₅ alkylene-—N(CH₃)-; -(C₃₋₆ cycloalkylene)—NH—; —C(O)NH-(C₁₋₅ alkylene)—NH—; —C(O)NH-(C₃₋₆ cycloalkylene)—NH—; -(C₁₋₅ alkylene)—O—(C₃₋₆cycloalkylene)—NH—; -(C₃₋₆ cycloalkenylene)—NH—; or 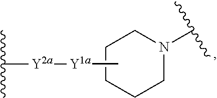
wherein Y¹a is a bond, —O—, —NH—, —NHC(O)—, —C(O)NH—, or C₁₋₃ alkylene; and Y^{2a} is a bond, —O—, —NH—, —NHC(O)—, —C(O)NH—, or C₁₋₃ alkylene.

54. The combination of any one of claims 30 to 53, wherein Y is selected from the group consisting

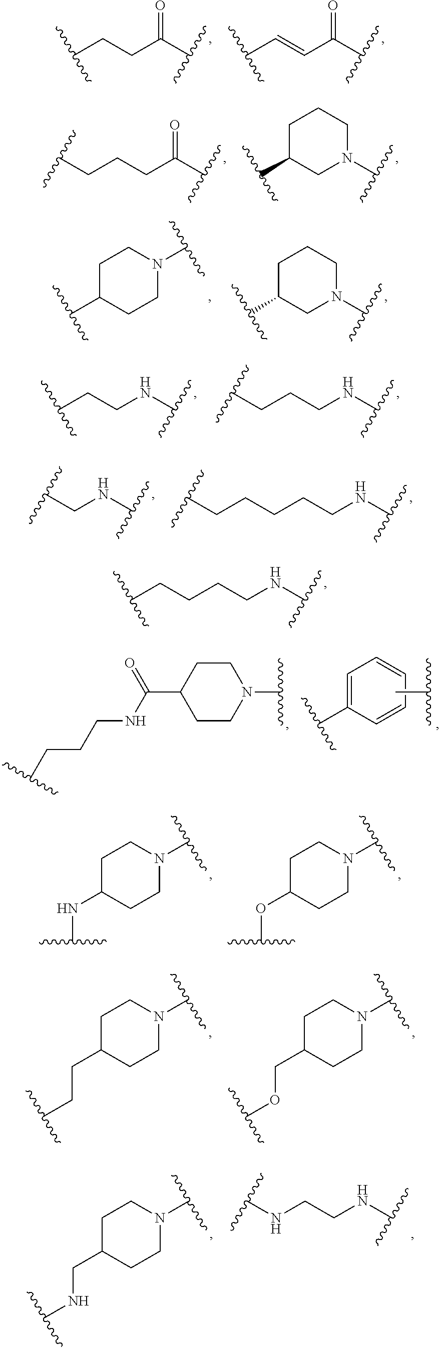

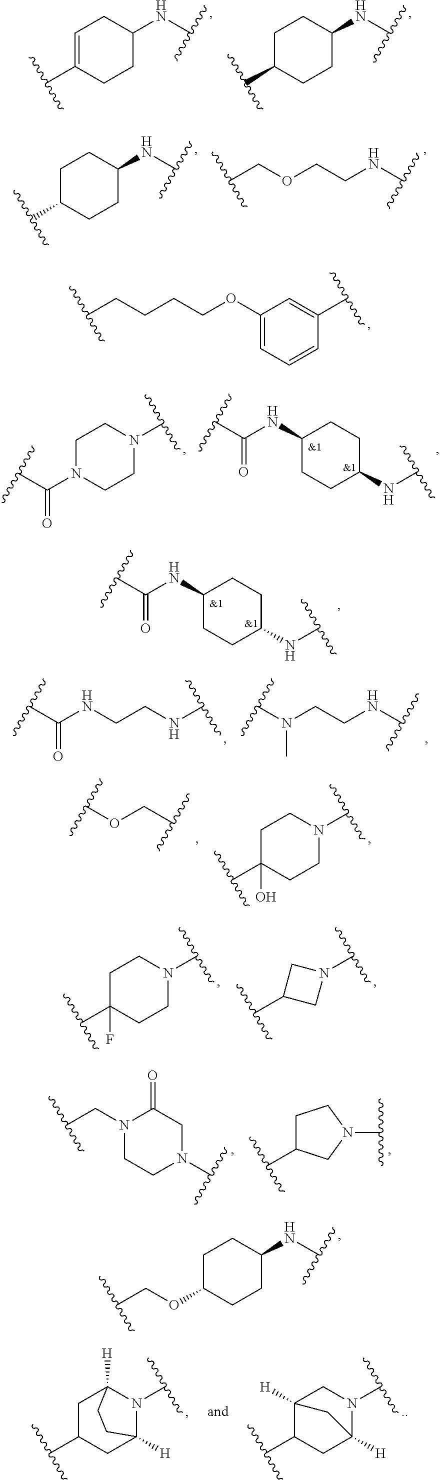

55. The combination of any one of claims 30 to 53, wherein Y is a bond or

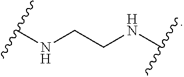 [SMILES: *NCCN*]

56. The combination of any one of claims 30 to 54, wherein Y is selected from the group consisting of:

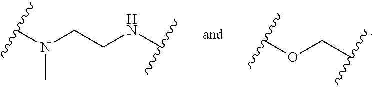

57. The combination of any one of claims 30 to 56,, wherein T is: 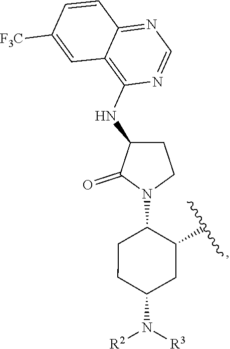
wherein R² is hydrogen or C₁₋₄ alkyl; and R³ is hydrogen or C₁₋₄ alkyl.

58. The combination of claim 57, wherein T is:

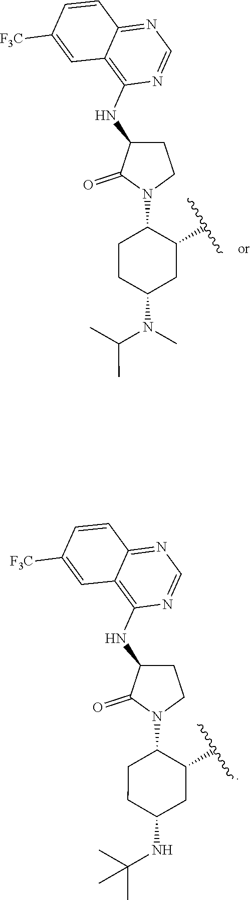 [SMILES: *[C@@H]1C[C@H](N(C)C(C)C)CC[C@@H]1N1CC[C@H](Nc2ncnc3ccc(C(F)(F)F)cc23)C1=O ; *[C@@H]1C[C@H](NC(C)(C)C)CC[C@@H]1N1CC[C@H](Nc2ncnc3ccc(C(F)(F)F)cc23)C1=O]

59. The combination of any one of claims 30 to 56, wherein T is selected from the group consisting of:

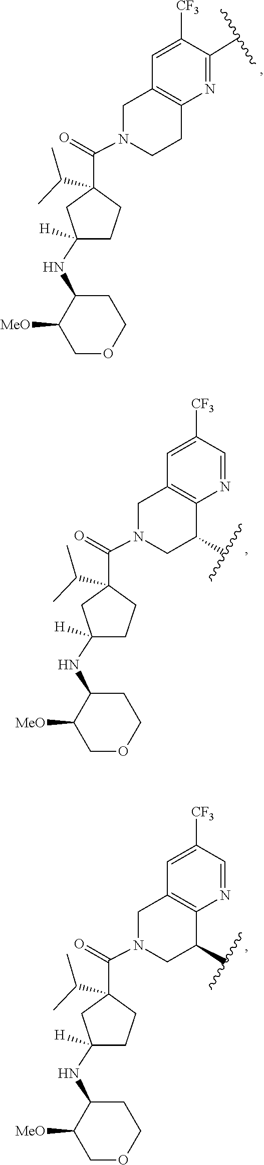 [SMILES: *c1nc2c(cc1C(F)(F)F)CN(C(=O)[C@@]1(C(C)C)CC[C@@H](N[C@H]3CCOC[C@H]3OC)C1)CC2 ; *[C@@H]1CN(C(=O)[C@@]2(C(C)C)CC[C@@H](N[C@H]3CCOC[C@H]3OC)C2)Cc2cc(C(F)(F)F)cnc21 ; *[C@H]1CN(C(=O)[C@@]2(C(C)C)CC[C@@H](N[C@H]3CCOC[C@H]3OC)C2)Cc2cc(C(F)(F)F)cnc21]

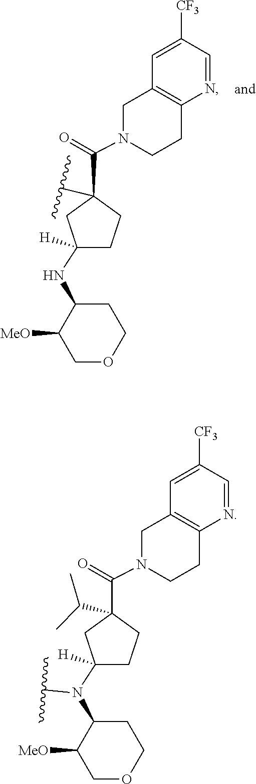 [SMILES: *[C@]1(C(=O)N2CCc3ncc(C(F)(F)F)cc3C2)CC[C@@H](N[C@H]2CCOC[C@H]2OC)C1 ; "and" ; *N([C@@H]1CC[C@@](C(=O)N2CCc3ncc(C(F)(F)F)cc3C2)(C(C)C)C1)[C@H]1CCOC[C@H]1OC]

60. The combination of any one of claims 30 to 56, wherein T is:

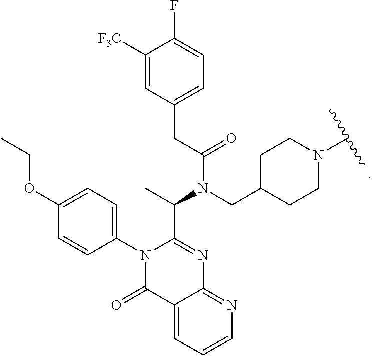 [SMILES: *N1CCC(CN(C(=O)Cc2ccc(F)c(C(F)(F)F)c2)[C@H](C)c2nc3ncccc3c(=O)n2-c2ccc(OCC)cc2)CC1]

61. The combination of any one of claims 30 to 56, wherein T is:

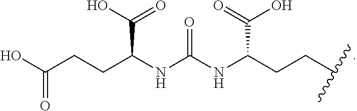 [SMILES: *CC[C@H](NC(=O)N[C@@H](CCC(=O)O)C(=O)O)C(=O)O]

62. The combination of any one of claims 30 to 56, wherein T is:

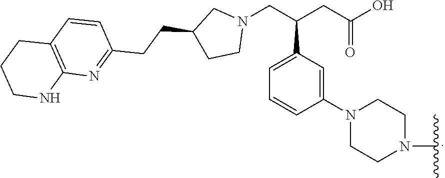 [SMILES: *N1CCN(c2cccc([C@H](CC(=O)O)CN3CC[C@@H](CCc4ccc5c(n4)NCCC5)C3)c2)CC1]

63. The combination of any one of claims 30 to 56, wherein T is:

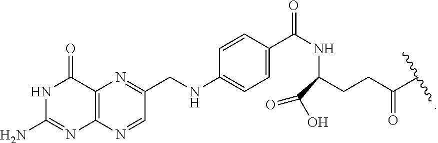 [SMILES: *C(=O)CC[C@H](NC(=O)c1ccc(NCc2cnc3nc(N)[nH]c(=O)c3n2)cc1)C(=O)O]

64. The combination of any one of claims 30 to 56, wherein T is selected from the group consisting of: 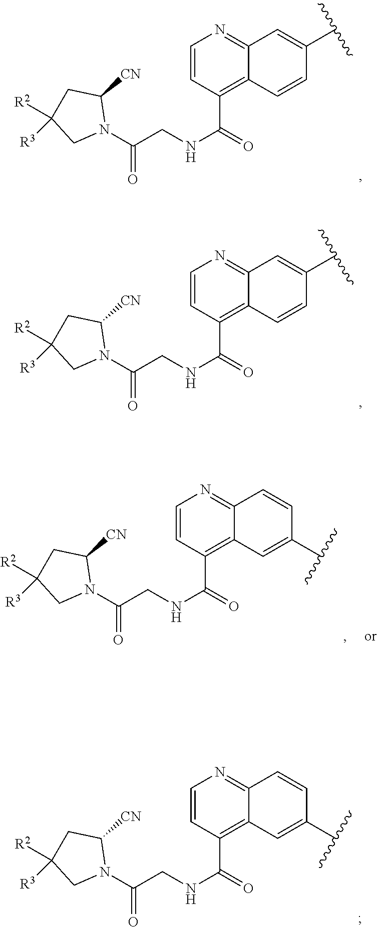
and
R² and R³ are each independently F or H.

65. The combination of any one of claims 30 to 56, wherein T is: 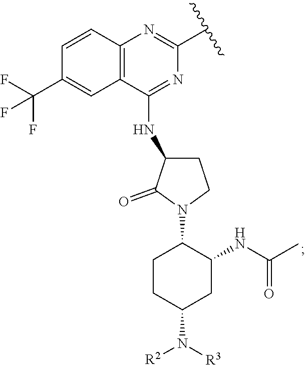
R² is hydrogen or C₁₋₄ alkyl; and
R³ is hydrogen or C₁₋₄ alkyl.

66. The combination of any one of claims 30 to 56, wherein T is: 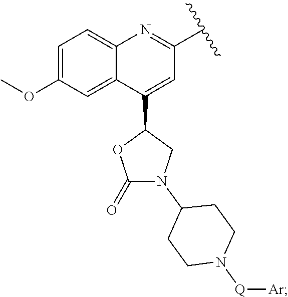
Q is C₁₋₅ alkylene, wherein 0, 1, or 2 methylene units are replaced with —O—; and
Ar is an optionally substituted 5- to 1O-membered aromatic ring or 9- or 1O-membered unsaturated fused bicyclic ring.

67. The combination of any one of claims 30 to 56, wherein the target of the target binding moiety T is selected from G protein-coupled receptor (GPCRs), enzymes, ion channels, proteases, and receptors.

68. The combination of any one claims 30 to 56, wherein the target of the target binding moiety T is present on a surface of a pathogenic immune cell, a tumor cell or cancer cell, or a stromal cell.

69. The combination of any one of claims 30 to 56, wherein the target of the target binding moiety T is present on the surface of a pathogenic agent selected from a virus or a bacterial cell.

70. The combination of any one claims 30 to 56, wherein the target of the target binding moiety T is present on a surface of monocytic myeloid-derived suppressor cells (mMDSCs), T regulatory cells (Tregs), neutrophils, macrophages, B regulatory cells (Bregs), CD8 regulatory cells (CD8regs), exhausted T cells, polymorphonuclear myeloid derived suppressor cells (PMN-MDSCs), or cancer-associated fibroblasts (CAFs).

71. The combination of any one of claims 30 to 56, wherein the target of the target binding moiety T is a chemokine receptor (CCR).

72. The combination of any one of claims 30 to 56, wherein the target of the target binding moiety T is selected from CCR1, CCR2, CCR3, or CCR5.

73. The combination of any one of claims 30 to 56, wherein the target of the target binding moiety T is selected from C-C motif chemokine receptor (CCR)₂ (CCR2), CCR1, CCR3, CCR4, CCR5, CCR6, CCR7, CCR8, CCR9, CCR10, C-X-C motif chemokine receptor 1 (CXCR1), C-X-C motif chemokine receptor 2 (CXCR2), C-X-C motif chemokine receptor 3 (CXCR3), C-X-C motif chemokine receptor 4 (CXCR4), C-X-C motif chemokine receptor 5 (CXCR5), C-X-C motif chemokine receptor 6 (CXCR6), atypical chemokine receptor 3 (ACKR3), integrin avB6, fibroblast activation protein-alpha (FAPα), prostate specific membrane antigen (PSMA), folate receptor (folate receptor 1 or folate receptor beta), complement C₃a receptor 1 (C₃AR1), complement C5a receptor 1 (C5AR1), G protein-coupled receptor (GPR)₆₅ (GPR65), GRP132, GPR84, GPR183, GPR35, GPR42, cholecystokinin A receptor (CCKAR), leukotriene B4 receptor (LTB4R), somatostatin receptor 2 (SSTR2), free fatty acid receptor 1 (FFAR1), purinergic receptor P2Y² (P2RY2), prostaglandin D2 receptor (PTGDR), calcitonin receptor (CALCR), CD38, purinergic receptor P2X 7 (P2RX7), integrin subunit alpha V (ITGAV), integrin subunit alpha 5 (ITGA5), integrin subunit beta 1 (ITGB1), integrin subunit beta 6 (ITGB6), integrin subunit beta 3 (ITGB3) prostaglandin D2 receptor 2 (PTGDR2), gastrin releasing peptide receptor (GRPR), MER proto-oncogene tyrosine kinase (MERTK), C-X3-C motif chemokine receptor 1 (CX3CR1), oxidized low density lipoprotein receptor 1 (OLR1), plasminogen activator urokinase receptor (PLAUR), carbonic anhydrase 9 (CA9), carbonic anhydrase 12 (CA12), mas -related G-protein coupled receptor member X² (MRGPRX2), heat shock protein 90 alpha family class A member 1 (HSP90AA1), dipeptidyl peptidase 4 (DPP4), formyl peptide receptor 2 (FPR2), and succinate receptor 1 (SUCNR1).

74. A method of treating and/or preventing a disease or disorder in a patient in need thereof, the method comprising: administering to the patient a therapeutically effective amount of the combination of any one of claims 7 and 29 to 73, wherein the disease or disorder is selected from a cancer, an inflammatory disease, an autoimmune disease, a viral infection, or a bacterial infection.

75. The method of claim 74, wherein the disease or disorder is mediated by chemokine receptor 2 (CCR2) and/or is associated with CCR2-positive pathogenic cells.

76. The method of claim 74, wherein the disease or disorder is mediated by C-X-C motif chemokine receptor 3 (CXCR3) and/or is associated with CXCR3-positive pathogenic cells.

77. The method of claim 74, wherein the disease or disorder is mediated by PSMA and/or is associated with PSMA-positive pathogenic cells.

78. The method of claim 74, wherein the disease or disorder is mediated by integrin αVβ6 and/or is associated with integrin αVβ6-positive pathogenic cells.

79. The method of claim 74, wherein the disease or disorder is mediated by folate receptor a (FRα) and/or folate receptor β (FRβ) and/or is associated with FRα- and/or FRβ-positive pathogenic cells.

80. The method of claim 74, wherein the disease or disorder is mediated by fibroblast activation protein (FAP) and/or is associated with FAP-positive pathogenic cells.

81. The method of claim 74, wherein the disease or disorder is mediated by chemokine receptor 8 (CCR8) and/or is associated with CCR8-positive pathogenic cells.

82. The method of any one of claims 74 to 81, wherein the disease is a cancer that is a solid tumor.

83. The method of any one of claims 74 to 82, wherein the cancer is selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, brain cancer, endocrine cancer, testicular cancer, bladder cancer, bone cancer, esophogeal cancer, gastric cancer, renal cell cancer, melanoma cancer, thyroid cancer, or breast cancer.

84. The method of claim 74 or 76, wherein the disease is an autoimmune or inflammatory disease selected from vitiligo and type 1 diabetes.

85. The method of any one of claims 74 to 84, wherein the compound and the antibody-drug conjugate are administered simultaneously, or wherein the compound and the bispecific antibody or bispecific antigen binding fragment thereof are administered simultaneously.

86. The method of any one of claims 74 to 84, wherein the compound and the antibody-drug conjugate are administered sequentially, or wherein the compound and the bispecific antibody or bispecific antigen binding fragment thereof are administered sequentially.

87. A method of increasing cell killing of target-expressing cells, the method comprising:
contacting the cells with an effective amount of the combination of any one of claims 7 and 29 to 73, wherein the target-binding moiety of the compound binds the target expressed on the cells.

88. A method of depleting target-expressing cells, the method comprising: contacting the cells with an effective amount of the combination of any one of claims 7 and 29 to 73, wherein the target-binding moiety of the compound binds the target expressed on the cells.

89. The method of claim 87 or 88, wherein the target-expressing cells are myeloid-derived suppressor cells (MDSCs), T regulatory cells (Tregs), neutrophils, macrophages, B regulatory cells (Bregs), CD8 regulatory cells (CD8regs), exhausted T cells, or cancer-associated fibroblasts (CAFs).

90. The method of any one of claims 87 to 89, wherein the target-expressing cells are CCR2-expressing cells.

91. The method of claim 87 or 88, wherein the target-expressing cells are CXCR3-expressing cells.

92. The method of claim 87 or 88, wherein the target-expressing cells are PSMA-expressing cells.

93. The method of claim 87 or 88, wherein the target-expressing cells are integrin αVβ6-expressing cells.

94. The method of claim 87 or 88, wherein the target-expressing cells are FRα- and/or FRβ-expressing cells.

95. The method of claim 87 or 88 wherein the target-expressing cells are FAP-expressing cells.

96. The method of claim 87 or 88, wherein the target-expressing cells are CCR8-expressing cells.
